(19) United States
(12) Patent Application Publication
(10) Pub. No.: US 2022/0281982 A1
(43) Pub. Date: Sep. 8, 2022

(54) BISPECIFIC ANTIBODY CAR CELL IMMUNOTHERAPY

(71) Applicant: CytoImmune Therapeutics, Inc., Wilmington, DE (US)

(72) Inventors: Michael Caligiuri, Wilmington, DE (US); Jianhua Yu, Wilmington, DE (US)

(73) Assignee: CytoImmune Therapeutics, Inc., Wilmington, DE (US)

(21) Appl. No.: 17/690,813

(22) Filed: Mar. 9, 2022

Related U.S. Application Data
(63) Continuation of application No. PCT/US2020/049998, filed on Sep. 9, 2020.
(60) Provisional application No. 62/898,503, filed on Sep. 10, 2019.

(51) Int. Cl. C07K 16/28; C07K 14/705; C07K 14/725; C07K 14/715; C12N 15/86

(52) U.S. Cl. CPC C07K 16/2851; C07K 16/2878; C07K 16/2803; C07K 14/70517; C07K 14/70521; C07K 14/7051; C07K 14/7155; C12N 15/86; C07K 2319/33; A61K 2039/505

(57) ABSTRACT

Described herein are single vectors that, when expressed in cytolytic immune cells, results in both the expression of (1) a CAR targeting tumor-associated antigens and (2) secretion of a bispecific antibody that on one end recognizes NKG2D expressed on both innate and antigen specific cytolytic immune cells and on the other end targets tumor associated antigens. Unexpectedly, these modifications to the T cells result in enhanced survival and proliferation in vivo. Thus, therapeutic and diagnostic uses are disclosed.

### CROSS-REFERENCE TO RELATED APPLICATIONS

This application claims priority under 35 U.S.C. § 119(e) of U.S. Provisional Application No. 62/898,503, filed Sep. 10, 2019, the contents of which is incorporated by reference in its entireties into the present application.

### SEQUENCE LISTING

The instant application contains a Sequence Listing which has been submitted electronically in ASCII format and is hereby incorporated by reference in its entirety. Said ASCII copy, created on Sep. 9, 2020, is named 113086-0113_ST25.txt and is 134,572 bytes in size.

### TECHNICAL FIELD

The present disclosure relates generally to the field of human immunology, specifically cancer immunotherapy.

### BACKGROUND

The following discussion of the background of the invention is merely provided to aid art to the present invention.

Current cancer treatment regimens including chemotherapies, immunomodulatory drugs¹⁴, monoclonal antibodies¹⁵, and autologous or allogeneic transplantation. These therapies often lead to remission, but nearly all patients eventually relapse and succumb to death due to return of the disease. Thus, there is an unmet need for new therapies, including new combination immunotherapies for relapsed and/or refractory disease. This disclosure addresses these limitations and provides related advantages as well.

### SUMMARY OF THE DISCLOSURE

Chimeric antigen receptor (CAR) T cells have been used successfully in the clinic for the treatment of both hematological malignancies and solid tumors, and have recently been approved by the U.S. FDA¹⁻⁴. Bispecific antibodies (BsAb) have also been approved by the FDA for cancer treatment and are being used as an alternative immunotherapeutic approach to CAR T cell therapy⁵. However, CAR- and BsAb-based cancer immunotherapies still need improvement for five important reasons. First, in some cases, CAR T cells cannot be expanded in vivo and cannot survive for a sufficient period of time to initiate tumor lysis in patients⁶. It has been reported that the efficacy of CAR T cells correlates with the quantity and duration of CAR T cell presence in vivo⁶⁻⁸. Second, tumor cells can shed targeted antigens to evade therapy, especially when only a single antigen is targeted. Third, in addition to being costly and time-consuming to manufacture, BsAb have a short half-life and to date have not been shown to be curative^{9,10}. Fourth, combination therapy of CAR T cells with BsAb targeting two distinct tumor associated antigens could be a good approach; however, producing each individually ex vivo would be labor intensive and costly; engineering T cells to express both a CAR and a BsAb (such as, a bispecific T cell engager, i.e., BiTE, or a bispecific natural killer cell engager, i.e., BiKE) within a single construct, of which the BsAb engages all cytolytic effector cells has not yet been reported or shown to be additive or synergistic, or to enhance T cell survival in vivo. Finally, current approaches to CAR immunotherapy mainly focus on one immune cell type, i.e., T cells, excluding all other cytolytic effector cells in the innate and adaptive arms of the immune system. NKG2D is a c-lectin type of receptor that is expressed on virtually all cytolytic effector cells in both the innate and adaptive arms of the immune system^{11,12}

This disclosure provides a platform to resolve these issues, in part or in full, by engineering T cells and/or Natural killer (NK) cells infected with a single vector delivering these two modes of therapy, i.e., producing a T cell or an NK cell whose CAR is targeting one specific tumor-associated antigen and which expresses and/or secretes BsAb targeting another specific tumor-associated antigen.

Thus, in one aspect, provided herein are bispecific antibody-chimeric antigen receptors (BsAb-CAR, referred to herein as a BsAb-CAR construct or a CAR construct) comprising, or alternatively consisting essentially of, or yet further consisting of: (1) a chimeric antigen receptor (CAR) comprising, or alternatively consisting essentially of, or yet further consisting of: (a) an antigen binding domain of a cancer or tumor targeting antibody; (b) a hinge domain, (c) a transmembrane domain, and (d) an intracellular domain; and (2) a bispecific antibody comprising, or alternatively consisting essentially of, or yet further consisting of an antigen binding domain of an antibody directed to a cancer or tumor antigen expressed on the same cancer or tumor cell to which the CAR binds, and an anti-NKG2D antigen binding domain. In one aspect, the intracellular domain comprises one or more, or two or more costimulatory regions that can comprise or consisting essentially of a CD28 costimulatory signaling region and/or a 4-1BB costimulatory domain. The bispecific antibody portion of the CAR can further comprise, or consist essentially of, or yet further consist of a signaling domain located prior to the anti-NKG2D antigen binding domain. Polynucleotides and vectors encoding these constructs are further provided herein as well as the polypeptides encoded by them. In one aspect, the CAR and the BsAb are expressed from a single contiguous polynucleotide and the cancer or tumor targeting antibody is expressed on the cell surface of the host cell and the BsAb component is secreted by the cell expressing the CAR. Polynucleotides encoding these constructs are further provided herein as well as the polypeptides encoded by them in vitro or in vivo.

In certain embodiments, the antigen binding domain of the tumor targeting antibody comprises, or alternatively consists essentially of, or further consists of one or more of: a heavy chain variable region and a light chain variable region, an scFv fragment, and/or an Fe fragment that are optionally linked by a linker peptide. In some embodiments, the heavy and/or light chain variable region comprises, or alternatively consists essentially of, or further consists of the relevant CDR regions of an antibody directed to any one of B-cell maturation antigen (BCMA) and/or SLAMF7 (also known as CS1 or CD319), and/or an equivalent of each thereof. In one aspect, the tumor targeting antibody targets BCMA and the BsAb comprises an antigen binding fragment of an anti-CS1 antibody. In some embodiments, the heavy chain and/or light chain variable region comprises, or alternatively consists essentially of, or further consists of the amino acid sequence of an antibody any one of B-cell maturation antigen (BCMA) and/or SLAMF7 (also known as CS1 or CD319), and/or an equivalent of each thereof. In one aspect, this construct further comprises, or alternatively consists essentially of, or yet further consists of a signaling domain. Polynucleotides and vector encoding these constructs are further provided herein as well as the polypeptides encoded by them in vitro or in vivo.

In certain embodiments, the CAR, further comprises, or alternatively further consists essentially of, or yet further consists of, a linker polypeptide located between the heavy chain variable region and the light chain variable region and/or the ScFv and Fc regions and/or the antigen binding domains. In one aspect, the linker polypeptide is located between the antibody fragments of anti-NKG2D and anti-CS1. In certain embodiments, the linker is a glycine-serine linker. In further embodiments, the linker polypeptide comprises, or alternatively consists essentially of, or further consists of the sequence (glycine-serine)n wherein n is an integer from 1 to 6 (SEQ ID NO: 110), e.g. wherein n is 4. In another aspect, the polynucleotide comprises a sequence encoding a self-cleaving peptide such as a T2A peptide located between the CAR and the bispecific antibody, e.g., between the anti-BCMA CAR and the anti-NKG2D antigen binding domain. In one aspect, the BsAb-CAR further comprises a self-cleaving peptide such as for example a T2A peptide located between the anti-NKG2D and the second antigen binding domain that binds the cancer or tumor antigen, e.g., CS1, both of which are linked to a Fc fragment. In this case, a “knobs-into-holes” system can be added so that homodimer of the NKG2D-Fc or CS1-Fc will not happen while the heterodimer of the two will occur using the method described in Protein Engineering, Vol. 9(6):617-621 (1996). In one aspect, this CAR further comprises, or alternatively consisting essentially of, or yet further consisting of a signaling domain. In one aspect, the signaling domain, e.g., an IgG1 signal peptide, is located at the beginning of the NKG2D-tumor antigen portion of the BsAb-CAR. Other signal peptides are known in the art. Polynucleotides and vectors encoding these constructs are further provided herein as well as the polypeptides encoded by them in vitro or in vivo.

In one aspect, the BsAb-CAR comprises the structure shown in part in FIG. 1E, i.e., a single BsAb-CAR comprising or consisting essentially of (a) a BCMA antigen binding polypeptide, a self-cleaving peptide such as T2A, anti-NKG2D scFv and Fc polypeptides, a second self-cleaving peptide such as T2A, and anti-CS1 scFv and Fc polypeptides. In this case, a “knobs-into-holes” system can be added so that homodimer of the NKG2D-Fc or CS1-Fc will not happen while the heterodimer of the two will occur using the method described in Protein Engineering, Vol. 9(6):617-621 (1996). The CAR, such as an anti-BCMA CAR can further comprise a hinge domain, a transmembrane domain, and an intracellular domain. In one aspect, this Bs-Ab CAR construct further comprises, or alternatively consists essentially of, or yet further consists of a signaling domain. In one aspect, this Bs-Ab CAR construct further comprises, or alternatively consists essentially of, or yet further consists of signal peptide, e.g., an IgG1 signal peptide, optionally located at the beginning of the anti-NKG2D-anti-tumor-antigen portion of the BsAb-CAR construct. Other signal peptides are known in the art. Further provided are polynucleotides and vectors encoding these BsAb-CAR constructs as well as the polypeptides encoded by them in vitro or in vivo.

In one aspect, the BsAb-CAR comprises the structure shown in part in FIG. 1F, i.e., a single CAR comprising or consisting essentially of (a) a BCMA antigen binding polypeptide, a self-cleaving peptide such as T2A, anti-NKG2D scFv and Fc polypeptides, an HMA polypeptide, and anti-CS1 scFv and Fc polypeptides. The BCMA CAR can further comprise a hinge domain, a transmembrane domain, and an intracellular domain. In one aspect, this polypeptide further comprises, or alternatively consists essentially of, or yet further consists of a signaling domain. In one aspect, this construct further comprises, or alternatively consists essentially of, or yet further consists of a signal peptide, e.g., an IgG1 signal peptide, is located at the beginning of the NKG2D-tumor antigen portion of the BsAb-CAR. Other signal peptides are known in the art. Further provided are polynucleotides and vectors encoding these BsAb-CAR constructs as well as the polypeptides encoded by them in vitro or in vivo.

In one aspect, the BsAb-CAR comprises the structure shown in part in FIG. 1G, i.e., a single Bs-Ab CAR comprising or consisting essentially of (a) a BCMA antigen binding polypeptide, a self-cleaving peptide such as T2A, anti-NKG2D scFv and Fc polypeptides, a linker such as G4S polypeptide (SEQ ID NO: 134), and anti-CS1 scFv and Fc polypeptides. The BCMA CAR can further comprise a hinge domain, a transmembrane domain, and an intracellular domain. In one aspect, this polypeptide further comprises, or alternatively consists essentially of, or yet further consists of a signaling domain. In one aspect, this construct further comprises, or alternatively consists essentially of, or yet further consists of a signaling domain, e.g., an IgG1 signal peptide, is located at the beginning of the NKG2D-tumor antigen portion of the BsAb-CAR. Other signal peptides are known in the art. Further provided are polynucleotides and vectors encoding these BsAb-CAR constructs as well as the polypeptides encoded by them in vitro or in vivo.

In one aspect, provided herein is: (a) a CAR comprising, or alternatively consisting essentially of, or yet further consisting of (i) an antigen binding domain of a cancer or tumor targeting antibody that binds a cancer or tumor cell with the optional proviso that the cancer or tumor targeting antibody does not target a B-cell maturation antigen (BCMA), (ii) a hinge domain, (iii) a transmembrane domain, and (iv) an intracellular domain; and (b) a bispecific antibody comprising, or alternatively consisting essentially of, or yet further consisting of an antigen binding domain that recognizes and binds a NKG2D and an antigen binding domain of an antibody that binds a different antigen expressed by the cancer or tumor cell of the CAR. The CAR combined with the secreted BsAb are designed to reduce the chance of clonal resistance to one modality (e.g., the CAR). In addition, by using NKG2D, it brings to the tumor microenvironment a variety of cytolytic effects (in addition to the CAR), to the tumor bed (e.g., NK cells) and Applicant shows herein that this significantly adds to the tumor cell killing. This is unexpected from the knowledge of one of skill in the art. Tumor-specific combinations have been reported (Townsend et al. (2018), J. Exper. & Clin. Cancer Res. 37:163, and references cited therein) but heretofore, they have not been combined with an NKG2D element.

In one aspect, the hinge domain comprises, or alternatively consists essentially of, or yet further consists of, a CD8 α hinge domain. In one aspect, this polynucleotide further comprises, or alternatively consists essentially of, or yet further consists of a polynucleotide encoding a signaling domain. In one aspect, the signaling domain, e.g., an IgG1 signal peptide, is located at the beginning of the NKG2D-tumor antigen portion of the BsAb-CAR. Other signal peptides are known in the art.

In one aspect, the transmembrane domain of the CAR comprises, or consists essentially of, or yet further consists of, a CD8 α transmembrane domain. In another aspect, the intracellular domain comprises, or alternatively consists essentially of, or yet further consists of, one or more, or two or more costimulatory regions selected from a CD28 costimulatory signaling region, a 4-1BB costimulatory signaling region, an ICOS costimulatory signaling region, or an OX40 costimulatory region. In another aspect, the transmembrane domain comprises, or consists essentially of or yet further consists of a CD28 costimulatory signaling region and/or a 4-1BB costimulatory signaling region. In another aspect, the signaling domain comprises, or alternatively consists essentially of, or yet further consists of, a CD3 zeta signaling domain.

In some aspects, the bispecific antibody comprises, or consists essentially of, or yet further consists of a ligand of NKG2D, or an anti-NKG2D scFv, and/or an equivalent each thereof. In some aspects, the ligand of NKG2D, an anti-NKG2D scFv, CDR regions of an antibody that binds to NKG2D, and/or an equivalent of each thereof. In some aspects, the bispecific antibody comprises, or consists essentially of, or yet further consists of the heavy chain and light chain variable region of an antibody to NKG2D, and/or an equivalent of each thereof. In some aspects, the bispecific antibody comprises, or consists essentially of, or yet further consists of a single chain variable fragment (scFv) derived from an antibody to NKG2D, and/or an equivalent each thereof.

Non-limiting examples of antigens targeted by the CAR or cancer or tumor targeting antibody are selected from antibodies that bind to a target of the group: CD19, mesothelin, human epidermal growth factor receptor 2 (HER2), prostate stem cell antigen (PSCA), carcinoembryonic antigen (CEA), CD33, GTPase-activating protein (GAP), ganglioside G2 (GD2), CD5, prostate specific membrane antigen (PSMA), receptor tyrosine kinase-like orphan receptor 1 (ROR1), CD123, CD70, CD38, mucin 1, (Muc1), ephrin type-A receptor 2 precursor (EphA2), epidermal growth factor receptor variant III (EGFRVIII), interleukin 13 receptor alpha 2 (IL13Ra2), CD133, glypican 3 (GPC3), epithelial cell adhesion molecule precursor (EpCam), fibroblast activation protein alpha (FAP), vascular endothelial growth factor receptor 2 (VEGFR2), cancer/testis (CT), guanylyl cyclase C (GUCY2C), tumor-associated glycoprotein-72 (TAG-72), thymidine kinase 1 (TK1), and hypoxanthine guanine phosphoribosyltransferase (HPRT1). Additional tumor targeting antibodies are known in the art.

In one aspect, the CAR recognizes and binds FLT3 and the bispecific antibody binds CD123, and is useful to treat AML. In another aspect, the CAR recognizes and binds EGFR and the bispecific antibody binds IL13Ra2 and is used to treat glioblastoma (GBM).

Further provided are polynucleotides encoding the above noted CAR constructs as well as the polypeptides encoded by them in vitro or in vivo. In one aspect, the polynucleotide encodes: (a) a CAR comprising, or alternatively consisting essentially of, or yet further consisting of: (i) an antigen binding domain of a cancer or tumor targeting antibody with the proviso that the cancer or tumor targeting antibody does not target a B-cell maturation antigen (BCMA); (ii) a hinge domain; (iii) a transmembrane domain; (iv) and an intracellular domain; and (b) a polynucleotide encoding a bispecific antibody comprising, or alternatively consisting essentially of, or yet further consisting of an antigen binding domain that recognizes and binds a NKG2D and polynucleotide that recognizes and binds an antigen on the tumor or cancer cell that is a different target than the CAR. In one aspect, the polynucleotide encodes a CD8 α hinge domain. In one aspect, this polynucleotide further comprises, or alternatively consisting essentially of, or yet further consisting of a polynucleotide encoding a signaling domain.

In one aspect, the polynucleotide further encodes a transmembrane domain that comprises, or consists essentially of, or yet further consists of, a CD8 α transmembrane domain. In another aspect, the polynucleotide further encodes an intracellular domain that comprises, or alternatively consists essentially of, or yet further consists of, a CD28 costimulatory signaling region and/or a 4-1BB costimulatory signaling region. In a further aspect, the polynucleotide further comprises a polynucleotide that encodes a CD3 zeta signaling domain.

In some aspects, the polynucleotide encodes a bispecific antibody that comprises, or consists essentially of, or yet further consists of a ligand of NKG2D, or an anti-NKG2D scFv, and/or an equivalent each thereof. In some aspects, the ligand of NKG2D or an anti-NKG2D scFv that comprises, or consists essentially of, or yet further consists of, CDR regions of an antibody that binds to NKG2D, and/or an equivalent of each thereof. In some aspects, the polynucleotide encodes a bispecific antibody that comprises, or alternatively consists essentially of, or yet further consists of, a heavy chain variable region and a light chain variable region of an antibody directed to (such as recognizing and binding) NKG2D, and/or an equivalent of each thereof. In some aspects, the polynucleotide encodes a bispecific antibody that comprises, or consists essentially of, or yet further consists of, a single chain variable fragment (scFv) derived from an antibody directed to (such as recognizing and binding) NKG2D, and/or an equivalent thereof.

Non-limiting examples of antibodies or antigen binding domains encoded by the polynucleotides are selected from those that bind an antigen selected from the group: FLT3, EGFR, CD123, IL13Ra2, CD19, mesothelin, human epidermal growth factor receptor 2 (HER2), prostate stem cell antigen (PSCA), carcinoembryonic antigen (CEA), CD33, GTPase-activating protein (GAP), ganglioside G2 (GD2), CD5, prostate specific membrane antigen (PSMA), receptor tyrosine kinase-like orphan receptor 1 (ROR1), CD123, CD70, CD38, mucin 1, (Muc1), ephrin type-A receptor 2 precursor (EphA2), epidermal growth factor receptor variant III (EGFRVIII), interleukin 13 receptor alpha 2 (IL13Ra2), CD133, glypican 3 (GPC3), epithelial cell adhesion molecule precursor (EpCam), fibroblast activation protein alpha (FAP), vascular endothelial growth factor receptor 2 (VEGFR2), cancer/testis (CT), guanylyl cyclase C (GUCY2C), tumor-associated glycoprotein-72 (TAG-72), thymidine kinase 1 (TK1), and hypoxanthine guanine phosphoribosyltransferase (HPRT1). Additional tumor targeting antibodies are known in the art.

In some aspects, the polynucleotide of (a) and (b) are contiguous. Non-limiting examples of such are shown in FIGS. 1E-1G and FIG. 3A.

This disclosure also provides a vector that comprises, or alternatively consists essentially of, or yet further consists of any one of the preceding polynucleotides. In some aspects, the polynucleotide or vector further comprises, or consists essentially of, or yet further consists of, regulatory elements to drive expression of the polynucleotide and/or the CAR and/or bispecific antibodies, such as a promoter and/or an enhancer element. In some aspects, the vector is a plasmid or a viral vector, non-limiting examples of such are selected from the group of a retroviral vector, a lentiviral vector, an adenoviral vector, and an adeno-associated viral vector.

Also provided herein is an isolated cell comprising, or alternatively consisting essentially of, or yet further consisting of, any one of the preceding vectors, CARs, and/or polynucleotides as described herein. The cell can be a prokaryotic cell (e.g., a bacterial cell) or a eukaryotic cell. Non-limiting examples of eukaryotic cells include, but are not limited to a yeast cell, an animal cell, a mammalian cell, a bovine cell, a feline cell, a canine cell, a murine cell, an equine cell, or a human cell. In some aspects, the eukaryotic cell, mammalian or human cell is an immune cell, optionally a T-cell, a B cell, a NK cell, a dendritic cell, a myeloid cell, a monocyte, or a macrophage. In some aspects, the isolated cell expresses the CAR and secretes the bispecific antibody.

Further provided herein are compositions comprising a carrier and any one of the preceding polynucleotides and/or any one of the preceding vectors and/or any one of the preceding isolated cells. In one aspect, the carrier is a pharmaceutically acceptable carrier.

In some aspects, the disclosure is drawn to an isolated complex comprising any one of the preceding isolated cells bound to a cancer or tumor cell, wherein the cancer or tumor cell is bound to the isolated cell by the antigen binding domain of the antigen or tumor targeting antibody expressed by the CAR- or BsAb-encoding polynucleotide.

In some aspects, the disclosure is drawn to a method of producing a CAR-expressing cell comprising transducing an isolated cell with any one of the preceding polynucleotides. In some aspects, the CAR expressing cell is selected from a group consisting of T-cells, B cells, NK cells, dendritic cells, myeloid cells, monocytes, or macrophages.

In some aspects, the disclosure is drawn to a method of inhibiting the growth of a cancer cell or tumor expressing a cancer or tumor antigen or tissue comprising the cancer or tumor cell, comprising, or alternatively consisting essentially of, or yet further consisting of, contacting the cancer cell, tumor or tissue with any one of the preceding CARs and bispecific antibodies and/or isolated cells expressing the CARs and/or bispecific antibodies. In some aspects, the contacting is in vitro or in vivo. In some aspects, the contacting is in vivo and the isolated cells are autologous or allogeneic to a subject being treated. In some aspects, the contacting is in vivo and the isolated cells are allogenic to a subject being treated. The contacting in vivo can be by administration of the cells to the subject in need of such treatment. As is apparent to the skilled artisan, the antigen binding domain of the tumor or cancer antigen is selected to bind to and treat or inhibit the growth of the cancer or tumor cell.

In some aspects, this disclosure provides a method of inhibiting the growth of a cancer cell or tumor expressing a cancer or tumor antigen or tissue comprising the cancer or tumor cell by administering to a subject an effective amount of the CAR and bispecific antibody and/or host cell expressing the BsAb-CAR as described herein and a cytoreductive therapy or chemotherapy or therapy that upregulates the expression of a target antigen of the antigen binding domain of the CAR. In some aspects, the cytoreductive therapy comprises, or alternatively consists essentially of, or yet further consists of one or more of a chemotherapy, cryotherapy, hyperthermia, targeted therapy, and/or radiation therapy.

In some aspects, provided herein is a method for one or more of: inhibiting the growth of a cancer or tumor, inhibiting metastasis of a cancer or a tumor, or treating a cancer or a tumor, in a subject in need thereof, comprising, or consisting essentially of, or yet further consisting of administering any one of the preceding CARs and bispecific antibodies, or isolated cells expressing the CAR and bispecific antibodies to the subject. In some aspects, the isolated cell is autologous or allogeneic to the subject in need. In some aspects, the isolated cell is allogenic to the subject in need. As is apparent to the skilled artisan, the antigen binding domain of the tumor or cancer antigen of the CAR is selected to bind to and treat or inhibit the growth of the cancer or tumor cell.

In some aspects, the above methods further comprise, or alternatively consist essentially of, or yet further consists of administering to the subject cytoreductive therapy or chemotherapy or therapy that upregulates the expression of a target antigen. In some aspects, the cytoreductive therapy comprises, or consists essentially of, or consists of, one or more of chemotherapy, cryotherapy, hyperthermia, targeted therapy, and/or radiation therapy.

Also provided herein is a kit comprises, or alternatively consists essentially of, or yet further consists of, one or more of a CAR, a BsAb-CAR, a polynucleotide, a cell, or a composition as disclosed herein and optionally, instructions for making or using the same. The kit optionally further comprises the compositions to assay a patient sample for the phenotype of the cancer or tumor in a patient sample such that the appropriate CAR or BsAb-CAR can be selected to treat the cancer or tumor.

In further aspects, provided herein are isolated nucleic acids that encode, in one construct, the CAR and bispecific antibody as disclosed above (“BsAb-CAR construct”). In one aspect, the isolated nucleic acid encodes an antigen binding fragment that targets a cancer or tumor associated antigen other than BCMA and a bispecific antibody, e.g., one scFv targeting an antigen on an AML or GBM, (e.g., FLT-3, EGFR, CD123 or IL13Ra2) and one scFv from an anti-NKG2D antibody, joined together by a nucleic acid encoding a non-immunogenic protein linker such as from human muscle aldolase (HMA). An exemplary BsAb-CAR vector is shown in FIG. 3. The vectors optionally comprise regulatory sequences such as promoters, enhancers, and viral long terminal repeats (LTRs).

Some aspects of the disclosure relate to a method of producing a CAR expressing cell or a CAR expressing cell that secretes BsAb, the method comprising, or alternatively consisting essentially of, or yet further consisting of transducing an isolated cell with the nucleic acid sequence encoding a CAR and BsAb or the isolated nucleic acid encoding the BsAb-CAR, as described herein.

In a further aspect, the method further comprises selecting and isolating the cell expressing the CAR or BsAb-CAR. In a further aspect, the cell is a eukaryotic cell such as a mammalian cell, e.g., a human cell such as a T-cell, a B cell, a NK cell, a dendritic cell, a myeloid cell, a monocyte, a macrophage, any subsets thereof, or any other immune cell. The cells can be transduced using the viral vectors as described herein or alternatively using technology described in Riet et al. (2013) Meth. Mol. Biol. 969:187-201 entitled “Nonviral RNA transfection to transiently modify T cell with chimeric antigen receptors for adoptive therapy.”

In certain embodiments, the method further comprises, or alternatively consists essentially of, or yet further consists of transducing the cell with an isolated polynucleotide comprising, or alternatively consisting essentially of, or yet further consisting of a polynucleotide encoding a bispecific antibody, which optionally recognizes and binds NKG2D. In some embodiments, the bispecific antibody comprises, or alternatively consists essentially of, or further consists of an NKG2D ligand optionally codon optimized ligand. In certain embodiments, the bispecific antibody comprises, or alternatively consists essentially of, or further consists of the relevant CDR regions of an antibody directed to NKG2D, optionally codon optimized, or an equivalent of each thereof. In some embodiments, the bispecific antibody comprises, or alternatively consists essentially of, or further consists of the heavy chain and/or light chain variable region of an antibody directed to NKG2D, that are optionally codon optimized and/or an equivalent of each thereof. In some embodiments, the bispecific antibody comprises a single chain variable fragment (scFv) derived from an antibody directed to NKG2D, that is optionally codon optimized, and/or an equivalent each thereof. The cells can be transduced using the viral vectors, e.g., lentiviral vectors, as described herein or alternatively using technology described in Riet et al. (2013) Meth. Mol. Biol. 969:187-201 entitled “Nonviral RNA transfection to transiently modify T cell with chimeric antigen receptors for adoptive therapy.”

In certain embodiments, the method of producing a CAR or BsAb-CAR expressing cell further comprises, or alternatively consists essentially of, or yet further consists of activating and expanding the population of CAR expressing cells. Certain aspects of the present disclosure relate to an isolated, activated population of cells comprising, or alternatively consisting essentially of, or yet further consisting of a CAR or BsAb-CAR. In certain embodiments, the cells are one or more of T-cells, B cells, NK cells, dendritic cells, myeloid cells, monocytes, macrophages, any subsets thereof, or any other immune cells.

Aspects of the disclosure relate to a method of inhibiting the growth of a tumor expressing a cancer or tumor antigen, by contacting the tumor with an effective amount of the isolated cells or compositions disclosed above. The contacting can be in vitro or in vivo. When the contacting is in vitro, the method can be used to test personalized therapy against a patient's tumor or to assay for combination therapies. When the contacting is in vivo, the method is useful to inhibit the growth of the tumor or cancer cell in a subject in need thereof, such as a human patient suffering from cancer and the patient receives an effective amount of the cells. In certain embodiments, the tumor is a solid tumor. An effective amount is administered alone or in combination with other therapies as described herein. In certain embodiments, the cancer/tumor targeted is a solid tumor or a cancer affecting the blood and/or bone marrow, e.g., multiple myeloma (MM), acute myeloid leukemia (AML), or glioblastoma (GBM). In certain embodiments, the isolated cells are autologous to the subject being treated. In another aspect, the cells are allogeneic to the subject being treated. In another aspect, the method further comprises, or consists essentially of, or yet further consists of, administering to the subject an effective amount of a cytoreductive therapy. In a further aspect, the method further comprises the steps of isolating the cells to be administered to the subject, transducing the cells with an effective amount of an isolated nucleic acid encoding a CAR or BsAb-CAR as described herein, culturing the cells to obtain a population of CAR or BsAb-CAR encoding cells, that are optionally expanded and activated and then administering the cells to the patient.

The above compositions and methods are unique and overcome the limitation of the state of the art in that they provide a CAR cell that simultaneously secretes a NKG2D-based BsAb targeting a second tumor-associated antigen, wherein the cancer or tumor targeting antibody of the CAR does not target a B-cell maturation antigen (BCMA). The disclosed CAR NKGD2D-based BsAb is exemplary only. This approach can be modified for any number of tumor antigens, as known in the art, e.g., EGFR, FLT-3, IL13Ra2, EGFRVIII; CD70, mesothelin, CD123, CD19, CEA, CD133, Her2, see Townsend et al. (2018) J. Exp. & Clinical Cancer Res. 37:163. In a further aspect, the cancer or tumor targeting antibody of the CAR does not target one or more of a B-cell maturation antigen (BCMA), EGFRVIII; CD70, mesothelin, CD123, CD19, CEA, CD133, Her2.

An exemplary construct was tested in a multiple myeloma model (MM). MM is a malignancy characterized by an accumulation of clonal plasma cells¹³. As noted above, current treatment regimens including chemotherapies, immunomodulatory drugs¹⁴, monoclonal antibodies¹⁵, and autologous or allogeneic transplantation often lead to remission, but nearly all patients eventually relapse and succumb to death due to return of the disease. Thus, there is an unmet need for new therapies, including new combination immunotherapies for relapsed and/or refractory MM, solid tumors and other cancers.

As a component of the innate immune system, natural killer (NK) cells play an important role in preventing tumor growth¹⁶, but NK cell anti-tumor activity has been found to be dampened in many MM patients¹⁷. Adoptive transfer of activated or allogeneic NK cells produce effective anti-tumor responses in the treatment of a number of hematological malignancies, including MM^{18,19}, and solid tumors. However, in many cases, NK cell-mediated antitumor responses are weak, which may result from NK cell expression of inhibitory receptors, poor capacity for survival, or limited migration of effector cells into tumor sites²⁰⁻²² Meanwhile, as a part of adaptive immunity, T cells can migrate efficiently into various tissues, and tend to proliferate well in response to antigen stimulation. However, T cells have strict specificities dictated by antigen-specific T-cell receptors (TCR). Thus, a method to engage both T cells and NK cells, and thus overcome their own limitations would be of great benefit for cancer immunotherapy. This disclosure provides this benefit.

### BRIEF DESCRIPTION OF THE DRAWINGS

FIGS. 1A-1G show the results of engineering T cells to express BCMA CAR and anti-NKG2D-anti-CS1 bispecific fusion protein individually or in combination that is presented as an example of the disclosed technology. (FIG. 1A) Schematic representation of the BCMA CAR lentiviral constructs containing a scFv against BCMA linked to CD28 and CD3zeta (ζ) endodomains. The expression of the transgene was traced by GFP expression driven by an EF1alpha (α) promoter. LTR, long terminal repeats; SP, signal peptide; VH, variable H chain; L, linker; VL, variable L chain. MyC, MyC tag; Hinge, Hinge Chain; CD28, a T cell co-stimulatory molecule; CD3ζ, CD3 zeta chain. (FIG. 1B) Schematic diagram of lentiviral construct for mammalian expression of anti-NKG2D-anti-CS1 bispecific antibody (BsAb). The anti-NKG2D-anti-CS1 BsAb consisted of an anti-NKG2D scFv, which was composed of VH and VL linked together by a linker (L), and an anti-CS1 scFv. Expression of the BsAb is driven by a CMV promoter flanked by lentiviral LTR. (FIG. 1C) PBMC (peripheral blood mononuclear cells) from healthy donors were activated with CD3 and CD28 beads and transduced with the pCDH empty vector (EV), BCMA CAR, anti-NKG2D-anti-CS1 BsAb. The activated T cells were also sequentially transduced with BsAb and BCMA-CAR and these transduced cells were named “BsAb-BCMA seq. trans. T”. GFP-positive cells were sorted, and cells were stained with biotin labeled goat anti-mouse Fab specific or isotype-matched control antibody, followed by streptavidin and CD3 antibody staining. (FIG. 1D) Supernatant of unmodified T cells, BsAb T cells or BsAb-BCMA seq. trans. T cells were collected, and individual cell lysates were subjected to immunoblot analysis with anti-6x His-tag antibody (“6x His” disclosed as SEQ ID NO: 111). FIGS. 1E to 1G show alternative BsAb-CAR BCMA-NKG2A-CS1 constructs. FIG. 1G discloses “G4S” as SEQ ID NO: 134.

FIGS. 2A-2E show that BsAb-BCMA seq. trans. T cells possess higher capacity of cytotoxicity and IFN-gamma (γ) production than BCMA-CAR T cells or BsAb T cells in response. (FIG. 2A) Flow cytometric analysis of BCMA and CS1 expression on the surface of MM cell lines. Three MM cell lines (MM1.S, H929, and RPMI-8226) and one chronic myelogenous leukemia cell line (K562) were stained with anti-CS1 mAb antibody (upper panel) or anti-BCMA mAb (lower panel), different colors were used to distinguish the three MM cell lines, MM.1S (green), H929 (red), and RPMI-8226 (gray) as well as the K562 cell line (blue) or isotype-matched control antibody (black solid line and open area). (FIG. 2B) ⁵¹Cr-labeled MM1.S, H929, RPMI-8226 MM cell lines and the K562 cell line (5×10³ for each cell line) were co-cultured with unmodified T cells (T, black solid line), empty vector-transduced T cells (EV T, black dotted line), T cells expressing anti-NKG2D-anti-CS1 BsAb (BsAb T, red solid line), BCMA CAR (BCMA CAR T, green solid line), and BsAb-BCMA seq. trans. T cells (blue solid line) at the indicated E:T ratios for 4 hours, and target lysis (⁵¹Cr release) was measured. BsAb-BCMA seq. trans. T vs BCMA CAR T, *p<0.05, **p<0.01; seq. trans. T vs BsAb T, #p<0.05, ##p<0.01. K562 cells as BCMA⁻CS1⁻ negative control. (FIG. 2C) unmodified T cells (white square), EV T cells (gray shadow square), BsAb T cells (red square), BCMA CAR T cells (green square) or BsAb-BCMA seq. trans. T cells (blue square) 2×10⁵ were cultured alone (no target) or stimulated with an equal number of MM.1S, H929, or RPMI-8226 MM cells expressing different levels of CS1 and BCMA or BCMA⁻CS1⁻K562 cells for 24 hours, and the supernatants were collected to measure IFN-γ secretion by ELISA. *p<0.05, **p<0.01, n.s. no significant difference. (FIG. 2D and FIG. 2E) Cells were treated as described in (FIG. 2C), and IL-2 or TNF-alpha (α) secretion in cell-free supernatants was determined by ELISA, respectively. **p<0.01, n.s. no significant difference.

FIGS. 3A-3H show the generation of an exemplary BsAb-CAR vector containing both BCMA CAR and anti-NKG2D-anti-CS1 bispecific antibody (BsAb) in the same construct and functional examination of T cells transduced with this construct. (FIG. 3A) Schematic representation of a generated lentiviral vector expressing both BCMA CAR and anti-NKG2D-anti-CS1 BsAb (referred hereafter as BsAb-CAR). T2A, a self-cleaving 2A gene. As described herein, the vector comprises a polynucleotide encoding an antigen binding domain that binds to a cancer or tumor antigen other than an antigen selected from BMCA, EGFRVIII; CD70, mesothelin, CD123, CD19, CEA, CD133, or Her2. (FIG. 3B) Supernatants and cell lysates of empty vector (EV)-transduced T cells or BsAb-CAR T cells were subjected to immunoblot analysis with an anti-6x His-tag antibody (“6x His” disclosed as SEQ ID NO: 111). (FIG. 3C) ⁵¹Cr-labeled MM1.S cells (5×10³) were co-cultured with unmodified T cells (T, black solid line), empty vector-transduced T cells (EV T, black dotted line) or BsAb-CAR T cells (purple line) at the indicated E:T rations for 4 hours, and target lysis (⁵¹Cr release) was measured. (FIG. 3D) Unmodified—(black solid line), EV—(black dotted line), BsAb—(red line), BCMA CAR—(green solid line) or BsAb-CAR (purple solid line)—transduced CD8 (+) T cells isolated from healthy donors were co-cultured with ⁵¹Cr-labeled MM1.S MM cells (5×10³) at the indicated E:T ratios for 4 hours, and target lysis (⁵¹Cr release) was measured. (FIG. 3E) 4-hour ⁵¹Cr release assays with MM.1S MM cells at an E:T ratio of 10:1. To assess this anti-tumor effect in the presence of different quantities of normal (uninfected) human PBMCs, PBMCs were added at a quantity of 1-fold, 10-fold, 100-fold, and 200-fold of the MM.1S MM target cells. (FIG. 3F) ⁵¹Cr release assays of unmodified T cells (black square), EV T cells (pattern square), BsAb T cells (red square), BCMA-CAR T cells (green square), or BsAb-CAR T cells (purple square) against MM.1S MM target cells at an E:T ratio of 5:1. As noted in the figure, the incubation time of effector cells and MM.1S MM target cells is 4-hr for PBMC, NK and NKT cells (left panel) and 16-hr for CD3⁺T cells, CD8⁻T cells, Vγ9Vδ2 T cells or CD4⁺T cells. *p<0.05, **p<0.01, n.s. no significant difference. (FIG. 3G) Control of co-culture of EV T cells (GFP, green) and MM.1S MM cells (red) after 1 hour, confocal microscopy analysis of synapses was determined (scale 10 μL, upper panel; scale 20 μL, lower panel); No synapses are noted, even at higher power shown in the lower panel. (FIG. 3H) One hour co-culture of BsAb-CAR T cells (E: GFP, green) and MM.1S MM cells (T: red); E/T synapses were observed and indicated by the arrows in all frames (S1 shows the same conjugated E/T pair in each frame moving left to right, as does S2, and S3). The frame on the top, left shows a bright field (Bf, scale 10 μL); the top, middle frame shows an immunofluorescence image of BsAb-CAR T cells (GFP, green) and MM.1S MM cells (red) co-culture (scale 10 μL). The frame on the top, right is a merged image with additional anti-6x-His-tag (“6x-His” disclosed as SEQ ID NO: 111) identifying the BsAb (blue, scale 10 μL). The bottom three rows demonstrate the three individual E/T conjugates (S1, S2, and S3) visualized in an enlarged field (scale 20 μL).

FIGS. 4A-4D show that overexpression of BCMA and CS1 in K562 cells triggers enhanced cytotoxicity and cytokine secretion after recognition by BsAb-CAR T cells. (FIG. 4A) Flow cytometric analysis of K562 cells overexpressing CS1 and BCMA (K562-CS1-BCMA, gray shadow) or an empty vector control (K562-PCDH, black solid line) after the cells were stained with a CS1 (left panel) or BCMA (right panel) or IgG isotype control (black dotted line in each panel) antibody. (FIG. 4B) Cytotoxicity of empty vector (EV)- or BsAb-CAR-transduced T cells against K562-CS1-BCMA and K562-PCDH cells, determined by 4-hour ⁵¹Cr release assays. K562-CS1-BCMA or K562-PCDH cells were incubated with EV T cells or BsAb-CAR T cells at indicated E:T ratios. **p<0.01 (K562-CS1-BCMA+BsAb T cells vs. K562-PCDH+BsAb T cells). (FIG. 4C) EV T cells or BsAb-CAR T cells (1×10⁵) were cultured alone or stimulated with an equal number of either K562-CS1-BCMA or K562-PCDH cells. Supernatants from cultures were used to determine IFN-γ secretion by ELISA. **p<0.01. (FIG. 4D) Cells were treated as in (FIG. 4C) and IL-2 secretion in cell-free supernatants was determined by ELISA. **p<0.01.

FIGS. 5A-5E show that secreted anti-NKG2D-anti-CS1 BsAb enhances CAR T cell proliferation through NKG2D signaling. (FIG. 5A) Medium color following culture of unmodified T cells (1), 2-EV T cells (2), BsAb T cells (3), BCMA CAR T cells (4), BsAb-CAR T cells (5) or naïve T cells (6) (non-proliferate control) were displayed in upper panel. The bar graph provides statistical analyses of total cell number included 6 individual samples for each group. **p<0.01 (group 5 vs. groups 1, 2, 4 and group 3 vs. groups 1, 2, 4). To document T cell proliferation or lack thereof, violet cell tracker was used and shown as V450 dilution that is displayed by histograms in the lower panel. (FIG. 5B) Five day-old culture medium of unmodified T cells, EV T cells, and BCMA CAR T cells in the presence or absence of cell-free supernatants of BsAb-CAR T cells from (FIG. 5A), designated as 1+, 2+, 4+ or 1, 2, 4, respectively. Cells were enumerated, and data were presented as a bar graph (top). **p<0.01 (4+ vs. 4, 2+ vs. 2, 1+ vs. 1). Violet cell tracker was shown as V450 dilution that displayed by histograms in the lower panel (bottom). (FIG. 5C) Two-day-old culture medium is shown. 1A—unmodified T cells, 2A—EV T cells, 3A—BsAb T cells, 4A—BCMA CAR T cells, and 5A—BsAb-CAR T cells. On day 0, NKG2D blockade antibody (20 μg/mL) was added into culture of 1B, 2B, 3B, 4B and 5B, while a nonreactive isotype control antibody (20 μg/mL) was added to 1A, 2A, 3A, 4A and 5A). Beneath these wells is flow cytometric staining for CD3, NKG2D, F(ab)₂ (which is denoted by “Fab” indicating expression of the CAR), and Ki67 to measure the cell proliferation. (FIG. 5D) Immunoblot analysis was performed to determine the phosphorylation (p) of AKT protein, and total AKT protein of 1A—Unmodified T, 2A—EV T, 3A—BsAb T, 4A—BCMA-CAR T, and 5A—BsAb-CAR T as well as 1B—Unmodified T cells+NKG2D blockade, 2A—EV T+NKG2D blockade, 3A—BsAb-T+NKG2D blockade, 4A—BCMA-CAR T+NKG2D blockade, and 5A—BsAb-CAR T+NKG2D blockade. (FIG. 5E) The same cells shown in (FIG. 5C) (1A, 2A, 3A, 4A and 5A) were also co-cultured with MM.1S MM cells for 48 hours. Flow cytometric analyses to assess cell proliferation was performed as described above in (FIG. 5C).

FIGS. 6A-6C show that secreted anti-NKG2D-anti-CS1 BsAb enhances CAR T cell survival through NKG2D signaling in vitro. (FIG. 6A) Five day-old culture media of 1—Un. (Unmodified) T+IL-2, 2—EV T+IL-2, 3—BsAb T+IL-2, 4—BCMA-CAR T+IL-2, 5—BsAb-CAR T+IL-2 were displayed. Flow cytometric staining for CD3 (1^{st} column), F(ab)₂ (2^{nd} column), and Ki67 to observe the cell proliferation (3^{rd} column), and Annexin V/Sytox Blue to observe the cell survival (4^{th} column). (FIG. 6B) Five day-old culture media (without IL-2) of 1—unmodified T, 2—EV T, 3—BsAb T, 4—BCMA-CAR T, and 5—BsAb-CAR T were shown in the upper panels. Flow cytometric staining for CD3 (1^{st} column) and Ki67 (2^{nd} column) to detect the cell proliferation. Annexin V/Sytox Blue was included to detect cell survival (3^{rd} column). (FIG. 6C) Statistical analyses of percentages of CD3, Ki67 proliferative cells, Annexin V(−) Sytox Blue(−) alive cells, Annexin V(+) apoptosis cells, and Annexin V(+) Sytox Blue(+) dead cells were displayed. Multiple t-test, compared each groups. **p<0.01.

FIGS. 7A-7D show BsAb-CAR transduced-T cells have better proliferation and survival capacity than BCMA-CAR T cells and control T cells in vivo. (FIG. 7A) Design of i.v. injection of unmodified T cells, EV T cells, BCMA-CAR T cells and BsAb-CAR T cells into immunodeficient NSG mice (a, upper). 3D histograms (lower panel, 1^{st} column) indicate the percentages of injected human CD3 T cells. The blue histograms are for the mice that had no T cell injection on day −1, the orange histograms represent 1 day after T cell injection, and the black histograms represent 14 days after T cell injection (red arrow points the BsAb-CAR T group). Contours indicate CD69 expression (orange for 1 day after i.v. injection, and black for 14 days after i.v. injection). The purple color histograms (4^{th} column) indicated percentages of the injected CD3 T cells 35 days after i.v. The purple contours are the combination of Ki67 and CD69 staining of 4 groups to reveal the cell proliferation. The red contours are the combination of Sytox Blue and Annexin V staining to reveal the cell apoptosis and cell death. S−/A− denotes Sytox Blue (−)/Annexin V(−), S−/A+ denotes Sytox Blue−/Annexin V+, and S+/A+ denotes Sytox Blue+/Annexin V+. (FIG. 7B, FIG. 7C) Statistical analysis of human CD3⁺ (FIG. 7B) and CD69⁻ (FIG. 7C) cells shown in (FIG. 7A). Unmodified T (empty rectangle), EV T (pattern filled rectangle), BCMA-CAR T (gray shadow rectangle), and BsAb-CAR T (black rectangle). Multiple t-test, compared each group. **p<0.01, n.s. no significant difference, n=5 mice per group. (FIG. 7D) Statistical analysis of the percentages of Ki67-CD69⁺ proliferative cells, Annexin V(−) Sytox Blue(−) alive cells, Annexin V(+) Sytox Blue(−) apoptosis cells, and Annexin V(+) Sytox Blue(+) dead cells. Multiple t-test, compared each group. **p<0.01, n.s. no significant difference, n=5/group.

FIGS. 8A-8C show BsAb-CAR T cells specifically recognize and eliminate CS1 or/and BCMA-expressing human primary multiple myeloma cells ex vivo. (FIG. 8A) Flow cytometric surface staining for CS1 and BCMA protein in CD138⁺ multiple myeloma tumor cells isolated from MM patients' bone marrow. Results from 8 patients are shown. Eight patients' MM cells were stained with PE-conjugated anti-CS1 mAb antibody (left panel) or APC-conjugated streptavidin with biotin-labeled anti-BCMA mAb (right panel). Various colors are used to indicate each of the 8 patients or isotype-matched control antibody (gray shadow). (FIG. 8B) 5×10³ CD138⁺ multiple myeloma tumor cells were co-cultured with EV T cells (black dotted line), BsAb T cells (red line), BCMA-CAR T cells (green line), or BsAb-CAR T cells (purple line) at the indicated E:T ratios for 4 hours, then specific lysis was determined using a standard ⁵¹Cr release assay. Representing data from Patient sample 1 and Patient sample 4 are shown. Eight patients' data were analyzed and shown as individual values for each patient (right panel). (FIG. 8C) Transduced T cells as indicated were co-cultured with CD138⁺ multiple myeloma tumor cells at an E:T ratio of 1:1 for 24 hours, and IFN-γ secretion was measured in cell-free supernatants via ELISA.

FIGS. 9A-9C show that BsAb-CAR T cells are superior to suppress in vivo MM growth and prolong survival of mice bearing MM or being re-challenged with tumor cells. (FIG. 9A) Bioluminescence imaging was shown for five representative mice bearing MM.1S tumors from each indicated group. NSG mice were intravenously inoculated with 8×10⁶ MM.1S cells expressing luciferase (day 0). On days 10, 17 and 24 after tumor implantation, each mouse received an i.v. injection with either saline (control group) or, 10×10⁶ EV T cells, BsAb T cells, BSMA CAR T cells, BsAb-BCMA seq. trans. T cells, or BsAb-CAR T cells, respectively (upper panel, experiment schedule). Images on the row were taken on day 10 after tumor implantation, just before infusion of engineered T cells or control T cells. Images in the middle row were taken on day 24, after mice already undergone treatment twice (on day 10, 17) and just prior to the third treatment. Images in the bottom row show mice on day 31, after 3 rounds of treatment (on day 10, 17, and 24). (FIG. 9B) On day 80 after tumor implantation, peripheral blood (PBL) were collected from survived mice (3 mice of BCMA CAR T cells treated group, 4 mice of BCMA seq. trans. T cells treated group, and 5 mice of BsAb-CAR T cells treated group). PBL total cell numbers were calculated (left panel). Flow cytometric staining with FITC-conjugated anti-human CD45 mAb antibody and APC-conjugated streptavidin with biotin-labeled goat anti-mouse (Fab)₂ polyclonal antibody or normal polyclonal goat immunoglobulin G (IgG) antibody. The percentages of Fab(+) cells and numbers were calculated as shown in the middle. (FIG. 9C) Kaplan-Meier survival curve of MM.1S-bearing mice treated with various transduced-T cells, saline (black solid line), EV T cells (black dotted line), BsAb T cells (red line), BCMA CAR T cells (green line), BCMA seq. trans. T cells (blue line), and BsAb-CAR T cells (purple dotted line). The gray dotted vertical line with arrow indicated the day 80 when mice were re-challenged with 4×10⁶ MM.1S cells.

FIGS. 10A-10D show that BsAb-CAR T cells more effectively than BCMA-CAR T cells suppress in vivo MM growth and prolong survival of MM tumor-bearing mice in the presence of adoptively transferred human PBMC. (FIG. 10A) Bioluminescence imaging was shown for three representative mice bearing MM.1S tumors from each indicated group. NSG mice were intravenously inoculated with 8×10⁶ MM.1S cells expressing luciferase (day 0). On days 10, 17 and 24 after tumor implantation, each mouse received an i.v. injection with either saline (control group), BSMA-CAR T cells, or BsAb-CAR T cells. Myeloid cells-depleted PBMC from the same donor were i.v. injected to mice on day 10 after tumor implantation (upper panel, experiment schedule). Images on the first column were taken on day 10 after tumor implantation, just prior to infusion of engineered T cells or control T cells. Images in the second column were taken on day 19, after the mice already undergone treatment twice (on day 10, 17) and just before the third treatment was administered. Images in the third column show mice on day 28, after 3 rounds of treatment (on day 10, 17, and 24). Images in the fourth column show mice on day 37. (FIG. 10B) Blood was collected from survived BsAb-CAR T cells injected mice (n=5), and untreated NSG mice (None, n=5). Flow cytometric staining for CD19/20(+) human plasma cells, CD56(+) NK cells, and CD3(+) T cells. Green color of contour that gated on human CD3(+)F(ab)₂(+) indicate the percentage of CAR expression within survived human T cells. (FIG. 10C) Statistical analysis of the percentages of human CD19/20(+) plasma cells, CD56(+) NK cells, CD3(+) T cells, and CD3(+)F(ab)₂(+) survived CAR T cells. (FIG. 10D) Kaplan-Meier survival curve of MM.1S-bearing mice treated with various transduced-T cells, saline (black solid line), BCMA-CAR T cells (green line), or BsAb-CAR T cells (purple dotted line). P<0.0001, BsAb-CAR T cells vs. BCMA-CAR T cells.

FIGS. 11A-11C. (FIG. 11A) 3D rainbow dots flow cytometric map (basic on CD3 staining, CD3 positive cells show the yellow and green color, and CD3 negative cells show the dark blue and purple color) displays the percentages of CD3(+) T cells (black circle, yellow color with green color), γδ T cells (yellow color alone), NKT cells (orange circle, yellow color with green color), and NK cells (blue circle, dark blue and purple color). 2D contour maps show the details of 3D map. (FIG. 11B) Flow cytometric staining of NKG2D surface expression in T cells, CD8⁺ T cells, pan γδ T cells, Vγ9Vδ2 T cells, NKT cells, and NK cells. Data presented are representative of PBMC from 10 healthy donors. (FIG. 11C) Statistical analyses of percentages of NKG2D⁺ cells in (B). n=10.

FIGS. 12A-12B. (FIG. 12A) 4-hour 51Cr release assays at the E:T ratio of 10:1 [E, effector cells of unmodified T cells (black solid line) or EV—(black dotted line), BsAb—(red line), BCMA-CAR—(green line), or BsAb-CAR-transduced T cells (purple line)]. Different quantities of human PBMC at 1-fold, 10-fold, 100-fold, or 200-fold over target cells. Specific lysis curve of one representative experiment of three are shown in A and the summary data of three are shown in B. (FIG. 12B) Statistical analyses of ⁵¹Cr release assays results of (a), multiple t-test, *p<0.05, **p<0.01, n.s. no significant difference, 3-time repetition.

FIGS. 13A-13D. (FIG. 13A) CD3(+) T cells, CD8(+) cytotoxic T cells, CD4(+) T cells, γδ T cells, NKT cells, and NK cells were isolated from leukopacks ordered from the American Red Cross. Black color contour map for primed T shows combinational staining of CD3 and pan αβ TCR. Brown and green color contour maps show sorted CD8(+) and CD4(+) T cells, respectively. (FIG. 13B) Activated human NK cells were stained with CD3 and CD56. (FIG. 13C) Sorted pan γδ T cells were stained with CD3 and pan γγ TCR antibodies. Black color contour maps for activated Vγ9γδ2 TCR T cells were performed by combinational staining of CD3, CD56, Vγ9 and Vδ2. (FIG. 13D) Freshly FACS-sorted human CD3(+)CD56(+) NKT cells were stained with CD3 and CD56.

FIGS. 14A-14E. (FIG. 14A) ⁵¹Cr release assays of unmodified T cells (black solid line), EV T cells (black dotted line), BsAb T cells (red line), BCMA-CAR T cells (green line) or BsAb-CAR T cells (purple line) at an E:T ratio of 5:1 at different time points, including 2 h, 4 h, 8 h, and 16 h. No additional PBMC were added. (FIG. 14B, FIG. 14C, FIG. 14D, FIG. 14E) Above described cytotoxicity assays were repeated in the presence or absence of bulk T cells or individual T cell subsets including primed CD3(+) T cells, CD8(+) T cells, CD4(+) T cells, and Vγ9Vδ2 T cells. The ratio of effector cells to target MM.1S cells is 5:1 for all experiments.

FIGS. 15A-15J show confocal microscopic analysis following 24 hour co-culture of either BsAb-CAR T cells (green) or EV T cells (green) with MM.1S MM cells (red). (FIG. 15A-FIG. 15F) Co-culture of BsAb-CAR T cells with MM.1S MM cells for 24 hours shows elimination of MM.1S MM cells. (FIG. 15G-FIG. 15J) Co-Culture of EV T cells with MM.1S MM cells for 24 hours shows persistence of MM.1 S MM cells. Bf=Bright field; Scale, 10 μL.

FIG. 16 shows the generation of K562 cells stably expressing the CS1 and BCMA genes. The left pseudo color flow map indicates the control of un-transduced K562 cells. The middle pseudo color flow map indicates FACS-sorted pCDH-CS1-GFP lentivirus-infected K562 cells. The third pseudo color flow map indicates FACS-sorted CS1(+)BCMA(+) K562 cells.

FIGS. 17A-17D. (FIG. 17A) 48 hours' culture. 1A—unmodified T cells, 2A—EV T cells, 3A—BsAb T cells, 4A—BCMA CAR T cells and 5A—BsAb-CAR T cells. Flow cytometric staining for CD3 and NKG2D to observe CD3(+) populations (blue flames), CD3(+)NKG2D(+) populations (red flame), and CD3(+)NKG2D(−) populations (green flames). (FIG. 17B) On day 0, NKG2D blockade antibody (20 μg/mL) was added in to (FIG. 17A) culture and named as 1B, 2B, 3B, 4B and 5B. After 48 hours, flow cytometric staining for CD3 and F(ab)₂ to observe the cell population. CD3(+) populations (blue flames). (FIG. 17C) 48 hours' culture media from (FIG. 17A) were shown. On day 0, CS1 blocking antibody (20 μg/mL) was added into (FIG. 17A) culture and named as 1C, 2C, 3C, 4C and 5C. After 48 hours, flow cytometric analysis was performed after for staining cells with anti-CD3, anti-F(ab)₂, anti-NKG2D, and anti-Ki67 to observe cell proliferations. Cells were gated on CD3(+) and CD3(+)NKG2D(+) populations (red flame), CD3(+)NKG2D(−) populations (green flames) were indicated. Red color dots flow maps indicated the proliferation of NKG2D(+)Fab(+) (upper three) or NKG2D(+)Fab(−) (lower two) cells. Green color dots flow maps indicate proliferation of NKG2D(−)Fab(+) (upper three) or NKG2D(−)Fab(−) (lower two) cells. Fab denotes anti-F(ab)₂ staining for CAR expression. (FIG. 17D) Cells from 1A, 2A, 3A, 4A and 5A in (FIG. 17A) were co-cultured with MM. S tumor cells for 48 hours. Flow cytometric analyses were performed after staining cells with anti-CD3, anti-F(ab)₂, and anti-NKG2D antibodies. Fab denotes F(ab)2 staining, which indicates CAR expression.

FIG. 18 provides supplemental data to the data in FIG. 7. In FIG. 7, FIG. 3D histograms (1^{st} column) of human CD3 T cells percentages are shown. These are the original pseudo color flow maps. The blue color frame indicated the human CD3 percentages of the mice that had no T cell injection on day −1 (1st column). The orange color frame represents anti-human CD3(+) cells in mice one day after T cell injection, and CAR expression were detected by flow cytometric analysis after staining with anti-F(ab)₂ (2^{nd}, 3^{rd} column). The black color frame shows anti-human CD3(+) cells in mice 14 days after infusion with engineered or control human T cell (4^{th} column). The purple color frame shows anti-human CD3(+) cells in mice 35 days after infusion with engineered or control human T cell (5^{th} column). Statistical analysis for CD3(+) human cells is shown in FIG. 7.

FIGS. 19A-19B show the effects of depletion of myeloid cells in human healthy donor's PBMC to avoid GVHD when injected to NSG mice. (FIG. 19A) Ficoll-Paque PLUS isolated human PBMC were stained. Number 1 indicated lymphocyte percentages, 2 indicated granulocyte percentages, and 3 indicated monocyte percentages. CD11c, CD14, CD33 and CD66b were stained to check the percentages of myeloid cells in healthy donor's PBMC before FACS sorted. (FIG. 19B) After sorting, CD11c, CD14, CD33 and CD66b were stained as above described, pseudo color low maps were displayed.

FIG. 20 provides an assessment of cytotoxicity of human BsAb-CAR T cells against autologous PBMC, T cells, NK cells, and plasma cells by a standard 4 h-⁵¹Cr release assay. PBMC were isolated by Ficoll-Paque PLUS gradient centrifugation. CD3(+) T cells, CD56(+) NK cells, and CD19/20(+) plasma cells were FACS-sorted from PBMC. EV T cells were used as control in the cytotoxicity assay.

FIGS. 21A-21D. (FIG. 21A) Different time points (12 h, 24 h, 48 h, 72 h, and 96 h) of BsAb BCMA-CAR (BsAb-CAR T) lentivirus-infected healthy donor's primed T cells. The same view of BF (Bright field) GFP (green) is shown. (FIG. 21B) Immunoblotting with anti-His-tag to show secretion of BsAb in the supernatant of BsAb-CAR T cells. FIG. 21B discloses “His 6x” as SEQ ID NO: 111. (FIG. 21C) Flow cytometric staining for BsAb-CAR T lentivirus-infected primed T cells. GFP-positive cells were sorted, and cells were stained with biotin labeled goat anti-mouse Fab specific or isotype-matched control antibody, followed by streptavidin and CD3 antibody staining. (FIG. 21D) ⁵¹Cr-labeled H929, RPMI-8226 and K562 target cell lines (5×10³) were co-cultured with unmodified T cells (black solid line), empty vector-transduced T cells (EV T, black dotted line), or BsAb-CAR T cells (purple line) at the indicated E:T ratios for 4 hours. Target lysis (⁵¹Cr release) was measured. BsAb-CAR T vs unmodified T or EV T, **P<0.01, Repeated for three times. BCMA(−)CS1(−) negative K562 served as negative control target cells.

### DETAILED DESCRIPTION

It is to be understood that the present disclosure is not limited to particular aspects described, as such may, of course, vary. It is also to be understood that the terminology used herein is for the purpose of describing particular aspects only, and is not intended to be limiting, since the scope of the present disclosure will be limited only by the appended claims. Throughout this disclosure, various technical publications are referenced by an Arabic numeral. The complete citations for these publications can be found immediately preceding the claims and are incorporated herein by reference.

Both B-cell maturation antigen (BCMA) and SLAMF7 (CS1 or CD319) have been shown to be excellent MM tumor antigen targets, while NKG2D is a superb immune cell target. NKG2D, a receptor, is expressed on virtually all cytolytic immune cells, including NK cells, NKT cells, CD8(+) T cells, and γδ T cells²³. BCMA is a member of the tumor necrosis factor receptor superfamily (TNFRSF17 or CD269), is selectively induced during plasma cell differentiation and is nearly absent on naïve and memory B cells^{24,25} Adoptive transfer of anti-BCMA-CAR-expressing T cells has been reported as a promising new strategy for treating MM²⁶⁻²⁸. CS1 is another attractive tumor-associated target antigen in MM, because CS1 is highly and ubiquitously expressed on the surface of MM cells^{29,30} CS1 is expressed at low levels on NK cells and on a subset of activated CD8(+) T cells, but it is almost undetectable on myeloid cells and normal hematopoietic stem cells³¹. A therapeutic monoclonal antibody against CS1 has been approved by the FDA for the treatment of MM³². Applicant's published research showed that genetic modification of T cells or NK cells redirected toward CS1 enhanced eradication of myeloma cells^{29,30}. A recent preclinical study showed that patient MM cells subjected to CS1 CAR T cell treatment were effectively eradicated, while normal NK and T cells with low levels of CS1 expression remained unaffected (Gogishvili, 2017 Blood. 2017 Dec. 28;130(26):2838-2847. doi: 10.1182/blood-2017-04-778423. Epub 2017 Oct. 31). NKG2D, an activating receptor, is expressed on a variety of innate and adaptive cytolytic cells as mentioned above^{11,33}. Triggering NKG2D can lead to activation of both innate and adaptive cellular immunity^{11,33}.

As disclosed herein, Applicants can engineer T cells to (1) express a specific second-generation CAR that binds to a cancer or tumor antigen that optionally binds other than BMCA, or in another aspect, none of EGFRVIII; CD70, mesothelin, CD123, CD19, CEA, CD133, or Her2 and (2) simultaneously secrete an anti-NKG2D-BsAb. Previous data suggest that these cells, hereafter referred to as BsAb-CAR T cells, represent a promising therapy for relapsed and/or refractory cancer, and can be a suitable platform for producing the next generation CAR-based cancer immunotherapy.

Such a combination of a CAR and an anti-NKG2D-based bispecific antibody as a single vector transduced into T cells has yet to be described in the literature. Applicants have demonstrated that this approach generates T cells (BsAb CAR T-cells) that function as potent cytolytic effector cells against tumors. Provided herein is a representative example of a T cell CAR directed against a well-known target in multiple myeloma (MM), called BCMA, and the anti-NKG2D-based bispecific antibody recognizes the well-described MM tumor antigen CS1 that brings it into close proximity of any innate or adaptive cytolytic effector cell bearing the NKG2D antigen. While Applicants present examples drawn to T-cell CAR directed against a well-known target in MM, BCMA, Applicants describe herein an expansion to a variety of complementary CARs and anti-NKG2D-based bispecific antibodies into a single vector to then infect T and NK cells for anti-tumor efficacy. Applicants further describe a modified approach that can be applied to CARs comprising an antigen binding domain of a cancer or tumor targeting antibody that bind to any number of cancer/tumor antigens known in the art, including, but not limited to: FLT-3, CD19, mesothelin, human epidermal growth factor receptor 2 (HER2), prostate stem cell antigen (PSCA), carcinoembryonic antigen (CEA), GTP-ase-activating protein (GAP), ganglioside G2 (GD2), CD5, prostate specific membrane antigen (PSMA), receptor tyrosine kinase-like orphan receptor 1 (ROR1), CD123, CD70, CD38, B cell maturation antigen (BCMA), mucin 1, (Muc1), ephrin type-A receptor 2 precursor (EphA2), epidermal growth factor receptor variant III (EGFRVIII), interleukin 13 receptor alpha 2 (IL13Ra2), CD133, glypican 3 (GPC3), epithelial cell adhesion molecule precursor (EpCam), fibroblast activation protein alpha (FAP), vascular endothelial growth factor receptor 2 (VEGFR2), cancer/testis (CT), guanylyl cyclase C (GUCY2C), tumor-associated glycoprotein-72 (TAG-72), thymidine kinase 1 (TK1), and hypoxanthine guanine phosphoribosyltransferase (HPRT1); see Townsend et al. (2018) J. Exp. & Clinical Cancer Res. 37:163.

As an example of this approach, a single vector delivering two complementary modalities directed against two distinct tumor associated antigens on MM cells, is superior to either modality alone and is superior to the use of T cells infected sequentially with the two separate constructs, one encoding the CAR and the other encoding the anti-NKG2D-based bispecific antibody. Further, when the T cell (infected with the experimental vector encoding both the CAR and the anti-NKG2D-based bispecific antibody) encounters the MM cell expressing both antigens, the BsAb CAR T cell undergoes both proliferation and enhanced survival in vitro and in vivo through NKG2D-mediated activation. Such results are altogether unexpected.

In sum, Applicants constructed BCMA CAR T cells that also secrete anti-NKG2D-anti-CS1 bispecific antibodies; these cells effectively target BCMA(+) and/or CS1(+) multiple myeloma (MM/i) cells. The secretion of anti-NKG2D-anti-CS1 bispecific antibodies by BCMA CAR T cells enhances both CAR T cell proliferation in vitro and CAR T cell survival and proliferation in vivo through NKG2D-mediated activation. BCMA CAR T cells secreting anti-NKG2D-anti-CS1 bispecific antibodies display significantly better in vitro and in vivo efficacy against tumor cell targets compared to single therapies with BCMA CAR T or with T cells secreting anti-NKG2D-anti-CS1 bispecific antibody alone. These results can be generalized to a variety of CARs employed in the same manner.

Unless defined otherwise, all technical and scientific terms used herein have the same meanings as commonly understood by one of ordinary skill in the art to which this technology belongs. Although any methods and materials similar or equivalent to those described herein can be used in the practice or testing of the present technology, the preferred methods, devices and materials are now described. All technical and patent publications cited herein are incorporated herein by reference in their entirety. Nothing herein is to be construed as an admission that the present technology is not entitled to antedate such disclosure by virtue of prior invention.

The practice of the present technology will employ, unless otherwise indicated, conventional techniques of tissue culture, immunology, molecular biology, microbiology, cell biology, and recombinant DNA, which are within the skill of the art. See, e.g., Green and Sambrook eds. (2012) Molecular Cloning: A Laboratory Manual, 4^{th} edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); MacPherson et al. (1995) PCR 2: A Practical Approach; McPherson et al. (2006) PCR: The Basics (Garland Science); Harlow and Lane eds. (1999) Antibodies, A Laboratory Manual; Greenfield ed. (2014) Antibodies, A Laboratory Manual; Freshney (2010) Culture of Animal Cells: A Manual of Basic Technique, 6^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Herdewijn ed. (2005) Oligonucleotide Synthesis: Methods and Applications; Hames and Higgins eds. (1984) Transcription and Translation; Buzdin and Lukyanov ed. (2007) Nucleic Acids Hybridization: Modern Applications; Immobilized Cells and Enzymes (IRL Press (1986)); Grandi ed. (2007) In Vitro Transcription and Translation Protocols, 2^{nd} edition; Guisan ed. (2006) Immobilization of Enzymes and Cells; Perbal (1988) A Practical Guide to Molecular Cloning, 2^{nd} edition; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Lundblad and Macdonald eds. (2010) Handbook of Biochemistry and Molecular Biology, 4^{th} edition; and Herzenberg et al. eds (1996) Weir's Handbook of Experimental Immunology, 5^{th} edition; and the more recent editions each thereof available at the time of filing.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

It is to be inferred without explicit recitation and unless otherwise intended, that when the present technology relates to a polypeptide, protein, polynucleotide or antibody, an equivalent or a biologically equivalent of such is intended within the scope of the present technology.

### Definitions

As it would be understood, the section or subsection headings as used herein is for organizational purposes only and are not to be construed as limiting and/or separating the subject matter described.

As used in the specification and claims, the singular form “a”, “an”, and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this disclosure or process steps to produce a composition or achieve an intended result. Embodiments defined by each of these transition terms are within the scope of this disclosure.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

As used herein, the term “animal” refers to living multi-cellular vertebrate organisms, a category that includes, for example, mammals and birds. The term “mammal” includes both human and non-human mammals.

The terms “subject,” “host,” “individual,” and “patient” are as used interchangeably herein to refer to human and veterinary subjects, for example, humans, animals, non-human primates, dogs, cats, sheep, mice, horses, and cows. In some embodiments, the subject is a human. In some embodiments, they refer to and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present disclosure is also useful for veterinary treatment of companion mammals, exotic animals and domesticated animals, including mammals, rodents. In one embodiment, the mammals include horses, dogs, and cats. In another embodiment of the present disclosure, the human is a fetus, an infant, a pre-pubescent subject, an adolescent, a pediatric patient, or an adult. In one aspect, the subject is pre-symptomatic mammal or human. In another aspect, the subject has minimal clinical symptoms of the disease. The subject can be a male or a female, adult, an infant or a pediatric subject. In an additional aspect, the subject is an adult. In some instances, the adult is an adult human, e.g., an adult human greater than 18 years of age.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins.

Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, murine or humanized non-primate antibodies), heteroconjugate antibodies (such as, bispecific antibodies). See also, Pierce Catalog and Handbook, 1994-1995 (Pierce Chemical Co., Rockford, Ill.); Owen et al., Kuby Immunology, 7^{th} Ed., W.H. Freeman & Co., 2013; Murphy, Janeway's Immunobiology, 8^{th} Ed., Garland Science, 2014; Male et al., Immunology (Roitt), 8^{th} Ed., Saunders, 2012; Parham, The Immune System, 4^{th} Ed., Garland Science, 2014.

As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

In terms of antibody structure, an immunoglobulin has heavy (H) chains and light (L) chains interconnected by disulfide bonds. There are two types of light chain, lambda (k) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopts a 3-sheet conformation and the CDRs form loops which connect, and in some cases form part of, the f-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located (heavy chain regions labeled CDHR and light chain regions labeled CDLR). Thus, a CDHR3 is the CDR3 from the variable domain of the heavy chain of the antibody in which it is found, whereas a CDLR1 is the CDR1 from the variable domain of the light chain of the antibody in which it is found. For example, a TNT antibody will have a specific VH region and the VL region sequence unique to the TNT relevant antigen, and thus specific CDR sequences. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

As used herein, a fragment crystallizable (Fc) region refers to the tail region of an antibody that stabilizes the antibody and optionally interacts with (such as binds) an Fc receptor on an immune cell or on a platelet or that binds a complement protein. In some embodiments, a Fc mutant may be used, such as comprising one or two or all three mutations of F234A, L235A and N297Q of human IgG4 Fc region in a Fc or an equivalent thereof at positions corresponding to those of human IgG4 Fc region, such as for SEQ ID NO: 81, the corresponding positions are amino acid (aa) 16, aa 17 and aa 79 of SEQ ID NO: 81.

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

In some embodiments, antigen of a binding moiety, such as an antibody, an antigen binding fragment thereof, or a CAR, may be provided herein in a format of “antigen” followed by the binding moiety (such as a BCMA CAR), or having “anti-” before the antigen and the binding moiety after the antigen (such as an anti-BCMA antibody), or the binding moiety followed by “to” or “directed to” and then the antigen (such as an antibody to CS1).

As used herein, the terms tumor associated antigen (TAA), cancer antigen, tumor antigen, cancer relevant antigen, and tumor relevant antigen are used interchangeably herein, referring to antigenic substance of a cancer or tumor cells. In some embodiments, a TAA presents on some tumor or cancer cells and also on some normal cells, optionally at a lower level. In some embodiments, a TAA only presents on a tumor or cancer cell but not on a normal cell. In some embodiments, a TAA is selected from FLT3, CD19, mesothelin, human epidermal growth factor receptor 2 (HER2), prostate stem cell antigen (PSCA), carcinoembryonic antigen (CEA), CD33, GTPase-activating protein (GAP), ganglioside G2 (GD2), CD5, prostate specific membrane antigen (PSMA), receptor tyrosine kinase-like orphan receptor 1 (ROR1), CD123, CD70, CD38, mucin 1, (Muc1), ephrin type-A receptor 2 precursor (EphA2), epidermal growth factor receptor variant III (EGFRVIII), interleukin 13 receptor alpha 2 (IL13Ra2), CD133, glypican 3 (GPC3), epithelial cell adhesion molecule precursor (EpCam), fibroblast activation protein alpha (FAP), vascular endothelial growth factor receptor 2 (VEGFR2), cancer/testis (CT), guanylyl cyclase C (GUCY2C), tumor-associated glycoprotein-72 (TAG-72), thymidine kinase 1 (TK1), and hypoxanthine guanine phosphoribosyltransferase (HPRT1).

As used herein, the term “antigen binding domain” refers to any protein or polypeptide domain that can specifically bind to an antigen target.

As used herein, the term “autologous,” in reference to cells refers to cells that are isolated and infused back into the same subject (recipient or host). “Allogeneic” refers to non-autologous cells.

As used herein, the term “B cell,” refers to a type of lymphocyte in the humoral immunity of the adaptive immune system. B cells principally function to make antibodies, serve as antigen presenting cells, release cytokines, and develop memory B cells after activation by antigen interaction. B cells are distinguished from other lymphocytes, such as T cells, by the presence of a B-cell receptor on the cell surface. B cells may either be isolated or obtained from a commercially available source. Non-limiting examples of commercially available B cell lines include lines AHH-1 (ATCC® CRL-8146™), BC-1 (ATCC® CRL-2230™), BC-2 (ATCC® CRL-2231™), BC-3 (ATCC® CRL-2277™) CA46 (ATCC® CRL-1648™), DG-75 [D.G.-75] (ATCC® CRL-2625™), DS-1 (ATCC® CRL-11102™), EB-3 [EB3] (ATCC® CCL-85™), Z-138 (ATCC #CRL-3001), DB (ATCC CRL-2289), Toledo (ATCC CRL-2631), Pfiffer (ATCC CRL-2632), SR (ATCC CRL-2262), JM-1 (ATCC CRL-10421), NFS-5 C-1 (ATCC CRL-1693); NFS-70 C10 (ATCC CRL-1694), NFS-25 C-3 (ATCC CRL-1695), AND SUP-B15 (ATCC CRL-1929). Further examples include but are not limited to cell lines derived from anaplastic and large cell lymphomas, e.g., DEL, DL-40, FE-PD, JB6, Karpas 299, Ki-JK, Mac-2A Ply1, SR-786, SU-DHL-1, -2, -4, -5, -6, -7, -8, -9, -10, and -16, DOHH-2, NU-DHL-1, U-937, Granda 519, USC-DHL-1, RL; Hodgkin's lymphomas, e.g., DEV, HD-70, HDLM-2, HD-MyZ, HKB-1, KM-H2, L 428, L 540, L1236, SBH-1, SUP-HD1, SU/RH-HD-1. Non-limiting exemplary sources for such commercially available cell lines include the American Type Culture Collection, or ATCC, (http://www.atcc.org/) and the German Collection of Microorganisms and Cell Cultures (https://www.dsmz.de/).

As used herein, a “cancer” is a disease state characterized by the presence in a subject of cells demonstrating abnormal uncontrolled replication and in some aspects, the term may be used interchangeably with the term “tumor.” The term “cancer or tumor antigen” refers to an antigen known to be associated and expressed on the surface with a cancer cell or tumor cell or tissue, and the term “cancer or tumor targeting antibody” refers to an antibody that targets such an antigen.

The term “chimeric antigen receptor” (CAR), as used herein, refers to a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” or “intracellular signaling domain” means any oligopeptide or polypeptide known to function as a domain that transmits a signal to cause activation or inhibition of a biological process in a cell. In certain embodiments, the intracellular domain may comprise, alternatively consist essentially of, or yet further comprise one or more costimulatory signaling domains in addition to the primary signaling domain. The “transmembrane domain” means any oligopeptide or polypeptide known to span the cell membrane and that can function to link the extracellular and signaling domains. A chimeric antigen receptor may optionally comprise a “hinge domain” which serves as a linker between the extracellular and transmembrane domains. Non limiting examples of such domains are provided herein, e.g.:

Hinge domain: IgG1 heavy chain hinge coding
sequence:
(SEQ ID NO: 112)
CTCGAGCCCAAATCTTGTGACAAAACTCACACATGCCCACCGTGCCCG

Additional non-limiting example includes an IgG4 hinge region, IgD and CD8 domains, as known in the art.

Transmembrane domain: CD28 transmembrane region
coding sequence:
(SEQ ID NO: 113)
TTTTGGGTGCTGGTGGTGGTTGGTGGAGTCCTGGCTTGCTATAGCTTG
CTAGTAACAGTGGCCTTTATTATTTTCTGGGTG
Intracellular domain: 4-1BB co-stimulatory
signaling region coding sequence:
(SEQ ID NO: 114)
AAACGGGGCAGAAAGAAACTCCTGTATATATTCAAACAACCATTTATG
AGACCAGTACAAACTACTCAAGAGGAAGATGGCTGTAGCTGCCGATTT
CCAGAAGAAGAAGAAGGAGGATGTGAACTG
Intracellular domain: CD28 co-stimulatory
signaling region coding sequence:
(SEQ ID NO: 115)
AGGAGTAAGAGGAGCAGGCTCCTGCACAGTGACTACATGAACATGACT
CCCCGCCGCCCCGGGCCCACCCGCAAGCATTACCAGCCCTATGCCCCA
CCACGCGACTTCGCAGCCTATCGCTCC
Intracellular domain: CD3 zeta signaling region
coding sequence:
(SEQ ID NO: 116)
AGAGTGAAGTTCAGCAGGAGCGCAGACGCCCCCGCGTACCAGCAGGGC
CAGAACCAGCTCTATAACGAGCTCAATCTAGGACGAAGAGAGGAGTAC
GATGTTTTGGACAAGAGACGTGGCCGGGACCCTGAGATGGGGGGAAAG
CCGAGAAGGAAGAACCCTCAGGAAGGCCTGTACAATGAACTGCAGAAA
GATAAGATGGCGGAGGCCTACAGTGAGATTGGGATGAAAGGCGAGCGC
CGGAGGGGCAAGGGGCACGATGGCCTTTACCAGGGTCTCAGTACAGCC
ACCAAGGACACCTACGACGCCCTTCACATGCAGGCCCTGCCCCCTCGC
TAA

Further embodiments of each exemplary domain component include other proteins that have analogous biological function that share at least 70%, or alternatively at least 80% amino acid sequence identity, preferably 90% sequence identity, more preferably at least 95% sequence identity with the proteins encoded by the above disclosed nucleic acid sequences. Further, non-limiting examples of such domains are provided herein.

As used herein, the term “CD8 α hinge domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 α hinge domain sequence as shown herein. The example sequences of CD8 α hinge domain for human, mouse, and other species are provided in Pinto, R. D. et al. (2006) Vet. Immunol. Immunopathol. 110:169-177. The sequences associated with the CD8 α hinge domain are provided in Pinto, R. D. et al. (2006) Vet. Immunol. Immunopathol. 110:169-177. Non-limiting examples of such include:

Human CD8 alpha hinge domain:
(SEQ ID NO: 117)
PAKPTTTPAPRPPTPAPTIASQPLSLRPEACRPAAGGAVHTRGLDFAC
DIY.
Mouse CD8 alpha hinge domain:
(SEQ ID NO: 118)
KVNSTTTKPVLRTPSPVHPTGTSQPQRPEDCRPRGSVKGTGLDFACDI
Y.
Cat CD8 alpha hinge domain:
(SEQ ID NO: 119)
PVKPTTTPAPRPPTQAPITTSQRVSLRPGTCQPSAGSTVEASGLDLSC
DIY.

As used herein, the term “CD8 α transmembrane domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 α transmembrane domain sequence as shown herein. The fragment sequences associated with the amino acid positions 183 to 203 of the human T-cell surface glycoprotein CD8 alpha chain (GenBank Accession No: NP_001759.3), or the amino acid positions 197 to 217 of the mouse T-cell surface glycoprotein CD8 alpha chain (GenBank Accession No: NP_001074579.1), and the amino acid positions 190 to 210 of the rat T-cell surface glycoprotein CD8 alpha chain (GenBank Accession No: NP_113726.1) provide additional example sequences of the CD8 α transmembrane domain. The sequences associated with each of the listed accession numbers are provided as follows:

Human CD8 alpha transmembrane domain:
(SEQ ID NO: 120)
IYIWAPLAGTCGVLLLSLVIT.
Mouse CD8 alpha transmembrane domain:
(SEQ ID NO: 121)
IWAPLAGICVALLLSLIITLI.
Rat CD8 alpha transmembrane domain:
(SEQ ID NO: 122)
IWAPLAGICAVLLLSLVITLI.

As used herein, the term “CD28 transmembrane domain” refers to a specific protein fragment associated with this name and any other molecules that have analogous biological function that share at least 70%, or alternatively at least 80% amino acid sequence identity, at least 90% sequence identity, or alternatively at least 95% sequence identity with the CD28 transmembrane domain sequence as shown herein. The fragment sequences associated with the GenBank Accession Nos: XM_006712862.2 and XM_009444056.1 provide additional, non-limiting, example sequences of the CD28 transmembrane domain. The sequences associated with each of the listed accession numbers are provided herein.

As used herein, the term “4-1BB costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the 4-1BB costimulatory signaling region sequence as shown herein. Non-limiting example sequences of the 4-1BB costimulatory signaling region are provided in U.S. Publication 20130266551A1 (filed as U.S. application Ser. No. 13/826,258), such as the exemplary sequence provided below:

4-1BB costimulatory signaling region:
(SEQ ID NO: 123)
KRGRKKLLYIFKQPFMRPVQTTQEEDGCSCRFPEEEEGGCEL

As used herein, the term “CD28 costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the CD28 costimulatory signaling region sequence shown herein. The example sequences CD28 costimulatory signaling domain are provided in U.S. Pat. No. 5,686,281; Geiger, T. L. et al., Blood 98: 2364-2371 (2001); Hombach, A. et al., J Immunol 167: 6123-6131 (2001); Maher, J. et al. Nat Biotechnol 20: 70-75 (2002); Haynes, N. M. et al., J Immunol 169: 5780-5786 (2002); Haynes, N. M. et al., Blood 100: 3155-3163 (2002). Non-limiting examples include residues 114-220 of the below CD28 Sequence: MLRLLLALNL FPSIQVTGNK ILVKQSPMLV AYDNAVNLSC KYSYNLFSRE FRASLIHKGLDSAVEVCVVYG NYSQQLQVYS KTGFNCDGKL GNESVTFYLQ NLYVNQTDIY FCKIEVMYPPPYLDNEKSNG TIIHVKGKHL CPSPLFPGPS KPFWVLVVVG GVLACYSLLVTVAFIIFWVR SKRSRLLHSD YMNMTPRRPG PTRKHYQPYA PPRDFAAYRS (SEQ ID NO: 124), and equivalents thereof.

As used herein, the term “ICOS costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the ICOS costimulatory signaling region sequence as shown herein. Non-limiting example sequences of the ICOS costimulatory signaling region are provided in U.S. Publication 2015/0017141A1 the exemplary polynucleotide sequence provided below.

ICOS costimulatory signaling region coding
sequence:
(SEQ ID NO: 125)
ACAAAAAAGA AGTATTCATC CAGTGTGCAC GACCCTAACG
GTGAATACAT GTTCATGAGA GCAGTGAACA CAGCCAAAAA
ATCCAGACTC ACAGATGTGA CCCTA

As used herein, the term “OX40 costimulatory signaling region” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the OX40 costimulatory signaling region sequence as shown herein. Non-limiting example sequences of the OX40 costimulatory signaling region are disclosed in U.S. Publication 2012/20148552A1, and include the exemplary sequence provided below.

OX40 costimulatory signaling region coding sequence:

AGGGACCAG AGGCTGCCCC CCGATGCCCA CAAGCCCCCT GGGGGAGGCA GTTTCCGGAC CCCCATCCAA GAGGAGCAGG CCGACGCCCA CTCCACCCTG GCCAAGATC (SEQ ID NO: 126), and equivalents thereof.

As used herein, the term “CD28 costimulatory signaling region” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the CD28 costimulatory signaling region sequence shown herein. The example sequences CD28 costimulatory signaling domain are provided in U.S. Pat. No. 5,686,281; Geiger, T. L. et al. (2001) Blood 98: 2364-2371; Hombach, A. et al. (2001) J Immunol 167: 6123-6131; Maher, J. et al. (2002) Nat Biotechnol 20: 70-75; Haynes, N. M. et al. (2002) J Immunol 169: 5780-5786 (2002); Haynes, N. M. et al. (2002) Blood 100: 3155-3163. Non-limiting examples include residues 114-220 of the below and the sequence encoded:

CD28 Sequence:
(SEQ ID NO: 124)
MLRLLLALNL FPSIQVTGNK ILVKQSPMLV AYDNAVNLSC
KYSYNLFSRE FRASLHKGLDSAVEVCVVYG NYSQQLQVYS
KTGFNCDGKL GNESVTFYLQ NLYVNQTDIY
FCKIEVMYPPPYLDNEKSNG TIIHVKGKHL CPSPLFPGPS
KPFWVLVVVG GVLACYSLLVTVAFIIFWVR SKRSRLLHSD
YMNMTPRRPG PTRKHYQPYA PPRDFAAYRS,

and equivalents thereof.

As used herein, the term “CD3 zeta signaling domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD3 zeta signaling domain sequence as shown herein. Non-limiting example sequences of the CD3 zeta signaling domain are provided in U.S. application Ser. No. 13/826,258, e.g.:

(SEQ ID NO: 3)
RVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTA
TKDTYDALHMQALPPR

As used herein, the term NKG2D refers to an activating receptor that has recently generated considerable interest. A number of NKG2D target ligands have been identified. The most intriguing of these are a pair of closely related proteins called MICA and MICB (major histocompatibility complex (MHC) class I chain-related).

Immunological & Biological Reagents (see linscottsdirectory.com, noted above).

As used herein, the term “FLT3” refers to a receptor-type tyrosine-protein kinase FLT3 associated with this name, any of its alternate names (Fms-Related Tyrosine Kinase, Stem Cell Tyrosine Kinase, Fms-Like Tyrosine Kinase, FL Cytokine Receptor, CD135 Antigen, EC 2.7.10.1, CD135, FLK-2, STK1, FLK2, Growth Factor Receptor Tyrosine Kinase Type III, Receptor-Type Tyrosine-Protein Kinase FLT3, Fetal Liver Kinase 2, Fetal Liver Kinase-2, EC 2.7.10, FLT-3, STK-1) or UniProt Accession No. P36888 and any other molecules that have analogous biological function that share at least 80% amino acid sequence identity, preferably 90% sequence identity, or alternatively at least 95% sequence identity with FLT3 and any variant or isoform thereof. Monoclonal antibodies that specifically bind FLT3 are commercially available from, for example, Becton Dickinson Biosciences and other commercial sources, e.g., those listed at the web address: biocompare.com/Search-Antibodies/?search=FLT3&said=0. Methods to prepare antigen binding fragments are known in the art. The antigen binding domains may be from any appropriate species, e.g., sheep or human.

Non-limiting examples of FLT3 include:

### Human FLT3 Isoform 1,

MPALARDGGQLPLLVVFSAMIFGTITNQDLPVIKCVLINHKNNDSSVGKSSSYPMVS ESPEDLGCALRPQSSGTVYEAAAVEVDVSASITLQVLVDAPGNISCLWVFKHSSLN CQPHFDLQNRGVVSMVILKMTETQAGEYLLFIQSEATNYTILFTVSIRNTLLYTLRR PYFRKMENQDALVCISESVPEPIVEWVLCDSQGESCKEESPAVVKKEEKVLHELFG TDIRCCARNELGRECTRLFTIDLNQTPQTTLPQLFLKVGEPLWIRCKAVHVNHGFGL TWELENKALEEGNYFEMSTYSTNRTMIRILFAFVSSVARNDTGYYTCSSSKHPSQSA LVTIVEKGFINATNSSEDYEIDQYEEFCFSVRFKAYPQIRCTWTFSRKSFPCEQKGLD NGYSISKFCNHKHQPGEYIFHAENDDAQFTKMFTLNIRRKPQVLAEASASQASCFSD GYPLPSWTWKKCSDKSPNCTEEITEGVWNRKANRKVFGQWVSSSTLNMSEAIKGF LVKCCAYNSLGTSCETILLNSPGPFPFIQDNISFYATIGVCLLFIVVLTLLICHKYKKQ FRYESQLQMVQVTGSSDNEYFYVDFREYEYDLKWEFPRENLEFGKVLGSGAFGKV MNATAYGISKTGVSIQVAVKMLKEKADSSEREALMSELKMMTQLGSHENIVNLLG ACTLSGPIYLIFEYCCYGDLLNYLRSKREKFHRTWTEIFKEHNFSFYPTFQSHPNSSM PGSREVQIHPDSDQISGLHGNSFHSEDEIEYENQKRLEEEEDLNVLTFEDLLCFAYQV AKGMEFLEFKSCVHRDLAARNVLVTHGKVVKICDFGLARDIMSDSNYVVRGNARL PVKWMAPESLFEGIYTIKSDVWSYGILLWEIFSLGVNPYPGIPVDANFYKLIQNGFK MDQPFYATEEIYIIMQSCWAFDSRKRPSFPNLTSFLGCQLADAEEAMYQNVDGRVS ECPHTYQNRRPFSREMDLGLLSPQAQVEDS (SEQ ID NO: 127), and optionally an equivalent thereof.

Human FLT3 Isoform 2:
(SEQ ID NO: 128)
MPALARDGGQLPLLVVFSAMIFGTITNQDLPVIKCVLINHKNNDSSVG
KSSSYPMVSESPEDLGCALRPQSSGTVYEAAAVEVDVSASITLQVLVD
APGNISCLWVFKHSSLNCQPHFDLQNRGVVSMVILKMTETQAGEYLLF
IQSEATNYTILFTVSIRNTLLYTLRRPYFRKMENQDALVCISESVPEP
IVEWVLCDSQGESCKEESPAVVKKEEKVLHELFGTDIRCCARNELGRE
CTRLFTIDLNQTPQTTLPQLFLKVGEPLWIRCKAVHVNHGFGLTWELE
NKALEEGNYFEMSTYSTNRTMIRILFAFVSSVARNDTGYYTCSSSKHP
SQSALVTIVEKGFINATNSSEDYEIDQYEEFCFSVRFKAYPQIRCTWT
FSRKSFPCEQKGLDNGYSISKFCNHKHQPGEYIFHAENDDAQFTKMFT
LNIRRKPQVLAEASASQASCFSDGYPLPSWTWKKCSDKSPNCTEEITE
GVWNRKANRKVFGQWVSSSTLNMSEAIKGFLVKCCAYNSLGTSCETIL
LNSPGPFPFIQDNISFYATIGVCLLFIVVLTLLICHKYKKQFRYESQL
QMVQVTGSSDNEYFYVDFREYEYDLKWEFPRENLEFGKVLGSGAFGKV
MNATAYGISKTGVSIQVAVKMLKEKADSSEREALMSELKMMTQLGSHE
NIVNLLGACTLSGPIYLIFEYCCYGDLLNYLRSKREKFHRTWTEIFKE
HNFSFYPTFQSHPNSSMPGSREVQIHPDSDQISGLHGNSFHSEDEIEY
ENQKRLEEEEDLNVLTFEDLLCFAYQVAKGMEFLEFKSARLPVKWMAP
ESLFEGIYTIKSDVWSYGILLWEIFSLGVNPYPGIPVDANFYKLIQNG
FKMDQPFYATEEIYIIMQSCWAFDSRKRPSFPNLTSFLGCQLADAEEA
MYQNVDGRVSECPHTYQNRRPFSREMDLGLLSPQAQVEDS,

and optionally an equivalent thereof.

As used herein, the term FLT3-1 in some aspects refers to an antibody comprising an amino acid sequence with CDRs that share at least 70%, or alternatively at least 80% amino acid sequence identity, preferably 90% sequence identity, more preferably at least 95% sequence identity with any one of the CDRs encoded in the heavy and light chain polynucleotide sequences disclosed herein below, preferably at least one of the CDR3 regions, most preferably both of the CDR3 regions, disclosed below. The amino acid sequences of said CDR regions are also disclosed herein below.

FLT3-1 Heavy Chain Variable Region Polynucleotide Sequence:

CAGGTCCAACTGCAGCAGCCTGGGGCTGAGCTTGTGAAGCCTGGGGCTTCATTG AAGCTGTCCTGCAAGTCTTCCGGGTACACCTTCACCAGCTACTGGATGCACTGG GTGAGGCAGAGGCCTGGACATGGCCTTGAGTGGATCGGAGAGATTGATCCTTCT GACAGTTATAAAGACTACAATCAGAAGTTCAAGGACAAGGCCACATTGACTGT GGACAGATCCTCCAACACAGCCTACATGCACCTCAGCAGCCTGACATCTGATGA CTCTGCGGTCTATTATTGTGCAAGAGCGATTACGACGACCCCCTTTGACTTCTGG GGCCAAGGCACCACTCTCACAGTCTCCTCA (SEQ ID NO: 105), and optionally an equivalent thereof.

FLT3-1 Light Chain Variable Region Polynucleotide Sequence:

GATATTGTGCTAACTCAGTCTCCAGCCACCCTGTCTGTGACTCCAGGAGATAGC GTCAGTCTTTCCTGCAGGGCCAGCCAGAGTATTAGCAACAACCTACACTGGTAT CAACAAAAATCACATGAGTCTCCAAGGCTTCTCATCAAGTATGCTTCCCAGTCC ATCTCTGGGATCCCCTCCAGGTTCAGTGGCAGTGGATCAGGGACAGATTTCACT CTCAGTATCAACAGTGTGGAGACTGAAGATTTTGGAGTGTATTTCTGTCAACAG AGTAACACCTGGCCGTACACGTTCGGAGGGGGGACCAAGCTGGAAATAAAACG G (SEQ ID NO: 103), and optionally an equivalent thereof.

FLT3-1 CDHR1:

SYWMH (SEQ ID NO: 21), and optionally an equivalent thereof.

FLT3-1 CDHR2:

EIDPSDSYKDYNQKFKD (SEQ ID NO: 23), and optionally an equivalent thereof.

FLT3-1 CDHR3:

AITTTPFDF (SEQ ID NO: 25), and optionally an equivalent thereof.

FLT3-1 CDLR1:

RASQSISNNLH (SEQ ID NO: 15), and optionally an equivalent thereof.

FLT3-1 CDLR2:

YASQSIS (SEQ ID NO: 17), and optionally an equivalent thereof.

FLT3-1 CDLR3:

QQSNTWPYT (SEQ ID NO: 19), and optionally an equivalent thereof.

Further non-limiting examples of FLT3 CDR domain amino acid sequences are described in Tables 1-4 of the US Patent Application No.: US20180346601, Table V of US Patent Application No.: US20180037657, Table 10 of US Patent Application No.: US20170037149, Table V of US Patent Application No.: US20160272716, Tables 1-3 of US Patent Application No.: US20110091470 and Tables 1-3 of US Patent Application No.: US20090297529.

Non-limiting examples of FLT3 heavy chain variable region and light chain variable region amino acid sequences are described in Tables 1 and 3 of the US Patent Application No.: US20180346601, Table X of US Patent Application No.: US20180037657, Table 10 of US Patent Application No.: US20170037149 and Table VII of US Patent Application No.: US20160272716.

As used herein, the term FLT3-2 refers to an antibody comprising an amino acid sequence with CDRs that share at least 70%, or alternatively at least 80% amino acid sequence identity, preferably 90% sequence identity, more preferably at least 95% sequence identity with any one of the CDRs encoded in the heavy and light chain polynucleotide sequences disclosed herein below, preferably at least one of the CDR3 regions, most preferably both of the CDR3 regions, disclosed below. The amino acid sequences of said CDR regions are also disclosed herein below.

FLT3-2 Heavy Chain Variable Region Sequence:

CAGGTGCAGCTGAAGCAGTCAGGACCTGGCCTAGTGCAGCCCTCACAGAGCCT GTCCATCACCTGCACAGTCTCTGGTTTCTCATTAACTAACTATGGTTTACACTGG GTTCGCCAGTCTCCAGGAAAGGGCCTGGAGTGGCTGGGAGTGATATGGAGTGG TGGAAGCACAGACTATAATGCAGCTTTCATATCCAGACTGAGCATCAGCAAGG ACAACTCCAAGAGCCAAGTTTTCTTTAAAATGAACAGTCTGCAGGCTGATGACA CAGCCATATACTACTGTGCCAGAAAAGGAGGGATCTACTATGCTAACCATTACT ATGCTATGGACTACTGGGGTCAAGGAACCTCAGTCACCGTCTCCTCA (SEQ ID NO: 106), and optionally an equivalent thereof.

FLT3-2 Light Chain Variable Region Sequence:

GACATTGTGATGACACAGTCTCCATCCTCCCTGAGTGTGTCAGCAGGAGAGAAG GTCACTATGAGCTGCAAGTCCAGTCAGAGTCTGTTAAACAGTGGAAATCAAAA GAACTATATGGCCTGGTATCAGCAGAAACCAGGGCAGCCTCCTAAACTGTTGAT CTACGGGGCATCCACTAGGGAATCTGGGGTCCCTGATCGCTTCACAGGCAGTGG ATCTGGAACCGATTTCACTCTTACCATCAGCAGTGTGCAGGCTGAAGACCTGGC AGTTTATTACTGTCAGAATGATCATAGTTATCCGCTCACGTTCGGTGCTGGGAC CAAGCTGGAGCTGAAACGG (SEQ ID NO: 104), and optionally an equivalent thereof.

FLT3-2 CDHR1:

NYGLH (SEQ ID NO: 22), and optionally an equivalent thereof.

FLT3-2 CDHR2:

VIWSGGSTDYNAAFIS (SEQ ID NO: 24), and optionally an equivalent thereof.

FLT3-2 CDHR3:

GGIYYANHYYAMDY (SEQ ID NO: 26), and optionally an equivalent thereof.

FLT3-2 CDLR1:

KSSQSLLNSGNQKNYM (SEQ ID NO: 16), and optionally an equivalent thereof.

FLT3-2 CDLR2:

GASTRES (SEQ ID NO: 18), and optionally an equivalent thereof.

FLT3-2 CDLR3:

QNDHSYPLT (SEQ ID NO: 20), and optionally an equivalent thereof.

A non-limiting example of the FLT3 antibody comprise, or alternatively consist essentially of, or further consist of a heavy chain variable region comprising: a CDHR1 having the amino acid sequence (SYWMH, SEQ ID NO: 21) or (NYGLH, SEQ ID NO: 22) or an equivalent of each thereof, a CDHR2 having the amino acid sequence (EIDPSDSYKDYNQKFKD, SEQ ID NO: 23) or (VIWSGGSTDYNAAFIS, SEQ ID NO: 24) or an equivalent of each thereof, and a CDHR3 having the amino acid sequence (AITTTPFDF, SEQ ID NO: 25) or (GGIYYANHYYAMDY, SEQ ID NO: 26) or an equivalent of each thereof, and/or a light chain variable region comprising: a CDLR1 having the amino acid sequence (RASQSISNNLH, SEQ ID NO: 15) or (KSSQSLLNSGNQKNYM, SEQ ID NO: 16) or an equivalent of each thereof, a CDLR2 having the amino acid sequence (YASQSIS, SEQ ID NO: 17) or (GASTRES, SEQ ID NO: 18) or an equivalent of each thereof, and a CDLR3 having the amino acid sequence (QQSNTWPYT, SEQ ID NO: 19) or (QNDHSYPLT, SEQ ID NO: 20) or an equivalent of each thereof. In one aspect, the FLT3 CAR is driven by a EF1alpha promoter EF1α.

The acronym “EGFR” stands for Epidermal Growth Factor Receptor. EGFR also is known as ErbB-1 and HER1. It is the cell surface receptors of the epidermal growth factor family of cell surface receptors. The term “EGFR” also refers to a specific protein fragment associated with this name and any other molecules that have analogous biological function that share at least 70%, or alternatively at least 80% amino acid sequence identity, preferably 90% sequence identity, more preferably at least 95% amino acid sequence identity with any isoform of EGFR, as disclosed herein. Isoform 1 is the canonical sequence; thus, all positional information that follows refers to the amino acid sequence disclosed below.

EGFR Isoform 1, Uniprot P00533-1:
(SEQ ID NO: 129)
MRPSGTAGAALLALLAALCPASRALEEKKVCQGTSNKLTQLGTFEDHF
LSLQRMFNNCEVVLGNLEITYVQRNYDLSFLKTIQEVAGYVLIALNTV
ERIPLENLQIIRGNMYYENSYALAVLSNYDANKTGLKELPMRNLQEIL
HGAVRESNNPALCNVESIQWRDIVSSDFLSNMSMDFQNHLGSCQKCDP
SCPNGSCWGAGEENCQKLTKIICAQQCSGRCRGKSPSDCCHNQCAAGC
TGPRESDCLVCRKFRDEATCKDTCPPLMLYNPTTYQMDVNPEGKYSFG
ATCVKKCPRNYVVTDHGSCVRACGADSYEMEEDGVRKCKKCEGPCRKV
CNGIGIGEFKDSLSINATNIKHFKNCTSISGDLHILPVAFRGDSFTHT
PPLDPQELDILKTVKEITGFLLIQAWPENRTDLHAFENLEIIRGRTKQ
HGQFSLAVVSLNITSLGLRSLKEISDGDVIISGNKNLCYANTINWKKL
FGTSGQKTKIISNRGENSCKATGQVCHALCSPEGCWGPEPRDCVSCRN
VSRGRECVDKCNLLEGEPREFVENSECIQCHPECLPQAMNITCTGRGP
DNCIQCAHYIDGPHCVKTCPAGVMGENNTLVWKYADAGHVCHLCHPNC
TYGCTGPGLEGCPTNGPKIPSIATGMVGALLLLLVVALGIGLFMRRRH
IVRKRTLRRLLQERELVEPLTPSGEAPNQALLRILKETEFKKIKVLGS
GAFGTVYKGLWIPEGEKVKIPVAIKELREATSPKANKEILDEAYVMAS
VDNPHVCRLLGICLTSTVQLITQLMPFGCLLDYVREHKDNIGSQYLLN
WCVQIAKGMNYLEDRRLVHRDLAARNVLVKTPQHVKITDFGLAKLLGA
EEKEYHAEGGKVPIKWMALESILHRIYTHQSDVWSYGVTVWELMTFGS
KPYDGIPASEISSILEKGERLPQPPICTIDVYMIMVKCWMIDADSRPK
FRELIIEFSKMARDPQRYLVIQGDERMHLPSPTDSNFYRALMDEEDMD
DVVDADEYLIPQQGFFSSPSTSRTPLLSSLSATSNNSTVACIDRNGLQ
SCPIKEDSFLQRYSSDPTGALTEDSIDDTFLPVPEYINQSVPKRPAGS
VQNPVYHNQPLNPAPSRDPHYQDPHSTAVGNPEYLNTVQPTCVNSTFD
SPAHWAQKGSHQISLDNPDYQQDFFPKEAKPNGIFKGSTAENAEYLRV
APQSSEFIGA

Binding sites include but are not limited to positions 745 and 855; active sites include but are not limited to position 837; and other sites of interest include but are not limited to position 1016. EGFR Isoform 2 (Uniprot P00533-2) has an FL to LS substitution at position 404 to 405 and is missing the region from position 406 to 1210. EGFR Isoform 4 (Uniprot P00533-4) has a C to S substitution at position 628 and is missing the region from position 629 to 1210. EGFR Isoform 3 (Uniprot P00533-3) differs from positons 628 to 705 and is missing the region from position 706 to 1210, in accordance with the sequence below.

EGFR Isoform 3, Uniprot P00533-3:
(SEQ ID NO: 130)
MRPSGTAGAALLALLAALCPASRALEEKKVCQGTSNKLTQLGTFEDHF
LSLQRMFNNCEVVLGNLEITYVQRNYDLSFLKTIQEVAGYVLIALNTV
ERIPLENLQIIRGNMYYENSYALAVLSNYDANKTGLKELPMRNLQEIL
HGAVRFSNNPALCNVESIQWRDIVSSDFLSNMSMDFQNHLGSCQKCDP
SCPNGSCWGAGEENCQKLTKIICAQQCSGRCRGKSPSDCCHNQCAAGC
TGPRESDCLVCRKFRDEATCKDTCPPLMLYNPTTYQMDVNPEGKYSFG
ATCVKKCPRNYVVTDHGSCVRACGADSYEMEEDGVRKCKKCEGPCRKV
CNGIGIGEFKDSLSINATNIKHFKNCTSISGDLHILPVAFRGDSFTHT
PPLDPQELDILKTVKEITGFLLIQAWPENRTDLHAFENLEIIRGRTKQ
HGQFSLAVVSLNITSLGLRSLKEISDGDVIISGNKNLCYANTINWKKL
EGTSGQKTKIISNRGENSCKATGQVCHALCSPEGCWGPEPRDCVSCRN
VSRGRECVDKCNLLEGEPREFVENSECIQCHPECLPQAMNITCTGRGP
DNCIQCAHYIDGPHCVKTCPAGVMGENNTLVWKYADAGHVCHLCHPNC
TYGPGNESLKAMLFCLFKLSSCNQSNDGSVSHQSGSPAAQESCLGWIP
SLLPSEFQLGWGGCSHLHAWPSASVIITASSCH

EGFRvIII is a mutant form of EGFR that is reported to be expressed in a considerable proportion of patients with glioblastoma multiforme (GB). Gan et al. 205350-5370 report that the mutant form is expressed in other tumors as well. The term “mutant EGFR” may refer to EGFRvIII or a specific protein fragment associated with this name and any other molecules that have analogous biological function that share at least 70%, or alternatively at least 80% amino acid sequence identity, preferably 90% sequence identity, more preferably at least 95% amino acid sequence identity with the EGFRvIII, as shown herein or an equivalent thereof as further defined herein.

EGFRvIII, Uniprot P00533[30-297]:
(SEQ ID NO: 131)
MRPSGTAGAA LLALLAALCP ASRALEEKKV CQGTSNKLTQ
LGTFEDHFLS LQRMFNNCEV VLGNLEITYV QRNYDLSFLK
TIQEVAGYVL IALNTVERIPLENLQIIRGN MYYENSYALA
VLSNYDANKT GLKELPMRNL QEILHGAVRF SNNPALCNVE
SIQWRDIVSS DFLSNMSMDF QNHLGSCQKC
DPSCPNGSCWGAGEENCQKL TKIICAQQCS GRCRGKSPSD
CCHNQCAAGC TGPRESDCLV CRKFRDEATC KDTCPPLMLY
NPTTYQMDVN PEGKYSFGAT CVKKCPRNYV VTDHGSCVRA
CGADSYEMEE DGVRKCKKCE GPCRKVCNGI GIGEFKDSLS
INATNIKHFK NCTSISGDLH ILPVAFRGDS FTHTPPLDPQ
ELDILKTVKE ITGFLLIQAW PENRTDLHAF ENLEIIRGRT
KQHGQFSLAV VSLNITSLGL RSLKEISDGD VIISGNKNLC
YANTINWKKL FGTSGQKTKI ISNRGENSCK ATGQVCHALC
SPEGCWGPEP RDCVSCRNVS RGRECVDKCN LLEGEPREFV
ENSECIQCHP ECLPQAMNIT CTGRGPDNCI QCAHYIDGPH
CVKTCPAGVM GENNTLVWKY ADAGHVCHLC HPNCTYGCTG
PGLEGCPTNG PKIPSIATGM VGALLLLLVV ALGIGLFMRR
RHIVRKRTLR RLLQERELVE PLTPSGEAPN QALLRILKET
EFKKIKVLGS GAFGTVYKGL WIPEGEKVKI
PVAIKELREATSPKANKEIL DEAYVMASVD NPHVCRLLGI
CLTSTVQLIT QLMPFGCLLDYVREHKDNIG SQYLLNWCVQ
IAKGMNYLED RRLVHRDLAA RNVLVKTPQHVKITDFGLAK
LLGAEEKEYH AEGGKVPIKW MALESILHRI
YTHQSDVWSYGVTVWELMTF GSKPYDGIPA SEISSILEKG
ERLPQPPICT IDVYMIMVKC WMIDADSRPK FRELIIEFSK
MARDPQRYLV IQGDERMHLPLMDEEDMDDV VDADEYLIPQ
QGFFSSPSTS RTPLLSSLSA TSNNSTVACIDRNGLQSCPI
KEDSFLQRYS SDPTGALTED SIDDTFLPVP
EYINQSVPKRPAGSVQNPVY HNQPLNPAPS RDPHYQDPHS
TAVGNPEYLN TVQPTCVNSTFDSPAHWAQK GSHQISLDNP
DYQQDFFPKE AKPNGIFKGS TAENAEYLRVAPQSSEFIGA

The term “mutant EGFR” may also refer to a natural variant of any isoform of EGFR including but not limited variants with one or more of the following mutations: R to Q at position 98, P to R at position 266, G to D at position 428, R to K at position 521, V to I a position 674, E to A at position 709, E to G at position 709, E to K at position 709, G to A at position 719, G to C at position 719, G to D at position 719, G to S at position 719, G to S at position 724, E to K at position 734, ELREATS (SEQ ID NO: 172) to D at positions 746 to 752, ELREAT (SEQ ID NO: 173) to A at positions 746 to 751, a deletion from positions 746 to 750, a deletion at position 746, a deletion from positions 747 to 751, a deletion from positions 747 to 749, L to F at position 747, R to P at position 748, a deletion from positions 752 to 759, S to I at position 768, V to M at position 769, Q to R at position 787, T to M at position 790, L to V at position 833, V to L at position 834, H to L at position 835, L to V at position 838, L to M at position 858, L to R at position 858, L to Q at position 861, G to E at position 873, R to G at position 962, H to P at position 988, L to R at position 1034, A to V at position 1210, and/or a different amino acid substitution or deletion at any one of the specific positions.

In some embodiments, the heavy chain variable region of the antibody comprises, or consists essentially of, or consists of:

Q V Q L Q Q S G S EM A R P G A S V K L P C K A S G D T F T S Y W M H W V K Q R H G H G P E W I G N I Y P G S G G T N Y A E K F K N K V T LT V D R S S R T V Y M H L S R L T S E DS A V Y Y C T R S G G P Y F F D Y W G Q G T T L T V S S (SEQ ID NO: 45), or an equivalent thereof, or a polynucleotide encoded by the polypeptide:
GACATTCTAATGACCCAATCTCCACTCTCCCTGCCTGTCAGTCTTGGAGATCAA GCCTCCTACCTGCAAAGGCCAGGCCAGTCTCCAAAGCTCCTGATCTACAAAGTT TCCGACCGATTTTACCTGCAAAGGCCAGGCCAGTCTCCAAAGCTCCTGATCTAC AAAGTTTCCGACCGATTTTCTGGGGTCCCAGACAGGTTCAGTGGCAGTGGATCA GGGACAGATTTCACACTCAAGATCAGCAGAGTAGAGGCTGAGGATCTGGGAAT TTATTACTGCTTTCAAGGTTCACATATTCCTCCCACGTTCGGAGGGGGGACCAA GCTGGAAATCAAACGTGCGGCC (SEQ ID NO: 132), or an equivalent thereof.

The polypeptide or equivalents of each thereof, can be followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In other aspect, the LC variable region comprises, or alternatively consists essentially of, or yet further consists of:

D I L M T Q S P L S L P V S L G D Q A S I S C R S S Q N I V H N N G I T Y L E W Y L Q R P G Q S P K L L I Y K V S D R F S G V P D R F S G S G S G T D F T L K I S R V E A E D L G I Y Y C F Q G S H I P P T F G G G T K L E I K R A A (SEQ ID NO: 43), or an equivalent thereof, or a polypeptide encoded by the polynucleotide:
CAGGTCCAGCTGCAGCAGTCTGGGTCTGAGATGGCGAGGCCTGGAGCTTCAGT GAAGCTGCCCTGCAAGGCTTCTGGCGACACATTCACCAGTTACTGGATGCACTG GGTGAAGCAGAGGCATGGACATGGCCCTGAGTGGATCGGAAATATTTATCCAG GTAGTGGTGGTACTAACTACGCTGAGAAGTTCAAGAACAAGGTCACTCTGACTG TAGACAGGTCCTCCCGCACAGTCTACATGCACCTCAGCAGGCTGACATCTGAGG ACTCTGCGGTCTATTATTGTACAAGATCGGGGGGTCCCTACTTCTTTGACTACTG GGGCCAAGGCACCACTCTCACAGTCTCCTCC (SEQ ID NO: 133), or an equivalent thereof.

The polypeptide or equivalents of each thereof, can be followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

An equivalent thereof comprises an polypeptide having at least 80% amino acid identity to the CAR or a polypeptide that is encoded by a polynucleotide that hybridizes under conditions of high stringency to the complement of a polynucleotide encoding the CAR, wherein conditions of high stringency comprises incubation temperatures of about 55° C. to about 68° C.; buffer concentrations of about 0.1×SSC to about 0.1×SSC; formamide concentrations of about 55% to about 75%; and wash solutions of about 0.1×SSC, 0.1×SSC, or deionized water.

Alternative embodiments include one or more of the CDRs (e.g., CDR1, CDR2, CDR3) from the LC variable region with appropriate CDRs from other EGFR antibody CDRs. And equivalents of each thereof. Accordingly, and as an example, the CDR1 and CDR2 from the LC variable region can be combined with the CDR3 of another anti-EGFR antibody's LC variable region, and in some aspects, can include an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus. In another aspect, the EGFR CAR is the CAR disclosed in WO 2016/164370.

A “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. For example, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions disclosed herein. Aspects defined by each of these transition terms are within the scope of the present disclosure.

The term “consensus sequence” as used herein refers to an amino acid or nucleic acid sequence that is determined by aligning a series of multiple sequences and that defines an idealized sequence that represents the predominant choice of amino acid or base at each corresponding position of the multiple sequences. Depending on the sequences of the series of multiple sequences, the consensus sequence for the series can differ from each of the sequences by zero, one, a few, or more substitutions. Also, depending on the sequences of the series of multiple sequences, more than one consensus sequence may be determined for the series. The generation of consensus sequences has been subjected to intensive mathematical analysis. Various software programs can be used to determine a consensus sequence.

As used herein, the term “CRISPR” refers to a technique of sequence specific genetic manipulation relying on the clustered regularly interspaced short palindromic repeats pathway. CRISPR can be used to perform gene editing and/or gene regulation, as well as to simply target proteins to a specific genomic location. Gene editing refers to a type of genetic engineering in which the nucleotide sequence of a target polynucleotide is changed through introduction of deletions, insertions, or base substitutions to the polynucleotide sequence. In some aspects, CRISPR-mediated gene editing utilizes the pathways of nonhomologous end-joining (NHEJ) or homologous recombination to perform the edits. Gene regulation refers to increasing or decreasing the production of specific gene products such as protein or RNA.

The term “gRNA” or “guide RNA” as used herein refers to the guide RNA sequences used to target specific genes for correction employing the CRISPR technique. Techniques of designing gRNAs and donor therapeutic polynucleotides for target specificity are well known in the art. For example, Doench, J., et al. Nature biotechnology 2014; 32(12):1262-7, Mohr, S. et al. (2016) FEBS Journal 283: 3232-38, and Graham, D., et al. Genome Biol. 2015; 16: 260. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspects, a gRNA is synthetic (Kelley, M. et al. (2016) J of Biotechnology 233 (2016) 74-83). As used herein, a biological equivalent of a gRNA includes but is not limited to polynucleotides or targeting molecules that can guide a Cas9 or equivalent thereof to a specific nucleotide sequence such as a specific region of a cell's genome.

“Cytoreductive therapy,” as used herein, includes but is not limited to chemotherapy, cryotherapy, and radiation therapy. Agents that act to reduce cellular proliferation are known in the art and widely used. Chemotherapy drugs that kill cancer cells only when they are dividing are termed cell-cycle specific. These drugs include agents that act in S-phase, including topoisomerase inhibitors and anti-metabolites.

Topoisomerase inhibitors are drugs that interfere with the action of topoisomerase enzymes (topoisomerase I and II). During the process of chemo treatments, topoisomerase enzymes control the manipulation of the structure of DNA necessary for replication, and are thus cell cycle specific. Examples of topoisomerase I inhibitors include the camptothecan analogs listed above, irinotecan and topotecan. Examples of topoisomerase II inhibitors include amsacrine, etoposide, etoposide phosphate, and teniposide.

Antimetabolites are usually analogs of normal metabolic substrates, often interfering with processes involved in chromosomal replication. They attack cells at very specific phases in the cycle. Antimetabolites include folic acid antagonists, e.g., methotrexate; pyrimidine antagonist, e.g., 5-fluorouracil, floxuridine, cytarabine, capecitabine, and gemcitabine; purine antagonist, e.g., 6-mercaptopurine and 6-thioguanine; adenosine deaminase inhibitor, e.g., cladribine, fludarabine, nelarabine and pentostatin; and the like.

Plant alkaloids are derived from certain types of plants. The vinca alkaloids are made from the periwinkle plant (Catharanthus rosea). The taxanes are made from the bark of the Pacific Yew tree (taxus). The vinca alkaloids and taxanes are also known as antimicrotubule agents. The podophyllotoxins are derived from the May apple plant. Camptothecan analogs are derived from the Asian “Happy Tree” (Camptotheca acuminata). Podophyllotoxins and camptothecan analogs are also classified as topoisomerase inhibitors. The plant alkaloids are generally cell-cycle specific.

Examples of these agents include vinca alkaloids, e.g., vincristine, vinblastine and vinorelbine; taxanes, e.g., paclitaxel and docetaxel; podophyllotoxins, e.g., etoposide and tenisopide; and camptothecan analogs, e.g., irinotecan and topotecan.

Cryotherapy includes, but is not limited to, therapies involving decreasing the temperature, for example, hypothermic therapy.

Radiation therapy includes, but is not limited to, exposure to radiation, e.g., ionizing radiation, UV radiation, as known in the art. Exemplary dosages include, but are not limited to, a dose of ionizing radiation at a range from at least about 2 Gy to not more than about 10 Gy and/or a dose of ultraviolet radiation at a range from at least about 5 J/m² to not more than about 50 J/m², usually about 10 J/m².

“Detectable label”, “label”, “detectable marker” or “marker” are used interchangeably, including, but not limited to radioisotopes, fluorochromes, chemiluminescent compounds, dyes, and proteins, including enzymes. Detectable labels can also be attached to a polynucleotide, polypeptide, antibody or composition described herein.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, 3-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁵I. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6th ed). Examples of luminescent probes include, but are not limited to, aequorin and luciferases.

As used herein, the term “immunoconjugate” comprises an antibody or an antibody derivative associated with or linked to a second agent, such as a cytotoxic agent, a detectable agent, a radioactive agent, a targeting agent, a human antibody, a humanized antibody, a chimeric antibody, a synthetic antibody, a semisynthetic antibody, or a multispecific antibody.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6th ed.).

In another aspect, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

An “effective amount” or “efficacious amount” refers to the amount of an agent, or combined amounts of two or more agents, that, when administered for the treatment of a mammal or other subject, is sufficient to effect such treatment for the disease. The “effective amount” will vary depending on the agent(s), the disease and its severity and the age, weight, etc., of the subject to be treated.

In some embodiments, the terms “first” “second” “third” “fourth” or similar in a component name are used to distinguish and identify more than one components sharing certain identity in their names. For example, “first TAA” and “second TAA” are used across the specification to distinguishing two TAAs, and in some embodiments, the first TAA is an TAA recognized and bound by a CAR as disclosed herein while the second TAA refers to the one recognized and bound by a bispecific antibody as disclosed herein.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “enhancer”, as used herein, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

In one aspect, the term “equivalent” or “biological equivalent” of an antibody means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

A “gene” refers to a polynucleotide containing at least one open reading frame (ORF) that is capable of encoding a particular polypeptide or protein after being transcribed and translated.

The term “express” refers to the production of a gene product.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

A “gene product” or alternatively a “gene expression product” refers to the amino acid (e.g., peptide or polypeptide) generated when a gene is transcribed and translated.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein).

Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical”, percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “isolated” as used herein refers to molecules or biologicals or cellular materials being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, the term “isolated cell” generally refers to a cell that is substantially separated from other cells of a tissue.

In some embodiments, the term “engineered” or “recombinant” refers to having at least one modification not normally found in a naturally occurring protein, polypeptide, polynucleotide, strain, wild-type strain or the parental host strain of the referenced species. In some embodiments, the term “engineered” or “recombinant” refers to being synthetized by human intervention.

“Immune cells” includes, e.g., white blood cells (leukocytes) which are derived from hematopoietic stem cells (HSC) produced in the bone marrow, lymphocytes (T cells, B cells, natural killer (NK) cells) and myeloid-derived cells (neutrophil, eosinophil, basophil, monocyte, macrophage, dendritic cells).

As used herein the terms “linker sequence” “linker peptide” and “linker polypeptide” are used interchangeably, relating to any amino acid sequence comprising from 1 to 10, or alternatively, 8 amino acids, or alternatively 6 amino acids, or alternatively 5 amino acids that may be repeated from 1 to 10, or alternatively to about 8, or alternatively to about 6, or alternatively about 5, or 4 or alternatively 3, or alternatively 2 times. For example, the linker may comprise up to 15 amino acid residues consisting of a pentapeptide repeated three times. In one aspect, the linker sequence is a (Glycine4Serine)3 (SEQ ID NO: 14) flexible polypeptide linker comprising three copies of gly-gly-gly-gly-ser (SEQ ID NO: 134).

A “normal cell corresponding to the tumor tissue type” refers to a normal cell from a same tissue type as the tumor tissue. A non-limiting example is a normal lung cell from a patient having lung tumor or lung tissue from a patient not having lung cancer, or a normal colon cell from a patient having colon tumor.

As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™), TALL-104 cytotoxic human T cell line (ATCC #CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PERO117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4; 11 (ATCC CRL-1873), CCRF-CEM (ATCC CRM-CCL-119); and cutaneous T-cell lymphoma lines, e.g., HuT78 (ATCC CRM-TIB-161), MJ[G11] (ATCC CRL-8294), HuT102 (ATCC TIB-162). Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (http://www.atcc.org/) and the German Collection of Microorganisms and Cell Cultures (https://www.dsmz.de/).

As used herein, the term “NK cell,” also known as natural killer cell, refers to a type of lymphocyte that originates in the bone marrow and play a critical role in the innate immune system. NK cells provide rapid immune responses against viral-infected cells, tumor cells or other stressed cell, even in the absence of antibodies and major histocompatibility complex on the cell surfaces. NK cells may either be isolated or obtained from a commercially available source. Non-limiting examples of commercial NK cell lines include lines NK-92 (ATCC® CRL-2407™), NK-92MI (ATCC® CRL-2408™). Further examples include but are not limited to NK lines HANK1, KHYG-1, NKL, NK-YS, NOI-90, and YT. Non-limiting exemplary sources for such commercially available cell lines include the American Type Culture Collection, or ATCC, (http://www.atcc.org/) and the German Collection of Microorganisms and Cell Cultures (https://www.dsmz.de/).

As used herein in reference to a regulatory polynucleotide, the term “operatively linked” refers to an association between the regulatory polynucleotide and the polynucleotide sequence to which it is linked such that, when a specific protein binds to the regulatory polynucleotide, the linked polynucleotide is transcribed.

As used herein, the term “overexpress” with respect to a cell, a tissue, or an organ expresses a protein to an amount that is greater than the amount that is produced in a control cell, a control issue, or an organ. A protein that is overexpressed may be endogenous to the host cell or exogenous to the host cell.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

In some embodiments, an equivalent to a reference nucleic acid, polynucleotide or oligonucleotide encodes the same sequence encoded by the reference. In some embodiments, an equivalent to a reference nucleic acid, polynucleotide or oligonucleotide hybridizes to the reference, a complement reference, a reverse reference, and/or a reverse-complement reference, optionally under conditions of high stringency. Additionally or alternatively, an equivalent nucleic acid, polynucleotide or oligonucleotide is one having at least 70%, or at least 75%, or at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence identity to the reference nucleic acid, polynucleotide, or oligonucleotide, or alternatively an equivalent nucleic acid hybridizes under conditions of high stringency to a reference polynucleotide or its complement. In one aspect, the equivalent must encode functional protein that optionally can be identified through one or more assays described herein. In another aspect, an equivalent has at least the 70%, or at least 75%, or at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence identity to the reference nucleic acid, polynucleotide, or oligonucleotide, or alternatively an equivalent nucleic acid hybridizes under conditions of high stringency to a reference polynucleotide or its complement.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting examples of promoters include the EF1alpha promoter and the CMV promoter. The EF1alpha sequence is known in the art (see, e.g., addgene.org/11154/sequences/;ncbi.nlm.nih.gov/nuccore/J04617, each last accessed on Mar. 13, 2019, and Zheng and Baum (2014) Int'l. J. Med. Sci. 11(5).404-408). The CMV promoter sequence is known in the art (see, e.g.,

snapgene.com/resources/plasmid-files/?set=basic_cloning_vectors&plasmid=CMV_promoter, last accessed on Mar. 13, 2019 and Zheng and Baum (2014), supra.). An example is:

### EF1 Alpha Promoter Sequence:

AAGGATCTGCGATCGCTCCGGTGCCCGTCAGTGGGCAGAGCGCACATCGCCCA CAGTCCCCGAGAAGTTGGGGGGAGGGGTCGGCAATTGAACGGGTGCCTAGAGA AGGTGGCGCGGGGTAAACTGGGAAAGTGATGTCGTGTACTGGCTCCGCCTTTTT CCCGAGGGTGGGGGAGAACCGTATATAAGTGCAGTAGTCGCCGTGAACGTTCT TTTTCGCAACGGGTTTGCCGCCAGAACACAGCTGAAGCTTCGAGGGGCTCGCAT CTCTCCTTCACGCGCCCGCCGCCCTACCTGAGGCCGCCATCCACGCCGGTTGAG TCGCGTTCTGCCGCCTCCCGCCTGTGGTGCCTCCTGAACTGCGTCCGCCGTCTAG GTAAGTTTAAAGCTCAGGTCGAGACCGGGCCTTTGTCCGGCGCTCCCTTGGAGC CTACCTAGACTCAGCCGGCTCTCCACGCTTTGCCTGACCCTGCTTGCTCAACTCT ACGTCTTTGTTTCGTTTTCTGTTCTGCGCCGTTACAGATCCAAGCTGTGACCGGC GCCTAC (SEQ ID NO: 148), and optionally, an equivalent thereof.

As used herein, the terms “T2A” and “2A peptide” are used interchangeably to refer to any 2A peptide or fragment thereof, any 2A-like peptide or fragment thereof, or an artificial peptide comprising the requisite amino acids in a relatively short peptide sequence (on the order of 20 amino acids long depending on the virus of origin) containing the consensus polypeptide motif D-V/I-E-X—N—P-G-P (SEQ ID NO: 174), wherein X refers to any amino acid generally thought to be self-cleaving.

IL3Ralpha (IL3Rα) or CD123 is a surface receptor overexpressed in several hematological malignancies. Initial targeting of IL3R was conducted using the natural ligand, IL-3. CD123 CAR cells have shown potent cytotoxicity against AML cell with mice and in humans. Townsend et al. (2018) J. Exper. & Clin. Cancer Res. 37:163, and references cited therein. Thus, CAR-able CD123 binding peptides and polynucleotides are known in the art.

IL13Ralpha2 (IL13Ra2) intends a cell marker expressed on glioblastoma cells. It is an IL-13 receptor that acts as a decoy by directly competing with IL13Rα1 receptor to elicit downstream STAT signaling. Townsend et al. (2018), supra. scFv-based CARS have been tested and have shown to be successful. Townsend et al. (2018), supra.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The term equivalent and biological equivalent are used interchangeably, for example when referring to a protein or polypeptide as a reference. In some embodiments, an equivalent protein or polypeptide is one having at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% sequence identity to the reference protein or polypeptide. In some embodiments, an equivalent protein or polypeptide has at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% sequence identity to a polypeptide or protein as disclosed herein. In some embodiments, an equivalent protein or polypeptide has at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% sequence identity to polypeptide or protein encoded by an equivalent polynucleotide as noted herein. In addition or alternatively, the equivalent of a polynucleotide would encode a protein or polypeptide of the same or similar function as the reference or parent polynucleotide. In some embodiments, the equivalent is a functional protein that optionally can be identified through one or more assays described herein. In another aspect, an equivalent has at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% sequence identity to the reference protein or polypeptide.

As used herein, an amino acid (aa) or nucleotide (nt) residue position in a sequence of interest “corresponding to” an identified position in a reference sequence refers to that the residue position is aligned to the identified position in a sequence alignment between the sequence of interest and the reference sequence. Various programs are available for performing such sequence alignments, such as Clustal Omega and BLAST.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

As used herein, the term “purification marker” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten. In some embodiments, the purification marker is a HA tag, optionally comprising, or alternatively consisting essentially of, or yet consisting of YPYDVPDYA (SEQ ID NO: 84).

As used herein, the term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

As used herein, the term “specific binding” means the contact between an antibody and an antigen with a binding affinity of at least 10⁻⁶ M. In certain aspects, antibodies bind with affinities of at least about 10⁻⁷ M, and preferably 10⁻⁸ M, 10⁻⁹ M, 10⁻¹⁰ M, 10⁻¹¹ M, or 10⁻¹² M.

A “solid tumor” is an abnormal mass of tissue that usually does not contain cysts or liquid areas. Solid tumors can be benign or malignant, metastatic or non-metastatic. Different types of solid tumors are named for the type of cells that form them. Examples of solid tumors include sarcomas, carcinomas, and lymphomas.

As used herein, the term “suicide gene” is a gene capable of inducing cell apoptosis; non-limiting examples include HSV-TK (Herpes simplex virus thymidine kinase), cytosine deaminase, nitroreductase, carboxylesterase, cytochrome P450 or PNP (Purine nucleoside phosphorylase), truncated EGFR, or inducible caspase (“iCasp”). Suicide genes may function along a variety of pathways, and, in some cases, may be inducible by an inducing agent such as a small molecule. For example, the iCasp suicide gene comprises portion of a caspase protein operatively linked to a protein optimized to bind to an inducing agent; introduction of the inducing agent into a cell comprising the suicide gene results in the activation of caspase and the subsequent apoptosis of said cell.

The term “transduce” or “transduction” as it is applied to the production of chimeric antigen receptor cells refers to the process whereby a foreign nucleotide sequence is introduced into a cell. In some embodiments, this transduction is done via a vector.

As used herein, “treating” or “treatment” of a disease in a subject and the like are used herein to mean obtaining a desired pharmacologic and/or physiologic effect. Examples of “treatment” include but are not limited to: preventing a disorder from occurring in a subject that may be predisposed to a disorder, but has not yet been diagnosed as having it; inhibiting a disorder, i.e., arresting its development; and/or relieving or ameliorating the symptoms of disorder. In one aspect, treatment is the arrestment of the development of symptoms of the disease or disorder, such as a cancer. In some embodiments, it refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Treatments containing the disclosed compositions and methods can be first line, second line, third line, fourth line, fifth line therapy and are intended to be used as a sole therapy or in combination with other appropriate therapies. When the disease is cancer, the following clinical end points are non-limiting examples of treatment:

reduction in tumor burden, slowing of tumor growth, longer overall survival, longer time to tumor progression, inhibition of metastasis or a reduction in metastasis of the tumor. In one aspect, the term “treatment” or “treating” excludes prevention or prophylaxis.

In one embodiment, the term “disease” or “disorder” as used herein refers to a cancer or tumor (which are used interchangeably), a status of being diagnosed with such disease, a status of being suspect of having such disease, or a status of at high risk of having such disease.

“Administration” or “delivery” of a cell or vector or other agent and compositions containing same can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician or in the case of animals, by the treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, intraperitoneal, infusion, nasal administration, inhalation, injection, and topical application.

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

As used herein, the term “vector” refers to a nucleic acid construct deigned for transfer between different hosts, including but not limited to a plasmid, a virus, a cosmid, a phage, a BAC, a YAC, etc. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827.

In aspects where gene transfer is mediated by a lentiviral vector, a vector construct refers to the polynucleotide comprising the lentiviral genome or part thereof, and a therapeutic gene. As used herein, “lentiviral mediated gene transfer” or “lentiviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, lentiviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral vectors, New York: Spring-Verlag Berlin Heidelberg.

Lentiviral vectors of this disclosure are based on or derived from oncoretroviruses (the sub-group of retroviruses containing MLV), and lentiviruses (the sub-group of retroviruses containing HIV). Examples include ASLV, SNV and RSV all of which have been split into packaging and vector components for lentiviral vector particle production systems. The lentiviral vector particle according to the disclosure may be based on a genetically or otherwise (e.g. by specific choice of packaging cell system) altered version of a particular retrovirus.

That the vector particle according to the disclosure is “based on” a particular retrovirus means that the vector is derived from that particular retrovirus. The genome of the vector particle comprises components from that retrovirus as a backbone. The vector particle contains essential vector components compatible with the RNA genome, including reverse transcription and integration systems. Usually these will include gag and pol proteins derived from the particular retrovirus. Thus, the majority of the structural components of the vector particle will normally be derived from that retrovirus, although they may have been altered genetically or otherwise so as to provide desired useful properties. However, certain structural components and in particular the env proteins, may originate from a different virus. The vector host range and cell types infected or transduced can be altered by using different env genes in the vector particle production system to give the vector particle a different specificity.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 sequentially numbered, AAV serotypes are known in the art. Non-limiting exemplary serotypes useful in the methods disclosed herein include any of the 11 serotypes, e.g., AAV2, AAV8, AAV9, or variant or synthetic serotypes, e.g., AAV-DJ and AAV PHP.B. The AAV particle comprises, alternatively consists essentially of, or yet further consists of three major viral proteins: VP1, VP2 and VP3. In one embodiment, the AAV refers to of the serotype AAV1, AAV2, AAV4, AAV5, AAV6, AAV7, AAV8, AAV9, AAV10, AAV11, AAV12, AAV13, AAV PHP.B, or AAV rh74. These vectors are commercially available or have been described in the patent or technical literature.

The terms or “acceptable,” “effective,” or “sufficient” when used to describe the selection of any components, ranges, dose forms, etc. disclosed herein intend that said component, range, dose form, etc. is suitable for the disclosed purpose.

The term “a regulatory sequence” “an expression control element” or “promoter” as used herein, intends a polynucleotide that is operatively linked to a target polynucleotide to be transcribed and/or replicated, and facilitates the expression and/or replication of the target polynucleotide. A promoter is an example of an expression control element or a regulatory sequence. Promoters can be located 5′ or upstream of a gene or other polynucleotide, that provides a control point for regulated gene transcription. Polymerase II and III are examples of promoters. The sequence of the MNDU3 promoter and the sequence of an exemplary CMV promoter are provided below.

A signal peptide, as used herein, refers to (sometimes referred to as signal sequence, targeting signal, localization signal, localization sequence, transit peptide, leader sequence or leader peptide) is a short peptide (usually 16-30 amino acids long) present at the N-terminus of the majority of newly synthesized proteins that are destined toward the secretory pathway. In some embodiments, the signal peptide is a secretary signal, such as an IL 2 signal peptide. See, for example, SEQ ID NO: 4. In some embodiments, the signal peptide directs a protein or polypeptide, for example, a transmembrane protein, to located in a cell membrane, such as on the cell surface. One such example is an IgG1 signal peptide. See for example, SEQ ID NO: 5.

A secretary signal intends a secretory signal peptide that allows the export of a protein from the cytosol into the secretory pathway. Proteins can exhibit differential levels of successful secretion and often certain signal peptides can cause lower or higher levels when partnered with specific proteins. In eukaryotes, the signal peptide is a hydrophobic string of amino acids that is recognized by the signal recognition particle (SRP) in the cytosol of eukaryotic cells. After the signal peptide is produced from a mRNA-ribosome complex, the SRP binds the peptide and stops protein translation. The SRP then shuttles the mRNA/ribosome complex to the rough endoplasmic reticulum where the protein is translated into the lumen of the endoplasmic reticulum. The signal peptide is then cleaved off the protein to produce either a soluble, or membrane tagged (if a transmembrane region is also present), protein in the endoplasmic reticulum. These are known in the art, and commercially available from vendors, e.g., Oxford Genetics.

As used herein, a cleavable peptide, which is also referred to as a cleavable linker, means a peptide that can be cleaved, for example, by an enzyme. One translated polypeptide comprising such cleavable peptide can produce two final products, therefore, allowing expressing more than one polypeptides from one open reading frame. One example of cleavable peptides is a self-cleaving peptide, such as a 2A self-cleaving peptide. 2A self-cleaving peptides, is a class of 18-22 aa-long peptides, which can induce the cleaving of the recombinant protein in a cell. In some embodiments, the 2A self-cleaving peptide is selected from P2A, T2A, E2A, F2A and BmCPV2A. See, for example, Wang Y, et al. 2A self-cleaving peptide-based multi-gene expression system in the silkworm Bombyx mori. Sci Rep. 2015; 5:16273. Published 2015 Nov. 5.

As used herein, a “pluripotent cell” defines a less differentiated cell that can give rise to at least two distinct (genotypically and/or phenotypically) further differentiated progeny cells. In another aspect, a “pluripotent cell” includes an Induced Pluripotent Stem Cell (iPSC) which is an artificially derived stem cell from a non-pluripotent cell, typically an adult somatic cell, that has historically been produced by inducing expression of one or more stem cell specific genes. Such stem cell specific genes include, but are not limited to, the family of octamer transcription factors, i.e. Oct-3/4; the family of Sox genes, i.e., Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e., OCT4, NANOG and REXI; or LIN28. Examples of iPSCs are described in Takahashi et al. (2007) Cell advance online publication 20 Nov. 2007; Takahashi & Yamanaka (2006) Cell 126:663-76; Okita et al. (2007) Nature 448:260-262; Yu et al. (2007) Science advance online publication 20 Nov. 2007; and Nakagawa et al. (2007) Nat. Biotechnol. Advance online publication 30 Nov. 2007.

An “induced pluripotent cell” intends embryonic-like cells reprogrammed to the immature phenotype from adult cells. Various methods are known in the art, e.g., “A simple new way to induce pluripotency: Acid.” Nature, 29 Jan. 2014 and available at sciencedaily.com/releases/2014/01/140129184445, last accessed on Feb. 5, 2014 and U.S. Patent Application Publication No. 2010/0041054. Human iPSCs also express stem cell markers and are capable of generating cells characteristic of all three germ layers.

As used herein, hematopoietic stem cells (HSCs) are cells, such as stem cells, that give rise to all types of blood cells, including but not limited to white blood cells, red blood cells, and platelets. Hematopoietic stem cells can be found in the peripheral blood and the bone marrow.

The term pharmaceutically acceptable carrier (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers suitable for use in the present disclosure include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

A population of cells intends a collection of more than one cell that is identical (clonal) or non-identical in phenotype and/or genotype. The population can be purified, highly purified, substantially homogenous or heterogeneous as described herein.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker.

As used herein the term “NKG2D” refers to a transmembrane protein belonging to the CD94/NKG2 family of C-type lectin-like receptors and encoded by the gene KLRK1 gene, which is located in the NK-gene complex and/or a biological equivalent thereof. Non-limiting exemplary sequences of this protein or the underlying gene may be found under Gene Cards ID: GC12M011728, HGNC: 18788, Entrez Gene: 22914, Ensembl: ENSG00000213809, OMIM: 611817, and UniProtKB: P26718, which are incorporated by reference herein.

As used herein the terms “BCMA” and “B-cell maturation antigen” are used interchangeably to refer to a protein belonging to the TNF superfamily which recognizes B-cell activating factor (BAFF) and encoded by the TNFRSF17 gene. Non-limiting exemplary sequences of this protein or the underlying gene may be found under Gene Cards ID: GC16P012058, HGNC: 11913, Entrez Gene: 608, Ensembl: ENSG00000048462, OMIM: 109545, and UniProtKB: Q02223, which are incorporated by reference herein.

As used herein the terms “SLAMF7,” “CS1,” and “CD319” are used interchangeably to refer to a protein known to be a robust marker to normal plasma cell and malignant plasma cells in multiple myeloma and encoded by the SLAMF7 gene. Non-limiting exemplary sequences of this protein or the underlying gene may be found under Gene Cards ID: GC01P160709, HGNC: 21394, Entrez Gene: 57823, Ensembl: ENSG00000026751, OMIM: 606625, and UniProtKB: Q9NQ25, which are incorporated by reference herein.

As used herein the terms “CD19,” and “B-lymphocyte antigen CD19” are used interchangeably to refer to a protein known to be a transmembrane protein that in humans is encoded by the gene CD19. In humans, CD19 is expressed in all B lineage cells, except for plasma cells, and in follicular dendritic cells. Due to its presence on all B cells, it is a biomarker for B lymphocyte development, lymphoma diagnosis can be utilized as a target for leukemia immunotherapies. Non-limiting exemplary sequences of this protein or the underlying gene may be found under Gene Cards ID: GC16PO28943, HGNC: 1633, Entrez Gene: 930, Ensembl: ENSG00000177455, OMIM: 107265, and UniProtKB: P15391, which are incorporated by reference herein.

As used herein the terms “HER2,” “HER2/neu,” “CD340,” and “ERBB2” are used interchangeably to refer to a protein known to be a member of the human epidermal growth factor receptor (HER/EGFR/ERBB) family. Amplification or over-expression of this oncogene has been shown to play an important role in the development and progression of certain aggressive types of breast cancer. The protein has become an important biomarker and target of therapy for many breast cancer patients. Non-limiting exemplary sequences of this protein or the underlying gene may be found under Gene Cards ID: GC17P039687, HGNC: 3430, Entrez Gene: 2064, Ensembl: ENSG000000141736, OMIM: 164870, and UniProtKB: P04626, which are incorporated by reference herein.

As used herein the terms “mesothelin” and “MSLN” are used interchangeably to refer to a protein that in humans is encoded by the MSLN gene. Mesothelin is a 40 kDa protein that is expressed in mesothelial cells. Mesothelin is over expressed in multiple types of tumors, including mesothelioma, ovarian cancer, pancreatic adenocarcinoma, lung adenocarcinoma, and cholangiocarcinoma. Non-limiting exemplary sequences of this protein or the underlying gene may be found under Gene Cards ID: GC16P001100, HGNC: 7371, Entrez Gene: 10232, Ensembl: ENSG000000102854, OMIM: 601051, and UniProtKB: Q13421, which are incorporated by reference herein.

As used herein the terms “PSCA” and “prostate stem cell antigen” are used interchangeably to refer to a protein that in humans is encoded by the PSCA gene. PSCA is a glycosylphosphatidylinositol-anchored cell membrane glycoprotein that is highly expressed in the prostate. PSCA is also expressed in the bladder, placenta, colon, kidney, and stomach. Non-limiting exemplary sequences of this protein or the underlying gene may be found under Gene Cards ID: GC08P142670, HGNC: 9500, Entrez Gene: 8000, Ensembl: ENSG000000167653, OMIM: 602470, and UniProtKB: 043653, which are incorporated by reference herein.

As used herein the terms “CEA” and “carcinoembryonic antigen” are used interchangeably to refer to a set of highly related glycoproteins involved in cellular adhesion. CEA is usually present at very low levels in the blood of healthy adults; however, the serum levels are raised in some types of cancer, which indicates that I can be used as a tumor marker in clinical tests. Immunologically, the CEA glycoproteins are characterized as members of the CD66 cluster of differentiation; the proteins include CD66a, CD66b, CD66c, CD66d, and CD66e.

Non-limiting exemplary sequences of CD66a, also known as carcinoembryonic antigen related cell adhesion molecule 1, or the underlying gene may be found under Gene Cards ID: GC19M042507, HGNC: 1814, Entrez Gene: 634, Ensembl: ENSG000000079385, OMIM: 109770, and UniProtKB: P13688, which are incorporated by reference herein.

Non-limiting exemplary sequences of CD66b, also known as carcinoembryonic antigen related cell adhesion molecule 8, or the underlying gene may be found under Gene Cards ID: GC19M042580, HGNC: 1820, Entrez Gene: 1088, Ensembl: ENSG00000124469, OMIM: 615747, and UniProtKB: P31997, which are incorporated by reference herein.

Non-limiting exemplary sequences of CD66c, also known as carcinoembryonic antigen related cell adhesion molecule 6, or the underlying gene may be found under Gene Cards ID: GC19P04150, HGNC: 1818, Entrez Gene: 4680, Ensembl: ENSG00000086548, OMIM: 163980, and UniProtKB: P40199, which are incorporated by reference herein.

Non-limiting exemplary sequences of CD66d, also known as carcinoembryonic antigen related cell adhesion molecule 3, or the underlying gene may be found under Gene Cards ID: GC19P041796, HGNC: 1815, Entrez Gene: 1084, Ensembl: ENSG00000170956, OMIM: 609142, and UniProtKB: P40198, which are incorporated by reference herein.

Non-limiting exemplary sequences of CD66e, also known as carcinoembryonic antigen related cell adhesion molecule 5, or the underlying gene may be found under Gene Cards ID: GC19P041709, HGNC: 1817, Entrez Gene: 1048, Ensembl: ENSG00000105388, OMIM: 114890, and UniProtKB: P06731, which are incorporated by reference herein.

As used herein the terms “GTPase-Activating Protein,” “GAP,” and “RAS P21 Protein Activator 1” are used interchangeably to refer to a cytoplasmic protein that is a member of the GAP1 family of GTPase-activating proteins. The protein stimulates the GTPase activity of normal RAS p21 but not its oncogenic counterpart. Acting as a suppressor of RAS function, the protein enhances the weak intrinsic GTPase activity of RAS proteins resulting in the inactive GDP-bound form of RAS, thereby allowing control of cellular proliferation and differentiation. Mutations leading to changes in the binding sites of either protein are associated with basal cell carcinomas. Non-limiting exemplary sequences of this protein or the underlying gene may be found under Gene Cards ID: GC05P087267, HGNC: 9871, Entrez Gene: 5921, Ensembl: ENSG000000145715, OMIM: 139150, and UniProtKB: P20936, which are incorporated by reference herein.

As used herein the terms “gd2,” “ganglioside G2,” and “ganglioside GD2” are used interchangeably to refer to a chemical that in humans is expressed on the plasma membranes of various types of malignant cells. The IUPAC name of GD2 is (2R,4R,5S,6S)-2-[3-[(2S,3S,4R,6S)-6-[(2S,3R,4R,5S,6R)-5-[(2S,3R,4R,5R,6R)-3-acetamido-4,5-dihydroxy-6-(hydroxymethyl)oxan-2-yl]oxy-2-[(2R,3S,4R,5R,6R)-4,5-dihydroxy-2-(hydroxymethyl)-6-[(E)-3-hydroxy-2-(octadecanoylamino)octadec-4-enoxy]oxan-3-yl]oxy-3-hydroxy-6-(hydroxymethyl)oxan-4-yl]oxy-3-amino-6-carboxy-4-hydroxyoxan-2-yl]-2,3-dihydroxypropoxy]-5-amino-4-hydroxy-6-(1,2,3-trihydroxypropyl)oxane-2-carboxylic acid.

As used herein the terms “CD5” and “T-cell surface glycoprotein CD5” are used interchangeably to refer to a protein that in humans is expressed on the surface of T cells. May T-cell neoplasms are reported to express CD5, and it is also found in chronic lymphocytic leukemia and mantle cell lymphoma. Non-limiting exemplary sequences of this protein or the underlying gene may be found under Gene Cards ID: GC11P061118, HGNC: 1685, Entrez Gene: 921, Ensembl: ENSG00000110448, OMIM: 153340, and UniProtKB: P06127, which are incorporated by reference herein.

As used herein the terms “prostate specific membrane antigen,” “PSMA,” “folate hydrolase 1,” and “FOLH1” are used interchangeably to refer to a protein that is a type II transmembrane glycoprotein belonging to the M28 peptidase family. In the prostate, the protein is up-regulated in cancerous cells and is used as an effective diagnostic and prognostic indicator of prostate cancer. Non-limiting exemplary sequences of this protein or the underlying gene may be found under Gene Cards ID: GC11M056090, HGNC: 3788, Entrez Gene: 2346, Ensembl: ENSG00000086205, OMIM: 600934, and UniProtKB: Q04609, which are incorporated by reference herein.

As used herein the terms “receptor tyrosine kinase-like orphan receptor 1” and “ROR1” are used interchangeably to refer to a protein that modulates neurite growth in the central nervous system. Increased expression is associated with B-cell chronic lymphocytic leukemia. Non-limiting exemplary sequences of this protein or the underlying gene may be found under Gene Cards ID: GC01P063774, HGNC: 10256, Entrez Gene: 4919, Ensembl: ENSG00000185483, OMIM: 602336, and UniProtKB: Q01973, which are incorporated by reference herein.

As used herein the terms “CD123,” “interleukin 3 receptor subunit alpha,” and “IL-3RA” are used interchangeably to refer to an interleukin 3 specific subunit of a heterodimeric cytokine receptor. Non-limiting exemplary sequences of this protein or the underlying gene may be found under Gene Cards ID: GC0XP001336, HGNC: 6012, Entrez Gene: 3563, Ensembl: ENSG00000185291, OMIM: 430000, and UniProtKB: P26951, which are incorporated by reference herein.

As used herein the terms “CD70” and “tumor necrosis factor ligand superfamily member 7” are used interchangeably to refer to a cytokine that belongs to the tumor necrosis factor ligand family. Non-limiting exemplary sequences of this protein or the underlying gene may be found under Gene Cards ID: GC19M006583, HGNC: 11937, Entrez Gene: 970, Ensembl: ENSG00000125726, OMIM: 602840, and UniProtKB: P32970, which are incorporated by reference herein.

As used herein the terms “CD38” and “ADP-ribosyl cyclase 1” are used interchangeably to refer to a non-lineage restricted, type-II transmembrane glycoprotein that synthesizes and hydrolyzes cyclic adinoside 5′-diphosphate-ribose, an intracellular calcium ion mobilizing messenger. Non-limiting exemplary sequences of this protein or the underlying gene may be found under Gene Cards ID: GC04P015779, HGNC: 1667, Entrez Gene: 952, Ensembl: ENSG0000004468, OMIM: 107270, and UniProtKB: P28907, which are incorporated by reference herein.

As used herein the terms “mucin 1” and “mucin 1” are used interchangeably to refer to a membrane-bound protein that is a member of the mucin family. Overexpression, aberrant intracellular localization, and changes in glycosylation of this protein have been associated with carcinomas. Non-limiting exemplary sequences of this protein or the underlying gene may be found under Gene Cards ID: GC01M155158, HGNC: 7508, Entrez Gene: 4582, Ensembl: ENSG00000185499, OMIM: 158340, and UniProtKB: P15941, which are incorporated by reference herein.

As used herein the terms “EPHA2” and “ephrin type-A receptor 2” are used interchangeably to refer to a protein that binds ephrin-A ligands. Non-limiting exemplary sequences of this protein or the underlying gene may be found under Gene Cards ID: GC01M016196, HGNC: 3386, Entrez Gene: 1969, Ensembl: ENSG00000142627, OMIM: 176946, and UniProtKB: P29317, which are incorporated by reference herein.

As used herein the terms “EGFRvIII” and “epidermal growth factor variant III” are used interchangeably to refer to the epidermal growth factor variant (vIII) that is the most common of the EGFR mutations, occurring in up to 30% of high-grade gliomas, especially glioblastoma multiforme. EGFRvIII arises from the deletion of exon 2-7 that leads to the formation of the constitutively activated mutant receptor incapable of binding to any known ligand.

As used herein the terms “IL13RA2” and “interleukin 13 receptor subunit alpha 2” are used interchangeably to refer to a subunit of the interleukin 13 receptor complex. Non-limiting exemplary sequences of this protein or the underlying gene may be found under Gene Cards ID: GC0XM115003, HGNC: 5975, Entrez Gene: 3598, Ensembl: ENSG00000123496, OMIM: 300130, and UniProtKB: Q14627, which are incorporated by reference herein.

As used herein the terms “CD133” and “prominin 1” are used interchangeably to refer to a pentaspan transmembrane glycoprotein. The protein localizes to membrane protrusions and is often expressed on adult stem cells, where it is thought to function in maintaining stem cell properties by suppressing differentiation. This protein is associated with several types of cancer. Non-limiting exemplary sequences of this protein or the underlying gene may be found under Gene Cards ID: GC04M015965, HGNC: 9454, Entrez Gene: 8842, Ensembl: ENSG00000007062, OMIM: 604365, and UniProtKB: 043490, which are incorporated by reference herein.

As used herein the terms “GPC3” and “glypican 3” are used interchangeably to refer to a cell surface heparin sulfate proteoglycan composed of a membrane-associated protein core substituted with a variable number of heparin sulfate chains. Non-limiting exemplary sequences of this protein or the underlying gene may be found under Gene Cards ID: GC0XM133535, HGNC: 4451, Entrez Gene: 2719, Ensembl: ENSG00000147257, OMIM: 300037, and UniProtKB: P51654, which are incorporated by reference herein.

As used herein the terms “EPCAM” and “epithelial cell adhesion molecule” are used interchangeably to refer to a carcinoma-associated antigen which is a member of a family that includes at least two type-I membrane proteins. This antigen is expressed on most normal epithelial cells and gastrointestinal carcinomas and functions as a homotypic calcium-independent adhesion molecule. Non-limiting exemplary sequences of this protein or the underlying gene may be found under Gene Cards ID: GC02P047345, HGNC: 11529, Entrez Gene: 4072, Ensembl: ENSG00000119888, OMIM: 185535, and UniProtKB: P16422, which are incorporated by reference herein.

As used herein the terms “FAP” and “fibroblast activated protein alpha” are used interchangeably to refer to a homodimeric integral membrane gelatinase belonging to the serine protease family. It is selectively expressed in reactive stromal fibroblasts of epithelial cancers, granulation tissues of healing wounds, and malignant cells of bone and soft tissue sarcomas. Non-limiting exemplary sequences of this protein or the underlying gene may be found under Gene Cards ID: GC02M162170, HGNC: 3590, Entrez Gene: 2191, Ensembl: ENSG00000078098, OMIM: 600403, and UniProtKB: Q12884, which are incorporated by reference herein.

As used herein the terms “VEGFR2,” “vascular endothelial growth factor receptor 2,” and “kinase insert domain receptor” are used interchangeably to refer to a protein that functions as the main mediator of VEGF-induced endothelial proliferation, survival, migration, tubular morphogenesis and sprouting. Non-limiting exemplary sequences of this protein or the underlying gene may be found under Gene Cards ID: GC04M055078, HGNC: 6307, Entrez Gene: 3791, Ensembl: ENSG00000128052, OMIM: 191306, and UniProtKB: P35968, which are incorporated by reference herein.

As used herein the terms “cancer/testis antigens” and “CT antigens” are used interchangeably to refer to a category of tumor antigens with normal expression restricted to male germ cells in the testis but not in adult somatic tissues. In malignancy, a variety of tumors may express CT antigens. Non-limiting exemplary CT antigens include: CTAG1B, CTAG2, CT45A2, CT45A5, CT45A6, CT45A3, CT45A1, CT47B1, CT47A11, CT47A6, CT47A7, CT45A7, CTAG1A, AT45A8, CT45A9, CT47A1, CT47A2, CT47A3, CT47A4, and CT47A8.

As used herein the terms “GUCY2C” and “guanylate cyclase 2C” are used interchangeably to refer to a transmembrane protein that functions as a receptor for endogenous peptides guanylin and uroguanylin. Non-limiting exemplary sequences of this protein or the underlying gene may be found under Gene Cards ID: GC12M014612, HGNC: 4688, Entrez Gene: 2984, Ensembl: ENSG00000070019, OMIM: 601330, and UniProtKB: P25092, which are incorporated by reference herein.

As used herein the terms “TAG72,” “CD247,” and “tumor-associated glycoprotein-72” are used interchangeably to refer to a T-cell receptor zeta. Non-limiting exemplary sequences of this protein or the underlying gene may be found under Gene Cards ID: GC01M167399, HGNC: 1677, Entrez Gene: 919, Ensembl: ENSG00000198821, OMIM: 186780, and UniProtKB: P20963, which are incorporated by reference herein.

As used herein the terms “TK1” and “thymidine kinase 1” are used interchangeably to refer to a cytosolic enzyme that catalyzes the addition of a gamma-phosphate group to thymidine. High levels of this protein have been used as a biomarker for diagnosing and categorizing many types of cancers. Non-limiting exemplary sequences of this protein or the underlying gene may be found under Gene Cards ID: GC12M014612, HGNC: 4688, Entrez Gene: 2984, Ensembl: ENSG00000070019, OMIM: 601330, and UniProtKB: P25092, which are incorporated by reference herein.

As used herein the terms “HPRT1” and “hypoxanthine phosphoribosyltransferase 1” are used interchangeably to refer to a transferase that catalyzes the conversion of hypoxanthine to inosine monophosphate and guanine to guanosine monophosphate via transfer of the 5-phosphoribosyl group from 5-phosphoribosyl 1-pyrophosphate. Non-limiting exemplary sequences of this protein or the underlying gene may be found under Gene Cards ID: GC0XP134460, HGNC: 5157, Entrez Gene: 3251, Ensembl: ENSG00000165704, OMIM: 308000, and UniProtKB: P00492, which are incorporated by reference herein.

The sequences associated with each of the above listed accession numbers and references are herein incorporated by reference.

### Modes for Carrying Out the Disclosure

Administration of immunoregulatory molecules has been pursued as a cancer therapeutic. However, due to severe side effects associated with systemic administration (Giovarelli, M. et al. (2000) J Immunol. 164:3200-3206; Lasek, W. et al. (2014) Cancer Immunol Immunother. 63:419-435), obtaining high concentrations of these immunoregulatory molecules in relevant tumors in order to achieve an effective immune response has been difficult.

Due to the unprecedented results being recently obtained in B-cell lymphomas and leukemia's using autologous treatment with genetically engineered chimeric antigen receptor (CAR) T-cells (Maude, S. L. et al. (2014) New Engl. J. Med. 371:1507-1517; Porter, D. L. et al. (2011) New Engl. J. Med. 365:725-733), a number of laboratories have begun to apply this approach to solid tumors including ovarian cancer, prostate cancer, and pancreatic tumors. CAR modified T-cells combine the HLA-independent targeting specificity of a monoclonal antibody with the cytolytic activity, proliferation, and homing properties of activated T-cells, but do not respond to checkpoint suppression. Because of their ability to kill antigen expressing targets directly, CAR T-cells are highly toxic to any antigen positive cells or tissues making it a requirement to construct CARs with highly tumor specific antibodies. To date, CAR modified T-cells to human solid tumors have been constructed against the α-folate receptor, mesothelin, and MUC-CD, PSMA, and other targets but most have some off-target expression of antigen in normal tissues. These constructs have not shown the same exceptional results in patients emphasizing the need for additional studies to identify new targets and methods of CAR T-cell construction that can be used against solid tumors.

Thus, this disclosure provides a chimeric antigen receptor (CAR) comprising a binding domain specific to a cancer or tumor antigen, that in some aspects, is the antigen binding domain of an antigen other than anti-BCMA antibody, and a bispecific antibody (such as a BiTE or a BiKE) or a fragment thereof, a polynucleotide or a vector encoding a BsAb-CAR construct, a BsAb-CAR expressing cell that targets a tumor or cancer antigen and secretes soluble antibody fragments, and methods and compositions relating to the use and production thereof.

### Chimeric Antigen Receptors and Uses Thereof

### Components

The present disclosure provides chimeric antigen receptors (CAR) that bind to a cancer or tumor antigen, the CAR comprising, or consisting essentially of, or consisting of, a cell activation moiety comprising an extracellular, transmembrane, and intracellular domain (also referred to herein as cytoplasmic domain). The extracellular domain comprises a target-specific binding element otherwise referred to as the antigen binding domain. The intracellular domain or cytoplasmic domain comprises one or more costimulatory signaling region(s) and a signaling domain, such as a CD3 zeta chain portion. The CAR may optionally further comprise a spacer domain of up to 300 amino acids, preferably 10 to 100 amino acids, more preferably 25 to 50 amino acids.

Additionally or alternatively, a CAR as disclosed herein comprises, or consists essentially of, or yet further consists of an antigen binding domain that recognizes and binds a tumor associated antigen (TAA) on a cancer cell, a hinge domain, a transmembrane domain and an intracellular/cytoplasmic domain. In some embodiments, the TAA is not a B-cell maturation antigen (BCMA), such as an EGFR (wildtype or EGFRvIII) or a FLT3. In some embodiments, the TAA is a BCMA.

Spacer Domain. The CAR may optionally further comprise a spacer domain of up to 300 amino acids, preferably 10 to 100 amino acids, more preferably 25 to 50 amino acids. For example, the spacer may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. A spacer domain may comprise, for example, a portion of a human Fc domain, a CH3 domain, or the hinge region of any immunoglobulin, such as IgA, IgD, IgE, IgG, or IgM, or variants thereof. For example, some embodiments may comprise an IgG4 hinge with or without a S228P, L235E, and/or N297Q mutation (according to Kabat numbering). Additional spacers include, but are not limited to, CD4, CD8, and CD28 hinge regions.

Signal Peptide. The CAR may optionally further comprise a signal peptide, optionally directing the CAR to be on cell surface of a CAR and/or BsAb-CAR expressing cell. In some embodiments, the signal peptide is located at the N terminus of the CAR, for example, on the N terminus side to the antigen binding domain of the CAR. In some embodiments, the signal peptide may be that of any immunoglobulin, such as IgA, IgD, IgE, IgG, or IgM. In one embodiment, the signal peptide is that of IgG1. In a further embodiment, the signal peptide comprises, or consists essentially of, or yet further consists of a sequence of SEQ ID NO: 5 or an equivalent thereof. In further embodiments, an equivalent of SEQ ID NO: 5 still directs the CAR to be located on cell surface of a CAR and/or BsAb-CAR expressing cell. Additionally or alternatively, an equivalent of SEQ ID NO: 5 is at least 80% (such as at least 85%, at least 90%, or at least 91%, or at least 92%, or at least 93%, or at least 94%, at least 95%, at least 96%, at least 97%, at least 98%, or at least 99%) identical to SEQ ID NO: 5. Methods of detecting whether a signal peptide directing a protein to be located on the cell surface are available to one of skill in the art, such as immunostaining and imaging a protein expressing cell.

Antigen Binding Domain. In certain embodiments, the present disclosure provides a CAR that comprises, or alternatively consists essentially of, or yet further consists of an antigen binding domain specific to a cancer or tumor antigen. The antigen binding domains can be from any appropriate species, e.g., murine, human or a humanized sequence.

In some embodiments, the antigen binding domain comprises, or alternatively consists essentially of, or yet consists of the antigen binding domain of an anti-BCMA antibody or an antibody that binds a BCMA-relevant antigen. Monoclonal antibodies that specifically bind these antigens are commercially available. The antigen binding domains can be from any appropriate species, e.g., murine, human or a humanized sequence. In one aspect, the antigen binding domain comprises the heavy chain variable region and the light chain variable region of an antibody directed to a cancer or tumor antigen other than a B-cell maturation antigen (BCMA). In another embodiment, the antigen binding domain comprises the heavy and light chain variable regions of an anti-SLAMF7 antibody (also known as anti-CS1 antibody or anti-CD319 antibody), and/or an equivalent of each thereof. In some embodiments, the antigen binding domain comprises, consists, or consists essentially of a fragment of a target-specific antibody (i.e., an antibody to an antigen other than a B-cell maturation antigen (BCMA) and/or SLAMF7 (also known as CS1 or CD319), and/or an equivalent of each thereof), for example, an scFv. An scFv region can comprise the variable regions of the heavy (VH) and light chains (VL) of immunoglobulins, connected with a short linker peptide. The linker peptide may be from 1 to 50 amino acids long, for instance, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. In some embodiments, the linker is glycine rich, although it may also contain serine or threonine.

In some embodiments of the present disclosure, an equivalent of the antigen binding domain of an anti-cancer or anti-tumor antibody includes one or more of the following characteristics:

(a) the light chain immunoglobulin variable region/domain sequence comprises one or more (such as 1, 2, 3 or more) CDRs that are at least 80% (such as at least 85%, at least 90%, or at least 91%, or at least 92%, or at least 93%, or at least 94%, at least 95%, at least 96%, at least 97%, at least 98%, or at least 99%) identical to a CDR of a light chain variable region/domain of any of the disclosed light chain sequences;

(b) the heavy chain immunoglobulin variable region/domain sequence comprises one or more CDRs (such as 1, 2, 3 or more) that are at least 80% (such as at least 85%, at least 90%, or at least 91%, or at least 92%, or at least 93%, or at least 94%, at least 95%, at least 96%, at least 97%, at least 98%, or at least 99%) identical to a CDR of a heavy chain variable domain of any of the disclosed heavy chain sequences;

(c) the light chain immunoglobulin variable region/domain sequence is at least 80% (such as at least 85%, at least 90%, or at least 91%, or at least 92%, or at least 93%, or at least 94%, at least 95%, at least 96%, at least 97%, at least 98%, or at least 99%) identical to a light chain variable region/domain of any of the disclosed light chain sequences;

(d) the heavy chain (HC) immunoglobulin variable region/domain sequence is at least 80% (such as at least 85%, at least 90%, or at least 91%, or at least 92%, or at least 93%, or at least 94%, at least 95%, at least 96%, at least 97%, at least 98%, or at least 99%) identical to a heavy chain variable region/domain of any of the disclosed heavy chain sequences;

(e) the light chain (LC) immunoglobulin variable region/domain sequence is at least 80% (such as at least 85%, at least 90%, or at least 91%, or at least 92%, or at least 93%, or at least 94%, at least 95%, at least 96%, at least 97%, at least 98%, or at least 99%) identical to a light chain variable region/domain of any of the disclosed light chain sequences; and

(f) the antibody binds an epitope that overlaps with an epitope bound by any of the disclosed sequences.

In further embodiments, any one or more of (a) to (e) still recognizes and binds the same epitope of the reference sequence as disclosed, and/or recognizes and binds an epitope that overlaps with an epitope bound by the reference sequence as disclosed.

Additional examples of antigen binding domain equivalents include peptide having at least 85%, or alternatively at least 90%, or alternatively at least 95%, or alternatively at least 97% amino acid identity to a reference peptide or polypeptide that is encoded by a polynucleotide that hybridizes under conditions of high stringency to the complement of a polynucleotide encoding the antigen binding domain. In some embodiments, conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

In some embodiments, the antigen binding domain of a CAR as disclosed herein recognizes and binds fins-like tyrosine kinase 3 (FLT3), i.e., is an anti-FLT3 antigen binding domain. In some embodiments, the anti-FLT3 antigen binding domain comprises, or alternatively consists essentially of, or yet consists of one or two or three or four or five or all six of the following CDRs: a CDRL1 comprising, or alternatively consisting essentially of, or yet consisting of RASQSISNNLH (SEQ ID NO: 15), KSSQSLLNSGNQKNYM (SEQ ID NO: 16), or an equivalent of each thereof; a CDRL2 comprising, or alternatively consisting essentially of, or yet consisting of YASQSIS (SEQ ID NO: 17), GASTRES (SEQ ID NO: 18), or an equivalent of each thereof, a CDRL3 comprising, or alternatively consisting essentially of, or yet consisting of QQSNTWPYT (SEQ ID NO: 19), QNDHSYPLT (SEQ ID NO: 20), or an equivalent of each thereof, a CDRH1 comprising, or alternatively consisting essentially of, or yet consisting of SYWMH (SEQ ID NO: 21), NYGLH (SEQ ID NO: 22), or an equivalent of each thereof, a CDRH2 comprising, or alternatively consisting essentially of, or yet consisting of EIDPSDSYKDYNQKFKD (SEQ ID NO: 23), VIWSGGSTDYNAAFIS (SEQ ID NO: 24), or an equivalent of each thereof, and a CDRH3 comprising, or alternatively consisting essentially of, or yet consisting of AITTTPFDF (SEQ ID NO: 25), GGIYYANHYYAMDY (SEQ ID NO: 26), or an equivalent of each thereof. In further embodiments, the CDR equivalent(s) thereof and/or the antigen binding domain comprising, or alternatively consisting essentially of, or yet consisting of the equivalent(s) recognize and bind FLT3. In some embodiments, the anti-FLT3 antigen binding domain comprises, or alternatively consists essentially of, or yet consists of a light chain variable region comprising, or alternatively consisting essentially of, or yet consisting of a sequence selected from

### DIVLTQSPATLSVTPGDSVSLSCRASQSISNNLHWYQQKSHESPRLLIKYASQSISGIP SRFSGSGSGTDFTLSINSVETEDFGVYFCQQSNTWPYTFGGGTKLEIKR (SEQ ID NO: 27),

DIVMTQSPSSLSVSAGEKVTMSCKSSQSLLNSGNQKNYMAWYQQKPGQPPKLLIY GASTRESGVPDRFTGSGSGTDFTLTISSVQAEDLAVYYCQNDHSYPLTFGAGTKLEL KR (SEQ ID NO: 28), or an equivalent of each thereof; and/or
a heavy chain variable region comprising, or alternatively consisting essentially of, or yet consisting of a sequence selected from

### QVQLQQPGAELVKPGASLKLSCKSSGYTFTSYWMHWVRQRPGHGLEWIGEIDPSD SYKDYNQKFKDKATLTVDRSSNTAYMHLSSLTSDDSAVYYCARAITTTPFDFWGQ GTTLTVSS (SEQ ID NO: 29),

QVQLKQSGPGLVQPSQSLSITCTVSGFSLTNYGLHWVRQSPGKGLEWLGVIWSGGS TDYNAAFISRLSISKDNSKSQVFFKMNSLQADDTAIYYCARKGGIYYANHYYAMD YWGQGTSVTVSS (SEQ ID NO: 30) or an equivalent of each thereof. In further embodiments, the variable equivalent(s) thereof and/or an antigen binding domain comprising, or alternatively consisting essentially of, or yet consisting of the equivalent(s) recognizes and binds FLT3.

In some embodiments, the antigen binding domain of a CAR as disclosed herein recognizes and binds epidermal growth factor receptor (EGFR), for example wildtype EGFR (EGFRwt) and/or a variant thereof, such as EGFRvIII. This antigen binding domain is also referred to herein as an anti-EGFR antigen binding domain. In some embodiments, the anti-EGFR antigen binding domain comprises, or alternatively consists essentially of, or yet consists of one or two or three or four or five or six of the following CDRs: a CDRL1 comprising, or alternatively consisting essentially of, or yet consisting of RSSQNIVHNNGITYLE (SEQ ID NO: 31), RASQGIRNNLA (SEQ ID NO: 32), or an equivalent of each thereof; a CDRL2 comprising, or alternatively consisting essentially of, or yet consisting of KVSDRFS (SEQ ID NO: 33), AASNLQS (SEQ ID NO: 34), or an equivalent of each thereof; a CDRL3 comprising, or alternatively consisting essentially of, or yet consisting of FQGSHIPPT (SEQ ID NO: 35), LQHHSYPLT (SEQ ID NO: 36), or an equivalent of each thereof; a CDRH1 comprising, or alternatively consisting essentially of, or yet consisting of GDTFTSY (SEQ ID NO: 37), GFTFSSY (SEQ ID NO: 38), or an equivalent of each thereof; a CDRH2 comprising, or alternatively consisting essentially of, or yet consisting of YPGSGG (SEQ ID NO: 39), SGSGGS (SEQ ID NO: 40), or an equivalent of each thereof; and a CDRH3 comprising, or alternatively consisting essentially of, or yet consisting of SGGPYFFDY (SEQ ID NO: 41), SSGWSEY (SEQ ID NO: 42), or an equivalent of each thereof. In further embodiments, the CDR equivalent(s) thereof and/or an antigen binding fragment comprising, or alternatively consisting essentially of, or yet consisting of the equivalent(s) recognize and bind EGFR. In some embodiments, the anti-EGFR antigen binding domain comprises, or alternatively consists essentially of, or yet consists of a light chain variable region comprising, or alternatively consisting essentially of, or yet consisting of a sequence selected from

### DILMTQSPLSLPVSLGDQASISCRSSQNIVHNNGITYLEWYLQRPGQSPKLLIYKVSD RFSGVPDRFSGSGSGTDFTLKISRVEAEDLGIYYCFQGSHIPPTFGGGTKLEIKRAA (SEQ ID NO: 43),

DIQMTQSPSSLSASVGDRVTITCRASQGIRNNLAWYQQKPGKAPKRLIYAASNLQS GVPSRFTGSGSGTEFTLIVSSLQPEDFATYYCLQHHSYPLTSGGGTKVEIKYAHNS (SEQ ID NO: 44), or an equivalent of each thereof and/or a heavy chain variable region comprising, or alternatively consisting essentially of, or yet consisting of a sequence selected from

### QVQLQQSGSEMARPGASVKLPCKASGDTFTSYWMHWVKQRHGHGPEWIGNIYPG SGGTNYAEKFKNKVTLTVDRSSRTVYMHLSRL TSEDSAVYYCTRSGGPYFFDYWG QGTTLTVSS (SEQ ID NO: 45),

EVQVLESGGGLVQPGGSLRLSCAASGFTFSSYAMSWVRQAPGKGLEWVSAISGSG GSTNYADSVKGRFTISRDNSKNTLYLQMNSLRAEDTAVYYCAGSSGWSEYWGQG TLVTVSS (SEQ ID NO: 46), or an equivalent of each thereof. In further embodiments, the variable region equivalent(s) thereof and/or an antigen binding fragment comprising, or alternatively consisting essentially of, or yet consisting of the equivalent(s) recognize and bind EGFR.

In some embodiments, the antigen binding domain of a CAR as disclosed herein recognizes and binds BCMA, i.e., an anti-BCMA antigen binding domain. In some embodiments, the antigen binding domain comprises, or alternatively consists essentially of, or yet consists of one or two or three or four or five or six of the following CDRs: a CDRL1 comprising, or alternatively consisting essentially of, or yet consisting of RASESVTILGSHLIH (SEQ ID NO: 47), SASQDISNYLN (SEQ ID NO: 48), RASESVTILGSHLIY (SEQ ID NO: 49), or an equivalent of each thereof; a CDRL2 comprising, or alternatively consisting essentially of, or yet consisting of LASNVQT (SEQ ID NO: 50), YTSNLHS (SEQ ID NO: 51), LASNVQT (SEQ ID NO: 52), or an equivalent of each thereof; a CDRL3 comprising, or alternatively consisting essentially of, or yet consisting of LQSRTIPRT (SEQ ID NO: 53), QQYRKLPWT (SEQ ID NO: 54), LQSRTIPRT (SEQ ID NO: 55), or an equivalent of each thereof, a CDRH1 comprising, or alternatively consisting essentially of, or yet consisting of GYTFTDY (SEQ ID NO: 56), GGTFSNY (SEQ ID NO: 57), GYTFRHY (SEQ ID NO: 58), or an equivalent of each thereof, a CDRH2 comprising, or alternatively consisting essentially of, or yet consisting of INTETRE (SEQ ID NO: 59), YRGHSD (SEQ ID NO: 60), NTESGV (SEQ ID NO: 61), or an equivalent of each thereof, and a CDRH3 comprising, or alternatively consisting essentially of, or yet consisting of DYSYAMDY (SEQ ID NO: 62), GAIYNGYDVLDN (SEQ ID NO: 63), DYLYSLDF (SEQ ID NO: 64), or an equivalent of each thereof. In further embodiments, the CDR equivalent(s) thereof and/or an antigen binding fragment comprising, or alternatively consisting essentially of, or yet consisting of the equivalent(s) recognize and bind BCMA. In some embodiments, the anti-BCMA antigen binding domain comprises, or alternatively consists essentially of, or yet consists of a light chain variable region comprising, or alternatively consisting essentially of, or yet consisting of a sequence selected from

### DIVLTQSPPSLAMSLGKRATISCRASESVTILGSHLIHWYQQKPGQPPTLLIQLASNV QTGVPARFSGSGSRTDFTLTIDPVEEDDVAVYYCLQSRTIPRTFGGGTKLEIK (SEQ ID NO: 65),

### DIQMTQSPSSLSASVGDRVTITCSASQDISNYLNWYQQKPGKAPKLLIYYTSNLHSG VPSRFSGSGSGTDFTLTISSLQPEDFATYYCQQYRKLPWTFGQGTKLEIKR (SEQ ID NO: 66),

DIVLTQSPPSLAMSLGKRATISCRASESVTILGSHLIYWYQQKPGQPPTLLIQLASNV QTGVPARFSGSGSRTDFTLTIDPVEEDDVAVYYCLQSRTIPRTFGGGTKLEIK (SEQ ID NO: 67), or an equivalent of each thereof and/or a heavy chain variable region comprising, or alternatively consisting essentially of, or yet consisting of a sequence selected from

### QIQLVQSGPELKKPGETVKISCKASGYTFTDYSINWVKRAPGKGLKWMGWINTETR EPAYAYDFRGRFAFSLETSASTAYLQINNLKYEDTATYFCALDYSYAMDYWGQGT SVTVSS (SEQ ID NO: 68),

### QVQLVQSGAEVKKPGSSVKVSCKASGGTFSNYWMHWVRQAPGQGLEWMGATYR GHSDTYYNQKFKGRVTITADKSTSTAYMELSSLRSEDTAVYYCARGAIYNGYDVL DNWGQGTLVTVSS (SEQ ID NO: 69),

QIQLVQSGPELKKPGETVKISCKASGYTFRHYSMNWVKQAPGKGLKWMGRINTES GVPIYADDFKGRFAFSVETSASTAYLVINNLKDEDTASYFCSNDYLYSLDFWGQGT ALTVSS (SEQ ID NO: 70), or an equivalent of each thereof. In further embodiments, the variable region equivalent(s) thereof and/or an antigen binding fragment comprising, or alternatively consisting essentially of, or yet consisting of the equivalent(s) recognize and bind BCMA.

In some embodiments, the antigen binding domain of a CAR as disclosed herein recognizes and binds CS1, i.e., an anti-CS1 antigen binding domain. In some embodiments, the anti-CS1 antigen binding domain comprises, or alternatively consists essentially of, or yet consists of one or two or three or four or five or six of the following CDRs: a CDRL1 comprising, or alternatively consisting essentially of, or yet consisting of KASQDVITGVA (SEQ ID NO: 71) or an equivalent thereof, a CDRL2 comprising, or alternatively consisting essentially of, or yet consisting of SASYRYT (SEQ ID NO: 72) or an equivalent thereof, a CDRL3 comprising, or alternatively consisting essentially of, or yet consisting of QQHYSTPLT (SEQ ID NO: 73) or an equivalent thereof,

a CDRH1 comprising, or alternatively consisting essentially of, or yet consisting of GYSFTTY (SEQ ID NO: 74) or an equivalent thereof, a CDRH2 comprising, or alternatively consisting essentially of, or yet consisting of HPSDSE (SEQ ID NO: 75) or an equivalent thereof, and a CDRH3 comprising, or alternatively consisting essentially of, or yet consisting of STMIATRAMDY (SEQ ID NO: 76) or an equivalent thereof. In further embodiments, the CDR equivalent(s) thereof and/or an antigen binding fragment comprising, or alternatively consisting essentially of, or yet consisting of the equivalent(s) recognize and bind CS1. In some embodiments, the anti-CS1 antigen binding domain comprises, or alternatively consists essentially of, or yet consists of a light chain variable region comprising, or alternatively consisting essentially of, or yet consisting of a sequence selected from

### SDIVMTQSQKSMSTSVGDRVSITCKASQDVITGVAWYQQKPGQSPKLLIYSASYRY TGVPDRFTGSGSGTDFTFTISNVQAEDLAVYYCQQHYSTPLTFGAGTKLELK (SEQ ID NO: 77),

### KLELKTGAGFTLPTSYHQQCYYVALDEAQVNSITFTFDTGSGSGTFRDPVGTYRYS ASYILLKPSQGPKQQYWAVGTIVDQSAKCTISVRDGVSTSMSKQSQTMVIDS (SEQ ID NO: 154),

DIVMTQSQKSMSTSVGDRVSITCKASQDVITGVAWYQQKPGQSPKLLIYSASYRYT GVPDRFTGSGSGTDFTFTISNVQAEDLAVYYCQQHYSTPLTFGAGTKLELK (SEQ ID NO: 78), or an equivalent of each thereof and/or a heavy chain variable region comprising, or alternatively consisting essentially of, or yet consisting of a sequence selected from

### SVTVSTGQGWYDMARTAIMTSRACYYVASDESTPSSLQMYATSSSKDVTLTAKDK FKQNLRTESDSPHIMGIWELGQGPRQKVWNMWYTTF SYGSAKCSLKVSAGPRVLE AGPQQLQVQS (SEQ ID NO: 79),

### SSVTVSTGQGWYDMARTAIMTSRACYYVASDESTPSSLQMYATSSSKDVTLTAKD KFKQNLRTESDSPHIMGIWELGQGPRQKVWNMWYTTFSYGSAKCSLKVSAGPRVL EAGPQQLQVQ (SEQ ID NO: 149),

### SQVQLQQPGAELVRPGASVKLSCKASGYSFTTYWMNWVKQRPGQGLEWIGMIHP SDSETRLNQKFKDKATLTVDKSSSTAYMQLSSPTSEDSAVYYCARSTMIATRAMDY WGQGTSVTVS (SEQ ID NO: 80),

QVQLQQPGAELVRPGASVKLSCKASGYSFTTYWMNWVKQRPGQGLEWIGMIHPS DSETRLNQKFKDKATLTVDKSSSTAYMQLSSPTSEDSAVYYCARSTMIATRAMDY WGQGTSVTVSS (SEQ ID NO: 150), or an equivalent of each thereof. In further embodiments, the variable region equivalent(s) thereof and/or an antigen binding fragment comprising, or alternatively consisting essentially of, or yet consisting of the equivalent(s) recognize and bind CS1.

In some embodiments, an equivalent of a reference protein or polypeptide, such as a CDR, a variable region, an antigen binding domain, or an antibody, is at least 80%, or at least 85%, or at least 90%, or at least 95%, or at least 96%, or at least 97%, or at least 98%, or at least 99% identical to the reference protein or polypeptide.

In some embodiments, an equivalent of a reference protein or polypeptide, such as a CDR, a variable region, an antigen binding domain, or an antibody, comprises, or alternatively consists essentially of, or yet consists of the amino acid sequence of C terminus to N terminus of the reference protein or polypeptide. For example, the reference protein or polypeptide comprising, or alternatively consisting essentially of, or yet consisting of KASQDVITGVA (SEQ ID NO: 71), and the equivalent thereof comprises, or alternatively consists essentially of, or yet consists of AVGTIVDQSAK (SEQ ID NO: 151). In the embodiments of a CDR, a variable region, an antigen binding domain, or an antibody as the reference protein or polypeptide, the equivalent still recognizes and binds the same epitope of the reference.

A therapeutic antibody does not guarantee its successful use in a chimeric antigen receptor (CAR) in view of their different mechanisms of action and safety profilings. They cannot predict each other even for targeting a same antigen. Antibodies take advantages of antigen binding to induce tumor apoptosis and/or Fc receptor binding to macrophages, natural killer (NK) cells and neutrophils to induce multifaceted immune responses. However, CAR immune cells (e.g., T cells and NK cells) utilize specific binding to a tumor antigen to subsequently induce immune cell activation via an artificial chimeric receptor. Although both CAR immune cells and antibodies can have off-target toxicities, specifically, antibodies can induce Fc-receptor-mediated toxicity (Schlothauer et al. Protein Eng. Des. Sel. 29, 457-466 (2016)), while CAR immune cells usually lead to lethal cytokine release syndrome (CRS) and neurological toxicities (Gupta et al. J Emerg Med. 2020 May 27;S0736-4679(20)30352-8). Thus, one of skill in the art cannot predict CAR efficacy and safety solely based on common antigen binding domain.

Although in some aspects, an scFv in a CAR can be derived from an antibody; to be functional, both are required to bind to a tumor antigen. However, the binding of an antibody to a tumor antigen is usually in a soluble form, while the binding of a scFv on CAR to a tumor antigen is from the surface of immune cells. There are at least three differences that determine these two types of interactions: 1) protein or peptide conformation requirement is different in the two settings; 2) in the antibody setting, a full-length antibody consisting of two Fab regions (each consisting of a part of heavy chain and a light chain) and a Fc on a heavy chain governs binding affinity and the binding is bivalent due to the two Fab regions, while for CAR, scFv, in which the one Fab region consisting of a heavy chain variable region (VH) and a light chain viable region (VL) that are linked to each other by a linker, is univalent binding; 3) due to univalent binding and a shorter length of scFv compared to its corresponding antibody, a CAR may have less binding affinity while less immunogenic in patients when a murine scFv is used for a CAR, which is the most cases of CARs on the market (Dotti et al. Immunol Rev 257, 107-126 (2014)). Thus, functionality of an antibody cannot predict that of a CAR. In fact, Haso et al. (Blood 121, 1165-1174 (2013)) recently demonstrated that increasing the binding affinity of a CAR using a high-affinity antibody-derived scFv failed to increase CAR activity in vitro and in vivo (Haso et al. Blood 121, 1165-1174 (2013) and Handgretinger et al. Blood 121, 1065-1066 (2013)).

In some embodiments, an antigen binding domain as disclosed herein further comprises a peptide linker, for example, between two CDRs and/or between its light chain variable region or the equivalent thereof and its heavy chain variable region or the equivalent thereof. In one embodiments, the peptide linker comprises, or alternatively consists essentially of, or yet consists of GGGGSGGGGSGGGGS (SEQ ID NO: 14) or an equivalent thereof. In some embodiments, an equivalent of SEQ ID NO: 14 may be from 1 to 50 amino acids long, for instance, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. In some embodiments, an equivalent of SEQ ID NO: 14 is glycine rich, although it may also contain serine or threonine. In some embodiments, an equivalent of SEQ ID NO: 14 comprises, or alternatively consists essentially of, or yet consists of (GGGGS)n, wherein n may be an integer of 1, or 2, or 3, or 4, or 5, or 6, or 7, or 8, or 9, or 10, or 11, or 12, or 13, or 14, or 15, or more (SEQ ID NOs: 134-135, 14 and 136-147).

Hinge Domain. The hinge domain may be derived either from a natural or from a synthetic source. In some embodiments, the hinge domain is derived from a cluster of differentiation protein, such as CD8, CD28, CD3, CD45, CD4, CD5, CDS, CD9, CD 16, CD22, CD33, CD37, CD64, CD80, CD86, CD 134, CD137, CD 154. In one embodiments, the hinge domain is a CD8 α hinge domain. In some embodiments, the hinge domain is derived from an immunoglobulin, such as IgA, IgD, IgE, IgG, or IgM. In one embodiment, the hinge domain is an IgG1 hinge domain. In a further embodiment, the IgG1 hinge domain comprises, or alternatively consists essentially of, or yet consists of LEPKSCDKTHTCPPCPDPKGT (SEQ ID NO: 1) or an equivalent thereof. In some embodiments, an equivalent of SEQ ID NO: 1 comprises 1, or 2, or 3, or 4, or 5, or 6, or 7, or 8, or 9, or 10, or 11, or 12, or 13, or 14, or 15, or more mutations compared to SEQ ID NO: 1.

Transmembrane Domain. The transmembrane domain may be derived either from a natural or from a synthetic source. Where the source is natural, the domain may be derived from any membrane-bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, and TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker. In some embodiments, the transmembrane domain comprises, or alternatively consists essentially of, or yet consists of a CD8 α transmembrane domain or a CD28 transmembrane domain.

Cytoplasmic/intracellular Domain. The cytoplasmic domain or intracellular domain of the CAR is responsible for activation of at least one of the traditional effector functions of an immune cell in which a CAR has been placed. In some embodiments, the cytoplasmic domain or intracellular domain comprises, or alternatively consists essentially of, or yet consists of an intracellular signaling domain. The intracellular signaling domain or in some embodiments, signaling domain, refers to a portion of a protein which transduces the effector function signal and directs the immune cell to perform its specific function. An entire signaling domain or a truncated portion thereof may be used so long as the truncated portion is sufficient to transduce the effector function signal. Cytoplasmic sequences of the TCR and co-receptors as well as derivatives or variants thereof can function as intracellular signaling domains for use in a CAR. Intracellular signaling domains of particular use in this disclosure may be derived from FcR, TCR, CD3, CDS, CD22, CD79a, CD79b, and CD66d.

In some embodiments, the signaling domain of the CAR can comprise, or alternatively consist essentially of, or yet consist of a CD3 ζ signaling domain. In some embodiments, the CD3 zeta signaling domain comprises, or alternatively consists essentially of, or yet consists of

RVKFSRSADAPAYQQGQNQLYNELNLGRREEYDVLDKRRGRDPEMGGKPRRKNP QEGLYNELQKDKMAEAYSEIGMKGERRRGKGHDGLYQGLSTATKDTYDALHMQ ALPPR (SEQ ID NO: 3) or an equivalent thereof. In further embodiments, the equivalent of SEQ ID NO: 3 may comprises 1, or 2, or 3, or 4, or 5, or 6, or 7, or 8, or 9, or 10, or 11, or 12, or 13, or 14, or 15, or more mutations compared to SEQ ID NO: 3 but is still capable of transducing the effector function signal and directing the immune cell to perform its specific function as SEQ ID NO: 3. Exemplified methods assessing such transduction can be found, for example, in Bridgeman J S, et al. Clin Exp Immunol. 2014 February;175(2):258-67.

Since signals generated through the TCR are alone insufficient for full activation of a T cell, a secondary or co-stimulatory signal may also be required. Thus, the intracellular region of a co-stimulatory signaling molecule, including but not limited the intracellular domains of the proteins CD27, CD28, 4-IBB (CD 137), OX40, CD30, CD40, PD-1, ICOS, lymphocyte function-associated antigen-1 (LFA-1), CD2, CD7, LIGHT, NKG2C, B7-H3, or a ligand that specifically binds with CD83, may also be included in the cytoplasmic domain of the CAR. For instance, a CAR may comprise one, two, or more co-stimulatory domains, in addition to a signaling domain (e.g., a CD3 ζ signaling domain). In some embodiments, the intracellular domain further comprises one or more or two or more costimulatory regions selected from a CD28 costimulatory signaling region, a 4-1BB costimulatory signaling region, an ICOS costimulatory signaling region, or an OX40 costimulatory region.

In some embodiments, the cell activation moiety of the chimeric antigen receptor is a T-cell signaling domain comprising, or alternatively consisting essentially of, or yet further consisting of, one or more proteins or fragments thereof selected from the group consisting of CD8 protein, CD28 protein, 4-1BB protein, OX40, CD30, CD40, PD-1, ICOS, LFA-1, CD2, CD7, CD27, LIGHT, NKG2C, B7-H3, and CD3-zeta protein.

In some embodiments, the cell activation moiety of the chimeric antigen receptor is a T-cell signaling domain comprising, or alternatively consisting essentially of, or yet further consisting of, one or more proteins or fragments thereof selected from the group consisting of CD8 protein, CD28 protein, 4-1BB protein, and CD3-zeta protein.

In specific embodiments, the CAR comprises, or alternatively consists essentially of, or yet consists of an antigen binding domain of a cancer or tumor targeting antibody, a CD8 α hinge domain, a CD8 α transmembrane domain, a costimulatory signaling region, and a CD3 zeta signaling domain. In further embodiments, the costimulatory signaling region comprises either or both a CD28 costimulatory signaling region and a 4-1BB costimulatory signaling region. In some embodiments, the CAR comprises, or alternatively consists essentially of, or yet consists of a CD28 transmembrane and cytoplasmic domain comprising

FWVLVVVGGVLACYSLLVTVAFIIFWVRSKRSRLLHSDYMNMTPRRPGPTRKHYQ PYAPPRDFAAYRS (SEQ ID NO: 2) or an equivalent thereof. In further embodiments, the equivalent of SEQ ID NO: 2 may comprises 1, or 2, or 3, or 4, or 5, or 6, or 7, or 8, or 9, or 10, or 11, or 12, or 13, or 14, or 15, or more mutations compared to SEQ ID NO: 32 but is still capable of functioning as a transmembrane domain and a costimulatory signaling region.

In some embodiments, a CAR and/or a cytoplasmic domain thereof as disclosed herein further comprise an IL2RP or a fragment thereof. In further embodiments, the fragments of IL2RP comprises, or alternatively consists essentially of, or yet consists of an JAK-STAT activation domain of the IL2RP, facilitating activation of the immune cell. In some embodiments, the CAR and/or the intracellular domain of the CAR further comprises an IL2R3 or a fragment thereof comprising an JAK-STAT activation domain. In some embodiments, the JAK-STAT activation domain comprises, or alternatively consists essentially of, or yet consists of a JAK binding domain (also known as a box-1 motif which allows for tyrosine kinase JAK association, for example JAK1) and/or a Signal Transducer and Activator of Transcription (STAT, such as STAT3 or STAT5) association motif. An example of the JAK binding domain can be found amino acid numbers 278 to 286 of NCBI RefSeq: NP 000869.1. In further embodiments, the intracellular domain further comprises an endogenous or exogenous JAK-binding motif and/or an endogenous or exogenous STAT association motif. In some embodiments, the exogenous STAT3 association motif is YXXQ (SEQ ID NO: 152), optionally YRHQ (SEQ ID NO: 153). Cells expressing such CAR cells show antigen-dependent JAK-STAT3/5 pathway activation, which promoted their proliferation and prevented terminal differentiation in vitro. Alternatively, JAK-STAT activation domain from a protein other than IL2RP may be used here as a substitution. Exemplified of such protein may include Erythropoietin receptor (EpoR), thrombopoietin receptor (TpoR), granulocyte macrophage colony stimulating factor receptor (GM-CSFR), or growth hormone receptor (GHR). See, for example, US20190359685A1 and Kagoya et al. Nat Med. 2018 March;24(3):352-359. doi: 10.1038/nm.4478. Epub 2018 Feb. 5.

Switch Mechanisms. In some embodiments, the CAR may also comprise a switch mechanism for controlling expression and/or activation of the CAR. For example, a CAR may comprise, consist, or consist essentially of an extracellular, transmembrane, and intracellular domain, in which the extracellular domain comprises a target-specific binding element that binds a label, binding domain, or tag that is specific for a molecule other than the target antigen that is expressed on or by a target cell (such as a cancer cell). In some embodiments, such label, binding domain or tag recognizes and binds the target antigen that is expressed on or by the target cell. In such embodiments, the specificity of the CAR is provided by a second construct that comprises, consists, or consists essentially of a target antigen binding domain and a domain on the CAR that is recognized by or binds to the label, binding domain, or tag. See, e.g., WO 2013/044225, WO 2016/000304, WO 2015/057834, WO 2015/057852, WO 2016/070061, U.S. Pat. No. 9,233,125, US 2016/0129109. In this way, a T-cell, NK cell or other immune cells that express the CAR can be administered to a subject, but it cannot bind a target antigen (i.e., BCMA) until a second composition comprising the label, binding domain, or tag, such as an BCMA-specific binding domain is administered.

CARs of the present disclosure may likewise require multimerization in order to active their function (see, e.g., US 2015/0368342, US 2016/0175359, US 2015/0368360) and/or an exogenous signal, such as a small molecule drug (US 2016/0166613, Yung et al., Science, 2015) in order to elicit an immune cell response, such as a T-cell response or a NK cell response.

Furthermore, the disclosed CARs can comprise a “suicide switch” (also referred to as a “suicide gene”) to induce cell death of the CAR cells following treatment (Buddee et al., PLoS One, 2013) or to downregulate expression of the CAR following binding to the target antigen (WO 2016/011210). A non-limiting exemplary suicide switch or suicide gene is iCasp. In some embodiments, a CAR and/or a cytoplasmic domain thereof as disclosed herein further comprise a suicide gene product. In further embodiments, the suicide gene product is selected from one or more of: HSV-TK (Herpes simplex virus thymidine kinase), cytosine deaminase, nitroreductase, carboxylesterase, cytochrome P450 or PNP (Purine nucleoside phosphorylase), truncated EGFR, or inducible caspase (“iCasp”).

In some embodiments, the CAR can further comprise a detectable marker or purification marker. In another aspect, the CARs as described herein are contained in a composition, e.g., a pharmaceutically acceptable carrier for diagnosis or therapy.

In certain embodiments, the antigen binding domain of the cancer- or tumor-targeting antibody of the CAR comprises, or alternatively consists essentially of, or further consists of a heavy chain variable region and a light chain variable region that are optionally linked by a linker peptide. In some embodiments, the heavy and/or light chain variable region comprises, or alternatively consists essentially of, or further consists of the relevant CDR regions of an antibody to antigen (such as those other than a B-cell maturation antigen (BCMA)), and/or SLAM/11F7 (also known as CS1 or CD319), and/or an equivalent of each thereof. In certain embodiments, the CAR further comprises, or alternatively further consists essentially of, or yet further consists of, a linker polypeptide located between the heavy chain variable region and the light chain variable region. In certain embodiments, the linker is a glycine-serine linker. In further embodiments, the linker polypeptide comprises, or alternatively consists essentially of, or further consists of the sequence (glycine-serine)n wherein n is an integer from 1 to 6 (SEQ ID NO: 110), wherein in one aspect, n is 4.

In certain embodiments, the antigen binding domain of the cancer- or tumor-targeting antibody of the CAR binds any cancer- or tumor-associated antigen, such as one or more selected from FLT3, CD19, mesothelin, human epidermal growth factor receptor 2 (HER2), prostate stem cell antigen (PSCA), carcinoembryonic antigen (CEA), GTP-ase-activating protein (GAP), ganglioside G2 (GD2), CD5, prostate specific membrane antigen (PSMA), receptor tyrosine kinase-like orphan receptor 1 (ROR1), CD123, CD70, CD38, B cell maturation antigen (BCMA), mucin 1, (Muc1), ephrin type-A receptor 2 precursor (EphA2), wildtype epidermal growth factor receptor (EGFRwt), epidermal growth factor receptor variant III (EGFRVIII), interleukin 13 receptor alpha 2 (IL13Ra2), CD133, glypican 3 (GPC3), epithelial cell adhesion molecule precursor (EpCam), fibroblast activation protein alpha (FAP), vascular endothelial growth factor receptor 2 (VEGFR2), cancer/testis (CT), guanylyl cyclase C (GUCY2C), tumor-associated glycoprotein-72 (TAG-72), thymidine kinase 1 (TK1), and hypoxanthine guanine phosphoribosyltransferase (HPRT1). These may comprise the heavy and light chains of the antigen binding domains, that alternatively consist of or comprise the CDRs of the antigen binding domains.

Also provided are polynucleotides (such as isolated and/or engineered polypeptides) and/or isolated nucleic acids that encode the CAR constructs. The polynucleotides and/or nucleic acids can further comprise the necessary regulatory sequences, e.g., a promoter for expression in a host cell, e.g., a mammalian or human host cell such as a T cell or an NK cell and/or enhancer elements. In some embodiments, the polynucleotides and/or nucleic acids further comprise a first regulatory sequence directing the expression of the CAR. In further embodiments, the regulatory sequences comprises one or more of the following: a promoter, an intron, an enhancer, or a polyadenylation signal. In some embodiments, the promoter is a CMV, MND, or an EF1alpha promoter. In further embodiments, the CAR polynucleotides and/or nucleic acids further encode a detectable or purification marker peptide (e.g., GFP) that may be regulated from a second regulatory sequence, such as a promoter element, e.g., CMV, MND, and EF1A promoters, located 5′ to the encoding polynucleotide. In some embodiments, the second promoter comprises an EF1 alpha promoter. As is apparent to the skilled artisan, the promoter(s) are selected for the host expression system and will vary with the host and the expression vector and intended use. In some embodiments, the polynucleotides and/or nucleic acids further comprise a suicide gene encoding a suicide gene product optionally selected from one or more of: HSV-TK (Herpes simplex virus thymidine kinase), cytosine deaminase, nitroreductase, carboxylesterase, cytochrome P450 or PNP (Purine nucleoside phosphorylase), truncated EGFR, or inducible caspase (“iCasp”). In further embodiments, the polynucleotides and/or nucleic acids further comprise a third regulatory sequence directing expression of the suicide gene product. In one embodiment, any one or two or three of the regulatory sequences is or are inducible or constitutively active in the cell. In one embodiment, any one or two or three of the regulatory sequences can be cell specific or tissue specific.

In some embodiments, the polynucleotides and/or nucleic acids comprise, or alternatively consist essentially of, or yet consist of one or more of the following: (I) a nucleotide sequence encoding a signal peptide, and wherein the nucleotide sequence comprises, or alternatively consists essentially of, or yet consists of

### ATGGGGTGGTCAAGCATTATTCTGTTTCTGGTCGCTACCGCTACAGGCGTCCAT (SEQ ID NO: 86),

ATGTACAGGATGCAACTCCTGTCTTGCATTGCACTAAGTCTTGCACTTGTCACA AACAGT (SEQ ID NO: 87), or an equivalent of each thereof,

(II) a nucleotide sequence encoding a linker peptide, and wherein the nucleotide sequence comprises, or alternatively consists essentially of, or yet consists of

(SEQ ID NO: 88)
GGTGGGGGCGGCTCTGGTGGCGGTGGCAGCGGCGGAGGTGGCAGT;

(III) a nucleotide sequence encoding a transmembrane and cytoplasmic domain, and wherein the nucleotide sequence comprises, or alternatively consists essentially of, or yet consists of

TTTTGGGTGCTGGTGGTGGTTGGTGGAGTCCTGGCTTGCTATAGCTTGCTAGTAA CAGTGGCCTTTATTATTTTCTGGGTGAGGAGTAAGAGGAGCAGGCTCCTGCACA GTGACTACATGAACATGACTCCCCGCCGCCCCGGGCCCACCCGCAAGCATTACC AGCCCTATGCCCCACCACGCGACTTCGCAGCCTATCGCTCC (SEQ ID NO: 89) or an equivalent thereof;

(IV) a nucleotide sequence encoding a signaling domain, and wherein the nucleotide sequence comprises, or alternatively consists essentially of, or yet consists of

AGAGTGAAGTTCAGCAGGAGCGCAGACGCCCCCGCGTACCAGCAGGGCCAGAA CCAGCTCTATAACGAGCTCAATCTAGGACGAAGAGAGGAGTACGATGTTTTGG ACAAGAGACGTGGCCGGGACCCTGAGATGGGGGGAAAGCCGAGAAGGAAGAA CCCTCAGGAAGGCCTGTACAATGAACTGCAGAAAGATAAGATGGCGGAGGCCT ACAGTGAGATTGGGATGAAAGGCGAGCGCCGGAGGGGCAAGGGGCACGATGG CCTTTACCAGGGTCTCAGTACAGCCACCAAGGACACCTACGACGCCCTTCACAT GCAGGCCCTGCCCCCTCGC (SEQ ID NO: 90) or an equivalent thereof;

(VI) a nucleotide sequence encoding a linker, and wherein the nucleotide sequence comprises, or alternatively consists essentially of, or yet consists of

CCGAGCGGCCAGGCGGGCGCGGCGGCATCGGAGTCCCTGTTTGTGTCAAATCA CGCCTAC (SEQ ID NO: 92) or an equivalent thereof;

(VII) a nucleotide sequence encoding an anti-BCMA light chain variable region, and wherein the nucleotide sequence is selected from:

GATATTGTTCTTACTCAATCACCCCCAAGCCTTGCGATGTCTCTTGGTAA ACGAGCGACAATTAGTTGTAGAGCTTCTGAAAGCGTAACTATTCTTGGGTCACA TCTTATTCATTGGTATCAACAAAAGCCGGGACAACCGCCTACACTCTTGATTCA ACTCGCGAGCAATGTTCAAACGGGTGTCCCTGCACGCTTTTCTGGGAGCGGTTC ACGAACAGATTTTACTCTCACGATTGATCCAGTCGAAGAAGATGATGTCGCTGT ATATTATTGTCTCCAAAGTAGGACAATACCAAGAACTTTTGGTGGTGGTACAAA ATTGGAAATTAAA (SEQ ID NO: 93),

GACATCCAGATGACCCAGAGCCCTAGCTCACTGAGCGCCAGCGTGGGCG ACAGGGTGACCATTACCTGCTCCGCCAGCCAGGACATCAGCAACTACCTGAACT GGTACCAGCAGAAGCCCGGCAAGGCCCCCAAGCTGCTGATCTACTACACCTCC AACCTGCACTCCGGCGTGCCCAGCAGGTTCAGCGGAAGCGGCAGCGGCACCGA TTTCACCCTGACCATCTCCAGCCTGCAGCCCGAGGACTTCGCCACCTACTACTG CCAGCAGTACAGGAAGCTCCCCTGGACTTTCGGCCAGGGCACCAAACTGGAGA TCAAGCGT (SEQ ID NO: 94),

GACATCGTGCTGACCCAGAGCCCCCCCAGCCTGGCCATGTCTCTGGGCA AGAGAGCCACCATCAGCTGCCGGGCCAGCGAGAGCGTGACCATCCTGGGCAGC CACCTGATCTACTGGTATCAGCAGAAGCCTGGCCAGCCCCCCACCCTGCTGATC CAGCTGGCTAGCAATGTGCAGACCGGCGTGCCCGCCAGATTCAGCGGCAGCGG CAGCAGAACCGACTTCACCCTGACCATCGACCCCGTGGAAGAGGACGACGTGG CCGTGTACTACTGCCTGCAGAGCCGGACCATCCCCCGGACCTTTGGCGGAGGAA CAAAGCTGGAAATCAAG (SEQ ID NO: 95),

GACATCGTGCTGACCCAGAGCCCCCCCAGCCTGGCCATGTCTCTGGGCAAGAGA GCCACCATCAGCTGCCGGGCCAGCGAGAGCGTGACCATCCTGGGCAGCCACCT GATCCACTGGTATCAGCAGAAGCCCGGCCAGCCCCCCACCCTGCTGATCCAGCT CGCCAGCAATGTGCAGACCGGCGTGCCCGCCAGATTCAGCGGCAGCGGCAGCA GAACCGACTTCACCCTGACCATCGACCCCGTGGAAGAGGACGACGTGGCCGTG TACTACTGCCTGCAGAGCCGGACCATCCCCCGGACCTTTGGCGGAGGCACCAAA CTGGAAATCAAG (SEQ ID NO: 96) or an equivalent of each thereof;

(VIII) a nucleotide sequence encoding an anti-BCMA heavy chain variable region, and wherein the nucleotide sequence is selected from:

CAGATCCAGCTGGTGCAGTCCGGCCCCGAGCTGAAGAAGCCCGGCGAGA CCGTGAAGATCTCCTGCAAGGCCTCCGGCTACACCTTCACCGACTACTCCATCA ACTGGGTGAAGCGGGCCCCCGGCAAGGGCCTGAAGTGGATGGGCTGGATCAAC ACCGAGACCCGGGAGCCCGCCTACGCCTACGACTTCCGGGGCCGGTTCGCCTTC TCCCTGGAGACCTCCGCCTCCACCGCCTACCTGCAGATCAACAACCTGAAGTAC GAGGACACCGCCACCTACTTCTGCGCCCTGGACTACTCCTACGCCATGGACTAC TGGGGCCAGGGCACCTCCGTGACCGTGTCCTCC (SEQ ID NO: 97),

CAGGTGCAGCTGGTCCAGAGCGGCGCCGAAGTGAAGAAGCCCGGCAGC TCCGTGAAAGTGAGCTGCAAGGCCAGCGGCGGCACCTTCAGCAACTACTGGAT GCACTGGGTGAGGCAGGCCCCCGGACAGGGCCTGGAGTGGATGGGCGCCACCT ACAGGGGCCACAGCGACACCTACTACAACCAGAAGTTCAAGGGCCGGGTGACC ATCACCGCCGACAAGAGCACCAGCACCGCCTACATGGAACTGAGCAGCCTCAG GAGCGAGGACACCGCTGTGTATTACTGCGCCAGGGGCGCCATCTACAACGGCT ACGACGTGCTGGACAACTGGGGCCAGGGCACACTAGTGACCGTGTCCAGC (SEQ ID NO: 98),

ATGGGATGGAGCTCTATCATCCTCTTCTTGGTAGCAACAGCTACAGGTGTCCAC CAGATTCAGCTGGTGCAGAGCGGCCCTGAGCTGAAGAAACCCGGCGAGACAGT GAAGATCAGCTGCAAGGCCTCCGGCTACACCTTCCGGCACTACAGCATGAACTG GGTGAAACAGGCCCCTGGCAAGGGCCTGAAGTGGATGGGCCGGATCAACACCG AGAGCGGCGTGCCCATCTACGCCGACGACTTCAAGGGCAGATTCGCCTTCAGCG TGGAAACCAGCGCCAGCACCGCCTACCTGGTGATCAACAACCTGAAGGACGAG GATACCGCCAGCTACTTCTGCAGCAACGACTACCTGTACAGCCTGGACTTCTGG GGCCAGGGCACCGCCCTGACCGTGTCCAGC (SEQ ID NO: 99), or
CAGATTCAGCTGGTGCAGAGCGGCCCTGAGCTGAAGAAACCCGGCGAGACAGT GAAGATCAGCTGCAAGGCCTCCGGCTACACCTTCACCGACTACAGCATCAACTG GGTGAAAAGAGCCCCTGGCAAGGGCCTGAAGTGGATGGGCTGGATCAACACCG AGACAAGAGAGCCCGCCTACGCCTACGACTTCCGGGGCAGATTCGCCTTCAGCC TGGAAACCAGCGCCAGCACCGCCTACCTGCAGATCAACAACCTGAAGTACGAG GACACCGCCACCTACTTTTGCGCCCTGGACTACAGCTACGCCATGGACTACTGG GGCCAGGGCACCAGCGTGACCGTGTCCAGC (SEQ ID NO: 100) or an equivalent of each thereof;

(XI) a nucleotide sequence encoding an anti-FLT3 light chain variable region, and wherein the nucleotide sequence comprises, or alternatively consists essentially of, or yet consists of

GATATTGTGCTAACTCAGTCTCCAGCCACCCTGTCTGTGACTCCAGGAGATAGC GTCAGTCTTTCCTGCAGGGCCAGCCAGAGTATTAGCAACAACCTACACTGGTAT CAACAAAAATCACATGAGTCTCCAAGGCTTCTCATCAAGTATGCTTCCCAGTCC ATCTCTGGGATCCCCTCCAGGTTCAGTGGCAGTGGATCAGGGACAGATTTCACT CTCAGTATCAACAGTGTGGAGACTGAAGATTTTGGAGTGTATTTCTGTCAACAG AGTAACACCTGGCCGTACACGTTCGGAGGGGGGACCAAGCTGGAAATAAAACG G (SEQ ID NO: 103),
GACATTGTGATGACACAGTCTCCATCCTCCCTGAGTGTGTCAGCAGGAGAGAAG GTCACTATGAGCTGCAAGTCCAGTCAGAGTCTGTTAAACAGTGGAAATCAAAA GAACTATATGGCCTGGTATCAGCAGAAACCAGGGCAGCCTCCTAAACTGTTGAT CTACGGGGCATCCACTAGGGAATCTGGGGTCCCTGATCGCTTCACAGGCAGTGG ATCTGGAACCGATTTCACTCTTACCATCAGCAGTGTGCAGGCTGAAGACCTGGC AGTTTATTACTGTCAGAATGATCATAGTTATCCGCTCACGTTCGGTGCTGGGAC CAAGCTGGAGCTGAAACGG (SEQ ID NO: 104) or an equivalent of each thereof,

(XII) a nucleotide sequence encoding an anti-FLT3 heavy chain variable region, and wherein the nucleotide sequence comprises, or alternatively consists essentially of, or yet consists of

CAGGTCCAACTGCAGCAGCCTGGGGCTGAGCTTGTGAAGCCTGGGGCTTCATTG AAGCTGTCCTGCAAGTCTTCCGGGTACACCTTCACCAGCTACTGGATGCACTGG GTGAGGCAGAGGCCTGGACATGGCCTTGAGTGGATCGGAGAGATTGATCCTTCT GACAGTTATAAAGACTACAATCAGAAGTTCAAGGACAAGGCCACATTGACTGT GGACAGATCCTCCAACACAGCCTACATGCACCTCAGCAGCCTGACATCTGATGA CTCTGCGGTCTATTATTGTGCAAGAGCGATTACGACGACCCCCTTTGACTTCTGG GGCCAAGGCACCACTCTCACAGTCTCCTCA (SEQ ID NO: 105),
CAGGTGCAGCTGAAGCAGTCAGGACCTGGCCTAGTGCAGCCCTCACAGAGCCT GTCCATCACCTGCACAGTCTCTGGTTTCTCATTAACTAACTATGGTTTACACTGG GTTCGCCAGTCTCCAGGAAAGGGCCTGGAGTGGCTGGGAGTGATATGGAGTGG TGGAAGCACAGACTATAATGCAGCTTTCATATCCAGACTGAGCATCAGCAAGG ACAACTCCAAGAGCCAAGTTTTCTTTAAAATGAACAGTCTGCAGGCTGATGACA CAGCCATATACTACTGTGCCAGAAAAGGAGGGATCTACTATGCTAACCATTACT ATGCTATGGACTACTGGGGTCAAGGAACCTCAGTCACCGTCTCCTCA (SEQ ID NO: 106) or an equivalent of each thereof;

(XIII) a nucleotide sequence encoding an anti-CS1 light chain variable region, and wherein the nucleotide sequence comprises, or alternatively consists essentially of, or yet consists of AAACTTGAGT TGAAGACCGG TGCCGGCTTC ACCTTACCGA CCAGTTATCA TCAACAATGC TATTACGTGG CCCTGGACGA AGCACAGGTG AATTCAATTA CGTTTACGTT TGATACCGGC TCTGGCAGCG GTACATTTCG TGATCCCGTG GGCACTTACC GCTATTCGGC GAGTTATATC TTGCTGAAAC CTTCCCAAGG TCCGAAACAG CAGTACTGGG CGGTTGGCAC CATTGTAGAC CAATCAGCCA AATGTACAAT CTCGGTTCGC GATGGTGTCA GTACGTCGAT GTCTAAGCAG TCACAGACAA TGGTTATCGA T (SEQ ID NO: 107) or an equivalent thereof;

(XIV) a nucleotide sequence encoding an anti-CS1 heavy chain variable region, and wherein the nucleotide sequence comprises, or alternatively consists essentially of, or yet consists of AGCGTTACCG TGAGTACAGG CCAGGGCTGG TATGACATGG CACGTACAGC CATCATGACC TCGCGCGCAT GTTACTACGT CGCGTCAGAT GAATCGACGC CTTCCTCGCT GCAAATGTAT GCAACCTCCA GCAGCAAAGA TGTTACCCTG ACCGCAAAGG ACAAGTTTAA ACAGAATTTG CGTACGGAGA GTGACTCCCC GCACATCATG GGAATCTGGG AGTTGGGTCA GGGGCCTCGT CAGAAGGTAT GGAACATGTG GTATACAACT TTTTCGTACG GCTCAGCAAA ATGCAGCTTG AAAGTGTCGG CAGGTCCGCG CGTGCTGGAG GCCGGTCCGC AGCAGCTGCA AGTCCAGTCT (SEQ ID NO: 108) or an equivalent thereof, and

(XV) a nucleotide sequence encoding a hinge domain, and wherein the nucleotide sequence comprises, or alternatively consists essentially of, or yet consists of CTCGAGCCCAAATCTTGTGACAAAACTCACACATGCCCACCGTGCCCGGATCCC AAAGGTACC (SEQ ID NO: 109) or an equivalent thereof.

In some embodiments, an equivalent of a reference nucleotide sequence encodes the same amino acid sequence of the reference. In some embodiments, an equivalent of a reference nucleotide sequence is at least 80% (such as at least 85%, at least 90%, or at least 91%, or at least 92%, or at least 93%, or at least 94%, at least 95%, at least 96%, at least 97%, at least 98%, or at least 99%) identical to the reference. Additionally or alternatively, an equivalent of a reference nucleotide sequence hybridizes under conditions of high stringency to the complement of the reference. In some embodiments, conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. Also provided is a polynucleotide that is reverse, complement, or reverse-complement to the polynucleotide as disclosed herein. In some embodiments, a polynucleotide as disclosed herein further comprise a detectable or purification marker and/or a sequence encoding a detectable or purification marker.

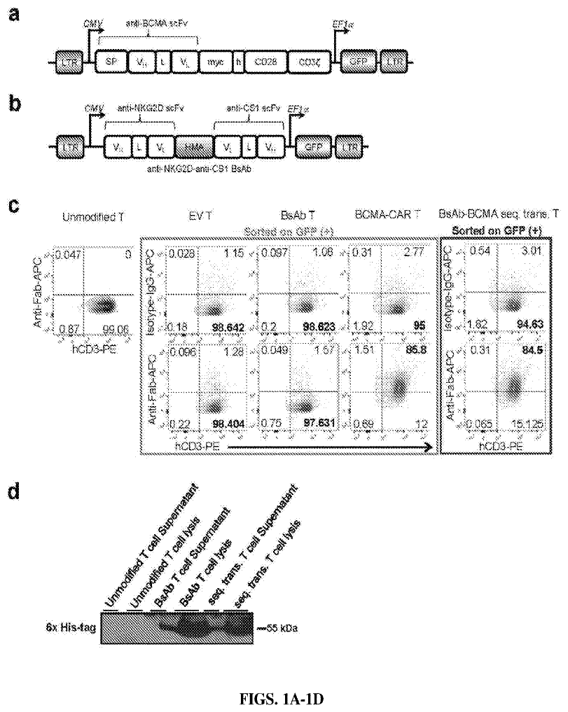

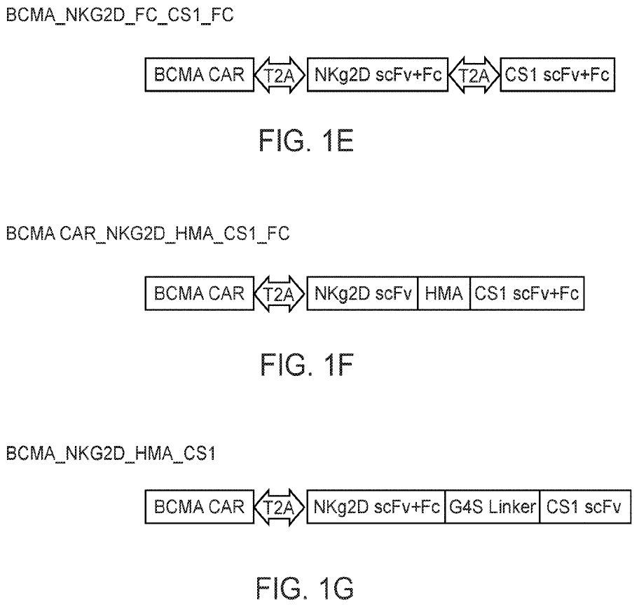

The polynucleotides and/or isolated nucleic acid can be inserted into a vector, such as an expression vector, e.g., a lentiviral vector or retroviral vector (between the 5′ and 3′ LTRs) or an adenovirus vector or any other vectors that can express a gene from. FIG. 1A is an exemplary construct of this disclosure. As is apparent, when used clinically in a human patient, marker or purification tags will be omitted from the construct. The cells can be transduced using the viral vectors as described herein or alternatively using technology described in Riet et al. (2013) Meth. Mol. Biol. 969:187-201 entitled “Nonviral RNA transfection to transiently modify T cell with chimeric antigen receptors for adoptive therapy.” In some embodiments, the vector is a non-viral vector, such as a plasmid. In some embodiments, the vector is a viral vector, optionally selected from the group of a retroviral vector, a lentiviral vector, an adenoviral vector, and an adeno-associated viral vector. Additionally or alternatively, the vector further comprises a regulatory sequence directing replication and/or expression of the CAR encoding polynucleotide and/or isolated nucleic acid. In further embodiments, the regulatory sequence comprises one or more of the following: a promoter, an intron, an enhancer, or a polyadenylation signal. In some embodiments, the vector further comprises a detectable or purification marker.

### Process for Preparing Antibodies for Use in the CAR Constructs

Antibodies for use in this disclosure can be purchased or prepared using methods known in the art and briefly described herein. If a new antigen is discovered, it will be necessary to manufacture antibodies and antigen binding domains of the antibodies. Their manufacture and uses are well known and disclosed in, for example, Harlow, E. and Lane, D., Antibodies: A Laboratory Manual, Cold Spring Harbor Laboratory Press, Cold Spring Harbor, N.Y., 1999. The antibodies may be generated using standard methods known in the art. Examples of antibodies include (but are not limited to) monoclonal, single chain, and functional fragments of antibodies.

Antibodies may be produced in a range of hosts, for example goats, rabbits, rats, mice, humans, and others. They may be immunized by injection with a target antigen or a fragment or oligopeptide thereof which has immunogenic properties, such as a C-terminal fragment a cancer or tumor relevant antigen or an isolated polypeptide, such as BCMA or NKG2D. Depending on the host species, various adjuvants may be added and used to increase an immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Among adjuvants used in humans, BCG (Bacille Calmette-Guerin) and Corynebacterium parvum are particularly useful. This this disclosure also provides the isolated polypeptide and an adjuvant.

In certain embodiments, the antibodies of the present disclosure are polyclonal, i.e., a mixture of plural types of antibodies having different amino acid sequences. In one embodiments, the polyclonal antibody comprises a mixture of plural types of antibodies having different CDRs. As such, a mixture of cells which produce different antibodies is cultured, and an antibody purified from the resulting culture can be used (see WO 2004/061104).

Monoclonal Antibody Production. Monoclonal antibodies to a cancer or tumor antigen may be prepared using any technique which provides for the production of antibody molecules by continuous cell lines in culture. Such techniques include, but are not limited to, the hybridoma technique (see, e.g., Kohler & Milstein, Nature 256: 495-497 (1975)); the trioma technique; the human B-cell hybridoma technique (see, e.g., Kozbor et al., Immunol. Today 4: 72 (1983)) and the EBV hybridoma technique to produce human monoclonal antibodies (see, e.g., Cole et al., in: MONOCLONAL ANTIBODIES AND CANCER THERAPY, Alan R. Liss, Inc., pp. 77-96 (1985)). Human monoclonal antibodies can be utilized in the practice of the present technology and can be produced by using human hybridomas (see, e.g., Cote et al., Proc. Natl. Acad. Sci. 80: 2026-2030 (1983)) or by transforming human B-cells with Epstein Barr Virus in vitro (see, e.g., Cole et al., in: MONOCLONAL ANTIBODIES AND CANCER THERAPY, Alan R. Liss, Inc., pp. 77-96 (1985)). For example, a population of nucleic acids that encode regions of antibodies can be isolated. PCR utilizing primers derived from sequences encoding conserved regions of antibodies is used to amplify sequences encoding portions of antibodies from the population and then reconstruct DNAs encoding antibodies or fragments thereof, such as variable domains, from the amplified sequences. Such amplified sequences also can be fused to DNAs encoding other proteins—e.g., a bacteriophage coat, or a bacterial cell surface protein—for expression and display of the fusion polypeptides on phage or bacteria. Amplified sequences can then be expressed and further selected or isolated based, e.g., on the affinity of the expressed antibody or fragment thereof for an antigen or epitope present on the BCMA relevant antigen polypeptide. Alternatively, hybridomas expressing monoclonal antibodies can be prepared by immunizing a subject, e.g., with an isolated polypeptide comprising, or alternatively consisting essentially of, or yet further consisting of, the amino acid sequence of the relevant antigen or a fragment thereof, and then isolating hybridomas from the subject's spleen using routine methods. See, e.g., Milstein et al., (Galfre and Milstein, Methods Enzymol 73: 3-46 (1981)). Screening the hybridomas using standard methods will produce monoclonal antibodies of varying specificity (i.e., for different epitopes) and affinity. A selected monoclonal antibody with the desired properties, e.g., a relevant antigen binding, can be (i) used as expressed by the hybridoma, (ii) bound to a molecule such as polyethylene glycol (PEG) to alter its properties, or (iii) a cDNA encoding the monoclonal antibody can be isolated, sequenced and manipulated in various ways. In one aspect, the monoclonal antibody is produced by a hybridoma which includes a B cell obtained from a transgenic non-human animal, e.g., a transgenic mouse, having a genome comprising a human heavy chain transgene and a light chain transgene fused to an immortalized cell. Hybridoma techniques include those known in the art and taught in Harlow et al., Antibodies: A Laboratory Manual Cold Spring Harbor Laboratory, Cold Spring Harbor, N.Y., 349 (1988); Hammerling et al., Monoclonal Antibodies And T-Cell Hybridomas, 563-681 (1981).

Phage Display Technique. As noted above, the antibodies of the present disclosure can be produced through the application of recombinant DNA and phage display technology. For example, BCMA antibodies, can be prepared using various phage display methods known in the art. In phage display methods, functional antibody domains are displayed on the surface of a phage particle which carries polynucleotide sequences encoding them. Phage with a desired binding property is selected from a repertoire or combinatorial antibody library (e.g., human or murine) by selecting directly with an antigen, typically an antigen bound or captured to a solid surface or bead. Phage used in these methods are typically filamentous phage including fd and M13 with Fab, Fᵥ or disulfide stabilized Fᵥ antibody domains are recombinantly fused to either the phage gene III or gene VIII protein. In addition, methods can be adapted for the construction of Fab expression libraries (see, e.g., Huse et al., Science 246: 1275-1281, 1989) to allow rapid and effective identification of monoclonal Fab fragments with the desired specificity for a relevant antigen polypeptide, e.g., a polypeptide or derivatives, fragments, analogs or homologs thereof. Other examples of phage display methods that can be used to make the isolated antibodies of the present disclosure include those disclosed in Huston et al., Proc. Natl. Acad. Sci. U.S.A., 85: 5879-5883 (1988); Chaudhary et al., Proc. Natl. Acad. Sci. U.S.A., 87: 1066-1070 (1990); Brinkman et al., J. Immunol. Methods 182: 41-50 (1995); Ames et al., J. Immunol. Methods 184: 177-186 (1995); Kettleborough et al., Eur. J. Immunol. 24: 952-958 (1994); Persic et al., Gene 187: 9-18 (1997); Burton et al., Advances in Immunology 57: 191-280 (1994); PCT/GB91/01134; WO 90/02809; WO 91/10737; WO 92/01047; WO 92/18619; WO 93/11236; WO 95/15982; WO 95/20401; WO 96/06213; WO 92/01047 (Medical Research Council et al.); WO 97/08320 (Morphosys); WO 92/01047 (CAT/MRC); WO 91/17271 (Affymax); and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743.

Methods useful for displaying polypeptides on the surface of bacteriophage particles by attaching the polypeptides via disulfide bonds have been described by Lohning, U.S. Pat. No. 6,753,136. As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)₂ fragments can also be employed using methods known in the art such as those disclosed in WO 92/22324; Mullinax et al., BioTechniques 12: 864-869 (1992); Sawai et al., AJRI 34: 26-34 (1995); and Better et al., Science 240: 1041-1043 (1988).

Generally, hybrid antibodies or hybrid antibody fragments that are cloned into a display vector can be selected against the appropriate antigen in order to identify variants that maintained good binding activity, because the antibody or antibody fragment will be present on the surface of the phage or phagemid particle. See, e.g., Barbas III et al., Phage Display, A Laboratory Manual (Cold Spring Harbor Laboratory Press, Cold Spring Harbor, N.Y., 2001). However, other vector formats could be used for this process, such as cloning the antibody fragment library into a lytic phage vector (modified T7 or Lambda Zap systems) for selection and/or screening.

Alternate Methods of Antibody Production. Antibodies may also be produced by inducing in vivo production in the lymphocyte population or by screening recombinant immunoglobulin libraries or panels of highly specific binding reagents (Orlandi et al., PNAS 86: 3833-3837 (1989); Winter, G. et al., Nature, 349: 293-299 (1991)).

Alternatively, techniques for the production of single chain antibodies may be used. Single chain antibodies (scFvs) comprise a heavy chain variable region and a light chain variable region connected with a linker peptide (typically around 5 to 25 amino acids in length). In the scFv, the variable regions of the heavy chain and the light chain may be derived from the same antibody or different antibodies. scFvs may be synthesized using recombinant techniques, for example by expression of a vector encoding the scFv in a host organism such as E. coli. DNA encoding scFv can be obtained by performing amplification using a partial DNA encoding the entire or a desired amino acid sequence of a DNA selected from a DNA encoding the heavy chain or the variable region of the heavy chain of the above-mentioned antibody and a DNA encoding the light chain or the variable region of the light chain thereof as a template, by PCR using a primer pair that defines both ends thereof, and further performing amplification combining a DNA encoding a polypeptide linker portion and a primer pair that defines both ends thereof, so as to ligate both ends of the linker to the heavy chain and the light chain, respectively. An expression vector containing the DNA encoding scFv and a host transformed by the expression vector can be obtained according to conventional methods known in the art.

Antigen binding fragments may also be generated, for example the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse et al., Science, 256: 1275-1281 (1989)).

Antibody Modifications. The antibodies of the present disclosure may be multimerized to increase the affinity for an antigen. The antibody to be multimerized may be one type of antibody or a plurality of antibodies which recognize a plurality of epitopes of the same antigen. As a method of multimerization of the antibody, binding of the IgG CH3 domain to two scFv molecules, binding to streptavidin, introduction of a helix-turn-helix motif and the like can be exemplified.

The antibody compositions disclosed herein may be in the form of a conjugate formed between any of these antibodies and another agent (immunoconjugate). In one aspect, the antibodies disclosed herein are conjugated to radioactive material. In another aspect, the antibodies disclosed herein can be bound to various types of molecules such as polyethylene glycol (PEG).

Antibody Screening. Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between the relevant antigen, or any fragment or oligopeptide thereof and its specific antibody. A two-site, monoclonal-based immunoassay utilizing monoclonal antibodies specific to two non-interfering relevant antigen epitopes may be used, but a competitive binding assay may also be employed (Maddox et al., J. Exp. Med., 158: 1211-1216 (1983)).

Antibody Purification. The antibodies disclosed herein can be purified to homogeneity. The separation and purification of the antibodies can be performed by employing conventional protein separation and purification methods.

By way of example only, the antibody can be separated and purified by appropriately selecting and combining use of chromatography columns, filters, ultrafiltration, salt precipitation, dialysis, preparative polyacrylamide gel electrophoresis, isoelectric focusing electrophoresis, and the like. Strategies for Protein Purification and Characterization: A Laboratory Course Manual, Daniel R. Marshak et al. eds., Cold Spring Harbor Laboratory Press (1996); Antibodies: A Laboratory Manual. Ed Harlow and David Lane, Cold Spring Harbor Laboratory (1988).

Examples of chromatography include affinity chromatography, ion exchange chromatography, hydrophobic chromatography, gel filtration chromatography, reverse phase chromatography, and adsorption chromatography. In one aspect, chromatography can be performed by employing liquid chromatography such as HPLC or FPLC.

In one aspect, a Protein A column or a Protein G column may be used in affinity chromatography. Other exemplary columns include a Protein A column, Hyper D, POROS, Sepharose F. F. (Pharmacia) and the like.

### Bispecific Antibodies

Also provided herein are bispecific antibody constructs that comprises any antibodies of NKG2D or of a tumor antigen, e.g., antigen binding domain of an anti-NKG2D antibody and a tumor targeting antigen binding domain of an antibody that binds to CST. In some embodiments, provided is a bispecific antibody comprising, or alternatively consisting essentially of, or yet consisting of an antigen binding domain that recognizes and binds a TAA on a cancer cell and an antigen binding domain the recognizes and binds NKG2D. In one embodiment, provided is a bispecific antibody comprising, or alternatively consisting essentially of, or yet consisting of an antigen binding domain that recognizes and binds CS1 (i.e., anti-CS1 antigen binding domain) and an antigen binding domain the recognizes and binds NKG2D (i.e., anti-NKG2D antigen binding domain). In some embodiments, the antigen binding domain binds an antigen present on the type of cancer or tumor cell that the antigen binding domain of the CAR binds, e.g., each binds a MM cell or a glioblastoma cell. The antigen binding domains can be from any appropriate species, e.g., murine, human or a humanized sequence. An example of such is described in PCT/US2016/018955, filed Feb. 22, 2016, incorporated herein by reference specifically including the polynucleotide and amino acid sequence of such.

Described herein as an example of this approach is a construct wherein at least one of the two antigen binding domains is specific for an antigen that is co-expressed with a predetermined first antigen. For example, in the case of MM, BCMA and CS1 are co-expressed on MM, and CS1 was selected to compliment the BCMA antigen binding domain of a CAR construct. Alternatively, BCMA can complement an anti-CS1 CAR.

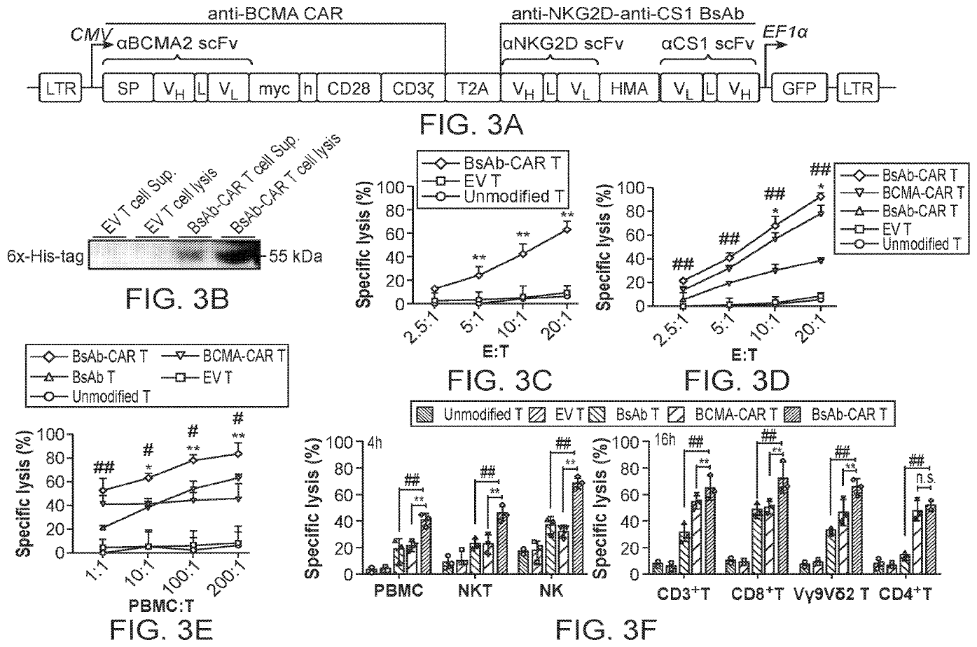

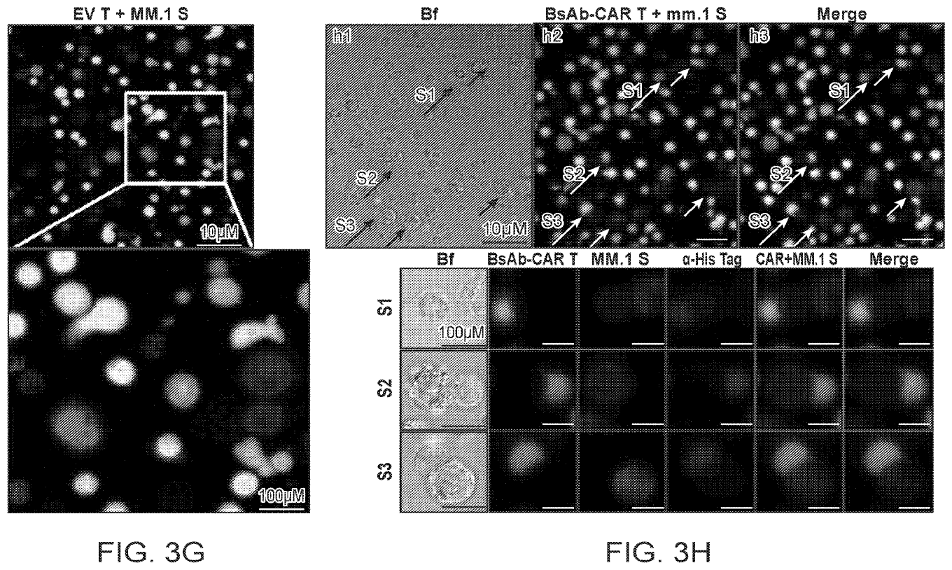

In a further aspect, the antigen binding domains comprise, or consist essentially of, or yet further consist of a scFv fragment that is optionally codon-optimized. In a further aspect, the bispecific antigen binding domains are the variable heavy and light chains that are joined by a peptide linker. In general, the antigen binding domains can be joined together by a peptide linker, e.g., a non-immunogenic protein linker derived from human muscle aldose (HMA)³⁵. An example of such is provided in FIG. 3A, and the bispecific components shown in FIG. 1E, 1F or 1G. As shown in these figures, the constructs can further comprise T2A, a self-cleaving 2A gene, an HMA polypeptide and/or a linker peptide, e.g., A G4S peptide linker (SEQ ID NOs: 14 and 134 to 147) (“G4S” disclosed as SEQ ID NO: 134).

In certain embodiments, the anti-TAA antigen binding domain of a bispecific antibody as disclosed herein recognizes and binds any cancer- or tumor-associated antigen, such as one or more selected from FLT3, CD19, mesothelin, human epidermal growth factor receptor 2 (HER2), prostate stem cell antigen (PSCA), carcinoembryonic antigen (CEA), GTP-ase-activating protein (GAP), ganglioside G2 (GD2), CD5, prostate specific membrane antigen (PSMA), receptor tyrosine kinase-like orphan receptor 1 (ROR1), CD123, CD70, CD38, B cell maturation antigen (BCMA), mucin 1, (Muc1), ephrin type-A receptor 2 precursor (EphA2), wildtype epidermal growth factor receptor (EGFRwt), epidermal growth factor receptor variant III (EGFRVIII), interleukin 13 receptor alpha 2 (IL13Ra2), CD133, glypican 3 (GPC3), epithelial cell adhesion molecule precursor (EpCam), fibroblast activation protein alpha (FAP), vascular endothelial growth factor receptor 2 (VEGFR2), cancer/testis (CT), guanylyl cyclase C (GUCY2C), tumor-associated glycoprotein-72 (TAG-72), thymidine kinase 1 (TK1), and hypoxanthine guanine phosphoribosyltransferase (HPRT1). These may comprise the heavy and light chains of the antigen binding domains, that alternatively consist of or comprise the CDRs of the antigen binding domains.

In some embodiments, the anti-TAA antigen binding domain of a bispecific antibody as disclosed herein recognizes and binds fins-like tyrosine kinase 3 (FLT3), i.e., is an anti-FLT3 antigen binding domain. In some embodiments, the anti-FLT3 antigen binding domain comprises, or alternatively consists essentially of, or yet consists of one or two or three or four or five or all six of the following CDRs: a CDRL1 comprising, or alternatively consisting essentially of, or yet consisting of SEQ ID NO: 15, SEQ ID NO: 16, or an equivalent of each thereof; a CDRL2 comprising, or alternatively consisting essentially of, or yet consisting of SEQ ID NO: 17, SEQ ID NO: 18, or an equivalent of each thereof, a CDRL3 comprising, or alternatively consisting essentially of, or yet consisting of SEQ ID NO: 19, SEQ ID NO: 20, or an equivalent of each thereof; a CDRH1 comprising, or alternatively consisting essentially of, or yet consisting of SEQ ID NO: 21, SEQ ID NO: 22, or an equivalent of each thereof, a CDRH2 comprising, or alternatively consisting essentially of, or yet consisting of SEQ ID NO: 23, SEQ ID NO: 24, or an equivalent of each thereof, and a CDRH3 comprising, or alternatively consisting essentially of, or yet consisting of SEQ ID NO: 25, SEQ ID NO: 26, or an equivalent of each thereof. In further embodiments, the CDR equivalent(s) thereof and/or the antigen binding domain comprising, or alternatively consisting essentially of, or yet consisting of the equivalent(s) recognize and bind FLT3. In some embodiments, the anti-FLT3 antigen binding domain comprises, or alternatively consists essentially of, or yet consists of a light chain variable region comprising, or alternatively consisting essentially of, or yet consisting of a sequence selected from SEQ ID NO: 27, SEQ ID NO: 28, or an equivalent of each thereof; and/or a heavy chain variable region comprising, or alternatively consisting essentially of, or yet consisting of a sequence selected from SEQ ID NO: 29, SEQ ID NO: 30 or an equivalent of each thereof. In further embodiments, the variable equivalent(s) thereof and/or an antigen binding domain comprising, or alternatively consisting essentially of, or yet consisting of the equivalent(s) recognizes and binds FLT3.

In some embodiments, the anti-TAA antigen binding domain of a bispecific antibody as disclosed herein recognizes and binds epidermal growth factor receptor (EGFR), for example wildtype EGFR (EGFRwt) and/or a variant thereof, such as EGFRvIII. This antigen binding domain is also referred to herein as an anti-EGFR antigen binding domain. In some embodiments, the anti-EGFR antigen binding domain comprises, or alternatively consists essentially of, or yet consists of one or two or three or four or five or six of the following CDRs: a CDRL1 comprising, or alternatively consisting essentially of, or yet consisting of SEQ ID NO: 31, SEQ ID NO: 32, or an equivalent of each thereof; a CDRL2 comprising, or alternatively consisting essentially of, or yet consisting of SEQ ID NO: 33, SEQ ID NO: 34, or an equivalent of each thereof; a CDRL3 comprising, or alternatively consisting essentially of, or yet consisting of SEQ ID NO: 35, SEQ ID NO: 36, or an equivalent of each thereof; a CDRH1 comprising, or alternatively consisting essentially of, or yet consisting of SEQ ID NO: 37, SEQ ID NO: 38, or an equivalent of each thereof; a CDRH2 comprising, or alternatively consisting essentially of, or yet consisting of SEQ ID NO: 39, SEQ ID NO: 40, or an equivalent of each thereof; and a CDRH3 comprising, or alternatively consisting essentially of, or yet consisting of SEQ ID NO: 41, SEQ ID NO: 42, or an equivalent of each thereof. In further embodiments, the CDR equivalent(s) thereof and/or an antigen binding fragment comprising, or alternatively consisting essentially of, or yet consisting of the equivalent(s) recognize and bind EGFR. In some embodiments, the anti-EGFR antigen binding domain comprises, or alternatively consists essentially of, or yet consists of a light chain variable region comprising, or alternatively consisting essentially of, or yet consisting of a sequence selected from SEQ ID NO: 43, SEQ ID NO: 44, or an equivalent of each thereof and/or a heavy chain variable region comprising, or alternatively consisting essentially of, or yet consisting of a sequence selected from SEQ ID NO: 45, SEQ ID NO: 46, or an equivalent of each thereof. In further embodiments, the variable region equivalent(s) thereof and/or an antigen binding fragment comprising, or alternatively consisting essentially of, or yet consisting of the equivalent(s) recognize and bind EGFR.

In some embodiments, the anti-TAA antigen binding domain of a bispecific antibody as disclosed herein recognizes and binds BCMA, i.e., an anti-BCMA antigen binding domain. In some embodiments, the antigen binding domain comprises, or alternatively consists essentially of, or yet consists of one or two or three or four or five or six of the following CDRs: a CDRL1 comprising, or alternatively consisting essentially of, or yet consisting of SEQ ID NO: 47, SEQ ID NO: 48, SEQ ID NO: 49, or an equivalent of each thereof, a CDRL2 comprising, or alternatively consisting essentially of, or yet consisting of SEQ ID NO: 50, SEQ ID NO: 51, SEQ ID NO: 52, or an equivalent of each thereof, a CDRL3 comprising, or alternatively consisting essentially of, or yet consisting of SEQ ID NO: 53, SEQ ID NO: 54, SEQ ID NO: 55, or an equivalent of each thereof, a CDRH1 comprising, or alternatively consisting essentially of, or yet consisting of SEQ ID NO: 56, SEQ ID NO: 57, SEQ ID NO: 58, or an equivalent of each thereof; a CDRH2 comprising, or alternatively consisting essentially of, or yet consisting of SEQ ID NO: 59, SEQ ID NO: 60, SEQ ID NO: 61, or an equivalent of each thereof; and a CDRH3 comprising, or alternatively consisting essentially of, or yet consisting of SEQ ID NO: 62, SEQ ID NO: 63, SEQ ID NO: 64, or an equivalent of each thereof. In further embodiments, the CDR equivalent(s) thereof and/or an antigen binding fragment comprising, or alternatively consisting essentially of, or yet consisting of the equivalent(s) recognize and bind BCMA. In some embodiments, the anti-BCMA antigen binding domain comprises, or alternatively consists essentially of, or yet consists of a light chain variable region comprising, or alternatively consisting essentially of, or yet consisting of a sequence selected from SEQ ID NO: 65, SEQ ID NO: 66, SEQ ID NO: 67, or an equivalent of each thereof and/or a heavy chain variable region comprising, or alternatively consisting essentially of, or yet consisting of a sequence selected from SEQ ID NO: 68, SEQ ID NO: 69, SEQ ID NO: 70, or an equivalent of each thereof. In further embodiments, the variable region equivalent(s) thereof and/or an antigen binding fragment comprising, or alternatively consisting essentially of, or yet consisting of the equivalent(s) recognize and bind BCMA.

In some embodiments, the anti-TAA antigen binding domain of a bispecific antibody as disclosed herein recognizes and binds CS1, i.e., an anti-CS1 antigen binding domain. In some embodiments, the anti-CS1 antigen binding domain comprises, or alternatively consists essentially of, or yet consists of one or two or three or four or five or six of the following CDRs: a CDRL1 comprising, or alternatively consisting essentially of, or yet consisting of SEQ ID NO: 71 or an equivalent thereof, a CDRL2 comprising, or alternatively consisting essentially of, or yet consisting of SEQ ID NO: 72 or an equivalent thereof, a CDRL3 comprising, or alternatively consisting essentially of, or yet consisting of SEQ ID NO: 73 or an equivalent thereof; a CDRH1 comprising, or alternatively consisting essentially of, or yet consisting of SEQ ID NO: 74 or an equivalent thereof, a CDRH2 comprising, or alternatively consisting essentially of, or yet consisting of SEQ ID NO: 75 or an equivalent thereof, and a CDRH3 comprising, or alternatively consisting essentially of, or yet consisting of SEQ ID NO: 76 or an equivalent thereof. In further embodiments, the CDR equivalent(s) thereof and/or an antigen binding fragment comprising, or alternatively consisting essentially of, or yet consisting of the equivalent(s) recognize and bind CS1. In some embodiments, the anti-CS1 antigen binding domain comprises, or alternatively consists essentially of, or yet consists of a light chain variable region comprising, or alternatively consisting essentially of, or yet consisting of a sequence selected from SEQ ID NO: 77, SEQ ID NO: 154, SEQ ID NO: 78, or an equivalent of each thereof and/or a heavy chain variable region comprising, or alternatively consisting essentially of, or yet consisting of a sequence selected from SEQ ID NO: 79, SEQ ID NO: 149, SEQ ID NO: 80, SEQ ID NO:150, or an equivalent of each thereof. In further embodiments, the variable region equivalent(s) thereof and/or an antigen binding fragment comprising, or alternatively consisting essentially of, or yet consisting of the equivalent(s) recognize and bind CS1.

In some embodiments, the anti-NKG2D antigen binding domain comprises, or alternatively consists essentially of, or yet consists of one or two or three or four or five or all six of the following complementarity-determining regions (CDRs): a light chain complementarity-determining region 1 (CDRL1) comprising, or alternatively consisting essentially of, or yet consisting of SGSSSNIGNNAVN (SEQ ID NO: 6) or an equivalent thereof, a light chain complementarity-determining region 2 (CDRL2) comprising, or alternatively consisting essentially of, or yet consisting of YDDLLPS (SEQ ID NO: 7) or an equivalent thereof, a light chain complementarity-determining region 3 (CDRL3) comprising, or alternatively consisting essentially of, or yet consisting of AAWDDSLNGPV (SEQ ID NO: 8) or an equivalent thereof, a heavy chain complementarity-determining region 1 (CDRH1) comprising, or alternatively consisting essentially of, or yet consisting of GFTFSSY (SEQ ID NO: 9) or an equivalent thereof, a heavy chain complementarity-determining region 2 (CDRH2) comprising, or alternatively consisting essentially of, or yet consisting of RYDGSN (SEQ ID NO: 10) or an equivalent thereof, and a heavy chain complementarity-determining region 3 (CDRH3) comprising, or alternatively consisting essentially of, or yet consisting of DRGLGDGTYFDY (SEQ ID NO: 11) or an equivalent thereof. In further embodiments, the CDR equivalent(s) thereof and/or an antigen binding fragment comprising, or alternatively consisting essentially of, or yet consisting of the equivalent(s) recognize and bind NKG2D. In some embodiments, the anti-NKG2D antigen binding domain comprises, or alternatively consists essentially of, or yet consists of a light chain variable region comprising, or alternatively consisting essentially of, or yet consisting of

QSALTQPASVSGSPGQSITISCSGSSSNIGNNAVNWYQQLPGKAPKLLIYYDDLLPSG VSDRFSGSKSGTSAFLAISGLQSEDEADYYCAAWDDSLNGPVFGGGTKLTVL (SEQ ID NO: 12), or an equivalent thereof; and/or a heavy chain variable region comprising, or alternatively consisting essentially of, or yet consisting of
QVQLVESGGGLVKPGGSLRLSCAASGFTFSSYGMHWVRQAPGKGLEWVAFIRYDG SNKYYADSVKGRFTISRDNSKNTLYLQMNSLRAEDTAVYYCAKDRGLGDGTYFD YWGQGTTVTVSS (SEQ ID NO: 13), or an equivalent thereof. In further embodiments, the variable region equivalent(s) thereof and/or an antigen binding fragment comprising, or alternatively consisting essentially of, or yet consisting of the equivalent(s) recognize and bind NKG2D.

In some embodiments, an equivalent of a reference protein or polypeptide, such as a CDR, a variable region, an antigen binding domain, or an antibody, is at least 80%, or at least 85%, or at least 90%, or at least 95%, or at least 96%, or at least 97%, or at least 98%, or at least 99% identical to the reference protein or polypeptide.

In some embodiments, an equivalent of a reference protein or polypeptide, such as a CDR, a variable region, an antigen binding domain, or an antibody, comprises, or alternatively consists essentially of, or yet consists of the amino acid sequence of C terminus to N terminus of the reference protein or polypeptide. Such equivalent is also referred to herein as a reverse sequence. For example, the reference protein or polypeptide comprising, or alternatively consisting essentially of, or yet consisting of KASQDVITGVA (SEQ ID NO: 71), and the equivalent thereof comprises, or alternatively consists essentially of, or yet consists of AVGTIVDQSAK (SEQ ID NO: 151). In the embodiments of a CDR, a variable region, an antigen binding domain, or an antibody as the reference protein or polypeptide, the equivalent still recognizes and binds the same epitope of the reference.

In some embodiments, an antigen binding domain as disclosed herein further comprises a peptide linker, for example, between two CDRs and/or between its light chain variable region or the equivalent thereof and its heavy chain variable region or the equivalent thereof. In one embodiments, the peptide linker comprises, or alternatively consists essentially of, or yet consists of GGGGSGGGGSGGGGS (SEQ ID NO: 14) or an equivalent thereof. In some embodiments, an equivalent of SEQ ID NO: 14 may be from 1 to 50 amino acids long, for instance, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. In some embodiments, an equivalent of SEQ ID NO: 14 is glycine rich, although it may also contain serine or threonine. In some embodiments, an equivalent of SEQ ID NO: 14 comprises, or alternatively consists essentially of, or yet consists of (GGGGS)n, wherein n may be an integer of 1, or 2, or 3, or 4, or 5, or 6, or 7, or 8, or 9, or 10, or 11, or 12, or 13, or 14, or 15, or more (SEQ ID NOs:134-135, 14 and 136-147).

In some embodiments, the bispecific antibody (BsAb) further comprises a signal peptide, optionally at its N terminus, optionally directing the bispecific antibody to be secreted out of a BsAb or BsAb-CAR expressing cell and/or facilitating secretion of the bispecific antibody, fragment or equivalent thereof. In some embodiments, the signal peptide is located at the N terminus of the bispecific antibody, for example, on the N terminus side to both of the antigen binding domains of the bispecific antibody. In some embodiments, the signal peptide is an IL2 signal peptide. In some embodiments, the signal peptide comprises, or consists essentially of, or yet further consists of MYRMQLLSCIALSLALVTNS (SEQ ID NO: 4) or an equivalent thereof. In further embodiments, an equivalent of SEQ ID NO: 4 still directs the bispecific antibody to be secreted out of a BsAb or BsAb-CAR expressing cell and/or facilitates secretion of the bispecific antibody, fragment or equivalent thereof. Additionally or alternatively, an equivalent of SEQ ID NO: 4 is at least 80% (such as at least 85%, at least 90%, or at least 91%, or at least 92%, or at least 93%, or at least 94%, at least 95%, at least 96%, at least 97%, at least 98%, or at least 99%) identical to SEQ ID NO: 4. Methods of detecting whether a signal peptide directing a protein to be secreted out of a BsAb or BsAb-CAR expressing cell are available to one of skill in the art, such as immunostaining revealing such protein in supernatant of the cell culture.

In some embodiments, the bispecific antibody (BsAb) further comprises a detectable or purification marker. In one embodiment, the detectable marker comprises, or alternatively consists essentially of, or yet consists of YPYDVPDYA (SEQ ID NO: 84).

In some embodiments, an antigen binding domain as disclosed herein further comprises a fragment crystallizable (Fc) region of an immunoglobulin, a mutant thereof, or an equivalent thereof. In some embodiments, the Fc region or a mutant thereof is a human Fc region or a mutant thereof. In some embodiments, the Fc region or a mutant thereof is an avian (such as chicken), or murine, or bovine, or swine, or camel, or llamas, or shark Fc region or a mutant thereof. In one embodiments, the Fc region comprises, or alternatively consists essentially of, or yet consists of

ESKYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLPS SIEKTISKAKGQPREPQVYTLPPSQEEMTKNQVSLTCLVKGFYPSDIAVEWESNGQP ENNYKTTPPVLDSDGSFFLYSRLTVDKSRWQEGNVFSCSVMHEALHNHYTQKSLS LSLGK (SEQ ID NO: 81), or an equivalent thereof. In some embodiments, the Fc region comprises, or alternatively consists essentially of, or yet consists of
ESKYGPPCPPCPAPEAAGGPSVFLFPPKPKDTLMISRTPEVTCVVVDVSQEDPEVQF NWYVDGVEVHNAKTKPREEQFQSTYRVVSVLTVLHQDWLNGKEYKCKVSNKGL PSSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 82) or an Fc equivalent having mutations at a position corresponding to amino acid (aa) 16, aa 17 and aa 79 of SEQ ID NO: 81. In further embodiments, the Fc equivalent, variant, or mutant binds an Fc receptor on an immune cell and/or on a platelet. Additionally or alternatively, the Fc equivalent, variant, or mutant binds a complement protein. In some embodiments, an equivalent comprises 1, or 2, or 3, or 4, or 5, or 6, or 7, or 8, or 9, or 10, or 11, or 12, or 13, or 14, or 15, or more mutations compared to its reference.

In some embodiments, the bispecific antibody further comprises a peptide linker between the two antigen binding domains. In some embodiments, the peptide linker is derived from human muscle aldolase (HMA), optionally comprising, or alternatively consisting essentially of, or yet consisting of PSGQAGAAASESLFVSNHAY (SEQ ID NO: 83). In one embodiments, the peptide linker comprises, or alternatively consists essentially of, or yet consists of GGGGSGGGGSGGGGS (SEQ ID NO: 14) or an equivalent thereof. In some embodiments, an equivalent of SEQ ID NO: 14 may be from 1 to 50 amino acids long, for instance, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. In some embodiments, an equivalent of SEQ ID NO: 14 is glycine rich, although it may also contain serine or threonine. In some embodiments, an equivalent of SEQ ID NO: 14 comprises, or alternatively consists essentially of, or yet consists of (GGGGS)n, wherein n may be an integer of 1, or 2, or 3, or 4, or 5, or 6, or 7, or 8, or 9, or 10, or 11, or 12, or 13, or 14, or 15, or more (SEQ ID NOs:134-135, 14 and 136-147). In some embodiments, the peptide linker here is a cleavable peptide. In further embodiments, the peptide linker here is a self-cleaving peptide. In yet further embodiments, the peptide linker here is a T2A peptide, optionally comprising, or alternatively consisting essentially of, or yet consisting of HVGSGEGRGSLLTCGDVEENPGP (SEQ ID NO: 85) or an equivalent thereof. In some embodiments, an equivalent of SEQ ID NO: 85 comprises 1, or 2, or 3, or 4, or 5, or 6, or 7, or 8, or 9, or 10, or 11, or 12, or 13, or 14, or 15, or more mutations compared to SEQ ID NO: 85 and is still capable of self-cleaving, and thus releasing two products from a single polypeptide. Methods of evaluating such self-cleavage are available for one of skill in the art, such as via western blot detecting molecular size of the products and/or polypeptides.

In some embodiments, the bispecific antibody comprises, or alternatively consists essentially of, or yet consists of, optionally from the N terminus to C terminus, an optional signal peptide, an anti-TAA heavy chain variable region, a first optional peptide linker, an anti-TAA light chain variable region, an optional Fc region or a mutant thereof or an equivalent of each thereof, a second optional peptide linker, an anti-NKG2D heavy chain variable region, a third optional peptide linker, an anti-NKG2D light chain variable region, an optional Fc region or a mutant thereof or an equivalent of each thereof.

In some embodiments, the bispecific antibody comprises, or alternatively consists essentially of, or yet consists of, optionally from the N terminus to C terminus, an optional signal peptide, an anti-TAA heavy chain variable region, a first optional peptide linker, an anti-TAA light chain variable region, an optional Fe region or a mutant thereof or an equivalent of each thereof, a second optional peptide linker, an anti-NKG2D light chain variable region, a third optional peptide linker, an anti-NKG2D heavy chain variable region, an optional Fc region or a mutant thereof or an equivalent of each thereof.

In some embodiments, the bispecific antibody comprises, or alternatively consists essentially of, or yet consists of, optionally from the N terminus to C terminus, an optional signal peptide, an anti-TAA light chain variable region, a first optional peptide linker, an anti-TAA heavy chain variable region, an optional Fc region or a mutant thereof or an equivalent of each thereof, a second optional peptide linker, an anti-NKG2D heavy chain variable region, a third optional peptide linker, an anti-NKG2D light chain variable region, an optional Fc region or a mutant thereof or an equivalent of each thereof.

In some embodiments, the bispecific antibody comprises, or alternatively consists essentially of, or yet consists of, optionally from the N terminus to C terminus, an optional signal peptide, an anti-TAA light chain variable region, a first optional peptide linker, an anti-TAA heavy chain variable region, an optional Fc region or a mutant thereof or an equivalent of each thereof, a second optional peptide linker, an anti-NKG2D light chain variable region, a third optional peptide linker, an anti-NKG2D heavy chain variable region, an optional Fc region or a mutant thereof or an equivalent of each thereof.

In some embodiments, variable regions of the antigen binding domain at the C terminus of the bispecific antibody comprise, or alternatively consist essentially of, or yet consist of a reversed sequence, i.e., C terminus to N terminus of the variable region sequence if present in a monospecific antibody. In further embodiments, the antigen binding domain at the N terminus of the bispecific antibody further comprises a Fc region or a mutant thereof or an equivalent of each thereof. In yet further embodiments, In further embodiments, the antigen binding domain at the C terminus of the bispecific antibody further does not comprise a Fc region or a mutant thereof or an equivalent of each thereof.

Also provided are polynucleotides (such as isolated and/or engineered polypeptides) and/or isolated nucleic acids that encode the bispecific antibodies. The polynucleotides and/or nucleic acids can further comprise the necessary regulatory sequences, e.g., a promoter for expression in a host cell, e.g., a mammalian or human host cell such as a T cell or an NK cell and/or an enhancer. In some embodiments, the polynucleotides and/or nucleic acids further comprise a first regulatory sequence directing the expression of the CAR. In further embodiments, the regulatory sequences comprises one or more of the following: a promoter, an intron, an enhancer, or a polyadenylation signal. In some embodiments, the promoter is a CMV or an EF1 alpha promoter. In further embodiments, the polynucleotide and/or isolated nucleic acid can further comprise polynucleotides encoding a detectable or purification marker such as GFP that may be located downstream from the BsAb coding polynucleotide and regulated from a separate regulatory element (a second regulatory sequence), e.g., a promoter optionally selected from an EF1alpha promoter. As is apparent to the skilled artisan, the promoter(s) are selected for the host expression system. As shown in FIG. 3A, the constructs can further comprise T2A, a self-cleaving 2A gene. In some embodiments, the polynucleotides and/or nucleic acids further comprise a suicide gene encoding a suicide gene product optionally selected from one or more of: HSV-TK (Herpes simplex virus thymidine kinase), cytosine deaminase, nitroreductase, carboxylesterase, cytochrome P450 or PNP (Purine nucleoside phosphorylase), truncated EGFR, or inducible caspase (“iCasp”). In further embodiments, the polynucleotides and/or nucleic acids further comprise a third regulatory sequence directing expression of the suicide gene product. In one embodiment, any one or two or three of the regulatory sequences is or are inducible or constitutively active in the cell. In one embodiment, any one or two or three of the regulatory sequences can be cell specific or tissue specific.

In some embodiments, the polynucleotides and/or nucleic acids comprise, or alternatively consist essentially of, or yet consist of one or more of the following:

(I) a nucleotide sequence encoding a signal peptide, and wherein the nucleotide sequence comprises, or alternatively consists essentially of, or yet consists of SEQ ID NO: 86, SEQ ID NO: 87, or an equivalent of each thereof;
(II) a nucleotide sequence encoding a linker peptide, and wherein the nucleotide sequence comprises, or alternatively consists essentially of, or yet consists of SEQ ID NO: 88;
(V) a nucleotide sequence encoding a cleavable peptide, and wherein the nucleotide sequence comprises, or alternatively consists essentially of, or yet consists of CACGTGGGTTCTGGAGAAGGACGCGGTTCCTTGTTGACGTGTGGCGATGTAGAG GAAAATCCGGGTCCA (SEQ ID NO: 91) or an equivalent thereof,
(VI) a nucleotide sequence encoding a linker, and wherein the nucleotide sequence comprises, or alternatively consists essentially of, or yet consists of SEQ ID NO: 92 or an equivalent thereof;
(VII) a nucleotide sequence encoding an anti-BCMA light chain variable region, and wherein the nucleotide sequence is selected from: SEQ ID NO: 93, SEQ ID NO: 94, SEQ ID NO: 95, SEQ ID NO: 96 or an equivalent of each thereof; (VIII) a nucleotide sequence encoding an anti-BCMA heavy chain variable region, and wherein the nucleotide sequence is selected from: SEQ ID NO: 97, SEQ ID NO: 98, SEQ ID NO: 99, or SEQ ID NO: 100 or an equivalent of each thereof;
(IX) a nucleotide sequence encoding an anti-NKG2D light chain variable region, and wherein the nucleotide sequence comprises, or alternatively consists essentially of, or yet consists of SEQ ID NO: 101 or an equivalent thereof;
(X) a nucleotide sequence encoding an anti-NKG2D heavy chain variable region, and wherein the nucleotide sequence comprises, or alternatively consists essentially of, or yet consists of SEQ ID NO: 102 or an equivalent thereof;
(XI) a nucleotide sequence encoding an anti-FLT3 light chain variable region, and wherein the nucleotide sequence comprises, or alternatively consists essentially of, or yet consists of SEQ ID NO: 103 SEQ ID NO: 104 or an equivalent of each thereof;
(XII) a nucleotide sequence encoding an anti-FLT3 heavy chain variable region, and wherein the nucleotide sequence comprises, or alternatively consists essentially of, or yet consists of SEQ ID NO: 105, SEQ ID NO: 106 or an equivalent of each thereof;
(XIII) a nucleotide sequence encoding an anti-CS1 light chain variable region, and wherein the nucleotide sequence comprises, or alternatively consists essentially of, or yet consists of SEQ ID NO: 107 or an equivalent thereof; and
(XIV) a nucleotide sequence encoding an anti-CS1 heavy chain variable region, and wherein the nucleotide sequence comprises, or alternatively consists essentially of, or yet consists of SEQ ID NO: 108 or an equivalent thereof.

In some embodiments, an equivalent of a reference nucleotide sequence encodes the same amino acid sequence of the reference. In some embodiments, an equivalent of a reference nucleotide sequence is at least 80% (such as at least 85%, at least 90%, or at least 91%, or at least 92%, or at least 93%, or at least 94%, at least 95%, at least 96%, at least 97%, at least 98%, or at least 99%) identical to the reference. Additionally or alternatively, an equivalent of a reference nucleotide sequence hybridizes under conditions of high stringency to the complement of the reference. In some embodiments, conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. Also provided is a polynucleotide that is reverse, complement, or reverse-complement to the polynucleotide as disclosed herein. In some embodiments, a polynucleotide as disclosed herein further comprise a detectable or purification marker and/or a sequence encoding a detectable or purification marker.

The polynucleotides and/or isolated nucleic acid can be inserted into a vector, such as an expression vector, e.g., a lentiviral vector, between the 5′ and 3′ LTRs. FIG. 1B is an exemplary lentiviral vector construct of this disclosure wherein in one aspect, the antigen binding domain of the anti-tumor or anti-cancer antibody is other than an anti-BMCA antibody. As is apparent to the skilled artisan, the constructs may not comprise a marker peptide or purification marker. In some embodiments, the vector is a non-viral vector, such as a plasmid. In some embodiments, the vector is a viral vector, optionally selected from the group of a retroviral vector, a lentiviral vector, an adenoviral vector, and an adeno-associated viral vector. Additionally or alternatively, the vector further comprises a regulatory sequence directing replication and/or expression of the antibody encoding polynucleotide and/or isolated nucleic acid. In further embodiments, the regulatory sequence comprises one or more of the following: a promoter, an intron, an enhancer, or a polyadenylation signal. In some embodiments, the vector further comprises a detectable or purification marker.

### BsAb-CAR Constructs

Thus, in one aspect, provided herein are BsAb-CARs (also referred to herein as the BsAb-CAR constructs, BsAb-CAR polypeptides, or polypeptides) comprising, or alternatively consisting essentially of, or yet further consisting of: (a) an antigen binding domain of a cancer or tumor targeting antibody; (b) a hinge domain; (c) a transmembrane domain; and (d) an intracellular domain and a bispecific antibody comprising, or alternatively consists essentially of, or yet consists of as one element, a NKG2D antigen binding domain and a second antigen binding domain selected to bind to the same cell type as the antigen binding domain of the CAR. In one aspect, provided is a polypeptide comprising, or alternatively consisting essentially of, or yet consisting of a CAR as disclosed herein and a bispecific antibody as disclosed herein. In one aspect, provided is a polypeptide comprising, or alternatively consisting essentially of, or yet consisting of (i) an amino acid sequence of a chimeric antigen receptor (CAR) comprising, or alternatively consisting essentially of, or yet consisting of (1) an antigen binding domain that recognizes and binds a first tumor associated antigen (TAA) on a cancer cell (first anti-TAA antigen binding domain) with the proviso that the first TAA is not a B-cell maturation antigen (BCMA); (2) a hinge domain; (3) a transmembrane domain; and (4) an intracellular domain; and (ii) a bispecific antibody comprising, or alternatively consisting essentially of, or yet consisting of (1) an antigen binding domain that recognizes and binds a second TAA on the cancer cell (second anti-TAA antigen binding domain); and (2) an antigen binding domain that recognizes and binds NKG2D (anti-NKG2D antigen binding domain). In another aspect, provided is a polypeptide comprising, or alternatively consisting essentially of, or yet consisting of (i) an amino acid sequence of a CAR comprising, or alternatively consisting essentially of, or yet consisting of (1) an antigen binding domain that recognizes and binds BCMA (anti-BCMA antigen binding domain); (2) a hinge domain; (3) a transmembrane domain; and (4) an intracellular domain; and (ii) a bispecific antibody comprising, or alternatively consisting essentially of, or yet consisting of (1) an antigen binding domain that recognizes and binds NKG2D (anti-NKG2D antigen binding domain) and (2) an antigen binding domain that recognizes and binds CS1 (anti-CS1 antigen binding domain). The CAR element can further comprise a signal peptide. A signal peptide also can be located before the BsAb portion of the BsAb-CAR construct. In some embodiments, the intracellular domain comprises one or more, or two or more costimulatory domains that can comprise, consist essentially of, or yet consist of a CD28 costimulatory signaling region and/or a 4-1BB costimulatory domain. In some embodiments, the first TAA and the second TAA are expressed on the same cancer or tumor type and/or cell.

In certain embodiments, the antigen binding domain of the tumor targeting antibody comprises, or alternatively consists essentially of, or further consists of a heavy chain variable region and a light chain variable region that are optionally linked by a linker peptide. In some embodiments, the heavy and/or light chain variable region comprises, or alternatively consists essentially of, or further consists of the relevant CDR regions of an antibody to any one of B-cell maturation antigen (BCMA) and/or SLAMF7 (also known as CS1 or CD319), and/or an equivalent of each thereof. In some embodiments, the tumor targeting antibody targets BCMA and the bispecific antibody targets or binds the same cell or tumor type, e.g., glioblastoma, MM or AML. In some embodiments, the heavy chain and/or light chain variable region comprises, or alternatively consists essentially of, or further consists of the amino acid sequence of an antibody to any one of B-cell maturation antigen (BCMA) and the bispecific comprises one or more of the CDRs, the heavy and light chains, or the scFv and/or Fv fragments of an anti-SLAMF7 antibody (also known as anti-CS1 or anti-CD319 antibody), and/or an equivalent of each thereof. In some embodiments, the CAR portion of the construct further comprises, or alternatively consisting essentially of, or yet further consisting of a signaling domain and/or a signal peptide. In some embodiments, the first TAA is BCMA and the second TAA is CST In some embodiments, the first TAA is CS1 and the second TAA is BCMA.

In some embodiments, the BsAb comprises an antigen binding domain of an anti-FLT3 antibody and the BsAb portion comprises an antigen binding domain of an anti-NKG2D and an antigen binding domain of an anti-CD123 antibody. This BsAb is a therapy for the treatment of AML and methods to make and use this BsAb CAR are within the scope of this disclosure. An effective amount of the BsAb is administered to a patient suffering from AML. These methods can be supplemented with appropriate diagnostic methods to diagnose AML and monitor treatment and toxicity. In some embodiments, the first TAA is FLT3 and the second TAA is CD123. In some embodiments, the first TAA is CD123 and the second TAA is FLT3.

In some embodiments, the BsAb comprises an antigen binding domain of an anti-EGFR antibody and the BsAb portion comprises an antigen binding domain of an anti-NKG2D and an antigen binding domain of an anti-IL13Ra2 antibody. This BsAb is a therapy for the treatment of glioblastoma and methods to make and use this BsAb CAR are within the scope of this disclosure. An effective amount of the BsAb is administered to a patient suffering from glioblastoma. Administration can be local (intracranial administration) or systemic. These methods can be supplemented with appropriate diagnostic methods to diagnose glioblastoma and monitor treatment and toxicity. In some embodiments, the first TAA is EGFR and the second TAA is IL13Ra2. In some embodiments, the first TAA is IL13Ra2 and the second TAA is EGFR.

In certain embodiments, the BsAb-CAR construct further comprises, or alternatively further consists essentially of, or yet further consists of, a linker polypeptide located between the heavy chain variable region and the light chain variable region. In some embodiments, a linker polypeptide is located between the antibody fragments of NDG2D and the other antigen binding portion, e.g., anti-CS1, anti-CD123 or anti-IL13Ra2. In certain embodiments, the linker is a glycine-serine linker. In further embodiments, the linker polypeptide comprises, or alternatively consists essentially of, or further consists of the sequence (glycine-serine)n wherein n is an integer from 1 to 6 (SEQ ID NO: 110), e.g. wherein n is 4. In some embodiments, the BsAB-CAR comprises a self-cleaving peptide such as a T2A peptide located between the antibody or antigen binding fragments that binds a cancer or tumor cell and the bispecific antibody, e.g., between the BMCA CAR and the NKG2D antigen binding domain. In some embodiments, the BsAb-CAR further comprises a self-cleaving peptide such as for example a T2A peptide located between the NKG2D and the second antigen binding domains that binds the cancer or tumor antigen, e.g., CS1. In some embodiments, this CAR element further comprises, or alternatively consisting essentially of, or yet further consisting of a signaling domain. In a further embodiment, a signaling peptide is located prior to the NKG2D antigen binding domain of the anti-NKG2D antibody. In some embodiments, this CAR element further comprises, or alternatively consisting essentially of, or yet further consisting of a signal peptide.

In some embodiments, either or both of the first TAA and the second TAA is or are selected from: FLT3, CD19, mesothelin, human epidermal growth factor receptor 2 (HER2), prostate stem cell antigen (PSCA), carcinoembryonic antigen (CEA), CD33, GTPase-activating protein (GAP), ganglioside G2 (GD2), CD5, prostate specific membrane antigen (PSMA), receptor tyrosine kinase-like orphan receptor 1 (ROR1), CD123, CD70, CD38, mucin 1, (Muc1), ephrin type-A receptor 2 precursor (EphA2), epidermal growth factor receptor variant III (EGFRVIII), interleukin 13 receptor alpha 2 (IL13Ra2), CD133, glypican 3 (GPC3), epithelial cell adhesion molecule precursor (EpCam), fibroblast activation protein alpha (FAP), vascular endothelial growth factor receptor 2 (VEGFR2), cancer/testis (CT), guanylyl cyclase C (GUCY2C), tumor-associated glycoprotein-72 (TAG-72), thymidine kinase 1 (TK1), and hypoxanthine guanine phosphoribosyltransferase (HPRT1).

In some embodiments, the BsAb-CAR construct (also referred to herein as a polypeptide) further comprises a peptide linker located between any two of the following: the CAR; the bispecific antibody; and an optional suicide gene product. In some embodiments, the BsAb-CAR construct further comprises a peptide linker between the CAR and the BsAb. In some embodiments, the peptide linker here is a cleavable peptide. In further embodiments, the peptide linker here is a self-cleaving peptide. In yet further embodiments, the peptide linker here is a T2A peptide, optionally comprising, or alternatively consisting essentially of, or yet consisting of HVGSGEGRGSLLTCGDVEENPGP (SEQ ID NO: 85) or an equivalent thereof. In some embodiments, an equivalent of SEQ ID NO: 85 comprises 1, or 2, or 3, or 4, or 5, or 6, or 7, or 8, or 9, or 10, or 11, or 12, or 13, or 14, or 15, or more mutations compared to SEQ ID NO: 85 and is still capable of self-cleaving, and thus releasing two products from a single polypeptide. Methods of evaluating such self-cleavage are available for one of skill in the art, such as via western blot detecting molecular size of the products and/or polypeptides.

In some embodiments, the BsAb-CAR construct (also referred to herein as a polypeptide) further comprises a detectable or purification marker.

Further provided are polynucleotides and vectors encoding these BsAb-CAR constructs.

In some embodiments, the BsAb-CAR comprises the structure shown in part in FIG. 1E, i.e., a single BsAb-CAR comprising or consisting essentially of a BCMA CAR, a self-cleaving peptide such as T2A, NKG2D scFv and Fc polypeptides, a second a self-cleaving peptide such as T2A, and CS1 scFv and Fc polypeptides. The BCMA CAR further comprises a hinge domain, a transmembrane domain, and an intracellular domain. In some embodiments, the CAR component further comprises, or alternatively consists essentially of, or yet further consists of a signaling domain. In some embodiment, the BsAb-CAR construct comprises, or alternatively consists essentially of, or yet further consists of a signal peptide that is located at the amino (N) terminus and/or ahead of anti-NKG2D antigen binding domain. An exemplary IgG1 signal peptide is provided herein but others are known in the art and can be substituted for this peptide. Further provided are polynucleotides and vectors encoding these BsAb-CAR constructs.

In some embodiments, the BsAb-CAR comprises the structure shown in part in FIG. 1F, i.e., a single BsAb-CAR comprising or consisting essentially of a BCMA antigen binding polypeptide, a self-cleaving peptide such as T2A, NKG2D scFv and Fc polypeptides, an HMA polypeptide, and CS1 scFv and Fc polypeptides. The BCMA CAR further comprises a hinge domain, a transmembrane domain, and an intracellular domain. In some embodiments, the BCMA CAR further comprises, or alternatively consists essentially of, or yet further consists of a signal peptide that is located at the amino (N) terminus or ahead of anti-NKG2D antigen binding domain. An exemplary IgG1 signal peptide is provided herein but others are known in the art and can be substituted for this peptide. Further provided are polynucleotides and vectors encoding these BsAb-CAR constructs.

In some embodiments, the BsAb-CAR comprises the structure shown in part in FIG. 1G, i.e., a single Bs-Ab CAR comprising or consisting essentially of (a) a BCMA antigen binding polypeptide, a self-cleaving peptide such as T2A, NKG2D scFv and Fc polypeptides, a linker such as G4S polypeptide (SEQ ID NO: 134), and CS1 scFv and Fc polypeptides. The BCMA further comprises or consists essentially of a hinge domain, a transmembrane domain, and an intracellular domain. In some embodiments, this polypeptide further comprises, or alternatively consists essentially of, or yet further consists of a signal peptide that is located at the amino terminus or ahead of anti-NKG2D antigen binding domain. An exemplary IgG1 signal peptide is provided herein but others are known in the art and can be substituted for this peptide. Further provided are polynucleotides and vectors encoding these BsAb-CAR constructs.

In further aspect, provided herein are polynucleotides (such as isolated and/or engineered polypeptides) and/or isolated nucleic acids that encode, in one construct, a CAR construct and a bispecific antibody as disclosed above (“BsAb-CAR construct”). The antigen binding domains can be from any appropriate species, e.g., murine, human or a humanized sequence. In one aspect, the polynucleotides and/or isolated nucleic acid encodes an antigen binding fragment that targets an cancer or tumor antigen other than BCMA and a bispecific antibody, e.g., one scFv from an anti-CS1 antibody and one scFv from an anti-NKG2D antibody, joined together by a non-immunogenic protein linker such as from human muscle aldose (HMA). In some embodiments, the polynucleotides and/or nucleic acid encoding the CAR construct is located 5′ to the nucleic acid encoding the BsAb. In further embodiments, a T2A coding element is located between the 5′ located CAR polynucleotide and the 3′ located BsAb. In some embodiments, the polynucleotides and/or nucleic acid encoding the CAR construct is located 3′ to the nucleic acid encoding the BsAb. In further embodiments, a T2A coding element is located between the 3′ located CAR polynucleotide and the 5′ located BsAb. The polynucleotides and/or nucleic acids can further comprise the necessary regulatory sequences, e.g., a promoter for expression in a host cell, e.g., a mammalian or human host cell such as a T cell or an NK cell. In some embodiments, the polynucleotides and/or nucleic acids further comprise a first regulatory sequence directing the expression of the BsAb-CAR construct. In further embodiments, the regulatory sequence comprises one or more of the following: a promoter, an intron, an enhancer, or a polyadenylation signal. In some embodiments, the promoter is an EF1a or a CMV promoter located 5′ to the polynucleotide encoding the CAR. In a further embodiment, the polynucleotides and/or isolated nucleic acid can further comprise a detectable or purification maker and/or a sequence encoding a detectable or purification marker, such as GFP that may be downstream from the BsAb polynucleotide and under the control of a separate regulatory element (a second regulatory sequence), e.g., a promoter optionally an EF1alpha promoter. As is apparent to the skilled artisan, the promoter(s) are selected for the host expression system. In some embodiments, the polynucleotides and/or nucleic acids further comprise a suicide gene encoding a suicide gene product optionally selected from one or more of: HSV-TK (Herpes simplex virus thymidine kinase), cytosine deaminase, nitroreductase, carboxylesterase, cytochrome P450 or PNP (Purine nucleoside phosphorylase), truncated EGFR, or inducible caspase (“iCasp”). In further embodiments, the polynucleotides and/or nucleic acids further comprise a third regulatory sequence directing expression of the suicide gene product. In one embodiment, any one or two or three of the regulatory sequences is or are inducible or constitutively active in the cell. In one embodiment, any one or two or three of the regulatory sequences can be cell specific or tissue specific.

In some embodiments, the polynucleotides and/or nucleic acids comprise, or alternatively consist essentially of, or yet consist of a CAR coding polynucleotide as disclosed herein and a bispecific antibody coding polynucleotide as disclosed herein. In some embodiments, the CAR coding polynucleotide and the bispecific antibody coding polynucleotide are contiguous. In some embodiments, the CAR coding polynucleotide and the bispecific antibody coding polynucleotide are linked together by a sequence encoding a cleavable peptide as disclosed herein. Also provided is a polynucleotide that is reverse, complement, or reverse-complement to the polynucleotide as disclosed herein. In some embodiments, a polynucleotide as disclosed herein further comprise a detectable or purification marker and/or a sequence encoding a detectable or purification marker.

In some embodiments relating to any disclosure herein, the regulatory sequences comprises, or alternatively consists essentially of, or yet consists of one or more of the following: a promoter, an intron, an enhancer, or a polyadenylation signal.

The polynucleotides and/or isolated nucleic acid can be inserted into a vector, such as an expression vector, e.g., a lentiviral vector, between the 5′ and 3′ LTRs. FIG. 3A is an exemplary lentiviral vector construct of this disclosure. As is apparent to the skilled artisan, the constructs may not comprise a marker peptide or purification marker. In some embodiments, the vector is a non-viral vector, such as a plasmid. In some embodiments, the vector is a viral vector, optionally selected from the group of a retroviral vector, a lentiviral vector, an adenoviral vector, and an adeno-associated viral vector. Additionally or alternatively, the vector further comprises a regulatory sequence directing replication and/or expression of the BsAb-CAR encoding polynucleotide and/or isolated nucleic acid. In further embodiments, the regulatory sequence comprises one or more of the following: a promoter, an intron, an enhancer, or a polyadenylation signal. In some embodiments, the vector further comprises a detectable or purification marker.

### Host Cells and Processes for Preparing CARs

Aspects of the present disclosure relate to a cell (which is also referred to herein as a host cell) for example, an isolated and/or engineered cell, comprising a CAR, a BsAb, and/or a BsAb-CAR, and methods of producing such cells. In one aspect, provided is an isolated or engineered cell comprising one or more of the following: a polypeptide as disclosed herein, a polynucleotide as disclosed herein, or a vector as disclosed herein. The cell is a prokaryotic or a eukaryotic cell. In some embodiments, the cell is a T-cell, a B cell, an NK cell, an NKT cell, a dendritic cell, a myeloid cell, a monocyte, a macrophage, any subsets thereof, or any other immune cell. In some embodiments, the cell is an immune cell optionally selected from a T-cell, a B cell, an NK cell, an NKT cell, a dendritic cell, a myeloid cell, a monocyte, a macrophage. In further embodiments, the immune cell is derived from hematopoietic stem cells (HSCs) and/or induced pluripotent stem cells (iPSCs). The eukaryotic cell can be from any preferred species, e.g., an animal cell, a mammalian cell such as a human, a bovine cell, a murine cell, an equine cell, a feline cell, or a canine cell. The cells may be derived from patients, donors, or cell lines, such as those available off-the-shelf. The cells can be autologous or allogeneic to the subject being treated. In some embodiments, the cell further comprise a detectable or purification marker. In some embodiments, the cell expresses a CAR as disclosed herein. Additionally or alternatively, the cell expresses a bispecific antibody as disclosed herein. In further embodiments, the cell secretes the bispecific antibody outside of the cell. Also provided is a cell population comprising, or alternatively consisting essentially of, or yet consisting of a cell as disclosed herein. In further embodiments, the cell population is substantially homogenous.

In specific embodiments, the isolated cell comprises, or alternatively consists essentially of, or yet further consists of an exogenous (for example to the cell or the species of the cell) CAR or a BsAb CAR comprising, or alternatively consisting essentially of, or yet further consisting of, an antigen binding domain of a cancer or tumor antibody, a CD8 α hinge domain, a CD8 α transmembrane domain, a CD28 costimulatory signaling region and/or a 4-1BB costimulatory signaling region, and a CD3 zeta signaling domain. In a separate embodiment, the isolated cell further comprises a BsAb as disclosed herein. In yet a further embodiment, the cell comprises the BsAb as disclosed herein. In certain embodiments, the isolated cell is a T-cell, e.g., an animal T-cell, a mammalian T-cell, a feline T-cell, a canine T-cell or a human T-cell. In certain embodiments, the isolated cell is an NK-cell, e.g., an animal NK-cell, a mammalian NK-cell, a feline NK-cell, a canine NK-cell or a human NK-cell. In certain embodiments, the isolated cell is an NKT-cell, e.g., an animal NKT-cell, a mammalian NKT-cell, a feline NKT-cell, a canine NKT-cell or a human NKT-cell. In certain embodiments, the isolated cell is a B-cell, e.g., an animal B-cell, a mammalian B-cell, a feline B-cell, a canine B-cell or a human B-cell. It is appreciated that the same or similar embodiments for each species apply with respect to dendritic cells, myeloid cells, monocytes, macrophages, any subsets of these or the T-cells, NK-cells, NTT-cells, and B-cells as described, and/or any other immune cells. In one aspect, the cell is a T cell that has been modified to remove CD52 expression using gene editing technology, e.g., CRISPR or TALEN.

In certain embodiments, methods of producing the BsAb, CAR, and/or CAR or BsAb-CAR expressing cells (optionally secreting the BsAb) are disclosed herein, the method comprising, or alternatively consisting essentially of, or yet further consisting of transducing a cell as described herein or a cell population as described herein, or a population of isolated cells with a nucleic acid sequence encoding the BsAb, the CAR, the BsAb and CAR, and/or BsAb CAR, such as a polynucleotide and/or vector as disclosed herein. In a further embodiment, a subpopulation of cells that have been successfully transduced with the nucleic acid sequence is selected. In some embodiments, the isolated cells are T-cells, an animal T-cell, a mammalian T-cell, a feline T-cell, a canine T-cell or a human T-cell, thereby producing the BsAb, the CAR, the BsAb and CAR and/or BsAb CAR T-cells. In certain embodiments, the isolated cell is an NK-cell, e.g., an animal NK-cell, a mammalian NK-cell, a feline NK-cell, a canine NK-cell or a human NK-cell, thereby producing the BsAb, the CAR, the BsAb and CAR and/or BsAb CAR NK-cells. In certain embodiments, the isolated cell is an NKT-cell, e.g., an animal NKT-cell, a mammalian NKT-cell, a feline NKT-cell, a canine NKT-cell or a human NKT-cell, thereby producing the BsAb, the CAR, the BsAb and CAR and/or BsAb CAR NKT-cells. In some embodiments, the isolated cells are B-cells, an animal B-cell, a mammalian B-cell, a feline B-cell, a canine B-cell or a human B-cell, thereby producing the BsAb, the CAR, the BsAb and CAR and/or BsAb CAR B-cells. It is appreciated that the same or similar embodiments for each species apply with respect to dendritic cells, myeloid cells, monocytes, macrophages, any subsets of these or the T-cells, NK-cells, and B-cells described, and/or any other immune cells. In some embodiments, the cell is selected from a Hematopoietic stem cell (HSC), an induced pluripotent stem cell (iPSCs), or an immune cell. In some embodiments, the cell population comprises, or alternatively consists essentially of, or yet consists of a Hematopoietic stem cell (HSC), an induced pluripotent stem cell (iPSCs), or an immune cell. In some embodiments, the immune cells is selected from a group consisting of T-cells, B cells, NK cells, NKT cells, dendritic cells, myeloid cells, monocytes, or macrophages. In some embodiments, the immune cell is derived from HSCs and/or iPSCs. In one embodiment, the cell is a T cell that has been modified to remove CD52 expression using gene editing technology, e.g., CRISPR or TALEN. In one aspect, the cells are autologous or allogenic to the subject being treated.

Sources of Isolated Cells. Prior to expansion and genetic modification of the cells disclosed herein, cells may be obtained from a subject—for instance, in embodiments involving autologous therapy—or a commercially available cell line or culture, or a stem cell such as an induced pluripotent stem cell (iPSC).

Cells can be obtained from a number of sources in a subject, including peripheral blood mononuclear cells, bone marrow, lymph node tissue, cord blood, thymus tissue, tissue from a site of infection, ascites, pleural effusion, spleen tissue, and tumors.

Methods of isolating relevant cells are well known in the art and can be readily adapted to the present application; an exemplary method is described in the examples below. Isolation methods for use in relation to this disclosure include, but are not limited to Life Technologies Dynabeads® System; STEMcell Technologies EasySep™, RoboSep™ RosetteSep™, SepMate™; Miltenyi Biotec MACS™ cell separation kits, and other commercially available cell separation and isolation kits. Particular subpopulations of immune cells may be isolated through the use of beads or other binding agents available in such kits specific to unique cell surface markers. For example, MACS™ CD4+ and CD8+ MicroBeads may be used to isolate CD4+ and CD8+ T-cells. Alternate non-limiting examples of cells that may be isolated according to known techniques include bulked T-cells, NK T-cells, and gamma delta T-cells.

Alternatively, cells may be obtained through commercially available cell cultures, including but not limited to, for T-cells, lines BCL2 (AAA) Jurkat (ATCC® CRL-2902™) BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™); for B cells, lines AHH-1 (ATCC® CRL-8146™), BC-1 (ATCC® CRL-2230™), BC-2 (ATCC® CRL-2231™), BC-3 (ATCC® CRL-2277™), CA46 (ATCC® CRL-1648™), DG-75 [D.G.-75] (ATCC® CRL-2625™), DS-1 (ATCC® CRL-11102™), EB-3 [EB3] (ATCC® CCL-85™), Z-138 (ATCC #CRL-3001), DB (ATCC CRL-2289), Toledo (ATCC CRL-2631), Pfiffer (ATCC CRL-2632), SR (ATCC CRL-2262), JM-1 (ATCC CRL-10421), NFS-5 C-1 (ATCC CRL-1693); NFS-70 C10 (ATCC CRL-1694), NFS-25 C-3 (ATCC CRL-1695), and SUP-B15 (ATCC CRL-1929); and, for NK cells, lines NK-92 (ATCC® CRL-2407™) NK-92MI (ATCC® CRL-2408™). Further examples include but are not limited to mature T-cell lines, e.g., Deglis, EBT-8, HPB-MLp-W, HUT 78, HUT 102, Karpas 384, Ki 225, My-La, Se-Ax, SKW-3, SMZ-1 and T34; immature T-cell lines, e.g., ALL-SIL, Bel3, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PERO117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4; 11 (ATCC CRL-1873), CCRF-CEM (ATCC CRM-CCL-119); cutaneous T-cell lymphoma lines, e.g., HuT78 (ATCC CRM-TIB-161), MJ[G11] (ATCC CRL-8294), HuT102 (ATCC TIB-162); B-cell lines derived from anaplastic and large cell lymphomas, e.g., DEL, DL-40, FE-PD, JB6, Karpas 299, Ki-JK, Mac-2A Ply1, SR-786, SU-DHL-1, -2, -4, -5, -6, -7, -8, -9, -10, and -16, DOHH-2, NU-DHL-1, U-937, Granda 519, USC-DHL-1, RL; Hodgkin's lymphomas, e.g., DEV, HD-70, HDLM-2, HD-MyZ, HKB-1, KM-H2, L 428, L 540, L1236, SBH-1, SUP-HD1, and SU/RH-HD-1; and NK lines such as HANK1, KHYG-1, NKL, NK-YS, NOI-90, and YT. Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HIMC-1 leukemia, KG-1 leukemia, U266 myeloma. Non-limiting exemplary sources for such commercially available cell lines include the American Type Culture Collection, or ATCC, (atcc.org/) and the German Collection of Microorganisms and Cell Cultures (dsmz.de/).

In some embodiments, T-cells expressing the disclosed CARs may be further modified to reduce or eliminate expression of endogenous TCRs. Reduction or elimination of endogenous TCRs can reduce off-target effects and increase the effectiveness of the T cells. T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs.

The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR. Even though some TCR complexes can be recycled to the cell surface when RNA interference is used, the RNA (e.g., shRNA, siRNA, miRNA, etc.) will prevent new production of TCR proteins resulting in degradation and removal of the entire TCR complex, resulting in the production of a T cell having a stable deficiency in functional TCR expression.

Expression of inhibitory RNAs (e.g., shRNA, siRNA, miRNA, etc.) in primary T cells can be achieved using any conventional expression system, e.g., a lentiviral expression system. Although lentiviruses are useful for targeting resting primary T cells, not all T cells will express the shRNAs. Some of these T cells may not express sufficient amounts of the RNAs to allow enough inhibition of TCR expression to alter the functional activity of the T cell. Thus, T cells that retain moderate to high TCR expression after viral transduction can be removed, e.g., by cell sorting or separation techniques, so that the remaining T cells are deficient in cell surface TCR or CD3, enabling the expansion of an isolated population of T cells deficient in expression of functional TCR or CD3.

Expression of CRISPR in primary T cells can be achieved using conventional CRISPR/Cas systems and guide RNAs specific to the target TCRs. Suitable expression systems, e.g. lentiviral or adenoviral expression systems are known in the art. Similar to the delivery of inhibitor RNAs, the CRISPR system can be used to specifically target resting primary T cells or other suitable immune cells for CAR cell therapy. Further, to the extent that CRISPR editing is unsuccessful, cells can be selected for success according to the methods disclosed above. For example, as noted above, T cells that retain moderate to high TCR expression after viral transduction can be removed, e.g., by cell sorting or separation techniques, so that the remaining T cells are deficient in cell surface TCR or CD3, enabling the expansion of an isolated population of T cells deficient in expression of functional TCR or CD3. It is further appreciated that a CRISPR editing construct may be useful in both knocking out the endogenous TCR and knocking in the CAR constructs disclosed herein. Accordingly, it is appreciated that a CRISPR system can be designed for to accomplish one or both of these purposes.

Vectors. CAR cells may be prepared using vectors. Aspects of the present disclosure relate to an isolated nucleic acid sequence encoding (i) a CAR or (ii) a polynucleotide encoding an immunoregulatory molecule and vectors comprising, or alternatively consisting essentially of, or yet further consisting of, an either one or both of these nucleic acids and/or complements and/or equivalents of each thereof.

In some embodiments, the isolated nucleic acid sequence encodes for the CAR component comprises, or alternatively consists essentially of, or yet further consists of an antigen binding domain of a cancer or tumor targeting antibody, a CD8 α hinge domain, a CD8 α transmembrane domain, a CD28 costimulatory signaling region and/or a 4-1BB costimulatory signaling region, and a CD3 zeta signaling domain. In specific embodiments, the isolated nucleic acid sequence comprises, or alternatively consisting essentially of, or yet further consisting of, sequences encoding (a) an antigen binding domain of a cancer or tumor targeting antibody followed by (b) a CD8 α hinge domain, (c) a CD8 α transmembrane domain followed by (d) a CD28 costimulatory signaling region and/or a 4-1BB costimulatory signaling region followed by (e) a CD3 zeta signaling domain.

In some embodiments, the isolated nucleic acid sequence encodes for the CAR component comprises, or alternatively consists essentially of, or yet further consists of, a Kozak consensus sequence upstream of the sequence encoding the antigen binding domain of the cancer or tumor targeting antibody.

In one aspect, the antigen binding domain targets BCMA, FLT3 or EGFR or alternatively CD19, CD123 or IL13Ra2. In one aspect, the antigen binding domain targets HER2. In one aspect, the antigen binding domain targets PSCA. In one aspect, the antigen binding domain targets a CEA. In one aspect, the antigen binding domain targets GAP. In one aspect, the antigen binding domain targets GD2. In one aspect, the antigen binding domain targets CD5. In one aspect, the antigen binding domain targets PSMA. In one aspect, the antigen binding domain targets ROR1. In one aspect, the antigen binding domain targets CD123. In one aspect, the antigen binding domain targets CD70. In one aspect, the antigen binding domain targets CD38. In one aspect, the antigen binding domain targets Muc1. In one aspect, the antigen binding domain targets EphA2. In one aspect, the antigen binding domain targets EGFRVIII. In one aspect, the antigen binding domain targets IL13Ra2. In one aspect, the antigen binding domain targets CD133. In one aspect, the antigen binding domain targets GPC3. In one aspect, the antigen binding domain targets EpCam. In one aspect, the antigen binding domain targets FAP. In one aspect, the antigen binding domain targets VEGFR2. In one aspect, the antigen binding domain targets a cancer/testis antigen. In one aspect, the antigen binding domain targets GUCY2C. In one aspect, the antigen binding domain targets TAG-72. In one aspect, the antigen binding domain targets TK1. In one aspect, the antigen binding domain targets HPRT1.

In some embodiments, the isolated nucleic acid comprises, or alternatively consists essentially of, or yet further consists of a polynucleotide encoding a bispecific antibody. In certain embodiments, the bispecific antibody, or alternatively consists essentially of, or further consists of the relevant CDR regions or scFv, or the scFv and Fv of an antibody to NKG2D, and optionally, the antigen binding domain of an anti-SLAMF7 (also known as CS1 or CD319), e.g. scFv and Fv or the scFv polypeptides that are optionally codon optimized, or an equivalent of each thereof. In certain embodiments, the bispecific antibody comprises, or alternatively consists essentially of, or further consists of the relevant CDR regions of an antibody to NKG2D and an antibody to SLAMF7 (also known as CS1 or CD319) (that are optionally codon optimized) or an equivalent of each thereof. In some embodiments, the bispecific antibody comprises, or alternatively consists essentially of, or further consists of the heavy chain and/or light chain variable region of an antibody to NKG2D and an antibody to SLAMF7 (also known as CS1 or CD319) (that are optionally codon optimized) and/or an equivalent of each thereof. In some embodiments, the bispecific antibody comprises a single chain variable fragment (scFv) derived from an antibody to NKG2D or a scFv and Fc fragments from an antibody to NKG2D and, optionally, a single chain variable fragment (scFv) and optionally scFv and Fc fragments of an antibody directed to SLAMF7 (also known as CS1 of CD319) (that are optionally codon optimized) and/or an equivalent each thereof. In some embodiments, the isolated nucleic acid comprises, or alternatively consists essentially of, or yet further consists of a polynucleotide sequence encoding the bispecific antibody operatively linked to a promoter that may be generated according to the method disclosed above.

In some embodiments, the isolated nucleic acid comprises a detectable label and/or a polynucleotide conferring antibiotic resistance. In one aspect, the label or polynucleotide are useful to select cells successfully transduced with the isolated nucleic acids.

In some embodiments, the isolated nucleic acid sequence is comprised within a vector. In certain embodiments, the vector is a plasmid. In other embodiments, the vector is a viral vector. Non-limiting examples of such include without limitation a retroviral vector, a lentiviral vector, an adenoviral vector, and an adeno-associated viral vector. In specific embodiments, the vector is a lentiviral vector.

The preparation of exemplary vectors and the generation of CAR expressing cells using said vectors is discussed in detail in the examples below. In summary, the expression of natural or synthetic nucleic acids encoding CARs or immunoregulatory molecules is typically achieved by operably linking a nucleic acid encoding the CAR polypeptide or portions thereof to a promoter, and incorporating the construct into an expression vector. A similar method may be used to construct the isolated nucleic acid sequence comprising a polynucleotide encoding an immunoregulatory molecule. The vectors can be suitable for replication and integration eukaryotes. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

In one aspect, the term “vector” intends a recombinant vector that retains the ability to infect and transduce non-dividing and/or slowly-dividing cells and integrate into the target cell's genome. In several aspects, the vector is derived from or based on a wild-type virus. In further aspects, the vector is derived from or based on a wild-type lentivirus. Examples of such include without limitation, human immunodeficiency virus (HIV), equine infectious anemia virus (EIAV), simian immunodeficiency virus (SIV) and feline immunodeficiency virus (FIV). Alternatively, it is contemplated that other retrovirus can be used as a basis for a vector backbone such murine leukemia virus (MLV). It will be evident that a viral vector according to the disclosure need not be confined to the components of a particular virus. The viral vector may comprise components derived from two or more different viruses, and may also comprise synthetic components. Vector components can be manipulated to obtain desired characteristics, such as target cell specificity.

The recombinant vectors of this disclosure are derived from primates and non-primates. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anemia virus (EIAV) and the more recently described feline immunodeficiency virus (FIV) and bovine immunodeficiency virus (BIV). Prior art recombinant lentiviral vectors are known in the art, e.g., see U.S. Pat. Nos. 6,924,123; 7,056,699; 7,419,829 and 7,442,551, incorporated herein by reference.

U.S. Pat. No. 6,924,123 discloses that certain retroviral sequence facilitate integration into the target cell genome. This patent teaches that each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome. The LTRs themselves are identical sequences that can be divided into three elements, which are called U3, R and U5. U3 is derived from the sequence unique to the 3′ end of the RNA. R is derived from a sequence repeated at both ends of the RNA, and U5 is derived from the sequence unique to the 5′ end of the RNA. The sizes of the three elements can vary considerably among different retroviruses. For the viral genome, the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome.

For the production of viral vector particles, the vector RNA genome is expressed from a DNA construct encoding it, in a host cell. The components of the particles not encoded by the vector genome are provided in trans by additional nucleic acid sequences (the “packaging system”, which usually includes either or both of the gag/pol and env genes) expressed in the host cell. The set of sequences required for the production of the viral vector particles may be introduced into the host cell by transient transfection, or they may be integrated into the host cell genome, or they may be provided in a mixture of ways. The techniques involved are known to those skilled in the art.

Retroviral vectors for use in this disclosure include, but are not limited to Invitrogen's pLenti series versions 4, 6, and 6.2 “ViraPower” system. Manufactured by Lentigen Corp.; pHIV-7-GFP, lab generated and used by the City of Hope Research Institute; “Lenti-X” lentiviral vector, pLVX, manufactured by Clontech; pLKO.1-puro, manufactured by Sigma-Aldrich; pLemiR, manufactured by Open Biosystems; and pLV, lab generated and used by Charite Medical School, Institute of Virology (CBF), Berlin, Germany.

Further methods of introducing exogenous nucleic acids into the art are known and include but are not limited to gene delivery using one or more of RNA electroporation, nanotechnology, sleeping beauty vectors, retroviruses, and/or adenoviruses.

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

Packaging vector and cell lines. The isolated nucleic acids can be packaged into a retroviral packaging system by using a packaging vector and cell lines. The packaging vector includes, but is not limited to retroviral vector, lentiviral vector, adenoviral vector, and adeno-associated viral vector. The packaging vector contains elements and sequences that facilitate the delivery of genetic materials into cells. For example, the retroviral constructs are packaging vectors comprising at least one retroviral helper DNA sequence derived from a replication-incompetent retroviral genome encoding in trans all virion proteins required to package a replication incompetent retroviral vector, and for producing virion proteins capable of packaging the replication-incompetent retroviral vector at high titer, without the production of replication-competent helper virus. The retroviral DNA sequence lacks the region encoding the native enhancer and/or promoter of the viral 5′ LTR of the virus, and lacks both the psi function sequence responsible for packaging helper genome and the 3′ LTR, but encodes a foreign polyadenylation site, for example the SV40 polyadenylation site, and a foreign enhancer and/or promoter which directs efficient transcription in a cell type where virus production is desired. The retrovirus is a leukemia virus such as a Moloney Murine Leukemia Virus (MMLV), the Human Immunodeficiency Virus (HIV), or the Gibbon Ape Leukemia virus (GALV). The foreign enhancer and promoter may be the human cytomegalovirus (HCMV) immediate early (IE) enhancer and promoter, the enhancer and promoter (U3 region) of the Moloney Murine Sarcoma Virus (MMSV), the U3 region of Rous Sarcoma Virus (RSV), the U3 region of Spleen Focus Forming Virus (SFFV), or the HCMV IE enhancer joined to the native Moloney Murine Leukemia Virus (MMLV) promoter. The retroviral packaging vector may consist of two retroviral helper DNA sequences encoded by plasmid based expression vectors, for example where a first helper sequence contains a cDNA encoding the gag and pol proteins of ecotropic MMLV or GALV and a second helper sequence contains a cDNA encoding the env protein. The Env gene, which determines the host range, may be derived from the genes encoding xenotropic, amphotropic, ecotropic, polytropic (mink focus forming) or 10A1 murine leukemia virus env proteins, or the Gibbon Ape Leukemia Virus (GALV env protein, the Human Immunodeficiency Virus env (gp160) protein, the Vesicular Stomatitus Virus (VSV) G protein, the Human T cell leukemia (HTLV) type I and II env gene products, chimeric envelope gene derived from combinations of one or more of the aforementioned env genes or chimeric envelope genes encoding the cytoplasmic and transmembrane of the aforementioned env gene products and a monoclonal antibody directed against a specific surface molecule on a desired target cell.

In the packaging process, the packaging vectors and retroviral vectors are transiently co-transfected into a first population of mammalian cells that are capable of producing virus, such as human embryonic kidney cells, for example 293 cells (ATCC No. CRL1573, ATCC, Rockville, Md.) to produce high titer recombinant retrovirus-containing supernatants. In another method of the disclosure this transiently transfected first population of cells is then co-cultivated with mammalian target cells, for example human lymphocytes, to transduce the target cells with the foreign gene at high efficiencies. In yet another method of the disclosure the supernatants from the above described transiently transfected first population of cells are incubated with mammalian target cells, for example human lymphocytes or hematopoietic stem cells, to transduce the target cells with the foreign gene at high efficiencies.

In another aspect, the packaging vectors are stably expressed in a first population of mammalian cells that are capable of producing virus, such as human embryonic kidney cells, for example 293 cells. Retroviral or lentiviral vectors are introduced into cells by either co-transfection with a selectable marker or infection with pseudotyped virus. In both cases, the vectors integrate. Alternatively, vectors can be introduced in an episomally maintained plasmid. High titer recombinant retrovirus-containing supernatants are produced.

Activation and Expansion of CAR Cells. Whether prior to or after genetic modification of the cells to express a desirable CAR, the cells can be activated and expanded using generally known methods such as those described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041 and references such as Lapateva et al. (2014) Crit Rev Oncog 19(1-2):121-32; Tam et al. (2003) Cytotherapy 5(3):259-72; Garcia-Marquez et al. (2014) Cytotherapy 16(11):1537-44. Stimulation with the tumor relevant antigen ex vivo can activate and expand the selected CAR expressing cell subpopulation. Alternatively, the cells may be activated in vivo by interaction with a tumor relevant antigen.

In the case of certain immune cells, additional cell populations, soluble ligands and/or cytokines, or stimulating agents may be required to activate and expand cells. The relevant reagents are well known in the art and are selected according to known immunological principles. For instance, soluble CD-40 ligand may be helpful in activating and expanding certain B-cell populations; similarly, irradiated feeder cells may be used in the procedure for activation and expansion of NK cells.

Methods of activating relevant cells are well known in the art and can be readily adapted to the present application; an exemplary method is described in the examples below. Isolation methods for use in relation to this disclosure include, but are not limited to Life Technologies Dynabeads® System activation and expansion kits; BD Biosciences Phosflow™ activation kits, Miltenyi Biotec MACS™ activation/expansion kits, and other commercially available cell kits specific to activation moieties of the relevant cell. Particular subpopulations of immune cells may be activated or expanded through the use of beads or other agents available in such kits. For example, α-CD3/α-CD28 Dynabeads® may be used to activate and expand a population of isolated T-cells.

### Methods of Use

Therapeutic Application. Method aspects of the present disclosure relate to methods for inhibiting the growth of a tumor or cancer cells, (e.g., MM, AML or GB cells) in vitro or in vivo and/or for treating a cancer patient in need thereof. In some embodiments, the tumor is a solid tumor. In some embodiments, the cancer is a cancer affecting blood and/or bone marrow, e.g., MM. In some embodiments, the cancer or tumor cell expresses or overexpresses a cancer or tumor antigen CS1. When practiced in vitro, the methods provide in vitro assays for precision medicine application and useful assays for testing new combination and therapies.

In one aspect, provided is a method of inhibiting the growth of a cancer cell expressing a tumor associated antigen (TAA) or a tissue comprising the cancer cell. The method comprises, or alternatively consists essentially of, or yet consists of contacting the cancer cell or the tissue with, optionally an efficient amount of, the isolated or engineered cell or a cell population as disclosed herein. In some embodiments, the contacting is in vitro or in vivo. In some embodiments, the contacting is in vivo and the isolated cells are autologous or allogeneic to a subject being treated. In some embodiments, the contacting is in vivo and the isolated cells are allogenic to a subject being treated. In some embodiments, the method further comprises contacting the cancer cell or the tissue with an effective amount of a cytoreductive therapy or a therapy that upregulates the expression of a TAA. In some embodiments, the cytoreductive therapy comprises chemotherapy, cryotherapy, hyperthermia, targeted therapy, and/or radiation therapy. In some embodiments, the cancer cell expresses the first TAA or BCMA as disclosed herein. Additionally or alternatively, the cancer cell expresses the second TAA or CS1 as disclosed herein.

In one aspect, provided is a method for one or more of inhibiting the growth of a cancer, inhibiting metastasis of a cancer, or treating a cancer, in a subject in need thereof. The method comprises, or alternatively consists essentially of, or yet consists of administering, for example an effective amount of, an isolated or engineered cell or a cell population as disclosed herein to the subject in need. In some embodiments, the isolated or engineered cell is autologous to the subject in need. In some embodiments, the isolated or engineered cell is allogenic to the subject in need. In some embodiments, the subject is selected for the therapy by determining expression of either or both of: the first TAA or BCMA as disclosed herein, and/or the second TAA or the CS1 as disclosed herein. In further embodiments, the expression is determined by contacting a sample of the subject with an antigen binding domain recognizing and binding the TAA or BCMA or CS1 in vitro or in vivo and detecting binding between the sample and the antigen binding domain. In yet further embodiments, the antigen binding domain further comprises a detectable marker. In some embodiments, the method further comprises administering to the subject an effective amount of a cytoreductive therapy or a therapy that upregulates the expression of a TAA. In some embodiments, the cytoreductive therapy comprises chemotherapy, cryotherapy, hyperthermia, targeted therapy, and/or radiation therapy.

In one aspect, provided is a method for treating a cancer in a subject selected for the treatment. The method comprises, or alternatively consists essentially of, or yet consists of administering, for example an effective amount of, an isolated or engineered cell or a cell population as disclosed herein to the subject. In some embodiments, the subject is selected if a cancer cell of the subject expresses either or both of: the first TAA or BCMA as disclosed herein, and/or the second TAA or the CS1 as disclosed herein. In some embodiments, the TAA expression is determined by contacting a sample of the subject with an antigen binding domain recognizing and binding the TAA or BCMA or CS1 in vitro or in vivo and detecting binding between the sample and the antigen binding domain. In further embodiments, the antigen binding domain further comprises a detectable marker. In some embodiments, the isolated or engineered cell is autologous to the subject in need. In some embodiments, the isolated or engineered cell is allogenic to the subject in need.

In certain embodiments, these methods comprise, or alternatively consist essentially of, or yet further consist of, administering to the subject or patient the isolated cell (e.g., an effective amount) comprising the CAR. In further embodiments, this isolated cell comprises or expresses a CAR and/or a bispecific antibody. In some embodiments, the antigen binding domain of the CAR comprises, or alternatively consists essentially of, or further consists of the relevant CDR regions of an antibody to BCMA or alternatively of an antibody other than to B-cell maturation antigen (BCMA) and/or SLAMF7 (also known as CS1 or CD319), and/or an equivalent of each thereof. In some embodiments, the antigen binding domain of the CAR comprises, or alternatively consists essentially of, or further consists of the heavy chain and/or light chain variable region of an antibody to an antigen other than BCMA and/or SLAMF7 (also known as CS1 or CD319), and/or an equivalent of each thereof. In still further embodiments, the isolated cell is a T-cell or an NK cell. In some embodiments, the bispecific antibody comprises, or alternatively consists essentially of, or further consists of an NKG2D ligand and, optionally, a SLAMF7 (also known as CS1 of CD319) ligand. In certain embodiments, the bispecific antibody comprises, or alternatively consists essentially of, or further consists of the relevant CDR regions of an antibody to NKG2D and, optionally, SLAMF7 (also known as CS1 or CD319), or an equivalent of each thereof. In some embodiments, the bispecific antibody comprises, or alternatively consists essentially of, or further consists of the heavy chain and/or light chain variable region of an antibody to NKG2D and, optionally, SLAMF7 (also known as CS1 or CD319), and/or an equivalent of each thereof. In some embodiments, the bispecific antibody comprises a single chain variable fragment (scFv) alone or in combination with an Fc fragment derived from an antibody to NKG2D and, optionally, a single chain variable fragment (scFv) alone or in combination with an Fc fragment of an antibody derived from SLAMF7 (also known as CS1 of CD319), and/or an equivalent each thereof. The methods can be combined with additional anti-cancer therapies and surgeries as determined by the treating physician or veterinarian.

In one aspect, provided herein are methods for administering to a subject in need thereof an isolated cell comprises or expresses a CAR and/or a bispecific antibody, wherein the BsAb comprises an antigen binding domain of an anti-FLT3 antibody and the BsAb portion comprises an antigen binding domain of an anti-NKG2D and an antigen binding domain of an anti-CD123 antibody. This BsAb is a therapy for the treatment of AML and methods to make and use this BsAb CAR are within the scope of this disclosure. An effective amount of the BsAb is administered to a patient suffering from AML. These methods can be supplemented with appropriate diagnostic methods to diagnose AML and monitor treatment and toxicity. The methods can be combined with additional anti-cancer therapies and surgeries as determined by the treating physician or veterinarian.

In another aspect, provided herein are methods for administering to a subject in need thereof an isolated cell comprises or expresses a CAR and/or a bispecific antibody, wherein the BsAb comprises an antigen binding domain of an anti-EGFR antibody and the BsAb portion comprises an antigen binding domain of an anti-NKG2D and an antigen binding domain of an anti-IL 13Ra2 antibody. This BsAb is a therapy for the treatment of glioblastoma and methods to make and use this BsAb CAR are within the scope of this disclosure. An effective amount of the BsAb is administered to a patient suffering from glioblastoma. Administration can be local (intracranial administration) or systemic. These methods can be supplemented with appropriate diagnostic methods to diagnose glioblastoma and monitor treatment and toxicity. The methods can be combined with additional anti-cancer therapies and surgeries as determined by the treating physician or veterinarian.

In one aspect, provided herein are methods for administering to a subject in need thereof an isolated cell that comprises or expresses a CAR and/or a bispecific antibody, wherein the BsAb the BsAb-CAR comprises the structure shown in part in FIG. 1E, i.e., a single BsAb-CAR comprising or consisting essentially of a BCMA CAR, a self-cleaving peptide such as T2A, NKG2D scFv and Fc polypeptides, a second a self-cleaving peptide such as T2A, and CS1 scFv and Fc polypeptides. The BCMA CAR further comprises a hinge domain, a transmembrane domain, and an intracellular domain. In one aspect, the CAR component further comprises, or alternatively consists essentially of, or yet further consists of a signaling domain that is located at the amino terminus or ahead of anti-NKG2D antigen binding domain. An exemplary IgG1 signal peptide is provided herein but others are known in the art and can be substituted for this peptide. This BsAb is a therapy for the treatment of MM and methods to make and use this BsAb CAR are within the scope of this disclosure. An effective amount of the BsAb is administered to a patient suffering from MM. These methods can be supplemented with appropriate diagnostic methods to diagnose MM and monitor treatment and toxicity. The methods can be combined with additional anti-cancer therapies and surgeries as determined by the treating physician or veterinarian.

In another aspect, provided herein are methods for administering to a subject in need thereof an isolated cell that comprises or expresses a CAR and/or a bispecific antibody, wherein the BsAb comprises the structure shown in part in FIG. 1F, i.e., a single BsAb-CAR comprising or consisting essentially of a BCMA antigen binding polypeptide, a self-cleaving peptide such as T2A, NKG2D scFv and Fc polypeptides, an HMA polypeptide, and CS1 scFv and Fc polypeptides. The BCMA CAR further comprises a hinge domain, a transmembrane domain, and an intracellular domain. In one aspect, the BCMA CAR further comprises, or alternatively consists essentially of, or yet further consists of a signaling domain that is located at the amino terminus or ahead of anti-NKG2D antigen binding domain. An exemplary IgG1 signal peptide is provided herein but others are known in the art and can be substituted for this peptide. This BsAb is a therapy for the treatment of MM and methods to make and use this BsAb CAR are within the scope of this disclosure. An effective amount of the BsAb is administered to a patient suffering from MM. These methods can be supplemented with appropriate diagnostic methods to diagnose MM and monitor treatment and toxicity. The methods can be combined with additional anti-cancer therapies and surgeries as determined by the treating physician or veterinarian.

In one aspect, provided herein are methods for administering to a subject in need thereof an isolated cell that comprises or expresses a CAR and/or a bispecific antibody, wherein the BsAb the BsAb-CAR comprises the structure shown in part in FIG. 1G, i.e., a single Bs-Ab CAR comprising or consisting essentially of (a) a BCMA antigen binding polypeptide, a self-cleaving peptide such as T2A, NKG2D scFv and Fc polypeptides, a linker such as G4S polypeptide (SEQ ID NO: 134), and CS1 scFv and Fc polypeptides. The BCMA further comprises or consists essentially of a hinge domain, a transmembrane domain, and an intracellular domain. In one aspect, this polypeptide further comprises, or alternatively consists essentially of, or yet further consists of a signaling domain that is located at the amino terminus or ahead of anti-NKG2D antigen binding domain. An exemplary IgG1 signal peptide is provided herein but others are known in the art and can be substituted for this peptide. This BsAb is a therapy for the treatment of MM and methods to make and use this BsAb CAR are within the scope of this disclosure. An effective amount of the BsAb is administered to a patient suffering from MM. These methods can be supplemented with appropriate diagnostic methods to diagnose MM and monitor treatment and toxicity. The methods can be combined with additional anti-cancer therapies and surgeries as determined by the treating physician or veterinarian.

In some embodiments, the isolated cell is autologous to the subject or patient being treated. In a further aspect, the tumor expresses a cancer or tumor antigen and the subject has been selected for the therapy by a diagnostic, such as use of an antibody that recognizes and binds the tumor or cancer relevant antigens targeted by the CARs. The subject is an animal, a mammal, a canine, a feline, a bovine, an equine, a murine or a human patient.

The CAR cells as disclosed herein may be administered either alone or in combination with the bispecific antibody disclosed herein or alternatively in one cell, as described herein, diluents, known anti-cancer therapeutics, and/or with other components such as cytokines or other cell populations that are immunoregulatory. They can be administered as a first line therapy, a second line therapy, a third line therapy, or further therapy. Non-limiting examples of additional therapies include cytoreductive therapy, such as radiation therapy, cryotherapy, or chemotherapy, or biologics. Further non-limiting examples include other relevant cell types, such as unmodified immune cells, modified immune cells comprising vectors expressing one or more immunoregulatory molecules, or CAR cells specific to a different antigen than those disclosed herein. As with the CAR cells of the present disclosure, in some embodiments, these cells may be autologous or allogenic. Appropriate treatment regimens will be determined by the treating physician or veterinarian.

Pharmaceutical compositions of the present disclosure may be administered in a manner appropriate to the disease to be treated or prevented. The quantity and frequency of administration will be determined by such factors as the condition of the patient, and the type and severity of the patient's disease, although appropriate dosages may be determined by clinical trials. In one aspect they are administered directly by direct injection or systemically such as intravenous injection.

Aspects of the disclosure provide an exemplary method for determining if a patient is likely to respond to, or is not likely to respond to, CAR therapy. The method comprises, or alternatively consists essentially of, or further consists of determining the presence or absence of necrosis in a tumor sample isolated from the patient and quantitating the amount of cancer or tumor cells expressing the cancer or tumor antigen. In certain embodiments, the method further comprises, or alternatively consists essentially of, or yet further consists of administering an effective amount of the CAR therapy to the patient that is determined likely to respond to the CAR therapy. The CAR therapy can be autologous or allogenic to the patient and the patient can be subject that suffers from a solid tumor, animal or human.

Techniques of histological staining for necrosis are well known in the art. For example, hematoxylin and eosin stains, also referred to as “H&E staining,” are a common technique for identifying the presence of necrosis in tissues, especially in tumorigenic or cancerous growth. Cytoplasmic H&E staining demonstrates increased eosinophilia, attributable in part to the loss of cytoplasmic RNA and in part to denatured cytoplasmic proteins. In necrotic tissue stains, the cytoplasm often appears “moth eaten” due to enzyme digestion of cytoplasmic organelles. Myelin figures, calcification, and evidence of phagocytosis into other cells are also hallmarks of necrotic tissues that can be detected by histological staining. Necrotic tissues also have specific hallmarks in nuclear staining often demonstrating karyolysis, pyknosis, and karyorrhexis as a result of cell death. Using microscopy and either manual or automated quantitation of such necrotic hallmarks, relevance of CAR therapy may be determined. Alternate means of detecting tumorigenic or cancerous growth or necrotic tissues in general, including but not limited to biomarker-based or imaging-based diagnostics, are also equally relevant to determining whether a patient will respond to certain types of CAR therapy, and may be used accordingly. As is apparent, the CAR-BsAb therapy is selected based on the genotype and/or phenotype of the cancer or tumor in the patient sample such that the antigen binding domain will target and treat the specific cancer or tumor.

### Carriers

Additional aspects of the disclosure relate to compositions comprising, or alternatively consisting essentially of, or yet further consisting of, a carrier and one or more of the products—e.g., a cell population as disclosed herein, a CAR, an isolated cell comprising a CAR, a polypeptide, a polynucleotide, an isolated nucleic acid, a vector, a cell, a cell population, and an isolated cell containing the CAR and/or the bispecific antibody as disclosed herein and/or nucleic acids encoding such—described in the embodiments disclosed herein. In some embodiments, the carrier is a pharmaceutically acceptable carrier. In further aspects, the composition may additionally comprise an immunoregulatory molecule and/or an isolated nucleic acid comprising a polynucleotide encoding a bispecific antibody. In certain embodiments, the bispecific antibody, or alternatively consists essentially of, or further consists of the relevant CDR regions of an antibody to BCMA and/or NKG2D, optionally, SLAMF7 (also known as CS1 or CD319), or an equivalent of each thereof. In some embodiments, the bispecific antibody comprises, or alternatively consists essentially of, or further consists of the heavy chain and/or light chain variable region of an antibody to NKG2D, optionally, SLAMF7 (also known as CS1 or CD319) (that are optionally codon optimized) and/or an equivalent of each thereof. In some embodiments, the bispecific antibody comprises a single chain variable fragment (scFv) alone or in combination with an the Fc fragment derived from an antibody to NKG2D, optionally, a single chain variable fragment (scFv) alone or in combination with an Fc fragment derived from SLAMF7 (also known as CS1 of CD319) (that are optionally codon optimized) and/or an equivalent each thereof.

Briefly, pharmaceutical compositions of the present disclosure including but not limited to any one of the claimed compositions as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for local or systemic administration, e.g, oral, intravenous, intracranial, topical, enteral, and/or parenteral administration. In certain embodiments, the compositions of the present disclosure are formulated for intravenous administration.

Administration of the cells or compositions can be effected in one dose, continuously or intermittently throughout the course of treatment and an effective amount to achieve the desired therapeutic benefit is provided. Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. Suitable dosage formulations and methods of administering the agents are known in the art. In a further aspect, the cells and composition of the disclosure can be administered in combination with other treatments.

The cells and populations of cell are administered to the host and/or subject using methods known in the art and described, for example, in PCT/US2011/064191. This administration of the cells or compositions of the disclosure can be done to generate an animal model of the desired disease, disorder, or condition for experimental and screening assays.

Also provided is an isolated complex comprising, or alternatively consisting essentially of, or yet consisting of either or both of the following: an isolated or engineered cell as disclosed herein bound to a cancer cell, and/or a polypeptide as disclosed herein bound to a cancer cell. In some embodiments, the cancer cell is bound to the isolated cell by the first anti-TAA antigen binding domain as disclosed herein or the anti-BCMA antigen binding domain as disclosed herein. In some embodiments, the cancer cell is bound to the polypeptide by the first anti-TAA antigen binding domain as disclosed herein or the anti-BCMA antigen binding domain as disclosed herein. Additionally or alternatively, the cancer cell is bound to the polypeptide by the second anti-TAA antigen binding domain as disclosed herein or the anti-CS1 antigen binding domain as disclosed herein.

### Combination Therapies

The compositions as described herein can be administered as first line, second line, third line, fourth line, or other therapy and can be combined with cytoreductive interventions. The can be administered sequentially or concurrently as determined by the treating physician. In some embodiments, they can be combined with therapies that may upregulate the expression of a tumor or other antigen to which the CAR and/or BsAb binds.

In some embodiments, some clinical drugs can increase targeted antigens. For example, CS1 surface expression can be increased by Lenalidomide, an immune modulator drug for multiple myeloma that is FDA-approved, see Wang et al. (2018) Clin. Cancer Res. January 1;24(1):106-119. Another example is the FDA-approved drug midostaurin that increases FLT3 expression when the CAR-BsAb targets a FLT3 antigen. In some embodiments, they can be combined with surgical removal of the cancer or tumor. In some embodiments, the cytoreductive therapy comprises, or alternatively consists essentially of, or yet consists of chemotherapy, cryotherapy, hyperthermia, targeted therapy, and/or radiation therapy.

### Kits

As set forth herein, the present disclosure provides methods for producing and administering CAR and/or BsAb CAR cells. In one particular aspect, the present disclosure provides kits for performing these methods as well as instructions for carrying out the methods of the present disclosure such as collecting cells and/or tissues, and/or performing the screen/transduction/etc., and/or analyzing the results.

In one aspect, the kit comprises, or alternatively consists essentially of, or yet further consists of, any one or more of: a polypeptide as disclosed herein, a polynucleotide as disclosed herein, a vector as disclosed herein, an isolated nucleic acids disclosed herein, a vector comprising said nucleic acid, a cell as disclosed herein, such as isolated allogenic cells, preferably T cells or NK cells, a cell population as disclosed herein, a composition as disclosed herein, an isolated complex as disclosed herein, and/or instructions optionally on the procuring of autologous cells from a patient. Such a kit may also comprise, or alternatively consist essentially of, or yet further comprise media and other reagents appropriate for the transduction and/or selection and/or activation and/or expansion of CAR and/or BsAb CAR expressing cells, such as those disclosed herein.

In one aspect the kit comprises, or alternatively consists essentially of, or yet further consists of, an isolated CAR and/or BsAb CAR expressing cell or population thereof. In some embodiments, the cells of this kit may require activation and/or expansion prior to administration to a subject in need thereof. In further embodiments, the kit may further comprise, or consist essentially of, media and reagents, such as those covered in the disclosure above, to activate and/or expand the isolated CAR and/or BsAb CAR expressing cell. In some embodiments, the cell is to be used for CAR therapy. In further embodiments, the kit comprises instructions on the administration of the isolated cell to a patient in need of CAR therapy.

The kits of this disclosure can also comprise, e.g., a buffering agent, a preservative or a protein-stabilizing agent. The kits can further comprise components necessary for detecting the detectable-label, e.g., an enzyme or a substrate. The kits can also contain a control sample or a series of control samples, which can be assayed and compared to the test sample. Each component of a kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

The following examples are illustrative of procedures which can be used in various instances in carrying the disclosure into effect.

### Example 1—BsAb CAR T-Cells Generation and Efficacy

Chimeric antigen receptor (CAR) T cells and bispecific antibodies (BsAb) are FDA-approved therapies and show impressive curative potential for cancer. However, in the majority of cases, neither have yet been shown to be curative. This could be due in part to the duration of the therapies, i.e., CAR T cells may not survive sufficiently long in vivo, and BsAb have a very short half-life with a costly and time-consuming manufacturing process, thus limiting their efficacy and broad application. Here, Applicants successfully created a platform to combine CAR T cell therapy with BsAb therapy, both of which have potential for long-lasting effects. Applicants tested this platform in the setting of multiple myeloma (MM), an incurable cancer with high rates of relapse following currently FDA-approved therapies. Applicants validated the concept utilizing two MM target antigens, CS1 and BCMA, and created a novel and effective single lentiviral construct to generate BsAb-CAR T cells expressing a BCMA CAR as well as secreting a soluble anti-NKG2D-anti-CS1 BsAb, with the former attacking BCMA(+) MM tumor cells and the latter engaging all NKG2D(+) cytolytic cells including CD8(+) T cells, γδ T cells, natural killer (NK) T cells, and NK cells, for the purpose of promoting their cytotoxicity activity against CS1(+) MM. This all-in-one, multifaceted immune modality provides two “living drugs” simultaneously, i.e., CAR T cells and BsAb, capturing both innate and adaptive immune effector cells directed at different target antigens on the same malignant population. Applicants found that, compared with BCMA CAR T cells or BsAb transduced-T cells, BsAb-CAR T cells secreted more IFN-γ and showed higher capacity for degranulation, while displaying enhanced cytotoxicity in vitro through targeting MM tumor cells, including MM cell lines and primary MM tumor cells. Ectopically forced expression of BCMA and CS1 in target cells lacking endogenous expression of these two antigens enhanced target cell lysis. Importantly, the anti-NKG2D-anti-CS1 BsAb secreted from the BCMA CAR T cells acts in an autocrine manner to trigger the BCMA CAR T cell proliferation in vitro and their enhanced proliferation and survival in in vivo, respectively, through activation of NKG2D signaling. These multipronged effects resulted in strong anti-tumor activity in vivo. Collectively, provided herein is evidence for generating next-generation cancer immunotherapy, with a capacity to combine CAR T cell therapy and anti-NKG2D bispecific antibody therapy into a single platform for increased duration and enhanced efficacy as well as the ability to capture specific anti-tumor activity of both innate and adaptive cytolytic effector cells.

Cell culture: Cell lines, MM.1S, H929, RPMI-8226 (human multiple myeloma cell lines), and K562 (human erythroleukemic cell line) were purchased from the ATCC (Manassas, Va., USA). These cells were cultured with RPMI 1640 media (Sigma, St. Louis, USA) containing 10% fetal bovine serum (FBS) (Invitrogen, CA, USA) and 1% Antibiotic-Antimycotic (Invitrogen). The 293T cell line, which was purchased from ATCC and used for lentiviral production, was cultured in DMEM (Sigma) plus the same supplements as in RPMI 1640. Human peripheral blood mononuclear cells (PBMCs) from healthy donors and M/M patients were isolated by Ficoll-Paque Plus (GE Healthcare Bio-Sciences, Pittsburgh, Pa.) density gradient centrifugation, following the manufacturer's instructions. Human CD56⁺ NK cells, CD3⁺CD56⁺ NKT cells, and CD3⁺γSTCR⁺ T cells were isolated using human NK, NKT and γδT cell isolation kits (MACS, Miltenyi Biotech, Auburn, Calif., USA), respectively, according to the manufacturer's instructions. Primary MM patient samples were provided by the Leukemia Tissue Bank Shared Resource of the OSU Comprehensive Cancer Center and James Cancer Hospital. All work with human subjects was performed according to a protocol approved by The Ohio State University Institutional Review Board.

Mice: Six- to 8-week-old NSG (NOD.Cg-Prkdcscid Il2rgtm1Wjl/SzJ) mice were purchased from Jackson Laboratories (Bar Harbor, Me., USA) and were used for all in vivo studies. All animal work was performed according to a protocol approved by The Ohio State University Animal Care and Use Committee. The progression of MM disease was closely monitored, and survival data were recorded. The mice were sacrificed upon observation of hind limb paralysis, lethargy, and obvious weight loss.

Generation of BCMA-CAR, anti-NKG2D-anti-CS1 BsAb, and BsAb-BCMA CAR-lentiviral constructs: For BCMA CAR, the BCMA coding domain sequences for variable regions of heavy (VH) and light (VL) chains were derived from a hybridoma and recombined using a linker. The VH-linker-VL fragment was incorporated in frame with the CD28-CD3zeta portion. The anti-BCMA-scFv-CD28-CD3zeta fragment was subcloned into the lentiviral vector pCDH to create a second-generation pCDH-BCMA CAR construct. To make the anti-NKG2D-anti-CS1 BsAb lentiviral construct, two codon-optimized single chain variable fragments (scFv) from an anti-CS1 monoclonal antibody²⁹ and an anti-NKG2D antibody joined together by a non-immunogenic protein linker derived from human muscle aldose³⁵ were cloned into the pCDH lentiviral vector. For “all in one” BsAb-BCMA CAR, the anti-BCMA-scFv-CD28-CD3ζ-T2A cassette was incorporated into the pCDH anti-CS1-NKG2D BsAb-EF1a-GFP to build a complete pCDH-BCMA CAR-T2A-BsAb-EF1a-GFP lentiviral construct. These methods used to generate BsAb-CARs as described herein.

Lentiviral production and transduction of T cells: Lentiviral transfection and infection were performed as described in a previously reported protocol^{36,37}.

Generation of K562 cells stably expressing the CS1 and BCMA genes: The full length pCDH-CMV-CS1-EF1α-GFP construct containing human CS1 coding sequences was previously reported³⁰. To produce lentivirus, 293T cells were co-transfected with the pCDH-CS1 plasmid or a pCDH empty vector plasmid plus the packaging plasmids pCMV-VSVG and pCMV-δr9 using Lipofectamine®2000 (Invitrogen). Then the lentiviral supernatants were harvested and used to infect K562 cells using a previously published protocol 36.38 GFP-positive cells were then sorted using an FACS Aria II cell sorter (BD Biosciences, San Jose, Calif., USA). BCMA-K562 are K562 cells transduced with a vector carrying the full-length BCMA cDNA. Lentivirus production, infection and sorting were performed using the methods described above. CS1⁺BCMA⁺K562 cells were generated by transducing a pCDH-CMV-BCMA-EF1α-GFP lentiviral construct to CS1-K562 cells described above. The double-transduced cells were stained with an APC-anti-BCMA mAb and then sorted for GFP⁺BCMA⁺ population. Before being used for experiments, these GFP⁺BCMA⁺ double positive cells were passed several times in culture to ensure the loss of anti-BCMA mAb-bound cells.

Flow Cytometry Analysis: Detection of CAR expression on the cell surface was performed as previously reported³⁰. Antibodies used in this study include: FITC and biotin-labeled goat anti-mouse (Fab)2 polyclonal antibody or normal polyclonal goat immunoglobulin G (IgG) antibody (Jackson ImmunoResearch), allophycocyanin (APC)-conjugated streptavidin (Jackson ImmunoResearch), PerCP/Cy5.5-conjugated streptavidin (Biolegend), PE, PerCP/Cy5.5 and BV421 anti-human CD3 (hCD3, clone UCHT1 and SK7, BD Biosciences), APC and PE anti-hCD56 (clone TULY56 and CMSSB, eBioscience), FITC and PC5.5 anti-TCR pan γ/δ (clone IMMU510, Beckman Coulter, Inc. CA, USA), PC5 anti-TCR pan α/β (clone IP26A, Beckman), FITC anti-TCR Vγ9 (clone IMMU 360, Beckman) and Pacific Blue anti-TCR Vδ2 (clone IMMU 389, Beckman), unconjugated and APC-anti hNKG2D (clone 1D11BD Biosciences), PE-Cy7 anti-hCD8 (clone SK1, BD Biosciences), APC-Cy7 anti-hCD4 (clone SK3, BD Biosciences), BV421 anti-human CD1d (clone CD1d42, BD OptiBuild), V450 anti-hCD11c (clone B-ly6, BD Horizon), APC-H7 anti-hCD19 (clone HIB19, BD Pharmingen), APC-H7 anti-hCD20 (clone 2H7, BD Biosciences), FITC anti-hCD45 (clone J.33, Beckman), biotin and FITC anti-hCD14 (clone 63D3 and M5E2, Biolegend), biotin and PE anti-hCD33 (clone P67.6, Biolegend, and WM53, BD Pharmingen), biotin anti-hCD66b (clone G10F5, Biolegend), unconjugated and PE-anti hCS1 (clone 162, eBioscience), APC-anti hBCMA (clone 19F2, Biolegend), PE-anti Ki67 (clone SolA15, BD Biosciences), PE-hCD69 (clone FN50, BD Biosciences). Cells were washed once with PBS containing 4% bovine serum albumin, stained with antibodies for 20 min at room temperature, and analyzed with a LSRII flow cytometer (BD Biosciences, San Jose, Calif., USA).

Immunoblotting: To detect intracellular expression and secretion of the bispecific antibody, BsAb-transduced T cells or BsAb-CAR T cells and cell-free supernatants from the culture of these cells were collected for immunoblotting using 6x-his-tagged mAb (“6x-his” disclosed as SEQ ID NO: 111) (clone 4A12E4, Invitrogen). Immunoblotting was performed according to a standard immunoblotting protocol that Applicants previously reported^{30,37}. For the detection of CAR expression, CAR-transduced T cells were lysed and proteins were extracted for immunoblotting, probing with mouse anti-human CD3ζ mAb (BD Pharmingen), as previously reported 3°.

Cytotoxicity Assay: cells were labeled with ⁵¹Cr and co-cultured with transduced T cells at various effector: target ratios (E:T) in the wells of 96-well V-bottom plates at 37° C. for 4 h, followed by harvesting supernatants to measure the release of ⁵¹Cr from target cells using TopCount counter (Canberra Packard). When studying the capacity of BsAb to engage PBMC, CD3⁺T cells, CD8⁺cytotoxic T cells, γδ T cells, NKT cells, and NK cells were isolated from leukopacks ordered from the American Red Cross. T cells were primed with Dynabeads™ Human T-Activator CD3/CD28 (4×10⁴/μL, Beads:T=1:1) with IL-2 (500 U/mL) and IL-15 (500 U/mL) for 5-14 days prior to the standard 4-hour ⁵¹Cr release assay described above. Human NK cells were activated by IL-2 (500 U/mL) for prior to cytotoxicity assay. Isolated human CD3⁻CD56⁺ NKT cells were activated by α-GalCer (α-Galactosylceramide, KRN7000, Enzo Biochem Inc. NY, USA. 100 ng/mL) with IL-2 (100 U/mL)³⁹ for 7-10 days. For CD3⁺γδTCR⁻ T cells activation, HMBPP ((E)-1-Hydroxy-2-methyl-2-butenyl 4-pyrophosphate, Sigma. 10 nM) with IL-2 (100 U/mL)^{40,41} were used and cultured for 14 days. The protocol for isolation of these cells from leukopacks was approved by The Ohio State University Institutional Review Board.

ELISA: The presence of human IFN-γ in culture supernatants was assayed by enzyme-linked immunosorbent assay (ELISA) using a kit from R&D Systems (Minneapolis, Minn., USA) according to the manufacturer's protocol. The levels of human IL-2 and TNF-α in culture supernatants were also assayed by ELISA kits (Thermo Fisher Scientific, MA, USA). For these ELISA analyses, 2.5×10⁵ cells of either a myeloma cell line or primary MM cells from patients were incubated with 2.5×10⁵ engineered or control T cells in 96-well V bottom plates for 24 h. Data were read at 450 nm using a Synergy HT microplate reader (Biotek, Winooski, Vt., USA).

In vivo treatment of MM-bearing mice and bioluminescence imaging: MM.1S myeloma cells expressing a firefly luciferase gene, MM.1S-GL3, have been previously described¹⁰. To build a xenograft orthotopic MM model, NSG mice (male) were injected with 8×10⁶ MM.1S-GL3 cells in 200 μL of saline through tail-vein i.v. on day 0. On days 10, 17 and 24, mice were administered (1) vehicle control (saline) or 10×10⁶ effector cells, including (2) empty-vector transduced T cells, (3) BsAb-transduced T cells, (4) BCMA-CAR-transduced T cells, (5) T cells sequentially transduced with BsAb and BCMA-CAR, or (6) BsAb-CAR-transduced T cells, by tail-vein i.v. injection, each in 200 μL saline. Ten days, 24 days, 31 days after inoculation with MM.1S-GL3 cells, D-luciferin (150 mg/kg body weight; Gold Biotechnology) was intraperitoneally injected to all mice. Imaging was performed using the In Vivo Imaging System (IVIS) with Living Image software (PerkinElmer). On day 80, we challenged the surviving mice by tail-vein i.v injection of 4×10⁶ MM1.S cells per mouse in 200 μl saline, Survival data were collected and closed on day 140. To investigate the effect of BsAb, the above experiment was repeated in the presence of PBMC with depletion of human myeloid cells. For this purpose, NSG mice were i.v. injected with 8×10⁶ MM.1S-GL3 cells in 200 mL of saline via tail vein on day 0. On day 10, the mice were administered with 3×10⁶ various transduced-T cells followed by 3×10⁶ CD33⁻CD14⁻CD66b⁻human PBMC, all i.v. On day 17 and 24, the mice received 3×10⁶ engineered T cells i.v. generated from the same donor. On day 10, day 19, day 28, and day 37, the mice were infused with D-luciferin and imaged as described above.

Immune-synapse detected by immunofluorescence microscopy: Primarily to label the secreted anti-NKG2D-anti-CS1 BsAb, supernatant from BsAb CAR T cells were collected and stained by 6x-His Tag mAb (“6x-His” disclosed as SEQ ID NO: 111) (clone 4E3D10H2/E3, Invitrogen) at a dilution of 1:500 for 1 h in 37° C. incubation and then labeled with Alexa Fluor®350 (blue, Thermo Fisher Scientific, MA, USA). MM.1S cells were harvested and incubated 45 min under growth conditions with CellTracker™ Deep Red Dye (20 μM, Thermo Fisher Scientific). To see the immune-synapse, BsAb-CAR T cells (GFP, green) or empty vector-transduced control T cells (GFP, green) were co-cultured with MM.1S cells (red) and His Tag labeled supernatant for 1 h or 24 h. Live-cell fluorescence imaging were observed by Zeiss Microscope Systems (Zeiss Axio Observer Z1, Carl Zeiss Inc., NY, USA). For video shooting, BsAb-CAR T cells (GFP, green) or empty vector-transduced T cells (GFP, green) were co-cultured with MM.1S cells (red) for 1 h, and microscopy was used for observing immune-synapse during a period of 2 h.

Results: Generation of primary T cells expressing BCMA-specific CAR and/or the anti-NKG2D-anti-CS1 bispecific antibody: Applicants generated a specific BCMA-CAR construct with a lentiviral vector backbone, which sequentially consists of a signal peptide (SP), a heavy chain variable region (VH), a glycine-serine (GS) linker, a light chain variable region (VL), a Myc tag, a hinge, CD28, and CD3C (FIG. 1A). Next, applicants designed the anti-NKG2D-anti-CS1 bispecific antibody (referred to as “BsAb”) construct with a lentiviral vector backbone. It consisted of two codon-optimized single chain variable fragments (scFv) from an anti-NKG2D antibody and an anti-CS1 monoclonal antibody, joined together by a non-immunogenic protein linker derived from human muscle aldose. Each scFv contains a corresponding heavy chain (VH) and light chain (VL) connected by a glycine-serine (GS) linker (FIG. 1B). The same donor T cells isolated from a healthy donor and activated by anti-human CD3/CD28 antibody beads were transduced with the empty vector (EV), the BsAb construct, BCMA-CAR construct, or first transduced with the BsAb construct followed sequentially by transduction with the BCMA-CAR construct (hereafter referred to as the BsAb-BCMA seq. trans. T construct). The expression of BCMA CAR on the cell surface was demonstrated by staining transduced T cells with anti-Fab, which detected expression of the scFv on more than 80% of FACS-enriched T cells transduced with either the BCMA CAR construct or the BsAb-BCMA seq. trans. T cell construct, whereas the expression remained almost undetectable on unmodified T cells, on EV-transduced T cells and on BsAb T cells (FIG. 1C). To determine whether BsAb T cells and the BsAb-BCMA seq. trans. T cells were successfully transduced, cell-free supernatants from a 4-day culture were harvested and cell pellets from a 4-day culture were lysed. Both the supernatants and cell lysates were then subjected to immunoblotting using a 6x-his tagged Ab (“6x-his” disclosed as SEQ ID NO: 111). Results showed that BsAb-T cells and BsAb-BCMA seq. trans. T cells produced both cellular and secreted BsAb while the controls from unmodified T cell supernatants and lysates, did not produce BsAb (FIG. 1D).

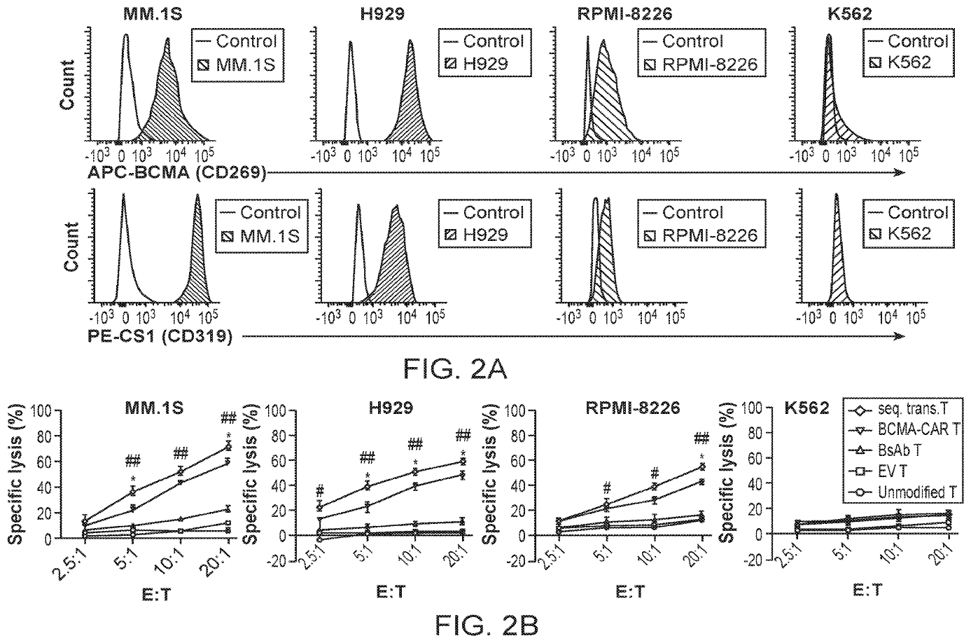

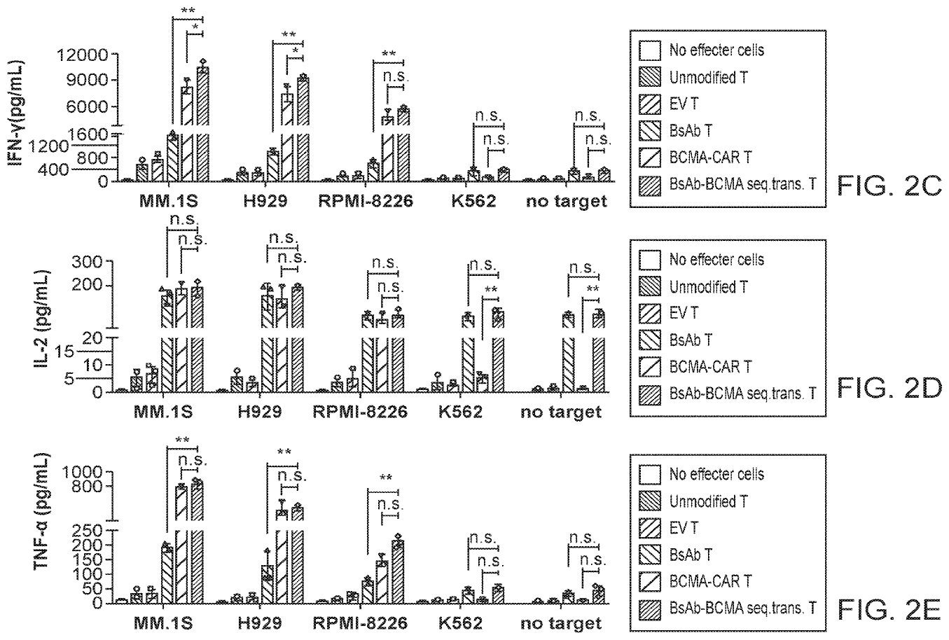

BsAb-BCMA seq. trans. T cells are more effective killers of MM than were T cells transduced with each vector alone in vitro: Since the BsAb contained an anti-NKG2D receptor portion and an anti-CS1 portion, applicants attempted to trigger NKG2D activation on the NKG2D⁺ cytolytic immune cells and tested whether it simultaneously engaged MM cells via the MM-associated antigen, CS1. Applicants first evaluated the surface expression of CS1 and BCMA in three commonly used MM cell lines MM.1S, H929, RPMI-8226 and a human erythroleukemic cell line, K562, by flow cytometric analysis. The results showed varied levels of BCMA and CS1 expression on the four MM cell lines. The MM1.S MM cell line has high levels of expression of both BCMA and CS1; the H929 MM cell line has high levels of BCMA and intermediate (int) levels of CS1 expression; and the RPMI-8226 MM cell line has intermediate levels of BCMA expression, while its CS1 expression is very low. As a negative control, the K562 erythroleukemia cell line did not express CS1 nor BCMA on the cell surface (FIG. 2A). To determine whether the aforementioned BsAb-BCMA seq. trans. T cells (with sequentially transduced BCMA CAR and anti-NKG2D-anti-CS1 BsAb) could lead to more efficient tumor cell lysis of the MM cell lines, a standard 4-hour ⁵¹Cr-release assay was performed, using the K562 erythroleukemia cell line as negative target control. Prior to generating a single construct-engineered BsAb-CAR T cell, Applicants compared MM tumor cell killing in five groups of T cells: (1) unmodified T cells, (2) empty vector (EV)-transduced T cells (EV T), (3) anti-NKG2D-anti-CS1-BsAb-transduced T cells (hereafter referred to as BsAb T), (4) BCMA-CAR-transduced T cells (hereafter referred to as BCMA-CAR T), and (5) anti-NKG2D-anti-CS1 BsAb and BCMA-CAR sequentially transduced T cells (referred to as BsAb-BCMA seq. trans. T). For the target MM cell lines BCMA^{high}CS1^{high} MM.1S and BCMA^{high}CS1^{int} H929, the BsAb-BCMA seq. trans. T cells produced significantly better killing than the BsAb T cells or BCMA-CAR T cells. For the target MM cell line BCMA^{int}CS1^{low} RPMI-8226, the BsAb-BCMA seq. trans. T cells performed better than BsAb T cells but not better than BCMA-CAR T cells. Importantly, either single or combination antigen targeting had no activity against the negative control K562 erythroleukemic target cell line (FIG. 2B). Of note, BCMA-CAR T cells were more effective at lysing MM target cells when compared to the effects of BsAb T cells, EV T cells, and unmodified T cells. Statistical analysis indicated that synergistic effects were observed for cytotoxicity assays conducted with the BsAb-BCMA seq. trans. T cell population of effector cells when the target MM cell lines expressed medium to high levels of CS1 and BCMA (i.e., MM1.S and H929), while this effect was not observed when target cells expressed low or medium levels of the two antigens (i.e., RPMI-8226) (FIG. 2B).

To further determine the activation of the above transduced-T cells upon recognition of MM cells that endogenously express CS1 and BCMA, the Applicants measured IFN-γ, IL-2 and TNF-α secretion via ELISA in supernatants from unmodified T cells, EV T cells, BCMA-CAR T cells, BsAb T cells, and BsAb-BCMA seq. trans. T cells. IFN-γ secretion from BsAb-BCMA seq. trans. T cells was significantly higher than BsAb T cells or BCMA CAR T cells alone when co-cultured with BCMA^{high}CS1^{high} MM.1S or BCMA^{high}CS1^{int} H929 MM cell lines. In the co-cultured conditions with the BCMA^{int}CS1^{low} RPMI-8226 MM cell line, IFN-γ secretion from BsAb-BCMA seq. trans. T cells was significantly higher than BsAb T cells but not significantly higher compared to BCMA T cells. In the co-culture condition with the CS1⁻BCMA⁻K562 erythroleukemia cell line or no tumor target cells, BsAb-BCMA seq. trans. T cells were not superior to single antigen targeting with either BsAb T cells or BCMA-CAR T cells (FIG. 2C). IL-2 production in BsAb T cells, BCMA-CAR T cells or BsAb-BCMA seq. trans. T cells was dramatically higher than unmodified T cells or EV T cells when co-cultured with MM.1S, H929 or RPMI-8226 MM cell lines, (FIG. 2D). Interestingly, even in co-cultures with the negative control K562 erythroleukemia cell line or no target cells, BsAb T cells and BsAb-BCMA seq. trans. T cells secreted a high level of IL-2 that was significantly higher than IL-2 secretion seen in BCMA CAR T cells (FIG. 2D), suggesting that at least in this instance, the effect appears to result from the presence of the secreted BsAb itself, rather than from the MVI target cells. TNF-α secretion was consistent with IFN-γ secretion (FIG. 2E). Overall, these results indicated that, compared with BsAb T cells or BCMA-CAR T cells, BsAb-BCMA seq. trans. T cells can more specifically recognize MM target cells, and become more activated after the recognition of these MM cells. Moreover, compared to other conditions, Applicants also noticed that the BsAb secreted from BsAb T cells or BsAb-BCMA seq. trans. T cells appeared to trigger T cell activation regardless of the presence or absence of MM cells, as evidenced by their highly abundant IL-2 production compared to T cells not expressing the BsAb construct (FIG. 2D). This suggested that the NKG2D receptor expressed on cytotoxic CD8(+) T cells was being activated by the presence of the secreted BsAb.

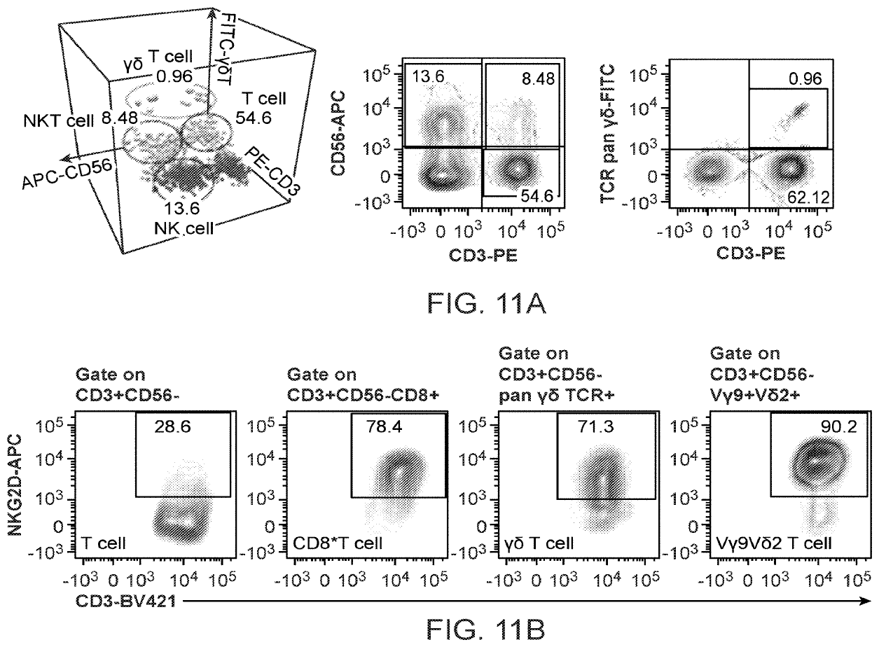

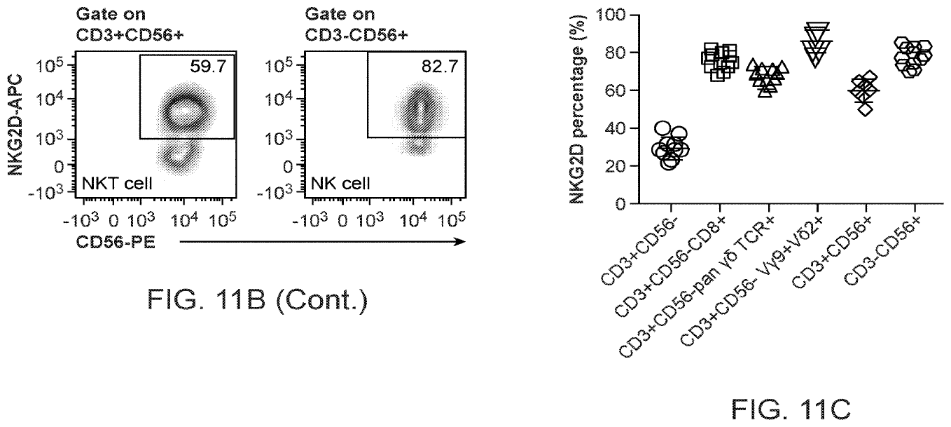

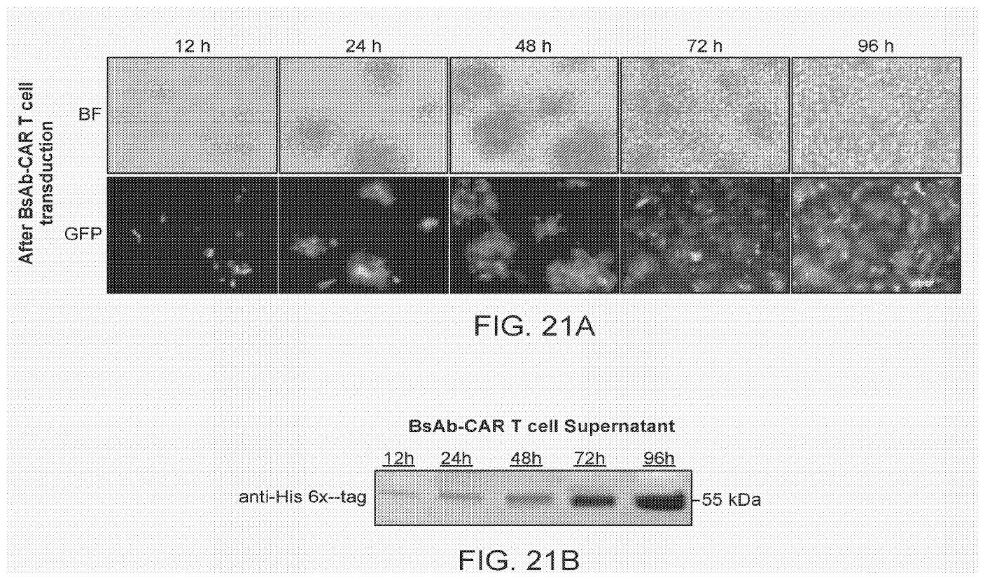

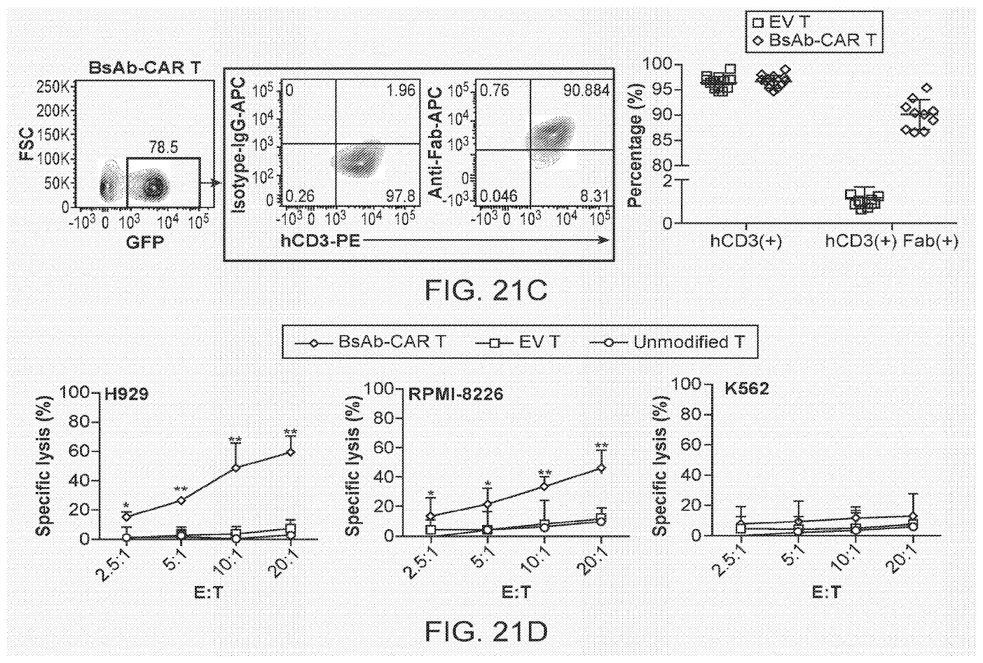

Generation of single construct-engineered BsAb-CAR T cells to target both BCMA and CS1 in MM: Applicants noted that T cells co-expressing BCMA-CAR and anti-NKG2D-anti-CS1 BsAb delivered by two separate constructs (i.e., BsAb-BCMA seq. trans. T cells) were superior at killing MM cells when compared to T cells expressing either BCMA-CAR or BsAb. A single construct expressing both a BsAb and a CAR (referred to hereafter as BsAb-CAR) would be more practical in (1) producing effective expression of both constructs in a single T cell; (2) decreasing manufacturing costs; and (3) saving time. Applicants therefore generated a single BsAb-CAR construct containing both parts in a lentiviral vector backbone connected by T2A (FIG. 3A). To generate primary T cells expressing BsAb-CAR, Applicants utilized the same method described above and then determined whether the BsAb-CAR-transduced T cells were successfully transduced. The surface expression of the CAR was confirmed by flow cytometric analysis (FIG. 21C). The BsAb fusion protein was successfully detected using a 6x-his-tagged Ab (“6x-his” disclosed as SEQ ID NO: 111) on day 4 in both cell lysates and in the serum-free-medium (FIG. 3B). Additionally, to dynamically measure BsAb secretion, applicants re-seeded the BsAb-CAR T cells in serum-free medium on day 5, and then collected cell-free supernatants at 12 h, 24 h, 48 h, 72 h and 96 h. The results showed that the BsAb secretion was potent, as expression started before 12 h and continued to increase in a time dependent manner (FIG. 21A-B). Applicants then demonstrated that unfractionated BsAb-CAR T cells showed significantly superior killing of the BCMA^{high}CS1^{high} MM cell line MM.1 S compared to killing by EV T cells and unmodified T cells (FIG. 3C). Similar results were obtained against the BCMA^{high}CS1^{int} H929 and BCMA^{int}CS1^{low} RPMI-8226 MM target cell lines, while there was no killing against the negative control K562 erythroleukemia cell line (FIG. 21D). As approximately 80% of CD8⁺ T cells have high surface density expression of NKG2D (vs. 30% of unfractionated T cells; FIG. 11A), Applicants transduced to highly enriched CD8⁺ T cells with the BsAb-CAR construct. Perhaps predictably, Applicants found these cells to have higher cytotoxicity than CD8⁺ BsAb T cells and CD8⁺ BCMA-CAR T cells as well as higher than two negative controls, unmodified T cells and EV T cells, against the BCMA^{high}CS1^{high} MM.1S MM cell line (FIG. 3D).

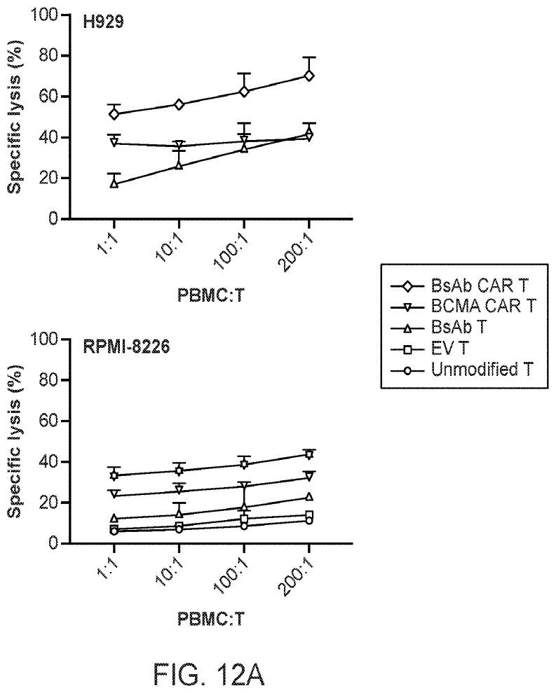

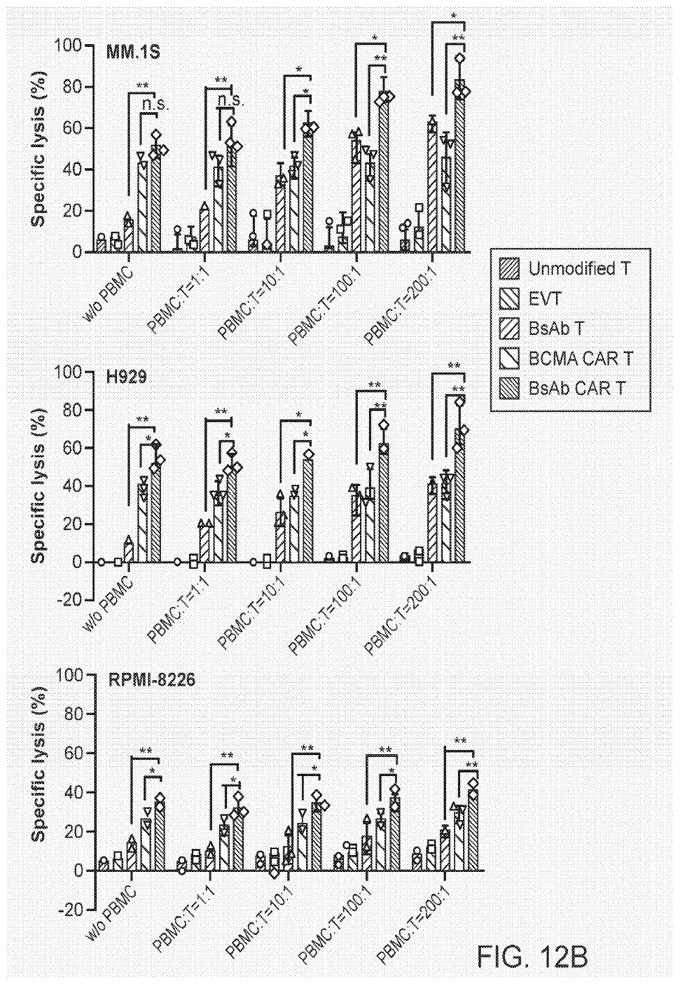

The BsAb secreted by CAR T cells requires two antigens to be functional: CS1 expressed on tumor cells, and NKG2D expressed on immune cells. Applicants first assessed the percentages of TCR pan α/β CD3⁻T, TCR pan γ/δ CD3⁺T, CD3⁺CD56⁺NKT, and CD3⁻CD56⁺NK cells among PBMC, which represent approximately 50%, 1%, 8%, and 15% of PBMC, respectively (FIG. 11A). Furthermore, Applicants confirmed NKG2D is expressed on approximately 30% of T cells, 80% of CD8⁺T cells, 70% of γδ T cells, 60% of NKT cells, and 90% NK cells. Of note, nearly 90% of Vγ9V52 T cells, which are a subset of γδ T cells, expressed NKG2D (FIGS. 11B and 11C). To determine whether anti-NKG2D-anti-CS1 BsAb triggers NKG2D⁺ immune cells, Applicants undertook 4-hour chromium-51 release assays as described above at the ratio of 10 Effector (transduced or unmodified T cells) to 1 target cells (MM.1 S), but added different quantities of human PBMC, i.e., 1-, 10-, 100-, or 200-fold of tumor cells. Applicants hypothesized that if the secreted BsAb from either the BsAb T cells and/or the BsAb-CAR T cells were recruiting non-transduced NKG2D+ cytolytic effector cells to the CS1+MM cell line target, the killing of the target would go up with greater dilution from the addition of non-transduced PBMC. In support of their hypothesis, as non-transduced PBMC were increased, only cultures containing transduced T cells that secreted the BsAb (i.e., BsAb T cells and BsAb-CAR T cells) showed increasing cytotoxicity against the BCMA^{high}CS1^{high} MM.1S MM cell line (FIG. 3E). Predictably, the effect was more modest against the BCMA^{high}CS1^{int} H929 cell line with lower expression of CS1 than the MM.1S MM cell line and was absent against the BCMA^{int}CS1^{low} RPMI-8226 MM target cell line, FIG. 12A, 12B).

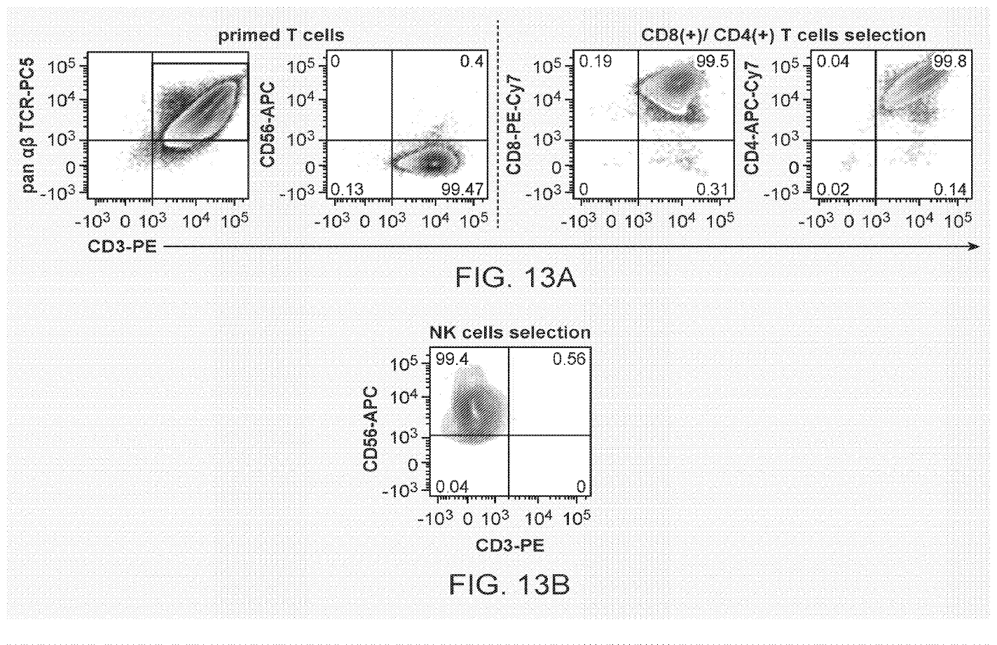

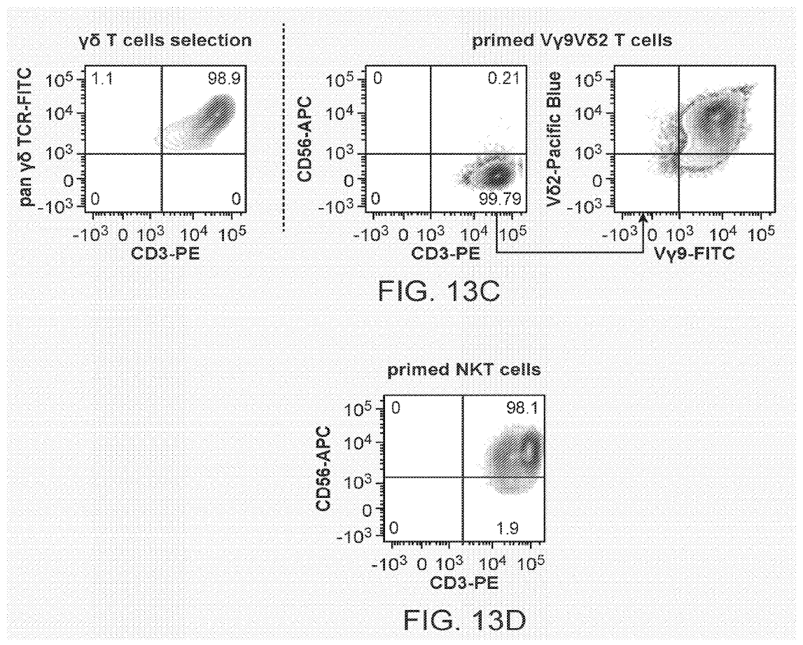

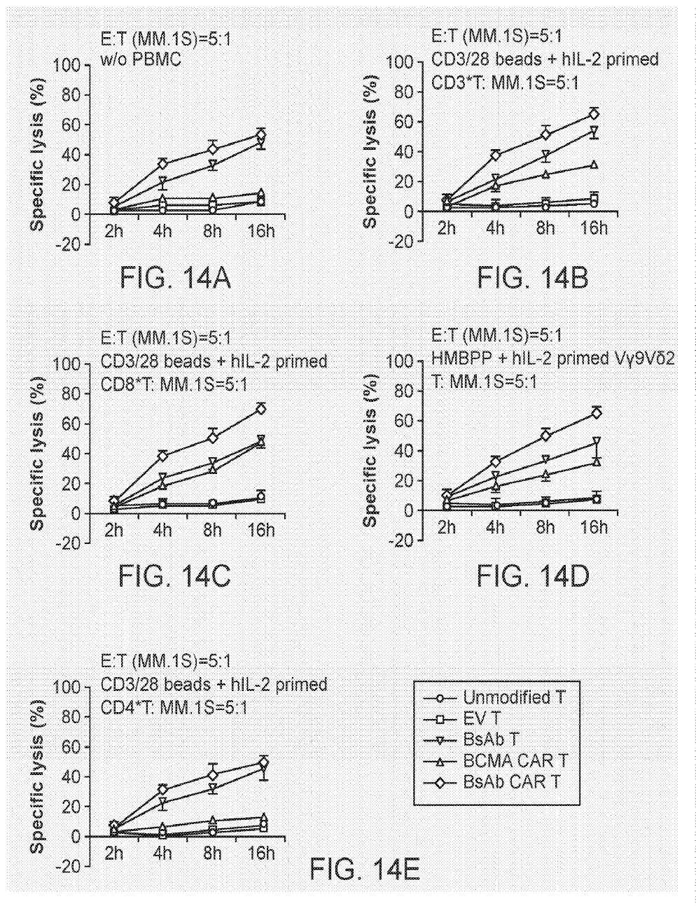

Applicants next enriched each subset of PBMC, i.e., NK cells, NKT cells, CD8⁺ T cells, and γ9Vδ2 T cells, to nearly 98% purity (FIG. 13), and priming them with IL-2, CalCer plus IL-2, CD3/CD28 Dynabeads plus IL-2, and HMBPP plus IL-2, respectively, and transduced each with one of the control or experimental vectors, followed by a 4 h (FIG. 3F, left) or 16 h (FIG. 3F, right) ⁵¹Cr-release cytotoxicity assay against the BCMA^{high}CS1^{high} MM.1S MM cell line at an E:T ratio of 5:1 for each. At four or more hours of incubation, the results showed that BsAb CAR T cells have significantly higher cytotoxicity than identical cells transduced with the other constructs, regardless of the T cell subset or the method of activation (FIGS. 3F and 14). Importantly, among the BsAb CAR T cell populations examined, compared to control cells, the activated CD4⁺ BsAb CAR T cells had the least increase in cytotoxic activity (FIG. 3F, right and FIG. 14), likely due at least in part to the relatively low surface density expression of NKG2D on this population.

To determine whether BsAb secreted by BsAb-CAR T cells can induce synapse formation between BsAb-CAR T cells and MM.1S MM cells, a confocal microscopy analysis was conducted after one hour of co-incubation. When a control of co-culture of EV T cells and MM.1S MM cells was observed, no synapses were seen (FIG. 3G). In contrast, synapses were observed during the co-culture of BsAb-CAR T cells and MM.1S MM cells (FIG. 3H).

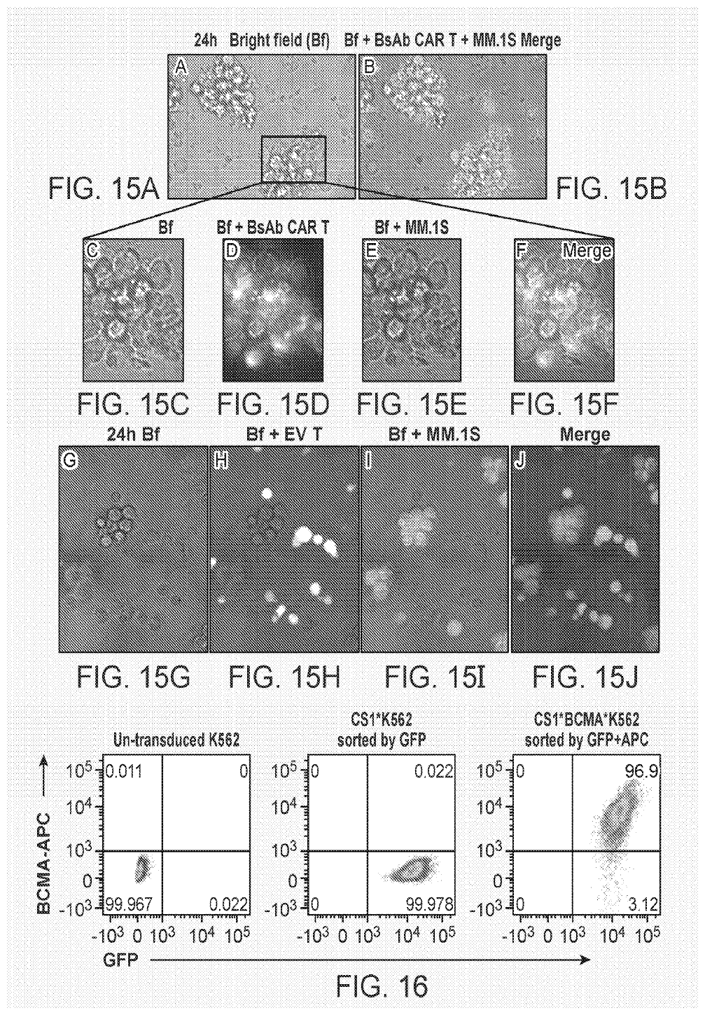

Further, when the 24 hours co-cultures of BsAb CAR-T cells (green) with the target MM. 1 S MM cells (red) were observed, Applicants noticed that only the BsAb CAR-T population (green) was present (FIG. 15A-F); yet in the co-culture of EV T cells and MM.1S MM cells, both the target MM.1S MM cells and the effector EV T cells (green) were still present (FIG. 15G-M).

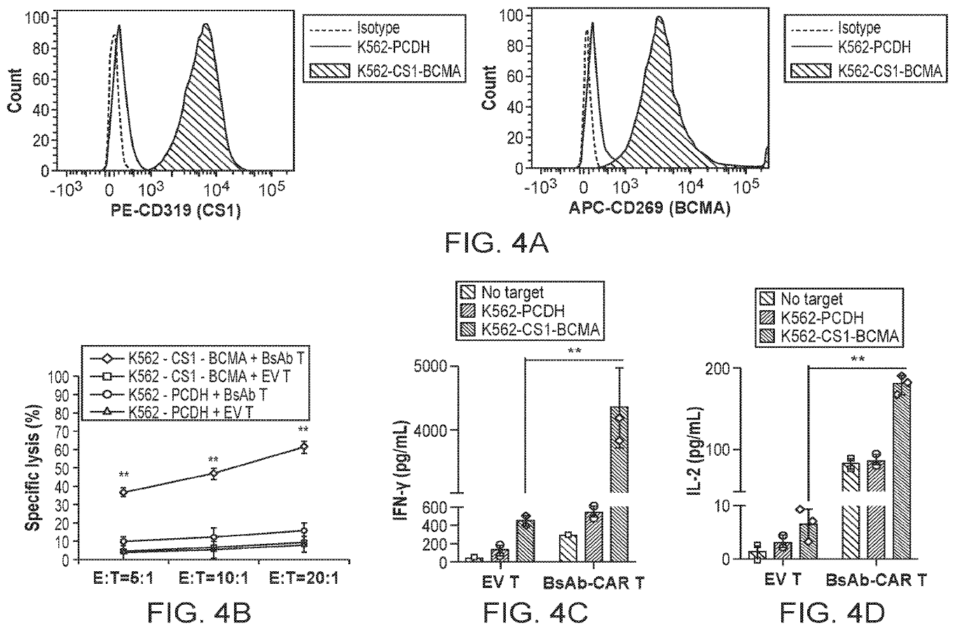

Functionally enhanced recognition and activation of BsAb-CAR T cells are CS1 and BCMA-dependent: To prove that enhanced cytotoxic effect of BsAb-CAR T cells depended on targeting tumor antigens, Applicants next explored whether forced overexpression of CS1 and BCMA in the BsAb-CAR T cell-resistant K562 cell line could lower its threshold for lysis against this effector population. For this purpose, Applicants sequentially transduced to K562 cells with lentiviruses encoding human CS1 and BCMA (or empty vector PCDH as control) to generate the K562 cell line ectopically expressing CST and BCMA (FIG. 16). After confirming the success of generating the target cell line (FIG. 4A), Applicants performed ⁵¹Cr release assays and the results indicated that overexpression of CS1 and BCMA in the K562 erythroleukemia cell line resulted in a significant increase in the cytotoxic activity of BsAb-CAR T cells against this K562 cell line as compared to the cytotoxicity using the EV T cells (FIG. 4B, purple lines).

Applicants next performed ELISA assays, which showed an increase of IFN-γ and IL-2 secretion in co-cultures of BsAb-CAR T cells and K562-CS1-BCMA cells, compared to co-cultures of EV T cells with K562-CST-BCMA cells (FIG. 4C, 4D). However, there was no difference in cytotoxicity, IFN-γ and IL-2 production between BsAb-CAR T cells and EV T cells when they were incubated with K562-PCDH (FIG. 4B-D). Collectively, these data suggested that the increased recognition, killing, and cytokine secretion of target cells by BsAb-CAR T cells occurred in a CS1 and BCMA-dependent manner.

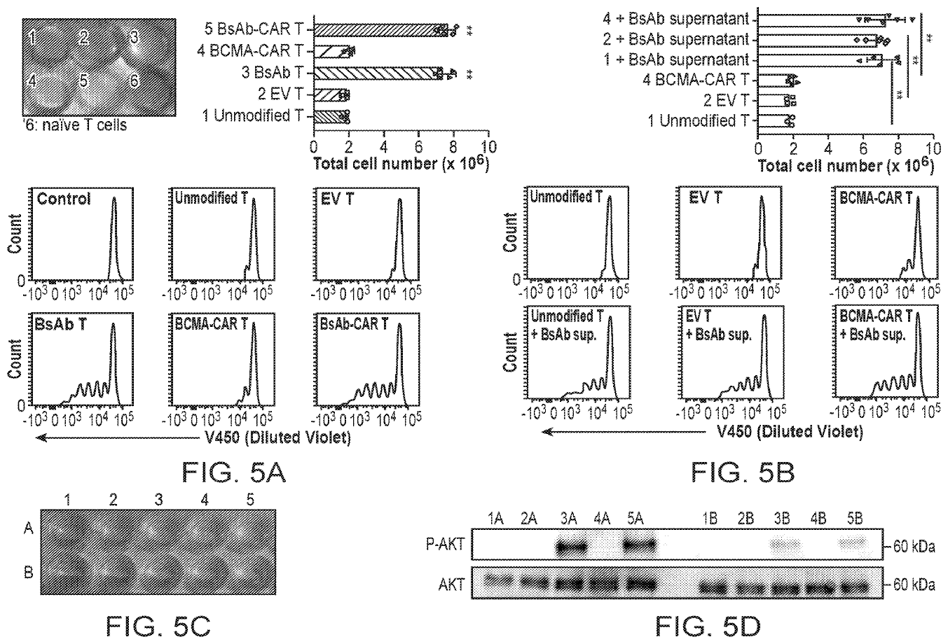

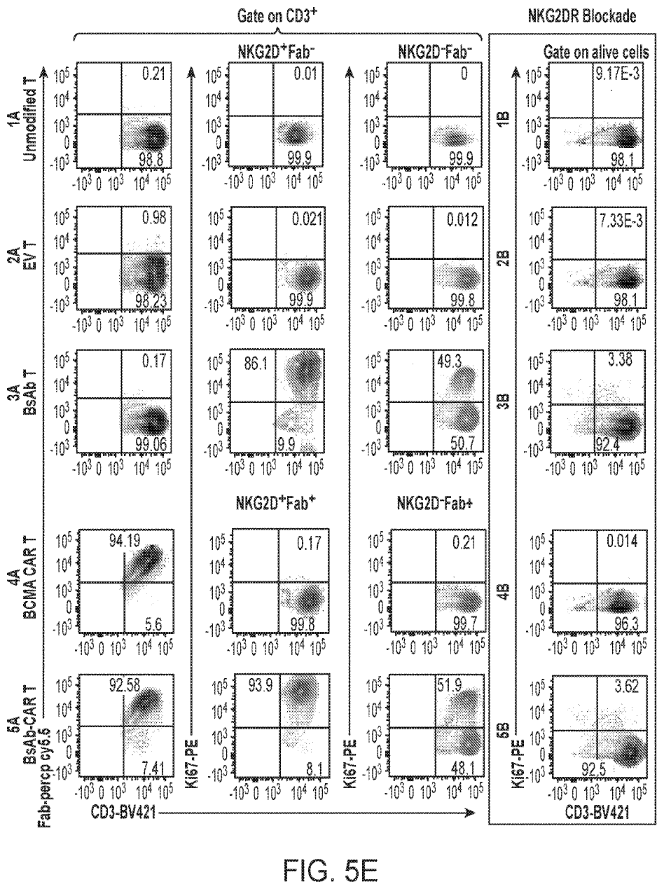

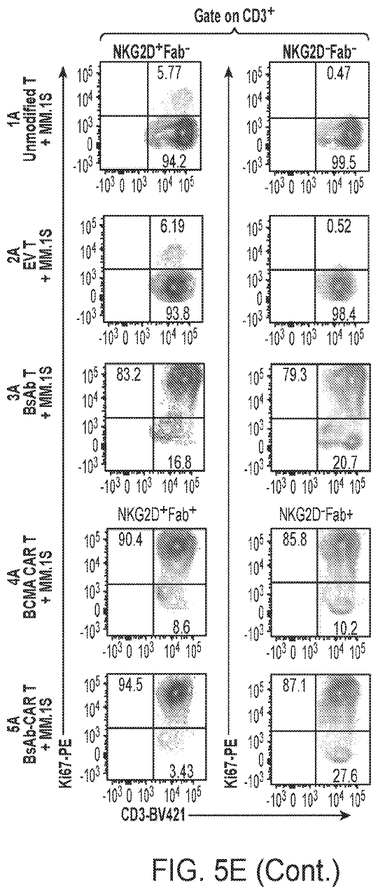

Secreted anti-NKG2D-anti-CS1 BsAb enhances CAR T cell proliferation in vitro and both survival and proliferation in vivo through NKG2D signaling: In some patients, CAR T cells do not survive very long, due to limited expansion and survival capacity, and this can limit the efficacy of these cells⁴². Unexpectedly, Applicants found that the culture medium of BsAb-transduced T cells and BsAb-CAR T cells was more acidotic compared to other conditions without BsAb at similar time points, suggesting a higher rate of cell metabolism (FIG. 5A, top). This observation is consistent with the data described above that these BsAb-transduced cells can secrete more cytokine even without target cells or with BCMA⁻CS1⁻ target cells (FIG. 2D). Applicants speculated that expression and secretion of the BsAb can induce T cell proliferation, survival, and/or its activation. Cell enumeration confirmed that, compared to other culture conditions, those containing T cells transduced with the BsAb or BsAb-CAR construct indeed contained a significantly higher quantity of cells (FIG. 5A, bar graph), which resulted from T cell proliferation as documented by the violet cell tracker and shown as V450 dilution displayed in histograms in the lower panel (FIG. 5A, histogram).

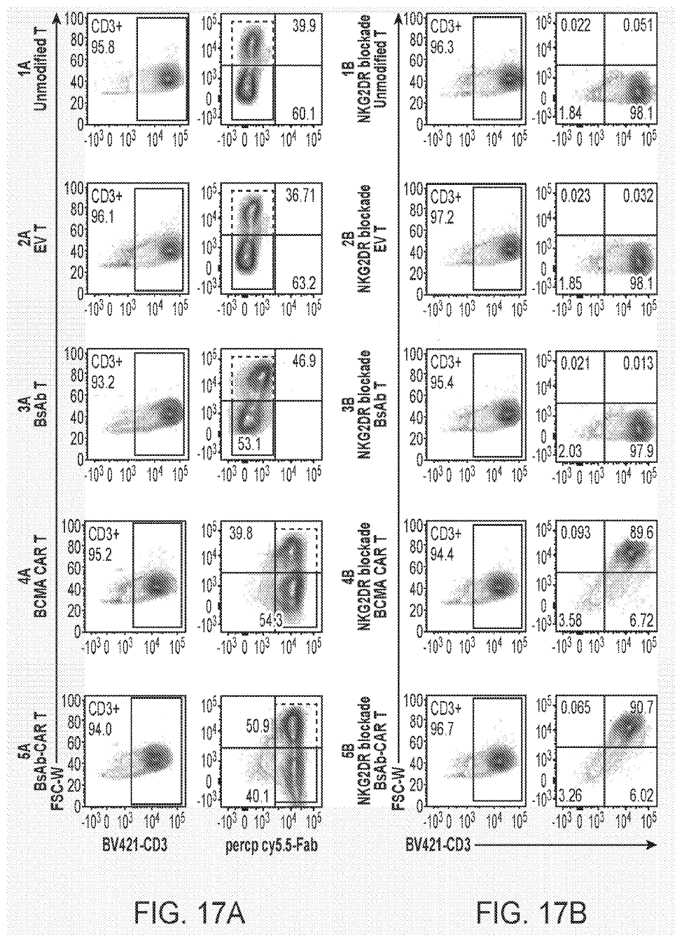

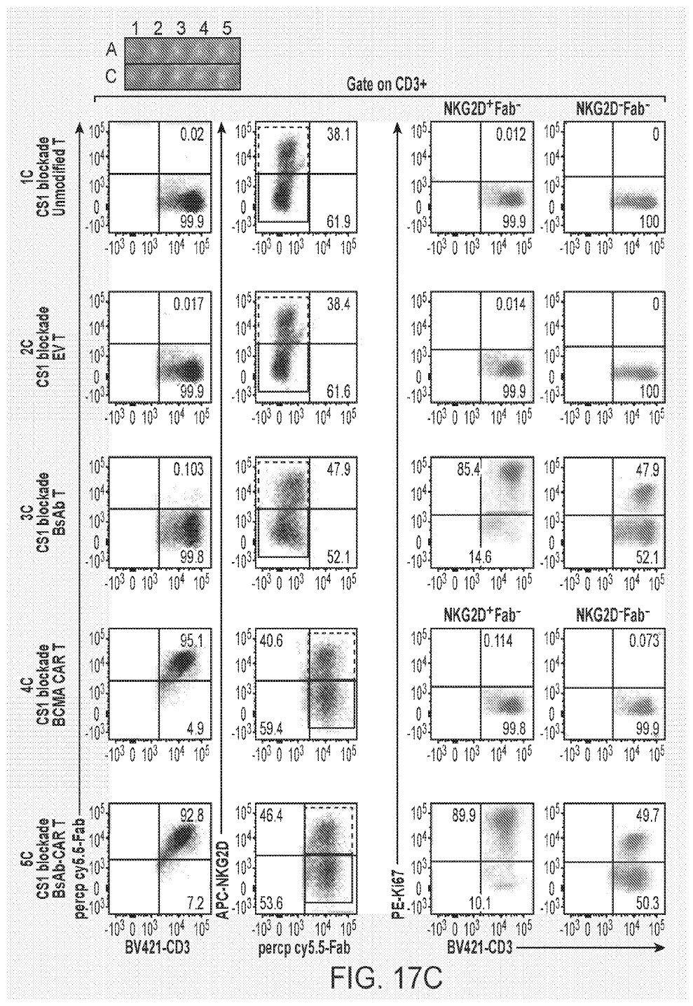

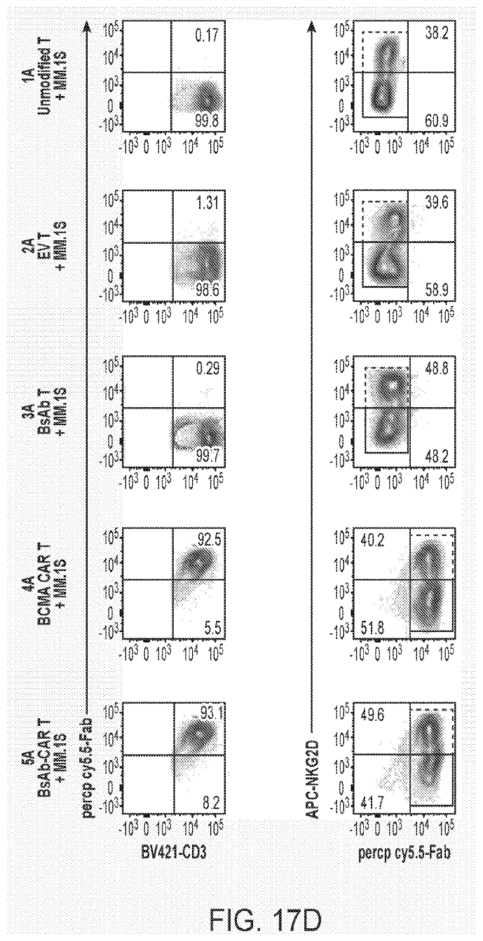

To confirm these unexpected results, Applicants added the supernatants from BsAb-CAR T cell cultures shown in FIG. 5A to cell culture conditions that were without BsAb, i.e., BCMA CAR T cells, EV T cells, and unmodified T cells. Indeed, adding supernatants from BsAb-CAR T cell cultures resulted in a significantly greater degree of cell metabolism after 5 days incubation, as evidenced by the yellow color of the medium as occurs in cell turnover, compared with cultures of BCMA CAR T cells, EV T cells, and unmodified T cells that were not supplemented with supernatants from BsAb-CAR T cell medium (not shown). Cell enumeration confirmed that, compared to other culture conditions, those supplement with medium from BsAb-CAR T cell cultures indeed contained a significantly higher quantity of cells (FIG. 5B, top), which resulted from T cell proliferation as documented by the violet cell tracker and shown as V450 dilution displayed in histograms in the lower panel (FIG. 5B, bottom). Ki67 staining indicated that, in the cultures containing either BsAb T cells or BsAb-CAR T cells, the vast majority of NKG2D⁺ cells were proliferating, as well as nearly half of NKG2D⁻ cells (FIG. 5C, and FIG. 17A), indicating that BsAb-activated NKG2D⁺ cells can in turn activate NKG2D⁻ cells, albeit to a lesser degree. Moreover, NKG2D blocking antibody mitigated the proliferative effects of the secreted BsAb (FIG. 5C blue frame, FIG. 17B). An immunoblot analysis was performed to determine the phosphorylation (p) of AKT protein, confirming that secreted BsAb can trigger NKG2D⁺ cell proliferation and activation under BsAb T and BsAb-CAR T cells culture conditions because these conditions have a higher level of p-AKT (FIG. 5D).

The data presented thus far supported the notion that activation of the NKG2D+ immune cells by the BsAb was occurring through the NKG2D pathway. The Applicants next asked if activation was occurring via CS1, which is expressed not only on MM cells, but also on NK, NKT, CD8⁺ T, and B cells or their subsets. (Veillette and Guo Crit Rev Oncol Hematol. 2013 October;88(1):168-77. doi: 10.1016/j.critrevonc.2013.04.003. Epub 2013 Jun. 2; Gogishvili et al. Blood. 2017 Dec. 28;130(26):2838-2847. doi: 10.1182/blood-2017-04-778423. Epub 2017 Oct. 31.) To determine whether secreted BsAb was also stimulating NKG2D⁺ immune cells via their expression of CS1 and via the CS1⁺ signaling pathway, Applicants next blocked this pathway using an anti-CS1 blocking antibody. Applicants found CS1 blockade did not affect the activation status of immune cells (FIG. 17C). Moreover, Applicants performed NKG2D and CS1 double blocking, and results demonstrated that there was no difference compared with NKG2D single blockade (data not shown). Collectively, these data suggest that activation of immune cells secreting BsAb only occurs through the NKG2D signaling pathway.

Based on the above findings, Applicants had anticipated that in co-cultures of the T cells with the target population, MM.1S MM cells, immune activation would not be strictly dependent on NKG2D, nor restricted to the NKG2D⁺ subset of T cells. For example, when BsAb T and BsAb-CAR T cells were co-cultured with MM.1S MM cells, both NKG2D⁺ (red in FIG. 5E; 83% to 94%) and NKG2D⁻ (green in FIG. 5E; 79% to 87%) fractions of CD3⁺ T cells showed extensive proliferation as measured by Ki67 staining. Likewise, for CD3⁺ T cells only expressing the BCMA CAR (denoted in FIG. 5E as Fab) both NKG2D⁺ (Red; 90.4%) and NKG2D⁻ (Green; 85.8%) fractions of CD3⁺ T cells showed extensive proliferation as measured by Ki67 staining, albeit slightly less than was seen with both NKG2D⁺ and NKG2D⁻ BsAb-CAR T cells.

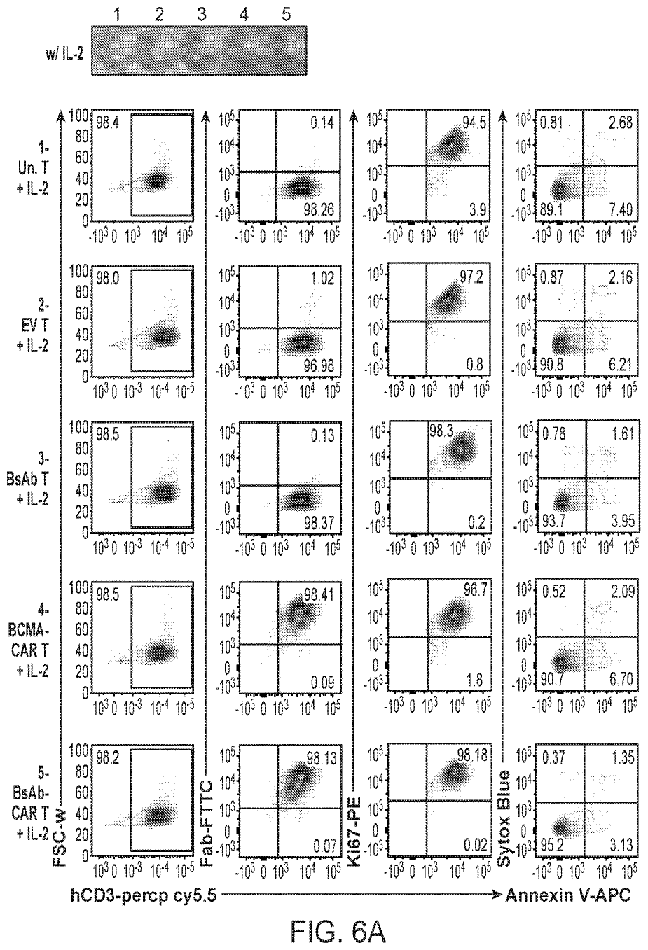

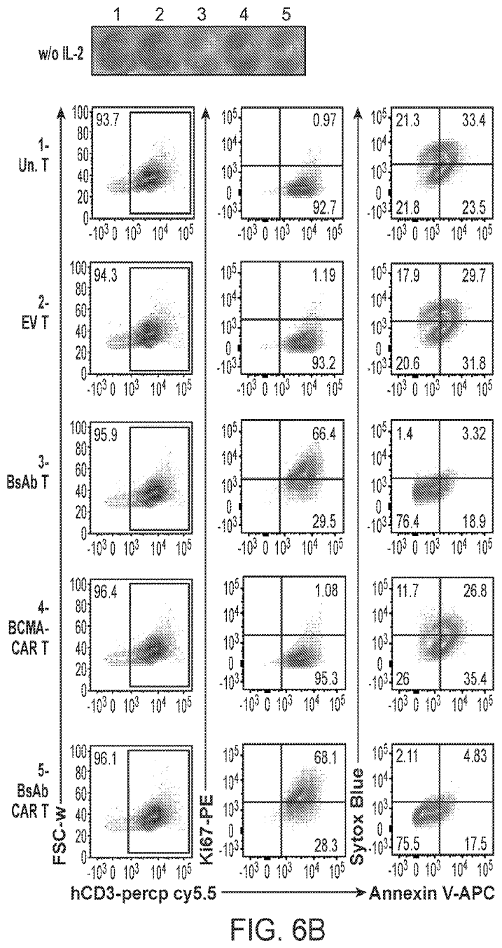

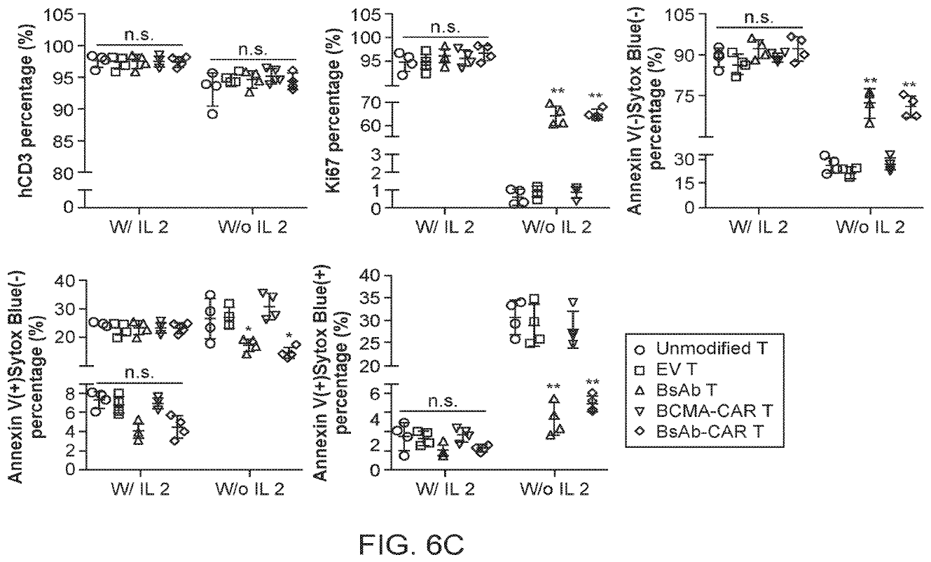

To investigate the ability of the transduced T cells to survive in vitro, Applicants cultured various transduced T cells in the presence or absence of IL-2. Under the IL-2 condition, all cells showed high Ki67 expression and low Annexin V and/or Sytox Blue expression (FIG. 6a). Interestingly, in IL-2 deficient condition (FIG. 6b), only the BsAb T cells and BsAb-CAR T cells (which secrete the BsAb and activate the T cells via NKG2D) showed better proliferation ability as about 80% Ki67 expression, which is consistent with data presented in FIG. 2D, and with the reduced cell apoptosis and death as illustrated by the low expression of Annexin V and/or Sytox Blue staining (FIG. 6b). These results are compared with the other three groups (unmodified T cells, EV T cells, and BCMA-CAR T cells, also in FIG. 6c. Therefore, BsAb-CAR T have enhanced cell proliferation and augmented cell survival, most likely via the NKG2D signaling pathway.

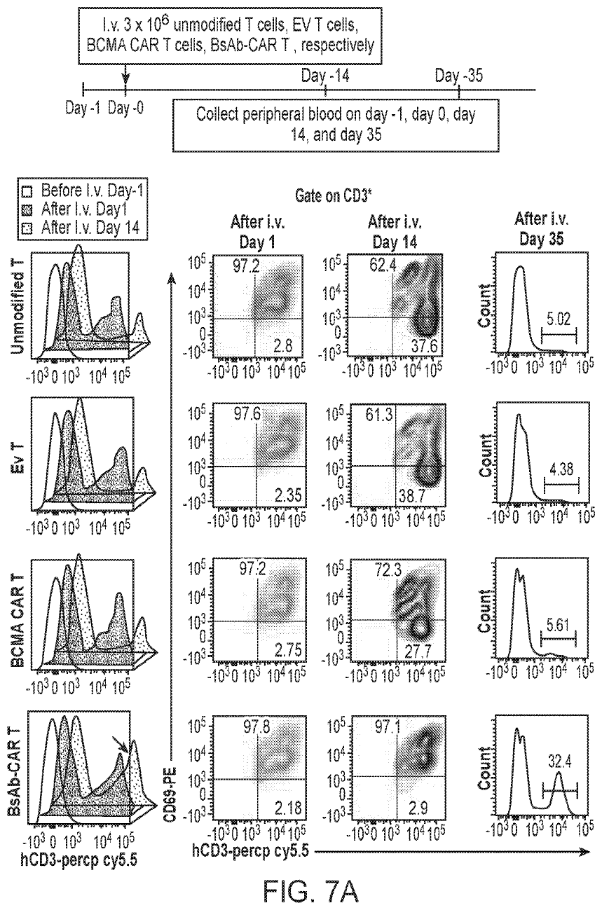

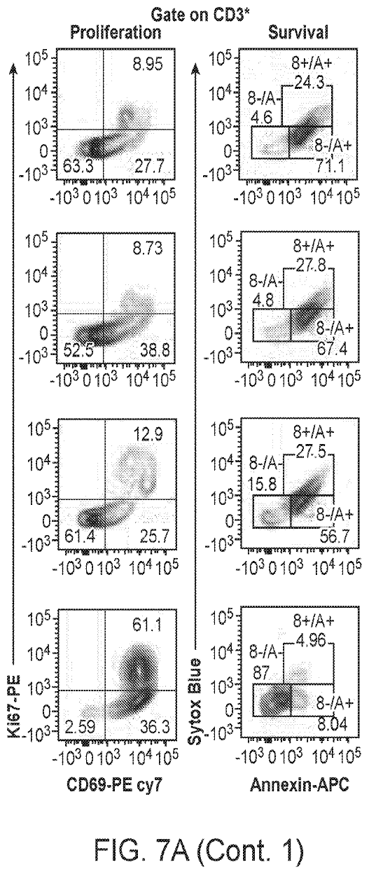

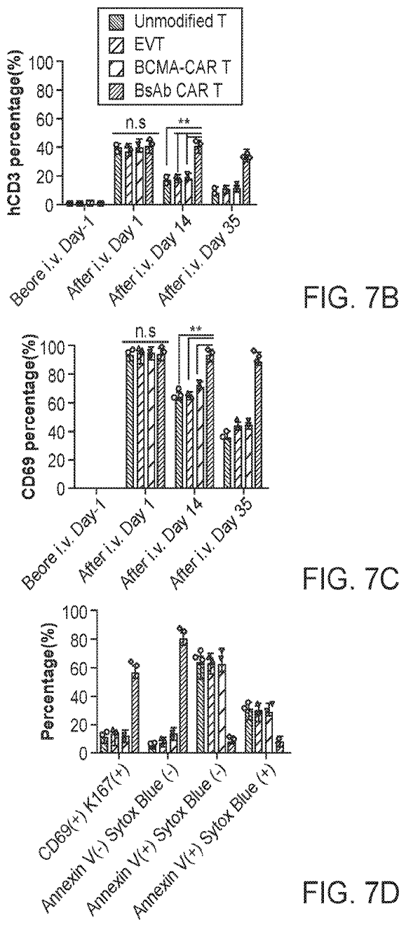

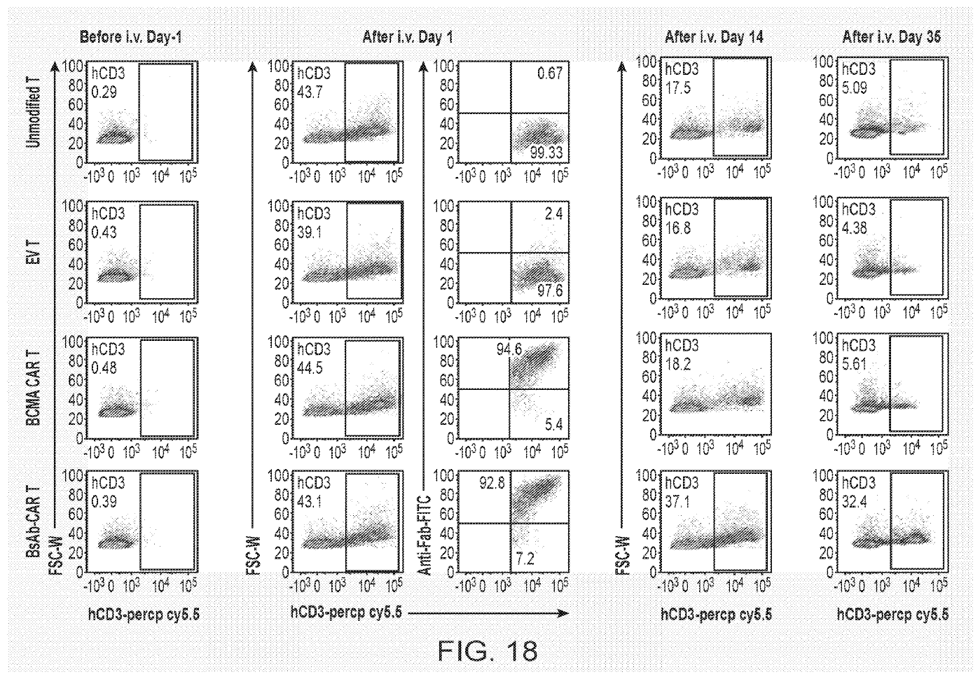

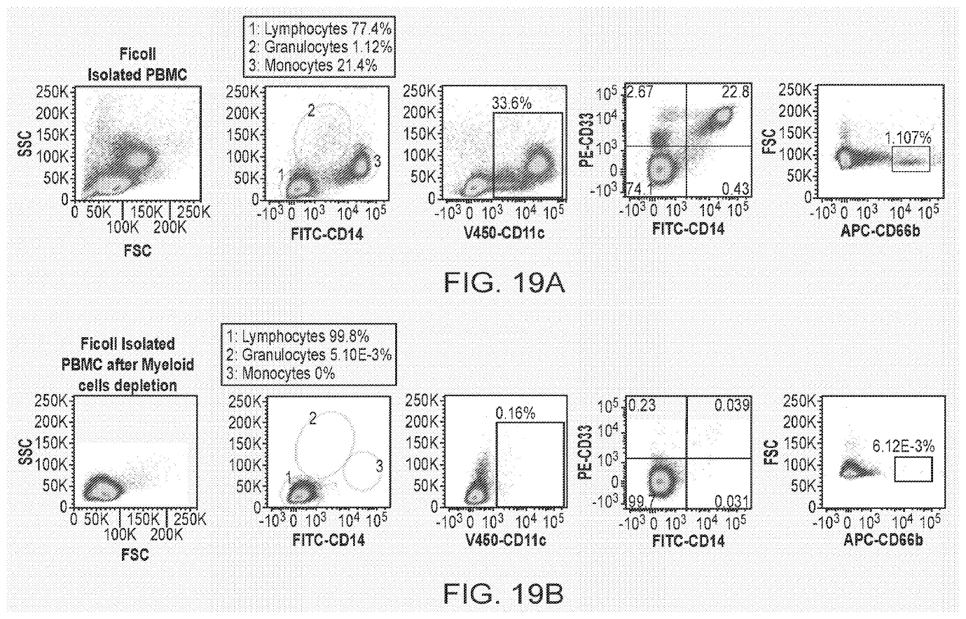

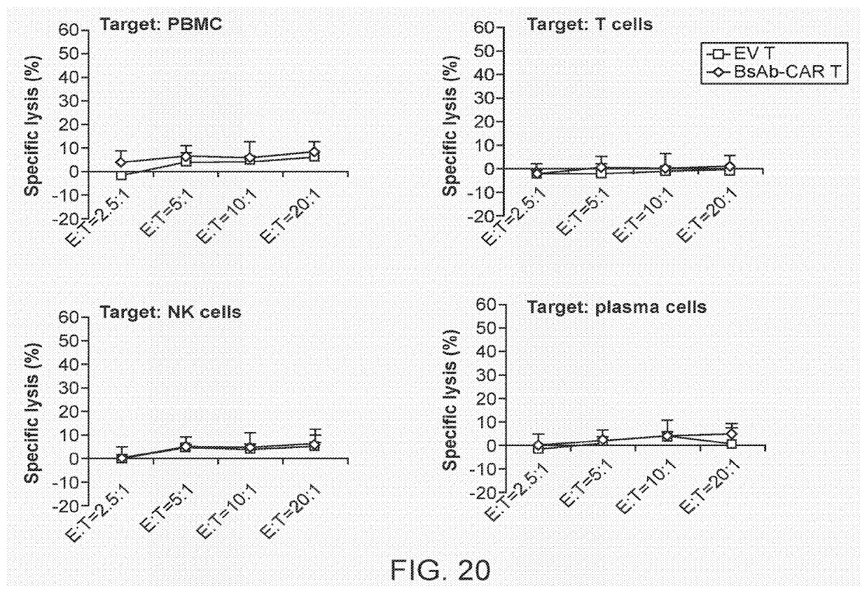

To compare the survival and proliferation of unmodified T cells, EV T cells, BCMA CAR T cells, and BsAb-CAR T cells in vivo, Applicants injected (i.v.) these human cells into immunodeficient NSG mice (FIG. 7A, upper). The background staining prior to i.v. injection of the human cells was also assessed on day −1 (FIG. 7A, and FIG. 18). On day +1 mice receiving each human T cell injection showed equal human CD3 expression, and the two CAR T cell populations were also identified by their F(ab)₂ expression. Moreover, the activation marker CD69 was detected on 97% of all four T cells populations isolated from the NSG mice (FIG. 7A, and FIG. 18). Interestingly, 14 days after the T cell infusions, only BsAb-CAR T cells group retained their high co-expression of CD3 and CD69 while there was substantial reduction of CD69 expression in the other three groups (FIG. 7A, and FIG. 18). Statistical analyses indicated that the day +14 percentage of both CD3 and CD3⁺CD69⁺ T cells in the BsAb-CAR T cell group were significantly higher than the other 3 groups (FIGS. 7B and 7C). Thirty-five days after the various transduced T cell injections, histograms showed that only the mice injected with BsAb-CAR T cells still possessed high percentages of hCD3 T cells (FIG. 7B). In vivo proliferation data shown that Ki67⁺CD69⁻ percentage was about 61.1% which was significantly higher than the three other groups that received non-transduced or transduced T cells (FIG. 7a purple court panels, FIG. 7c). Moreover, Applicants also determined the survival of BsAb-CAR T cells in vivo and observed that that the percentage of living (Annexin V⁻Sytox Blue⁻) BsAb-CAR T cells was approximately 87%, and the percentage of dead (Annexin V⁺Sytox Blue⁺) BsAb-CAR T cells was approximately 5%. Statistical analyses indicated BsAb-CAR T cells possessed a higher fraction of living cells and lower fraction of dead cells compared to the other three groups (FIG. 7D). Taking together, BsAb-CAR T cells not only show enhanced the cell proliferation via NKG2D signaling pathway in vitro, but also demonstrate enhanced proliferation and survival in vivo, when compared to the other groups of injected cells (including BCMA CAR T cells).

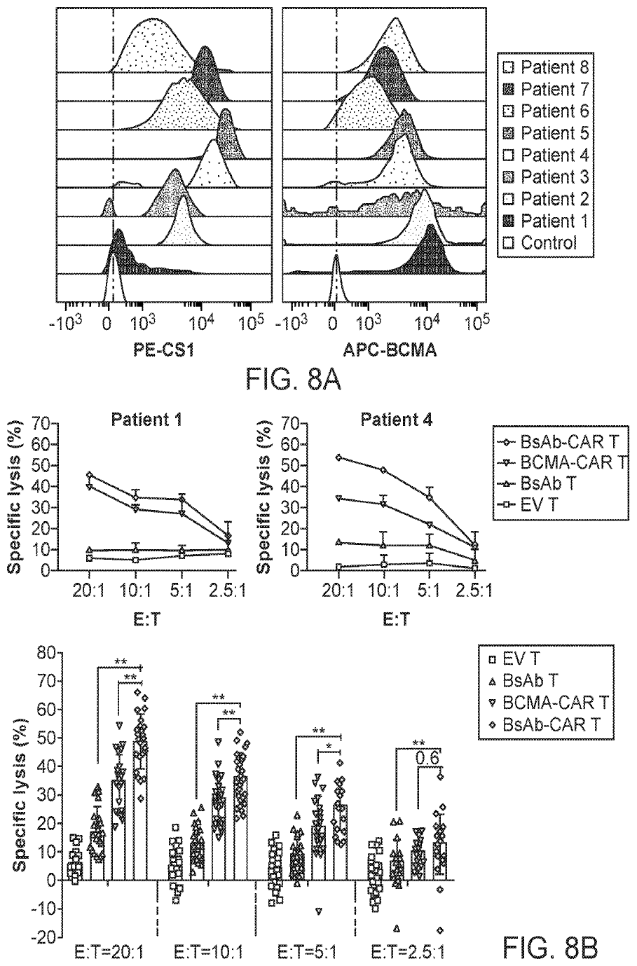

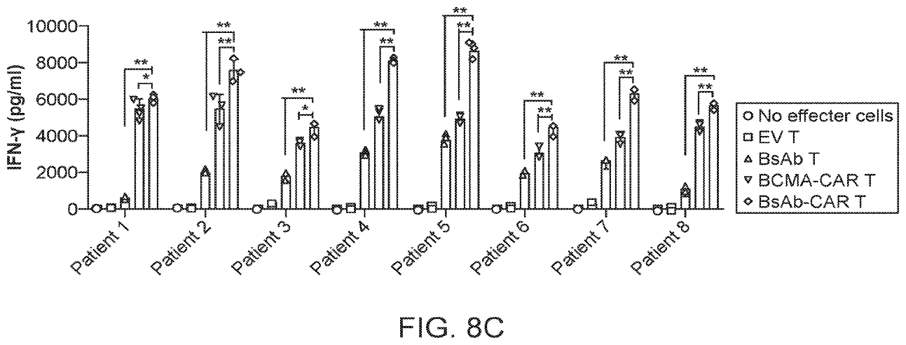

Improved recognition and killing of primary myeloma cells by BsAb-CAR T cells ex vivo: To assess the clinical relevance of the BsAb-CAR T cells, Applicants investigated whether they could efficiently recognize and kill MM cells isolated from patients and enhance IFN-γ production ex vivo. Primary CD138⁺ MM cells obtained from eight patients' bone marrow were isolated using positive magnetic selection, and flow cytometry was used to assess their surface expression of BCMA and CS1 (FIG. 8A). Using a ⁵¹Cr release assay performed in the absence of autologous PBMC, Applicants observed that MM cells from patients were highly resistant to EV-transduced T cell mediated lysis in all eight patients. Compared with BCMA CAR T cells or BsAb T cells, BsAb-CAR T cells showed significantly higher cytotoxicity in all eight patients that were tested, including patient 1 whose tumor cells had very low surface density expression of CS1. There is no significant difference in cytolytic activity between BsAb-CAR T cells and BsAb-BCMA seq. trans. T cells (FIG. 8B). Applicants also measured IFN-γ after 24 hours in a similar co-culture assay; BsAb-CAR T cells also secreted significantly higher levels of IFN-γ than EV-transduced T cells, BsAb T cells, or BCMA-CAR T cells (FIG. 8C). These findings demonstrate that BsAb-CAR T cells possess superb capacity to eradicate patient MM cells ex vivo.

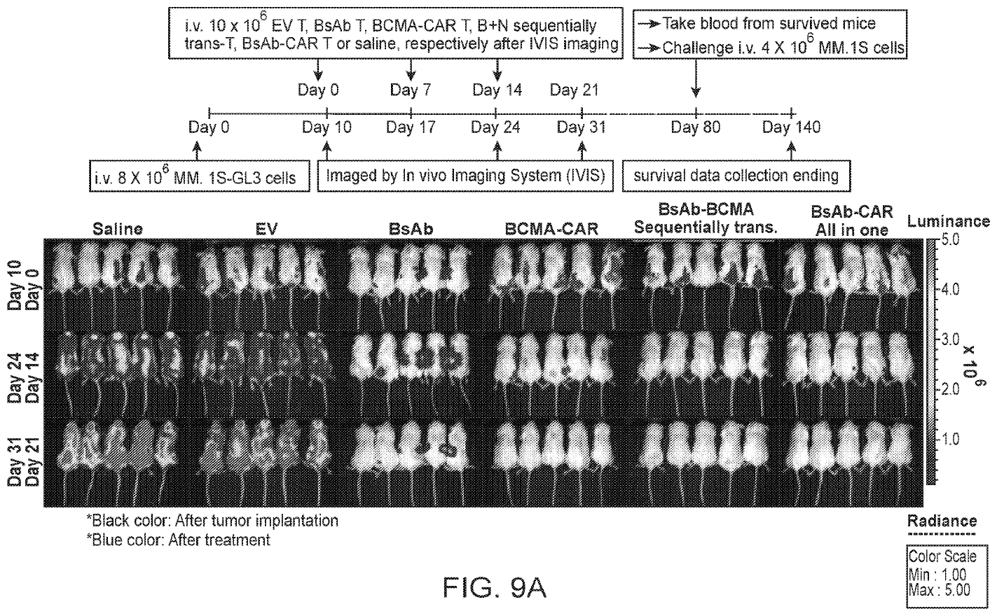

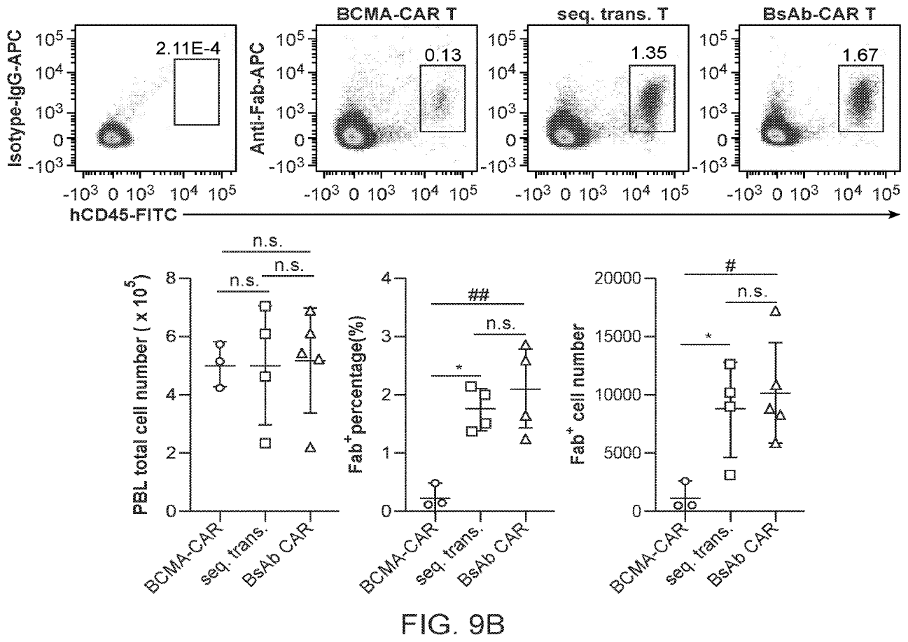

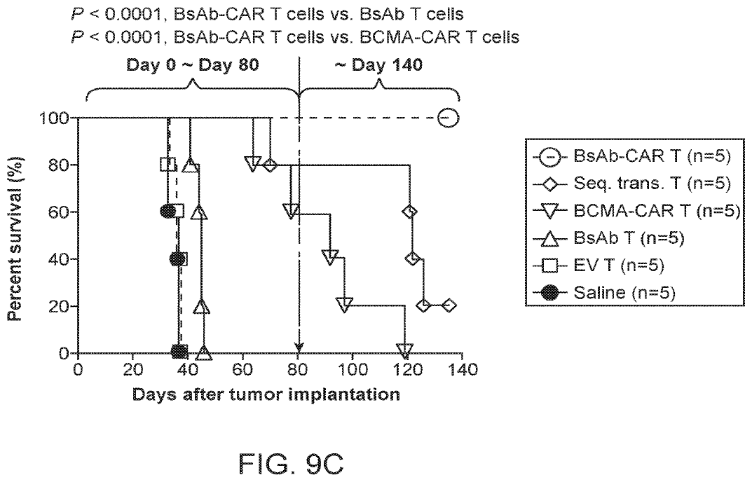

BsAb-CAR T cells inhibit MM tumor growth and prolong survival of tumor-bearing mice in an orthotopic xenograft MM model: To further address the potential therapeutic application of BsAb-CAR T cells, Applicants examined their antitumor activity in an MM.1 S MM-engrafted NSG mouse model. Intravenous injection of MM.1 S MM cells has been widely used to establish a mouse xenograft model of MM, because this can lead to bone marrow engraftment as well as consistent establishment of multifocal bone lytic lesions, which closely recapitulate human MM^{43,44}. To facilitate monitoring of tumor growth, Applicants engineered MM.1S MM cells to express both GFP and firefly luciferase by retroviral infection, and used iv. injection of 8×10⁶ of these MM cells to engraft NSG mice on day 0 as previously reported³⁰. These mice were then infused on three occasions (day 10, day 17, and day 24) with i.v. saline or 1×10⁷ EV T cells, 1×10⁷ BsAb T cells, 1×10⁷ BCMA CAR T cells, 1×10⁷ BsAb-BCMA seq. trans. T cells, or 1×10⁷ BsAb-CAR T cells. For mice that survived up to day 80, Applicants collected peripheral blood lymphocytes and re-challenged the nice with 1×10⁴ MM.1S MM cells. Bioluminescence imaging was used to monitor the MM.1 S MM growth and shows early disease progression up to day 31 in all six treatment groups (FIG. 9A). Using an anti-F(ab)₂ antibody to identify BCMA CAR T cells, Applicants noted on day 80 that the percentages of BsAb-BCMA seq. trans. T cells and BsAb-CAR T cells were significantly higher in the blood of MM.1S MM mice than were BCMA CAR T (FIG. 9B), while the total lymphocyte count in each mouse was similar (data not shown). Immunoblotting indicated the serum from day 80 MM.1S MM mice treated with BsAb-BCMA seq. trans. T cells or BsAb-CAR T cells still contained the secreted BsAb (not shown). By day 80, MM.1S MM mice treated with BsAb-BCMA seq. trans. T cells or with BsAb-CAR T cells had a significantly prolonged survival compared to similar mice treated with saline control, EV T cells or BsAb T cells. MM.1S MM mice treated with BCMA CAR T cells did slightly worse than MM.1S MM mice treated with BsAb-BCMA seq. trans. T cells or with BsAb-CAR T cells (FIG. 9D). On day 140 (60 days after the first tumor re-challenge), all five of the MM.1S MM mice treated with BsAb-CAR T group survived (FIG. 9D). These survival data following tumor re-challenge likely speak to the enhanced in vivo survival and enhanced in vivo proliferation of the BsAb-CAR T cells noted above in FIG. 7. Overall, these results indicate that BsAb-CAR T cells are superior to the other non-transduced or transduced T cell populations in their ability to inhibit MM tumor growth and prolong survival of tumor-bearing mice in orthotopic xenograft MM model.

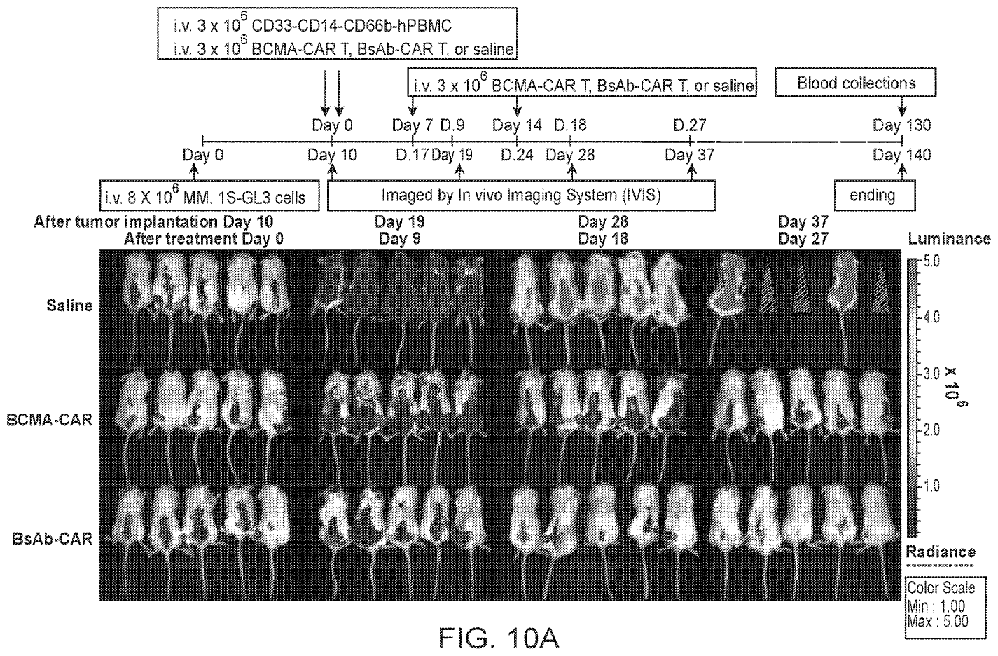

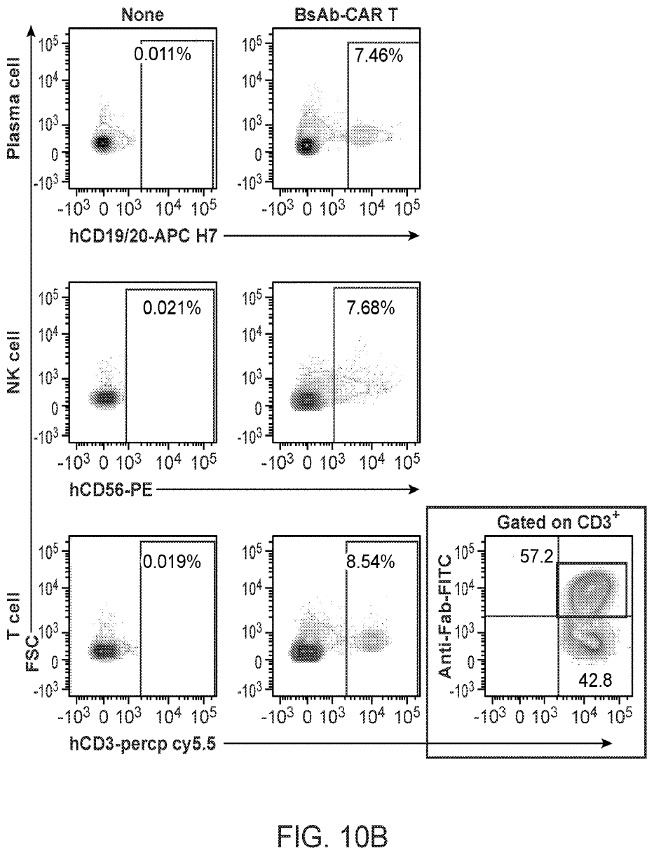

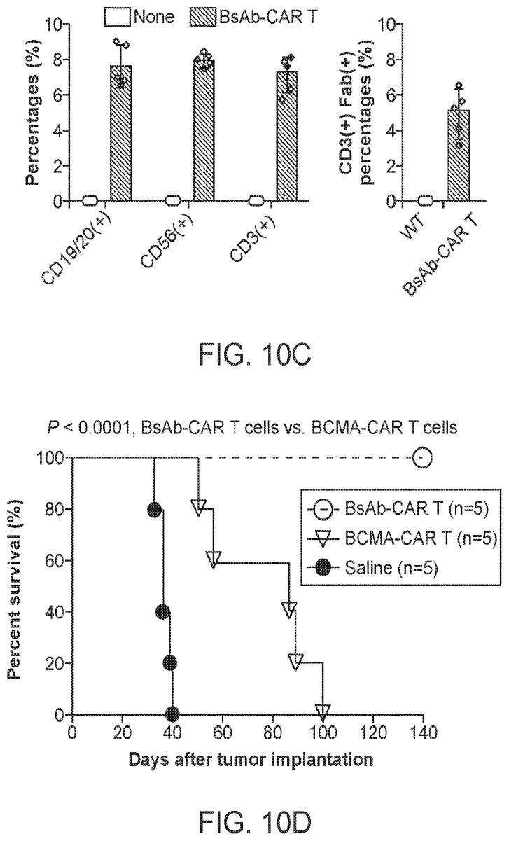

In order to determine the relevance of human non-transduced NKG2D+ lymphocytes in the orthotopic xenograft MM model (i.e., the in vivo counterpart to the in vitro experiment shown in FIG. 3E), Applicants next examined the anti-tumor efficacy of saline control, EV T cells, BCMA CAR T cells, or BsAb-CAR T cells in MM.1S MM-bearing NSG mice while co-injecting myeloid cell-depleted PBMC isolated from the same donor. The depletion of myeloid cells by sorting was undertaken to avoid GVHD (FIGS. 10A, 10B). The schema for injection of MM.1S MM cells, normal human lymphocytes, and various transduced T cells is shown in FIG. 10A, as is the imaging documenting MM progression through day 37. FIGS. 10B and 10C illustrate the percentage human lymphocytes detected in the blood of these mice. With additional time under these conditions, Applicants found a dramatic difference between mice treated with BCMA CAR T cells (that do not secrete the BsAb; 0% survival by day 100) and mice treated with BsAb-CAR T cells (that do secrete the BsAb; 100% survival on day 140; FIG. 10D). These data suggest that the more NKG2D+ immune cells that are involved, the better the efficacy of BsAb-CAR T cells will be for tumor eradication.

### Example 2—BsAb CAR T-Cells: Generation and Efficacy

The BsAb CAR of this disclosure comprise a CAR that recognizes and binds a first antigen while the BsAb recognizes and binds a second antigen. Both antigens are selected from the following list and different from each other: FLT3, CD19, mesothelin, human epidermal growth factor receptor 2 (HER2), prostate stem cell antigen (PSCA), carcinoembryonic antigen (CEA), GTP-ase-activating protein (GAP), ganglioside G2 (GD2), CD5, prostate specific membrane antigen (PSMA), receptor tyrosine kinase-like orphan receptor 1 (ROR1), CD123, CD70, CD38, B cell maturation antigen (BCMA), mucin 1, (Muc1), ephrin type-A receptor 2 precursor (EphA2), wildtype epidermal growth factor receptor (EGFRwt), epidermal growth factor receptor variant III (EGFRVIII), interleukin 13 receptor alpha 2 (IL13Ra2), CD133, glypican 3 (GPC3), epithelial cell adhesion molecule precursor (EpCam), fibroblast activation protein alpha (FAP), vascular endothelial growth factor receptor 2 (VEGFR2), cancer/testis (CT), guanylyl cyclase C (GUCY2C), tumor-associated glycoprotein-72 (TAG-72), thymidine kinase 1 (TK1), and hypoxanthine guanine phosphoribosyltransferase (HPRT1).

For the CAR, coding sequences for anti-first-TAA variable regions of heavy (VH) and light (VL) chains can be recombinantly produced or derived from a hybridoma and recombined using a linker. The VH-linker-VL fragment is incorporated in frame with the CD28-CD3zeta portion. The anti-first-TAA-scFv-CD28-CD3zeta fragment are subcloned into a lentiviral vector such as pCDH to create a second-generation pCDH-first-TAA CAR construct. To make the anti-NKG2D-anti-second-TAA BsAb lentiviral construct, two scFV or one or two codon-optimized single chain variable fragments (scFv) from an anti-second-TAA monoclonal antibody²⁹ and an anti-NKG2D antibody joined together by a non-immunogenic protein linker derived from human muscle aldose³⁵ are cloned into the pCDH lentiviral vector. For “all in one” BsAb-anti-first CAR, the anti-first-TAA-scFv-CD28-CD3ζ-T2A cassette is incorporated into the pCDH anti-second-TAA-NKG2D BsAb-EF1a (optional)-GFP (optional) to build a complete pCDH-anti-first-TAA-CAR-T2A-anti-second-TAA-BsAb-EF1a (optional)-GFP (optional) lentiviral construct. As is apparent to the skilled artisan, clinical constructs will not comprise the GFP and its promoter. These methods are used to generate BsAb-CARs as described herein.

Lentiviral transfection and infection are performed as described in a previously reported protocol.^{36,37}

To produce lentivirus, 293T cells are co-transfected with the pCDH-anti-first-TAA-CAR-T2A-anti-second-TAA-BsAb-EF1a-GFP plasmid or a pCDH empty vector plasmid plus the packaging plasmids pCMV-VSVG and pCMV-δr9 using Lipofectamine® 2000 (Invitrogen). Then the lentiviral supernatants are harvested and used to infect K562 cells using a previously published protocol 36.38 Transfected cells are isolated or GFP-positive cells are then sorted using an FACS Aria II cell sorter (BD Biosciences, San Jose, Calif., USA).

First-TAA-K562 K562 cells are transduced with a vector carrying the cDNA encoding the full-length first TAA. Lentivirus production, infection and sorting are performed using the methods described above. Second-TAA⁺first-TAA⁺K562 cells are generated by transducing a pCDH-CMV-second-TAA-EF1α-GFP lentiviral construct to first-TAA-K562 cells described above. The double-transduced cells are stained with an APC-anti-first-TAA mAb and then sorted for GFP⁺ second-TAA⁺ population. Before being used for experiments, these GFPrsecond-TAA⁺ double positive cells are passed several times in culture to ensure the loss of anti-first-TAA mAb-bound cells.

Flow cytometry analysis, immunoblotting, cytotoxicity assay, ELISA, in vivo treatment of MM-bearing mice and bioluminescence imaging, immune-synapse detected by immunofluorescence microscopy, cell culture, and mouse experiments are performed as described above.

Primary T cells expressing anti-first-TAA-specific CAR and/or the anti-NKG2D-anti-second-TAA bispecific antibody are generated. For example, a specific anti-first-TAA-CAR construct with a lentiviral vector backbone is generated, which sequentially consists of a signal peptide (SP), an anti-first-TAA heavy chain variable region (VH), a glycine-serine (GS) linker, an anti-first-TAA light chain variable region (VL), an optional Myc tag, a hinge, CD28 transmembrane domain and co-stimulatory domain, and CD3ζ intracellular signaling domain. Next, the anti-NKG2D-anti-second-TAA bispecific antibody (referred to as “BsAb”) construct is designed with a lentiviral vector backbone. It consists of single chain variable fragments (scFv) from an anti-NKG2D antibody and an anti-second-TAA monoclonal antibody, joined together by a non-immunogenic protein linker derived from human muscle aldose (HMA). Each scFv contains a corresponding heavy chain (VH) and light chain (VL) connected by a glycine-serine (GS) linker. The same donor T cells isolated from a healthy donor and activated by anti-human CD3/CD28 antibody beads are transduced with the empty vector (EV), the BsAb construct, anti-first-TAA-CAR construct, or first transduced with the BsAb construct followed sequentially by transduction with the anti-first-TAA-CAR construct. To determine whether the BsAb construct and the anti-first-TAA-CAR construct are successfully transduced, the expression of anti-first-TAA CAR on the cell surface is demonstrated by staining transduced T cells with anti-Fab. Further, cell-free supernatants from a 4-day culture are harvested and cell pellets from a 4-day culture are lysed. Both the supernatants and cell lysates are then subjected to immunoblotting using a 6x-his tagged Ab (“6x-his” disclosed as SEQ ID NO: 111). Production of both cellular and secreted BsAb indicates successful transduction of the BsAb construct.

The transduced T cells are then tested for effectiveness in killing a cancer cell, such as an MM cell, compared to T cells transduced with each vector alone in vitro. The surface expression of the first and second TAAs in cancer cells and/or cell lines are tested, for example, by flow cytometric analysis. A standard 4-hour ⁵¹Cr-release assay can be performed, using the cell line not expressing the first TAA or the second TAA as negative target control. Five groups of T cells are compared: (1) unmodified T cells, (2) empty vector (EV)-transduced T cells (EV T), (3) anti-NKG2D-anti-second-TAA-BsAb-transduced T cells (hereafter referred to as BsAb T), (4) anti-first-TAA-CAR-transduced T cells (hereafter referred to as anti-first-TAA-CAR T), and (5) anti-NKG2D-anti-second-TAA BsAb and anti-first-TAA-CAR sequentially transduced T cells (referred to as BsAb-anti-first-TAA-CAR seq. trans. T).

To further determine the activation of the above transduced-T cells upon recognition of cancer cells that endogenously express the first TAA and the second TAA, IFN-γ, IL-2 and TNF-α secretion is measured via ELISA in supernatants from unmodified T cells, EV T cells, anti-first-TAA-CAR T cells, BsAb T cells, and BsAb-anti-first-TAA-CAR seq. trans. The result can suggest that the NKG2D receptor expressed on cytotoxic CD8(+) T cells is being activated by the presence of the secreted BsAb.

Generation of single construct-engineered BsAb-CAR T cells to target both of the first TAA and the second TAA in a cancer cell, such as in MM: A single construct expressing both a BsAb and a CAR (referred to hereafter as BsAb-CAR) would be more practical in (1) producing effective expression of both constructs in a single T cell; (2) decreasing manufacturing costs; and (3) saving time. Applicants therefore generates a single BsAb-CAR construct containing both parts in a lentiviral vector backbone connected by T2A. To generate primary T cells expressing BsAb-CAR, the same method described above is utilized and then it is determined whether the BsAb-CAR-transduced T cells are successfully transduced. The surface expression of the CAR can be confirmed by flow cytometric analysis. The BsAb fusion protein can be successfully detected using a 6x-his-tagged Ab (“6x-his” disclosed as SEQ ID NO: 111) on day 4 in both cell lysates and in the serum-free-medium. Additionally, to dynamically measure BsAb secretion, the BsAb-CAR T cells are reseeded in serum-free medium on day 5, and then cell-free supernatants are collected at 12 h, 24 h, 48 h, 72 h and 96 h. Cytotoxicity of the unfractionated BsAb-CAR T cells is then tested, compared to target cell killing by EV T cells and unmodified T cells.

The BsAb secreted by CAR T cells requires two antigens to be functional: the second TAA expressed on tumor cells, and NKG2D expressed on immune cells. To determine whether anti-NKG2D-anti-second-TAA BsAb triggers NKG2D⁺ immune cells, 4-hour chromium-51 release assays are performed as described above at the ratio of 10 Effector (transduced or unmodified T cells) to 1 target cell, but added different quantities of human PBMC, i.e., 1-, 10-, 100-, or 200-fold of tumor cells. Without wishing to be bound by the theory and based on the results obtained in Example 1, it is hypothesized that if the secreted BsAb from either the BsAb T cells and/or the BsAb-CAR T cells are recruiting non-transduced NKG2D+ cytolytic effector cells to the second TAA+cancer cell line target, the killing of the target would go up with greater dilution from the addition of non-transduced PBMC.

Each subset of PBMC, i.e., NK cells, NKT cells, CD8⁺ T cells, and γ9Vδ2 T cells, is enriched to nearly 98% purity, primed with IL-2, CalCer plus IL-2, CD3/CD28 Dynabeads plus IL-2, and HMBPP plus IL-2, respectively, and transduced each with one of the control or experimental vectors, followed by a 4 h or 16 h ⁵¹Cr-release cytotoxicity assay against the cancer cell line at an E:T ratio of 5:1 for each.

To determine whether BsAb secreted by BsAb-CAR T cells can induce synapse formation between BsAb-CAR T cells and cancer cells, a confocal microscopy analysis is conducted after one hour of co-incubation. Co-localization is then observed.

To prove that enhanced cytotoxic effect of BsAb-CAR T cells depended on targeting tumor antigens, forced overexpression of the first TAA and the second TAA in the BsAb-CAR T cell-resistant K562 cell line is generated and tested for whether it can lower its threshold for lysis against this effector population. For this purpose, K562 cells are transduced with lentiviruses encoding the first TAA and the second TAA (or empty vector PCDH as control) to generate the K562 cell line ectopically expressing the first TAA and the second TAA. After confirming the success of generating the target cell line, ⁵¹Cr release assays are performed. ELISA assays are further performed to show whether there is an increase of IFN-γ and IL-2 secretion in co-cultures of BsAb-CAR T cells and K562-first-TAA-second-TAA cells, compared to co-cultures of EV T cells with K562-first-TAA-second-TAA.

Further, it is tested whether secreted anti-NKG2D-anti-second-TAA BsAb enhances CAR T cell proliferation in vitro and both survival and proliferation in vivo through NKG2D signaling. Cell numbers, cell proliferation, cell viability and metabolism are measured in the BsAb-CAR T cells cultured in culture medium of BsAb-transduced T cells compared to other conditions without BsAb at similar time points.

To investigate the ability of the transduced T cells to survive in vitro, various transduced T cells are cultured in the presence or absence of IL-2. Ki67, Annexin V and/or Sytox Blue expression are evaluated. To compare the survival and proliferation of unmodified T cells, EV T cells, anti-first-TAA CAR T cells, and BsAb-CAR T cells in vivo, these human cells are injected (i.v.) into immunodeficient NSG mice. The background staining prior to i.v. injection of the human cells is also assessed on day −1. CD3, F(ab)₂, the activation marker CD69 are assessed at various days, such as day 14 after injection and day 35 after injection.

Improved recognition and killing of primary cancer cells by BsAb-CAR T cells are tested ex vivo: To assess the clinical relevance of the BsAb-CAR T cells, it is investigated whether they can efficiently recognize and kill cancer cells isolated from patients and enhance IFN-γ production ex vivo. Primary cancer cells are obtained from patients and isolated using positive magnetic selection. Flow cytometry is used to assess their surface expression of the first TAA and the second TAA. A ⁵¹Cr release assay is performed in the absence of autologous PBMC. IFN-γ is measured after 24 hours in a similar co-culture assay.

It is also tested whether BsAb-CAR T cells inhibit cancer tumor growth and prolong survival of tumor-bearing mice in a cancer animal model, such as an orthotopic xenograft MM model as described herein. In order to determine the relevance of human non-transduced NKG2D+ lymphocytes in the cancer animal model, the anti-tumor efficacy is examined of saline control, EV T cells, anti-first-TAA CAR T cells, or BsAb-CAR T cells in cancer cell-bearing NSG mice while co-injecting myeloid cell-depleted PBMC isolated from the same donor. The depletion of myeloid cells by sorting is undertaken to avoid GVHD. Cancer progression is imaged through day 37 and longer, such as day 100, day 140 or longer. The percentage human lymphocytes is detected in the blood of these mice.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

### REFERENCES

1. Grupp, S. A., et al. Chimeric antigen receptor-modified T cells for acute lymphoid leukemia. The New England journal of medicine 368, 1509-1518 (2013).
2. Maude, S. L., et al. Chimeric antigen receptor T cells for sustained remissions in leukemia. N Engl J Med 371, 1507-1517 (2014).
3. Porter, D. L., Levine, B. L., Kalos, M., Bagg, A. & June, C. H. Chimeric antigen receptor-modified T cells in chronic lymphoid leukemia. The New England journal of medicine 365, 725-733 (2011).
4. Brown, C. E., et al. Regression of Glioblastoma after Chimeric Antigen Receptor T-Cell Therapy. N Engl J Med 375, 2561-2569 (2016).
5. Velasquez, M. P., Bonifant, C. L. & Gottschalk, S. Redirecting T cells to hematological malignancies with bispecific antibodies. Blood 131, 30-38 (2018).
6. Maude, S. & Barrett, D. M. Current status of chimeric antigen receptor therapy for haematological malignancies. Br J Haematol 172, 11-22 (2016).
7. Brentjens, R. J., et al. CD19-targeted T cells rapidly induce molecular remissions in adults with chemotherapy-refractory acute lymphoblastic leukemia. Sci Transl Med 5, 177ra138 (2013).
8. Porter, D. L., et al. Chimeric antigen receptor T cells persist and induce sustained remissions in relapsed refractory chronic lymphocytic leukemia. Sci Transl Med 7, 303ra139 (2015).
9. Oak, E. & Bartlett, N. L. Blinatumomab for the treatment of B-cell lymphoma.

Expert Opin Investig Drugs 24, 715-724 (2015).
10. von Stackelberg, A., et al. Phase I/Phase II Study of Blinatumomab in Pediatric Patients With Relapsed/Refractory Acute Lymphoblastic Leukemia. J Clin Oncol 34, 4381-4389 (2016).
11. Raulet, D. H. Roles of the NKG2D immunoreceptor and its ligands. Nat Rev Immunol 3, 781-790 (2003).
12. Groh, V., et al. Costimulation of CD8alphabeta T cells by NKG2D via engagement by MIC induced on virus-infected cells. Nat Immunol 2, 255-260 (2001).
13. Palumbo, A. & Anderson, K. Multiple myeloma. N Engl J Med 364, 1046-1060 (2011).
14. Dimopoulos, M. A., et al. Daratumumab, Lenalidomide, and Dexamethasone for Multiple Myeloma. N Engl J Med 375, 1319-1331 (2016).
15. van de Donk, N. W., Kamps, S., Mutis, T. & Lokhorst, H. M. Monoclonal antibody-based therapy as a new treatment strategy in multiple myeloma. Leukemia 26, 199-213 (2012).
16. Caligiuri, M. A. Human natural killer cells. Blood 112, 461-469 (2008).
17. Benson, D. M., et al. The PD-1/PD-L1 axis modulates the natural killer cell versus multiple myeloma effect: a therapeutic target for CT-011, a novel monoclonal anti-PD-1 antibody. Blood 116, 2286-2294 (2010).
18. Godfrey, J. & Benson, D. M. The role of natural killer cells in immunity against multiple myeloma. Leuk Lymphoma 53, 1666-1676 (2012).
19. Szmania, S., et al. Ex vivo-expanded natural killer cells demonstrate robust proliferation in vivo in high-risk relapsed multiple myeloma patients. J Immunother 38, 24-36 (2015).
20. Ruggeri, L., Mancusi, A., Capanni, M., Martelli, M. F. & Velardi, A. Exploitation of alloreactive NK cells in adoptive immunotherapy of cancer. Current opinion in immunology 17, 211-217 (2005).
21. Felgar, R. E. & Hiserodt, J. C. In vivo migration and tissue localization of highly purified lymphokine-activated killer cells (A-LAK cells) in tumor-bearing rats. Cell Immunol 129, 288-298 (1990).
22. Brand, J. M., et al. Kinetics and organ distribution of allogeneic natural killer lymphocytes transfused into patients suffering from renal cell carcinoma. Stem Cells Dev 13, 307-314 (2004).
23. Champsaur, M. & Lanier, L. L. Effect of NKG2D ligand expression on host immune responses. Immunological reviews 235, 267-285 (2010).
24. Avery, D. T., et al. BAFF selectively enhances the survival of plasmablasts generated from human memory B cells. J Clin Invest 112, 286-297 (2003).
25. Chiu, A., et al. Hodgkin lymphoma cells express TACI and BCMA receptors and generate survival and proliferation signals in response to BAFF and APRIL. Blood 109, 729-739 (2007).
26. Sidaway, P. Haematological cancer: Anti-BCMA CAR T cells show promise in IM. Nat Rev Clin Oncol 13, 530 (2016).
27. Carpenter, R. O., et al. B-cell maturation antigen is a promising target for adoptive T-cell therapy of multiple myeloma. Clinical cancer research: an official journal of the American Association for Cancer Research 19, 2048-2060 (2013).
28. Bellucci, R., et al. Graft-versus-tumor response in patients with multiple myeloma is associated with antibody response to BCMA, a plasma-cell membrane receptor. Blood 105, 3945-3950 (2005).
29. Chu, J., et al. CS1-specific chimeric antigen receptor (CAR)-engineered natural killer cells enhance in vitro and in vivo antitumor activity against human multiple myeloma.

Leukemia 28, 917-927 (2014).
30. Chu, J., et al. Genetic modification of T cells redirected toward CS1 enhances eradication of myeloma cells. Clinical cancer research: an official journal of the American Association for Cancer Research 20, 3989-4000 (2014).
31. Hsi, E. D., et al. CS1, a potential new therapeutic antibody target for the treatment of multiple myeloma. Clinical cancer research: an official journal of the American Association for Cancer Research 14, 2775-2784 (2008).
32. Raedler, L. A. Darzalex (Daratumumab): First Anti-CD38 Monoclonal Antibody Approved for Patients with Relapsed Multiple Myeloma. American health & drug benefits 9, 70-73 (2016).
33. Jamieson, A. M., et al. The role of the NKG2D immunoreceptor in immune cell activation and natural killing. Immunity 17, 19-29 (2002).
34. Vivier, E., Tomasello, E. & Paul, P. Lymphocyte activation via NKG2D: towards a new paradigm in immune recognition? Current opinion in immunology 14, 306-311 (2002).
35. Vallera, D. A., et al. A bispecific recombinant immunotoxin, DT2219, targeting human CD19 and CD22 receptors in a mouse xenograft model of B-cell leukemia/lymphoma. Clin Cancer Res 11, 3879-3888 (2005).
36. Yu, J., et al. Pro- and antiinflammatory cytokine signaling: reciprocal antagonism regulates interferon-gamma production by human natural killer cells. Immunity 24, 575-590 (2006).
37. Chen, L., et al. Targeting FLT3 by chimeric antigen receptor T cells for the treatment of acute myeloid leukemia. Leukemia 31, 1830-1834 (2017).
38. Becknell, B., et al. Efficient infection of human natural killer cells with an EBV/retroviral hybrid vector. Journal of immunological methods 296, 115-123 (2005).
39. Metelitsa, L. S., Weinberg, K. I., Emanuel, P. D. & Seeger, R. C. Expression of CD1d by myelomonocytic leukemias provides a target for cytotoxic NKT cells. Leukemia 17, 1068-1077 (2003).
40. Hintz, M., et al. Identification of (E)-4-hydroxy-3-methyl-but-2-enyl pyrophosphate as a major activator for human gammadelta T cells in Escherichia coli. FEBS letters 509, 317-322 (2001).
41. Cardone, J., et al. Complement regulator CD46 temporally regulates cytokine production by conventional and unconventional T cells. Nature immunology 11, 862-871 (2010).
42. D'Errico, G., Machado, H. L. & Sainz, B., Jr. A current perspective on cancer immune therapy: step-by-step approach to constructing the magic bullet. Clin Transl Med 6, 3 (2017).
43. Morales-Kastresana, A., Labiano, S., Quetglas, J. I. & Melero, I. Better performance of CARs deprived of the PD-1 brake. Clinical cancer research: an official journal of the American Association for Cancer Research 19, 5546-5548 (2013).
44. Zitvogel, L., Apetoh, L., Ghiringhelli, F. & Kroemer, G. Immunological aspects of cancer chemotherapy. Nature reviews. Immunology 8, 59-73 (2008).

PARTIAL SEQUENCE LISTING
IgG1 hinge domain:
(SEQ ID NO: 1)
LEPKSCDKTHTCPPCPDPKGT
CD28 transmembrane and cytoplasmic domain:
(SEQ ID NO: 2)
FWVLVVVGGVLACYSLLVTVAFIIFWVRSKRSRLLHSDYMNMTPRRPGPTRKHYQ
PYAPPRDFAAYRS
CD3 zeta signaling domain:
(SEQ ID NO: 3)
RVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQ
ALPPR
IL 2 signal peptide:
(SEQ ID NO: 4)
MYRMQLLSCIALSLALVTNS,
IgG1 signal peptide:
(SEQ ID NO: 5)
MGWSSIILFLVATATGVH
CDRs of anti-NKG2D antigen binding domain:
CDRL1:
(SEQ ID NO: 6)
SGSSSNIGNNAVN
CDRL2:
(SEQ ID NO: 7)
YDDLLPS
CDRL3:
(SEQ ID NO: 8)
AAWDDSLNGPV
CDRH1:
(SEQ ID NO: 9)
GFTFSSY
CDRH2:
(SEQ ID NO: 10)
RYDGSN
CDRH3:
(SEQ ID NO: 11)
DRGLGDGTYFDY
Anti-NKG2D light chain variable region:
(SEQ ID NO: 12)
QSALTQPASVSGSPGQSITISCSGSSSNIGNNAVNWYQQLPGKAPKLLIYYDDLLPSG
VSDRFSGSKSGTSAFLAISGLQSEDEADYYCAAWDDSLNGPVFGGGTKLTVL
Anti-NKG2D heavy chain variable region:
(SEQ ID NO: 13)
QVQLVESGGGLVKPGGSLRLSCAASGFTFSSYGMHWVRQAPGKGLEWVAFIRYDG
SNKYYADSVKGRFTISRDNSKNTLYLQMNSLRAEDTAVYYCAKDRGLGDGTYFD
YWGQGTTVTVSS
Peptide linker:
(SEQ ID NO: 14)
GGGGSGGGGSGGGGS
CDRs of anti-FLT3 antigen binding domain:
CDRL1:
(SEQ ID NO: 15)
RASQSISNNLH
CDRL2:
(SEQ ID NO: 17)
YASQSIS
CDRL3:
(SEQ ID NO: 19)
QQSNTWPYT
CDRH1:
(SEQ ID NO: 21)
SYWMH
CDRH2:
(SEQ ID NO: 23)
EIDPSDSYKDYNQKFKD
CDRH3:
(SEQ ID NO: 25)
AITTTPFDF
CDRs of anti-FLT3 antigen binding domain:
CDRL1:
(SEQ ID NO: 16)
KSSQSLLNSGNQKNYM;
CDRL2:
(SEQ ID NO: 18)
GASTRES
CDRL3:
(SEQ ID NO: 20)
QNDHSYPLT
CDRH1:
(SEQ ID NO: 22)
NYGLH
CDRH2:
(SEQ ID NO: 24)
VIWSGGSTDYNAAFIS
CDRH3:
(SEQ ID NO: 26)
GGIYYANHYYAMDY
Anti-FLT3 light chain variable region 1:
(SEQ ID NO: 27)
DIVLTQSPATLSVTPGDSVSLSCRASQSISNNLHWYQQKSHESPRLLIKYASQSISGIP
SRFSGSGSGTDFTLSINSVETEDFGVYFCQQSNTWPYTFGGGTKLEIKR
Anti-FLT3 heavy chain variable region 1:
(SEQ ID NO: 29)
QVQLQQPGAELVKPGASLKLSCKSSGYTFTSYWMHWVRQRPGHGLEWIGEIDPSD
SYKDYNQKFKDKATLTVDRSSNTAYMHLSSLTSDDSAVYYCARAITTTPFDFWGQ
GTTLTVSS
Anti-FLT3 light chain variable region 2:
(SEQ ID NO: 28)
DIVMTQSPSSLSVSAGEKVTMSCKSSQSLLNSGNQKNYMAWYQQKPGQPPKLLIY
GASTRESGVPDRFTGSGSGTDFTLTISSVQAEDLAVYYCQNDHSYPLTFGAGTKLEL
KR
Anti-FLT3 heavy chain variable region 2:
(SEQ ID NO: 30)
QVQLKQSGPGLVQPSQSLSITCTVSGFSLTNYGLHWVRQSPGKGLEWLGVIWSGGS
TDYNAAFISRLSISKDNSKSQVFFKMNSLQADDTAIYYCARKGGIYYANHYYAMD
YWGQGTSVTVSS
CDRs of anti-EGFR antigen binding domain:
CDRL1:
(SEQ ID NO: 31)
RSSQNIVHNNGITYLE
CDRL2:
(SEQ ID NO: 33)
KVSDRFS
CDRL3:
(SEQ ID NO: 35)
FQGSHIPPT
CDRH1:
(SEQ ID NO: 37)
GDTFTSY
CDRH2:
(SEQ ID NO: 39)
YPGSGG
CDRH3:
(SEQ ID NO: 41)
SGGPYFFDY
CDRs of anti-EGFR antigen binding domain:
CDRL1:
(SEQ ID NO: 32)
RASQGIRNNLA
CDRL2:
(SEQ ID NO: 34)
AASNLQS
CDRL3:
(SEQ ID NO: 36)
LQHHSYPLT
CDRH1:
(SEQ ID NO: 38)
GFTFSSY
CDRH2:
(SEQ ID NO: 40)
SGSGGS
CDRH3:
(SEQ ID NO: 42)
SSGWSEY
Anti-EGFR light chain variable region 1:
(SEQ ID NO: 43)
DILMTQSPLSLPVSLGDQASISCRSSQNIVHNNGITYLEWYLQRPGQSPKLLIYKVSD
RFSGVPDRFSGSGSGTDFTLKISRVEAEDLGIYYCFQGSHIPPTFGGGTKLEIKRAA
Anti-EGFR heavy chain variable region 1:
(SEQ ID NO: 45)
QVQLQQSGSEMARPGASVKLPCKASGDTFTSYWMHWVKQRHGHGPEWIGNIYPG
SGGTNYAEKFKNKVTLTVDRSSRTVYMHLSRLTSEDSAVYYCTRSGGPYFFDYWG
QGTTLTVSS
Anti-EGFR light chain variable region 2:
(SEQ ID NO: 44)
DIQMTQSPSSLSASVGDRVTITCRASQGIRNNLAWYQQKPGKAPKRLIYAASNLQS
GVPSRFTGSGSGTEFTLIVSSLQPEDFATYYCLQHHSYPLTSGGGTKVEIKYAHNS
Anti-EGFR heavy chain variable region 2:
(SEQ ID NO: 46)
EVQVLESGGGLVQPGGSLRLSCAASGFTFSSYAMSWVRQAPGKGLEWVSAISGSG
GSTNYADSVKGRFTISRDNSKNTLYLQMNSLRAEDTAVYYCAGSSGWSEYWGQG
TLVTVSS
CDRs of anti-BCMA antigen binding domain:
CDRL1:
(SEQ ID NO: 47)
RASESVTILGSHLIH
CDRL2:
(SEQ ID NO: 50)
LASNVQT
CDRL3:
(SEQ ID NO: 53)
LQSRTIPRT
CDRH1:
(SEQ ID NO: 56)
GYTFTDY
CDRH2:
(SEQ ID NO: 59)
INTETRE
CDRH3:
(SEQ ID NO: 62)
DYSYAMDY
CDRs of anti-BCMA antigen binding domain:
CDRL1:
(SEQ ID NO: 48)
SASQDISNYLN
CDRL2:
(SEQ ID NO: 51)
YTSNLHS
CDRL3:
(SEQ ID NO: 54)
QQYRKLPWT
CDRH1:
(SEQ ID NO: 57)
GGTFSNY
CDRH2:
(SEQ ID NO: 60)
YRGHSD
CDRH3:
(SEQ ID NO: 63)
GAIYNGYDVLDN
CDRs of anti-BCMA antigen binding domain:
CDRL1:
(SEQ ID NO: 49)
RASESVTILGSHLIY
CDRL2:
(SEQ ID NO: 52)
LASNVQT
CDRL3:
(SEQ ID NO: 55)
LQSRTIPRT
CDRH1:
(SEQ ID NO: 58)
GYTFRHY
CDRH2:
(SEQ ID NO: 61)
NTESGV
CDRH3:
(SEQ ID NO: 64)
DYLYSLDF
Anti-BCMA light chain variable region 1:
(SEQ ID NO: 65)
DIVLTQSPPSLAMSLGKRATISCRASESVTILGSHLIHWYQQKPGQPPTLLIQLASNV
QTGVPARFSGSGSRTDFTLTIDPVEEDDVAVYYCLQSRTIPRTEGGGTKLEIK
Anti-BCMA heavy chain variable region 1:
(SEQ ID NO: 68)
QIQLVQSGPELKKPGETVKISCKASGYTFTDYSINWVKRAPGKGLKWMGWINTETR
EPAYAYDFRGRFAFSLETSASTAYLQINNLKYEDTATYFCALDYSYAMDYWGQGT
SVTVSS
Anti-BCMA light chain variable region 2:
(SEQ ID NO: 66)
DIQMTQSPSSLSASVGDRVTITCSASQDISNYLNWYQQKPGKAPKLLIYYTSNLHSG
VPSRFSGSGSGTDFTLTISSLQPEDFATYYCQQYRKLPWTEGQGTKLEIKR
Anti-BCMA heavy chain variable region 2:
(SEQ ID NO: 69)
QVQLVQSGAEVKKPGSSVKVSCKASGGTFSNYWMHWVRQAPGQGLEWMGATYR
GHSDTYYNQKFKGRVTITADKSTSTAYMELSSLRSEDTAVYYCARGAIYNGYDVL
DNWGQGTLVTVSS
Anti-BCMA light chain variable region 3:
(SEQ ID NO: 67)
DIVLTQSPPSLAMSLGKRATISCRASESVTILGSHLIYWYQQKPGQPPTLLIQLASNV
QTGVPARFSGSGSRTDFTLTIDPVEEDDVAVYYCLQSRTIPRTFGGGTKLEIK
Anti-BCMA heavy chain variable region 3:
(SEQ ID NO: 70)
QIQLVQSGPELKKPGETVKISCKASGYTFRHYSMNWVKQAPGKGLKWMGRINTES
GVPIYADDFKGRFAFSVETSASTAYLVINNLKDEDTASYFCSNDYLYSLDFWGQGT
ALTVSS
CDRs of anti-CS1 antigen binding domain:
CDRL1:
(SEQ ID NO: 71)
KASQDVITGVA
CDRL2:
(SEQ ID NO: 72)
SASYRYT
CDRL3:
(SEQ ID NO: 73)
QQHYSTPLT
CDRH1:
(SEQ ID NO: 74)
GYSFTTY
CDRH2:
(SEQ ID NO: 75)
HPSDSE
CDRH3:
(SEQ ID NO: 76)
STMIATRAMDY
Anti-CS1 light chain variable region:
(SEQ ID NO: 77)
SDIVMTQSQKSMSTSVGDRVSITCKASQDVITGVAWYQQKPGQSPKLLIYSASYRY
TGVPDRFTGSGSGTDFTFTISNVQAEDLAVYYCQQHYSTPLTFGAGTKLELK
or
(SEQ ID NO: 154)
KLELKTGAGFTLPTSYHQQCYYVALDEAQVNSITFTFDTGSGSGTFRDPVGTYRYS
ASYILLKPSQGPKQQYWAVGTIVDQSAKCTISVRDGVSTSMSKQSQTMVIDS
or
(SEQ ID NO: 78)
DIVMTQSQKSMSTSVGDRVSITCKASQDVITGVAWYQQKPGQSPKLLIYSASYRYT
GVPDRFTGSGSGTDFTFTISNVQAEDLAVYYCQQHYSTPLTFGAGTKLELK.
Anti-CS1 heavy chain variable region:
(SEQ ID NO: 79)
SVTVSTGQGWYDMARTAIMTSRACYYVASDESTPSSLQMYATSSSKDVTLTAKDK
FKQNLRTESDSPHIMGIWELGQGPRQKVWNMWYTTFSYGSAKCSLKVSAGPRVLE
AGPQQLQVQS
or
(SEQ ID NO: 149)
SSVTVSTGQGWYDMARTAIMTSRACYYVASDESTPSSLQMYATSSSKDVTLTAKD
KFKQNLRTESDSPHIMGIWELGQGPRQKVWNMWYTTFSYGSAKCSLKVSAGPRVL
EAGPQQLQVQ,
or
(SEQ ID NO: 80)
SQVQLQQPGAELVRPGASVKLSCKASGYSFTTYWMNWVKQRPGQGLEWIGMIHP
SDSETRLNQKFKDKATLTVDKSSSTAYMQLSSPTSEDSAVYYCARSTMIATRAMDY
WGQGTSVTVS
or
(SEQ ID NO: 150)
QVQLQQPGAELVRPGASVKLSCKASGYSFTTYWMNWVKQRPGQGLEWIGMIHPS
DSETRLNQKFKDKATLTVDKSSSTAYMQLSSPTSEDSAVYYCARSTMIATRAMDY
WGQGTSVTVSS
human IgG4 Fc region:
(SEQ ID NO: 81)
ESKYGPPCPPCPAPEFLGGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVEWESNGQP
ENNYKTTPPVLDSDGSFFLYSRLTVDKSRWQEGNVFSCSVMHEALHNHYTQKSLS
LSLGK
human IgG4 Fc region equivalent having F234A, L235A and
N297Q mutations (i.e., mutations at aa 16, aa 17 and
aa 79 of SEQ ID NO: 81, respectively)
(SEQ ID NO: 82)
ESKYGPPCPPCPAPEAAGGPSVFLFPPKPKDTLMISRTPEVTCVVVDVSQEDPEVQF
NWYVDGVEVHNAKTKPREEQFQSTYRVVSVLTVLHQDWLNGKEYKCKVSNKGL
PSSIEKTISKAKGQPREPQVYTLPPSQEEMTKNQVSLTCLVKGFYPSDIAVEWESNG
QPENNYKTTPPVLDSDGSFFLYSRLTVDKSRWQEGNVFSCSVMHEALHNHYTQKS
LSLSLGK
Human muscle aldolase (HMA) peptide linker:
(SEQ ID NO: 83)
PSGQAGAAASESLFVSNHAY
Detectable marker:
(SEQ ID NO: 84)
YPYDVPDYA
T2A peptide:
(SEQ ID NO: 85)
HVGSGEGRGSLLTCGDVEENPGP
A nucleotide sequence encoding a signal peptide:
(SEQ ID NO: 86)
ATGGGGTGGTCAAGCATTATTCTGTTTCTGGTCGCTACCGCTACAGGCGTCCAT
A nucleotide sequence encoding a signal peptide:
(SEQ ID NO: 87)
ATGTACAGGATGCAACTCCTGTCTTGCATTGCACTAAGTCTTGCACTTGTCACA
AACAGT
A nucleotide sequence encoding a linker peptide:
(SEQ ID NO: 88)
GGTGGGGGCGGCTCTGGTGGCGGTGGCAGCGGCGGAGGTGGCAGT
A nucleotide sequence encoding a transmembrane and
cytoplasmic domain:
(SEQ ID NO: 89)
TTTTGGGTGCTGGTGGTGGTTGGTGGAGTCCTGGCTTGCTATAGCTTGCTAGTAA
CAGTGGCCTTTATTATTTTCTGGGTGAGGAGTAAGAGGAGCAGGCTCCTGCACA
GTGACTACATGAACATGACTCCCCGCCGCCCCGGGCCCACCCGCAAGCATTACC
AGCCCTATGCCCCACCACGCGACTTCGCAGCCTATCGCTCC
A nucleotide sequence encoding a signaling domain:
(SEQ ID NO: 90)
AGAGTGAAGTTCAGCAGGAGCGCAGACGCCCCCGCGTACCAGCAGGGCCAGAA
CCAGCTCTATAACGAGCTCAATCTAGGACGAAGAGAGGAGTACGATGTTTTGG
ACAAGAGACGTGGCCGGGACCCTGAGATGGGGGGAAAGCCGAGAAGGAAGAA
CCCTCAGGAAGGCCTGTACAATGAACTGCAGAAAGATAAGATGGCGGAGGCCT
ACAGTGAGATTGGGATGAAAGGCGAGCGCCGGAGGGGCAAGGGGCACGATGG
CCTTTACCAGGGTCTCAGTACAGCCACCAAGGACACCTACGACGCCCTTCACAT
GCAGGCCCTGCCCCCTCGC
or an equivalent thereof;
A nucleotide sequence encoding a cleavable peptide:
(SEQ ID NO: 91)
CACGTGGGTTCTGGAGAAGGACGCGGTTCCTTGTTGACGTGTGGCGATGTAGAG
GAAAATCCGGGTCCA
A nucleotide sequence encoding a linker:
(SEQ ID NO: 92)
CCGAGCGGCCAGGCGGGCGCGGCGGCATCGGAGTCCCTGTTTGTGTCAAATCA
CGCCTAC
A nucleotide sequence encoding an anti-BCMA light chain
variable region:
(SEQ ID NO: 93)
GATATTGTTCTTACTCAATCACCCCCAAGCCTTGCGATGTCTCTTGGTAAACGAG
CGACAATTAGTTGTAGAGCTTCTGAAAGCGTAACTATTCTTGGGTCACATCTTA
TTCATTGGTATCAACAAAAGCCGGGACAACCGCCTACACTCTTGATTCAACTCG
CGAGCAATGTTCAAACGGGTGTCCCTGCACGCTTTTCTGGGAGCGGTTCACGAA
CAGATTTTACTCTCACGATTGATCCAGTCGAAGAAGATGATGTCGCTGTATATT
ATTGTCTCCAAAGTAGGACAATACCAAGAACTTTTGGTGGTGGTACAAAATTGG
AAATTAAA
A nucleotide sequence encoding an anti-BCMA light chain
variable region:
(SEQ ID NO: 94)
GACATCCAGATGACCCAGAGCCCTAGCTCACTGAGCGCCAGCGTGGGCGACAG
GGTGACCATTACCTGCTCCGCCAGCCAGGACATCAGCAACTACCTGAACTGGTA
CCAGCAGAAGCCCGGCAAGGCCCCCAAGCTGCTGATCTACTACACCTCCAACCT
GCACTCCGGCGTGCCCAGCAGGTTCAGCGGAAGCGGCAGCGGCACCGATTTCA
CCCTGACCATCTCCAGCCTGCAGCCCGAGGACTTCGCCACCTACTACTGCCAGC
AGTACAGGAAGCTCCCCTGGACTTTCGGCCAGGGCACCAAACTGGAGATCAAG
CGT
A nucleotide sequence encoding an anti-BCMA light chain
variable region, i.e., Anti-BCMA sequence 1 scFv light chain:
(SEQ ID NO: 95)
GACATCGTGCTGACCCAGAGCCCCCCCAGCCTGGCCATGTCTCTGGGCAAGAGA
GCCACCATCAGCTGCCGGGCCAGCGAGAGCGTGACCATCCTGGGCAGCCACCT
GATCTACTGGTATCAGCAGAAGCCTGGCCAGCCCCCCACCCTGCTGATCCAGCT
GGCTAGCAATGTGCAGACCGGCGTGCCCGCCAGATTCAGCGGCAGCGGCAGCA
GAACCGACTTCACCCTGACCATCGACCCCGTGGAAGAGGACGACGTGGCCGTG
TACTACTGCCTGCAGAGCCGGACCATCCCCCGGACCTTTGGCGGAGGAACAAA
GCTGGAAATCAAG
A nucleotide sequence encoding an anti-BCMA light chain
variable region:
(SEQ ID NO: 96)
GACATCGTGCTGACCCAGAGCCCCCCCAGCCTGGCCATGTCTCTGGGCAAGAGA
GCCACCATCAGCTGCCGGGCCAGCGAGAGCGTGACCATCCTGGGCAGCCACCT
GATCCACTGGTATCAGCAGAAGCCCGGCCAGCCCCCCACCCTGCTGATCCAGCT
CGCCAGCAATGTGCAGACCGGCGTGCCCGCCAGATTCAGCGGCAGCGGCAGCA
GAACCGACTTCACCCTGACCATCGACCCCGTGGAAGAGGACGACGTGGCCGTG
TACTACTGCCTGCAGAGCCGGACCATCCCCCGGACCTTTGGCGGAGGCACCAAA
CTGGAAATCAAG
A nucleotide sequence encoding an anti-BCMA heavy chain
variable region:
(SEQ ID NO: 97)
CAGATCCAGCTGGTGCAGTCCGGCCCCGAGCTGAAGAAGCCCGGCGAGACCGT
GAAGATCTCCTGCAAGGCCTCCGGCTACACCTTCACCGACTACTCCATCAACTG
GGTGAAGCGGGCCCCCGGCAAGGGCCTGAAGTGGATGGGCTGGATCAACACCG
AGACCCGGGAGCCCGCCTACGCCTACGACTTCCGGGGCCGGTTCGCCTTCTCCC
TGGAGACCTCCGCCTCCACCGCCTACCTGCAGATCAACAACCTGAAGTACGAGG
ACACCGCCACCTACTTCTGCGCCCTGGACTACTCCTACGCCATGGACTACTGGG
GCCAGGGCACCTCCGTGACCGTGTCCTCC
A nucleotide sequence encoding an anti-BCMA heavy chain
variable region:
(SEQ ID NO: 98)
CAGGTGCAGCTGGTCCAGAGCGGCGCCGAAGTGAAGAAGCCCGGCAGCTCCGT
GAAAGTGAGCTGCAAGGCCAGCGGCGGCACCTTCAGCAACTACTGGATGCACT
GGGTGAGGCAGGCCCCCGGACAGGGCCTGGAGTGGATGGGCGCCACCTACAGG
GGCCACAGCGACACCTACTACAACCAGAAGTTCAAGGGCCGGGTGACCATCAC
CGCCGACAAGAGCACCAGCACCGCCTACATGGAACTGAGCAGCCTCAGGAGCG
AGGACACCGCTGTGTATTACTGCGCCAGGGGCGCCATCTACAACGGCTACGAC
GTGCTGGACAACTGGGGCCAGGGCACACTAGTGACCGTGTCCAGC
A nucleotide sequence encoding an anti-BCMA heavy chain
variable region, i.e., Anti-BCMA sequence 1 scFv heavy chain:
(SEQ ID NO: 99)
ATGGGATGGAGCTCTATCATCCTCTTCTTGGTAGCAACAGCTACAGGTGTCCAC
CAGATTCAGCTGGTGCAGAGCGGCCCTGAGCTGAAGAAACCCGGCGAGACAGT
GAAGATCAGCTGCAAGGCCTCCGGCTACACCTTCCGGCACTACAGCATGAACTG
GGTGAAACAGGCCCCTGGCAAGGGCCTGAAGTGGATGGGCCGGATCAACACCG
AGAGCGGCGTGCCCATCTACGCCGACGACTTCAAGGGCAGATTCGCCTTCAGCG
TGGAAACCAGCGCCAGCACCGCCTACCTGGTGATCAACAACCTGAAGGACGAG
GATACCGCCAGCTACTTCTGCAGCAACGACTACCTGTACAGCCTGGACTTCTGG
GGCCAGGGCACCGCCCTGACCGTGTCCAGC
A nucleotide sequence encoding an anti-BCMA heavy chain
variable region:
(SEQ ID NO: 100)
CAGATTCAGCTGGTGCAGAGCGGCCCTGAGCTGAAGAAACCCGGCGAGACAGT
GAAGATCAGCTGCAAGGCCTCCGGCTACACCTTCACCGACTACAGCATCAACTG
GGTGAAAAGAGCCCCTGGCAAGGGCCTGAAGTGGATGGGCTGGATCAACACCG
AGACAAGAGAGCCCGCCTACGCCTACGACTTCCGGGGCAGATTCGCCTTCAGCC
TGGAAACCAGCGCCAGCACCGCCTACCTGCAGATCAACAACCTGAAGTACGAG
GACACCGCCACCTACTTTTGCGCCCTGGACTACAGCTACGCCATGGACTACTGG
GGCCAGGGCACCAGCGTGACCGTGTCCAGC
A nucleotide sequence encoding an anti-NKG2D light chain
variable region:
(SEQ ID NO: 101)
CAGTCAGCGCTTACGCAGCCGGCGTCGGTGTCGGGTTCCCCGGGTCAGTCGATC
ACGATCAGCTGTAGTGGGAGCAGCTCCAACATCGGTAACAACGCAGTGAACTG
GTATCAGCAACTGCCGGGAAAAGCGCCGAAACTGCTGATTTACTATGATGATTT
GCTGCCAAGTGGAGTTAGTGACCGCTTTTCCGGCAGTAAATCGGGTACCTCGGC
TTTTCTGGCTATTTCGGGTCTCCAGAGCGAGGATGAAGCTGATTATTATTGCGCC
GCATGGGATGATAGCTTAAATGGCCCAGTTTTTGGCGGCGGTACTAAACTGACC
GTGCTG
A nucleotide sequence encoding an anti-NKG2D heavy chain
variable region:
(SEQ ID NO: 102)
CAAGTGCAGCTGGTTGAATCCGGTGGCGGTCTGGTCAAGCCGGGCGGCTCTTTG
CGTCTGAGCTGTGCCGCGTCGGGTTTTACCTTCAGCTCTTATGGTATGCATTGGG
TGCGTCAGGCGCCTGGCAAAGGTCTGGAGTGGGTTGCGTTCATCCGCTACGATG
GGTCTAACAAATATTATGCCGACTCAGTAAAAGGACGCTTCACTATTAGCCGCG
ACAATAGCAAAAATACCCTGTACCTGCAAATGAATAGCCTGCGCGCCGAAGAT
ACCGCCGTTTACTATTGCGCTAAAGATCGTGGCCTGGGTGATGGTACGTACTTC
GATTACTGGGGTCAGGGCACCACCGTTACCGTTAGTTCA
A nucleotide sequence encoding an anti-FLT3 light chain
variable region:
(SEQ ID NO: 103)
GATATTGTGCTAACTCAGTCTCCAGCCACCCTGTCTGTGACTCCAGGAGATAGC
GTCAGTCTTTCCTGCAGGGCCAGCCAGAGTATTAGCAACAACCTACACTGGTAT
CAACAAAAATCACATGAGTCTCCAAGGCTTCTCATCAAGTATGCTTCCCAGTCC
ATCTCTGGGATCCCCTCCAGGTTCAGTGGCAGTGGATCAGGGACAGATTTCACT
CTCAGTATCAACAGTGTGGAGACTGAAGATTTTGGAGTGTATTTCTGTCAACAG
AGTAACACCTGGCCGTACACGTTCGGAGGGGGGACCAAGCTGGAAATAAAACG
G
A nucleotide sequence encoding an anti-FLT3 light chain
variable region:
(SEQ ID NO: 104)
GACATTGTGATGACACAGTCTCCATCCTCCCTGAGTGTGTCAGCAGGAGAGAAG
GTCACTATGAGCTGCAAGTCCAGTCAGAGTCTGTTAAACAGTGGAAATCAAAA
GAACTATATGGCCTGGTATCAGCAGAAACCAGGGCAGCCTCCTAAACTGTTGAT
CTACGGGGCATCCACTAGGGAATCTGGGGTCCCTGATCGCTTCACAGGCAGTGG
ATCTGGAACCGATTTCACTCTTACCATCAGCAGTGTGCAGGCTGAAGACCTGGC
AGTTTATTACTGTCAGAATGATCATAGTTATCCGCTCACGTTCGGTGCTGGGAC
CAAGCTGGAGCTGAAACGG
A nucleotide sequence encoding an anti-FLT3 heavy chain
variable region:
(SEQ ID NO: 105)
CAGGTCCAACTGCAGCAGCCTGGGGCTGAGCTTGTGAAGCCTGGGGCTTCATTG
AAGCTGTCCTGCAAGTCTTCCGGGTACACCTTCACCAGCTACTGGATGCACTGG
GTGAGGCAGAGGCCTGGACATGGCCTTGAGTGGATCGGAGAGATTGATCCTTCT
GACAGTTATAAAGACTACAATCAGAAGTTCAAGGACAAGGCCACATTGACTGT
GGACAGATCCTCCAACACAGCCTACATGCACCTCAGCAGCCTGACATCTGATGA
CTCTGCGGTCTATTATTGTGCAAGAGCGATTACGACGACCCCCTTTGACTTCTGG
GGCCAAGGCACCACTCTCACAGTCTCCTCA
A nucleotide sequence encoding an anti-FLT3 heavy chain
variable region:
(SEQ ID NO: 106)
CAGGTGCAGCTGAAGCAGTCAGGACCTGGCCTAGTGCAGCCCTCACAGAGCCT
GTCCATCACCTGCACAGTCTCTGGTTTCTCATTAACTAACTATGGTTTACACTGG
GTTCGCCAGTCTCCAGGAAAGGGCCTGGAGTGGCTGGGAGTGATATGGAGTGG
TGGAAGCACAGACTATAATGCAGCTTTCATATCCAGACTGAGCATCAGCAAGG
ACAACTCCAAGAGCCAAGTTTTCTTTAAAATGAACAGTCTGCAGGCTGATGACA
CAGCCATATACTACTGTGCCAGAAAAGGAGGGATCTACTATGCTAACCATTACT
ATGCTATGGACTACTGGGGTCAAGGAACCTCAGTCACCGTCTCCTCA
A nucleotide sequence encoding an anti-CS1 light chain
variable region:
(SEQ ID NO: 107)
AAACTTGAGT TGAAGACCGG TGCCGGCTTC ACCTTACCGA CCAGTTATCA
TCAACAATGC TATTACGTGG CCCTGGACGA AGCACAGGTG AATTCAATTA
CGTTTACGTT TGATACCGGC TCTGGCAGCG GTACATTTCG TGATCCCGTG
GGCACTTACC GCTATTCGGC GAGTTATATC TTGCTGAAAC CTTCCCAAGG
TCCGAAACAG CAGTACTGGG CGGTTGGCAC CATTGTAGAC CAATCAGCCA
AATGTACAAT CTCGGTTCGC GATGGTGTCA GTACGTCGAT GTCTAAGCAG
TCACAGACAA TGGTTATCGA T
A nucleotide sequence encoding an anti-CS1 heavy chain
variable region:
(SEQ ID NO: 108)
AGCGTTACCG TGAGTACAGG CCAGGGCTGG TATGACATGG CACGTACAGC
CATCATGACC TCGCGCGCAT GTTACTACGT CGCGTCAGAT GAATCGACGC
CTTCCTCGCT GCAAATGTAT GCAACCTCCA GCAGCAAAGA TGTTACCCTG
ACCGCAAAGG ACAAGTTTAA ACAGAATTTG CGTACGGAGA GTGACTCCCC
GCACATCATG GGAATCTGGG AGTTGGGTCA GGGGCCTCGT CAGAAGGTAT
GGAACATGTG GTATACAACT TTTTCGTACG GCTCAGCAAA ATGCAGCTTG
AAAGTGTCGG CAGGTCCGCG CGTGCTGGAG GCCGGTCCGC AGCAGCTGCA
AGTCCAGTCT
A nucleotide sequence encoding a hinge domain:
(SEQ ID NO: 109)
CTCGAGCCCAAATCTTGTGACAAAACTCACACATGCCCACCGTGCCCGGATCCC
AAAGGTACC
peptide linker:
(SEQ ID NO: 110)
(glycine-serine)n
wherein n is an integer from 1 to 6
6x His tag:
(SEQ ID NO: 111)
His His His His His His
Hinge domain: IgG1 heavy chain hinge coding sequence:
(SEQ ID NO: 112)
CTCGAGCCCAAATCTTGTGACAAAACTCACACATGCCCACCGTGCCCG
CD28 transmembrane region coding sequence:
(SEQ ID NO: 113)
TTTTGGGTGCTGGTGGTGGTTGGTGGAGTCCTGGCTTGCTATAGCTTGCTAGTAA
CAGTGGCCTTTATTATTTTCTGGGTG
4-1BB co-stimulatory signaling region coding sequence:
(SEQ ID NO: 114)
AAACGGGGCAGAAAGAAACTCCTGTATATATTCAAACAACCATTTATGAGACC
AGTACAAACTACTCAAGAGGAAGATGGCTGTAGCTGCCGATTTCCAGAAGAAG
AAGAAGGAGGATGTGAACTG
CD28 co-stimulatory signaling region coding sequence:
(SEQ ID NO: 115)
AGGAGTAAGAGGAGCAGGCTCCTGCACAGTGACTACATGAACATGACTCCCCG
CCGCCCCGGGCCCACCCGCAAGCATTACCAGCCCTATGCCCCACCACGCGACTT
CGCAGCCTATCGCTCC
CD3 zeta signaling region coding sequence:
(SEQ ID NO: 116)
AGAGTGAAGTTCAGCAGGAGCGCAGACGCCCCCGCGTACCAGCAGGGCCAGAA
CCAGCTCTATAACGAGCTCAATCTAGGACGAAGAGAGGAGTACGATGTTTTGG
ACAAGAGACGTGGCCGGGACCCTGAGATGGGGGGAAAGCCGAGAAGGAAGAA
CCCTCAGGAAGGCCTGTACAATGAACTGCAGAAAGATAAGATGGCGGAGGCCT
ACAGTGAGATTGGGATGAAAGGCGAGCGCCGGAGGGGCAAGGGGCACGATGG
CCTTTACCAGGGTCTCAGTACAGCCACCAAGGACACCTACGACGCCCTTCACAT
GCAGGCCCTGCCCCCTCGCTAA
Human CD8 alpha hinge domain:
(SEQ ID NO: 117)
PAKPTTTPAPRPPTPAPTIASQPLSLRPEACRPAAGGAVHTRGLDFACDIY.
Mouse CD8 alpha hinge domain:
(SEQ ID NO: 118)
KVNSTTTKPVLRTPSPVHPTGTSQPQRPEDCRPRGSVKGTGLDFACDIY.
Cat CD8 alpha hinge domain:
(SEQ ID NO: 119)
PVKPTTTPAPRPPTQAPITTSQRVSLRPGTCQPSAGSTVEASGLDLSCDIY.
Human CD8 alpha transmembrane domain:
(SEQ ID NO: 120)
IYIWAPLAGTCGVLLLSLVIT.
Mouse CD8 alpha transmembrane domain:
(SEQ ID NO: 121)
IWAPLAGICVALLLSLIITLI.
Rat CD8 alpha transmembrane domain:
(SEQ ID NO: 122)
IWAPLAGICAVLLLSLVITLI.
4-1BB costimulatory signaling region:
(SEQ ID NO: 123)
KRGRKKLLYIFKQPFMRPVQTTQEEDGCSCRFPEEEEGGCEL
CD28 Sequence:
(SEQ ID NO: 124)
MLRLLLALNL FPSIQVTGNK ILVKQSPMLV
AYDNAVNLSC KYSYNLFSRE FRASLHKGLDSAVEVCVVYG NYSQQLQVYS
KTGFNCDGKL GNESVTFYLQ NLYVNQTDIY FCKIEVMYPPPYLDNEKSNG
TIIHVKGKHL CPSPLFPGPS KPFWVLVVVG GVLACYSLLVTVAFIIFWVR
SKRSRLLHSD YMNMTPRRPG PTRKHYQPYA PPRDFAAYRS
ICOS costimulatory signaling region coding sequence:
(SEQ ID NO: 125)
ACAAAAAAGA AGTATTCATC CAGTGTGCAC GACCCTAACG GTGAATACAT
GTTCATGAGA GCAGTGAACA CAGCCAAAAA ATCCAGACTC ACAGATGTGA
CCCTA
OX40 costimulatory signaling region coding sequence:
(SEQ ID NO: 126)
AGGGACCAG AGGCTGCCCC CCGATGCCCA CAAGCCCCCT GGGGGAGGCA
GTTTCCGGAC CCCCATCCAA GAGGAGCAGG CCGACGCCCA CTCCACCCTG
GCCAAGATC
Human FLT3 Isoform 1,
(SEQ ID NO: 127)
MPALARDGGQLPLLVVFSAMIFGTITNQDLPVIKCVLINHKNNDSSVGKSSSYPMVS
ESPEDLGCALRPQSSGTVYEAAAVEVDVSASITLQVLVDAPGNISCLWVFKHSSLN
CQPHFDLQNRGVVSMVILKMTETQAGEYLLFIQSEATNYTILFTVSIRNTLLYTLRR
PYFRKMENQDALVCISESVPEPIVEWVLCDSQGESCKEESPAVVKKEEKVLHELFG
TDIRCCARNELGRECTRLFTIDLNQTPQTTLPQLFLKVGEPLWIRCKAVHVNHGFGL
TWELENKALEEGNYFEMSTYSTNRTMIRILFAFVSSVARNDTGYYTCSSSKRPSQSA
LVTIVEKGFINATNSSEDYEIDQYEEFCFSVRFKAYPQIRCTWTFSRKSFPCEQKGLD
NGYSISKFCNHKHQPGEYIFHAENDDAQFTKMFTLNIRRKPQVLAEASASQASCFSD
GYPLPSWTWKKCSDKSPNCTEEITEGVWNRKANRKVFGQWVSSSTLNMSEAIKGF
LVKCCAYNSLGTSCETILLNSPGPFPFIQDNISFYATIGVCLLFIVVLTLLICHKYKKQ
FRYESQLQMVQVTGSSDNEYFYVDFREYEYDLKWEFPRENLEFGKVLGSGAFGKV
MNATAYGISKTGVSIQVAVKMLKEKADSSEREALMSELKMMTQLGSHENIVNLLG
ACTLSGPIYLIFEYCCYGDLLNYLRSKREKFHRTWTEIFKEHNFSFYPTFQSHPNSSM
PGSREVQIHPDSDQISGLHGNSFHSEDEIEYENQKRLEEEEDLNVLTFEDLLCFAYQV
AKGMEFLEFKSCVHRDLAARNVLVTHGKVVKICDFGLARDIMSDSNYVVRGNARL
PVKWMAPESLFEGIYTIKSDVWSYGILLWEIFSLGVNPYPGIPVDANFYKLIQNGFK
MDQPFYATEEIYIIMQSCWAFDSRKRPSFPNLTSFLGCQLADAEEAMYQNVDGRVS
ECPHTYQNRRPFSREMDLGLLSPQAQVEDS
Human FLT3 Isoform 2:
(SEQ ID NO: 128)
MPALARDGGQLPLLVVFSAMIFGTITNQDLPVIKCVLINHKNNDSSVGKSSSYPMVS
ESPEDLGCALRPQSSGTVYEAAAVEVDVSASITLQVLVDAPGNISCLWVFKHSSLN
CQPHFDLQNRGVVSMVILKMTETQAGEYLLFIQSEATNYTILFTVSIRNTLLYTLRR
PYFRKMENQDALVCISESVPEPIVEWVLCDSQGESCKEESPAVVKKEEKVLHELFG
TDIRCCARNELGRECTRLFTIDLNQTPQTTLPQLFLKVGEPLWIRCKAVHVNHGFGL
TWELENKALEEGNYFEMSTYSTNRTMIRILFAFVSSVARNDTGYYTCSSSKHPSQSA
LVTIVEKGFINATNSSEDYEIDQYEEFCFSVRFKAYPQIRCTWTFSRKSFPCEQKGLD
NGYSISKFCNHKHQPGEYIFHAENDDAQFTKMFTLNIRRKPQVLAEASASQASCFSD
GYPLPSWTWKKCSDKSPNCTEEITEGVWNRKANRKVFGQWVSSSTLNMSEAIKGF
LVKCCAYNSLGTSCETILLNSPGPFPFIQDNISFYATIGVCLLFIVVLTLLICHKYKKQ
FRYESQLQMVQVTGSSDNEYFYVDFREYEYDLKWEFPRENLEFGKVLGSGAFGKV
MNATAYGISKTGVSIQVAVKMLKEKADSSEREALMSELKMMTQLGSHENIVNLLG
ACTLSGPIYLIFEYCCYGDLLNYLRSKREKFHRTWTEIFKEHNFSFYPTFQSEIPNSSM
PGSREVQIHPDSDQISGLHGNSFHSEDEIEYENQKRLEEEEDLNVLTFEDLLCFAYQV
AKGMEFLEEKSARLPVKWMAPESLFEGIYTIKSDVWSYGILLWEIFSLGVNPYPGIP
VDANFYKLIQNGFKMDQPFYATEEIYIIMQSCWAFDSRKRPSFPNLTSFLGCQLADA
EEAMYQNVDGRVSECPHTYQNRRPFSREMDLGLLSPQAQVEDS
EGFR Isoform 1, Uniprot P00533-1:
(SEQ ID NO: 129)
MRPSGTAGAALLALLAALCPASRALEEKKVCQGTSNKLTQLGTFEDHFLSLQRMF
NNCEVVLGNLEITYVQRNYDLSFLKTIQEVAGYVLIALNTVERIPLENLQIIRGNMY
YENSYALAVLSNYDANKTGLKELPMRNLQEILHGAVRFSNNPALCNVESIQWRDIV
SSDFLSNMSMDFQNHLGSCQKCDPSCPNGSCWGAGEENCQKLTKIICAQQCSGRCR
GKSPSDCCHNQCAAGCTGPRESDCLVCRKFRDEATCKDTCPPLMLYNPTTYQMDV
NPEGKYSFGATCVKKCPRNYVVTDHGSCVRACGADSYEMEEDGVRKCKKCEGPC
RKVCNGIGIGEFKDSLSINATNIKHFKNCTSISGDLHILPVAFRGDSFTHTPPLDPQEL
DILKTVKEITGFLLIQAWPENRTDLHAFENLEIIRGRTKQHGQFSLAVVSLNITSLGL
RSLKEISDGDVIISGNKNLCYANTINWKKLFGTSGQKTKIISNRGENSCKATGQVCH
ALCSPEGCWGPEPRDCVSCRNVSRGRECVDKCNLLEGEPREFVENSECIQCHPECLP
QAMNITCTGRGPDNCIQCAHYIDGPHCVKTCPAGVMGENNTLVWKYADAGHVCH
LCHPNCTYGCTGPGLEGCPTNGPKIPSIATGMVGALLLLLVVALGIGLFMRRRHIVR
KRTLRRLLQERELVEPLTPSGEAPNQALLRILKETEFKKIKVLGSGAFGTVYKGLWI
PEGEKVKIPVAIKELREATSPKANKEILDEAYVMASVDNPHVCRLLGICLTSTVQLIT
QLMPFGCLLDYVREHKDNIGSQYLLNWCVQIAKGMNYLEDRRLVHRDLAARNVL
VKTPQHVKITDFGLAKLLGAEEKEYHAEGGKVPIKWMALESILHRIYTHQSDVWSY
GVTVWELMTFGSKPYDGIPASEISSILEKGERLPQPPICTIDVYMIMVKCWMIDADS
RPKFRELIIEFSKMARDPQRYLVIQGDERMHLPSPTDSNFYRALMDEEDMDDVVDA
DEYLIPQQGFFSSPSTSRTPLLSSLSATSNNSTVACIDRNGLQSCPIKEDSFLQRYSSD
PTGALTEDSIDDTFLPVPEYINQSVPKRPAGSVQNPVYHNQPLNPAPSRDPHYQDPH
STAVGNPEYLNTVQPTCVNSTFDSPAHWAQKGSHQISLDNPDYQQDFFPKEAKPNG
IFKGSTAENAEYLRVAPQSSEFIGA
EGFR Isoform 3, Uniprot P00533-3:
(SEQ ID NO: 130)
MRPSGTAGAALLALLAALCPASRALEEKKVCQGTSNKLTQLGTFEDHFLSLQRMF
NNCEVVLGNLEITYVQRNYDLSFLKTIQEVAGYVLIALNTVERIPLENLQIIRGNMY
YENSYALAVLSNYDANKTGLKELPMRNLQEILHGAVRFSNNPALCNVESIQWRDIV
SSDFLSNMSMDFQNHLGSCQKCDPSCPNGSCWGAGEENCQKLTKIICAQQCSGRCR
GKSPSDCCHNQCAAGCTGPRESDCLVCRKFRDEATCKDTCPPLMLYNPTTYQMDV
NPEGKYSFGATCVKKCPRNYVVTDHGSCVRACGADSYEMEEDGVRKCKKCEGPC
RKVCNGIGIGEFKDSLSINATNIKHFKNCTSISGDLHILPVAFRGDSFTHTPPLDPQEL
DILKTVKEITGFLLIQAWPENRTDLHAFENLEIIRGRTKQHGQFSLAVVSLNITSLGL
RSLKEISDGDVIISGNKNLCYANTINWKKLFGTSGQKTKIISNRGENSCKATGQVCH
ALCSPEGCWGPEPRDCVSCRNVSRGRECVDKCNLLEGEPREFVENSECIQCHPECLP
QAMNITCTGRGPDNCIQCAHYIDGPHCVKTCPAGVMGENNTLVWKYADAGHVCH
LCHPNCTYGPGNESLKAMLFCLFKLSSCNQSNDGSVSHQSGSPAAQESCLGWIPSLL
PSEFQLGWGGCSHLHAWPSASVIITASSCH
EGFRvIII, Uniprot P00533[30-297]
(SEQ ID NO: 131)
MRPSGTAGAA LLALLAALCP ASRALEEKKV CQGTSNKLTQ LGTFEDHFLS
LQRMFNNCEV VLGNLEITYV QRNYDLSFLK TIQEVAGYVL
IALNTVERIPLENLQIIRGN MYYENSYALA VLSNYDANKT GLKELPMRNL
QEILHGAVRF SNNPALCNVE SIQWRDIVSS DFLSNMSMDF QNHLGSCQKC
DPSCPNGSCWGAGEENCQKL TKIICAQQCS GRCRGKSPSD CCHNQCAAGC
TGPRESDCLV CRKFRDEATC KDTCPPLMLY NPTTYQMDVN PEGKYSFGAT
CVKKCPRNYV VTDHGSCVRA CGADSYEMEE DGVRKCKKCE GPCRKVCNGI
GIGEFKDSLS INATNIKHFK NCTSISGDLH ILPVAFRGDS FTHTPPLDPQ
ELDILKTVKE ITGFLLIQAW PENRTDLHAF ENLEIIRGRT KQHGQFSLAV
VSLNITSLGL RSLKEISDGD VIISGNKNLC YANTINWKKL FGTSGQKTKI
ISNRGENSCK ATGQVCHALC SPEGCWGPEP RDCVSCRNVS RGRECVDKCN
LLEGEPREFV ENSECIQCHP ECLPQAMNIT CTGRGPDNCI QCAHYIDGPH
CVKTCPAGVM GENNTLWVKY ADAGHVCHLC HPNCTYGCTG PGLEGCPTNG
PKIPSIATGM VGALLLLLVV ALGIGLFMRR RHIVRKRTLR RLLQERELVE
PLTPSGEAPN QALLRILKET EFKKIKVLGS GAFGTVYKGL WIPEGEKVKI
PVAIKELREATSPKANKEIL DEAYVMASVD NPHVCRLLGI CLTSTVQLIT
QLMPFGCLLDYVREHKDNIG SQYLLNWCVQ IAKGMNYLED RRLVHRDLAA
RNVLVKTPQHVKITDFGLAK LLGAEEKEYH AEGGKVPIKW MALESILBRI
YTHQSDVWSYGVTVWELMTF GSKPYDGIPA SEISSILEKG ERLPQPPICT
IDVYMIMVKC WMIDADSRPK FRELIIEFSK MARDPQRYLV
IQGDERMHLPLMDEEDMDDV VDADEYLIPQ QGFFSSPSTS RTPLLSSLSA
TSNNSTVACIDRNGLQSCPI KEDSFLQRYS SDPTGALTED SIDDTFLPVP
EYINQSVPKRPAGSVQNPVY HNQPLNPAPS RDPHYQDPHS TAVGNPEYLN
TVQPTCVNSTFDSPAHWAQK GSHQISLDNP DYQQDFFPKE AKPNGIFKGS
TAENAEYLRVAPQSSEFIGA
Polynucleotide encoding anti-EGFR heavy chain variable region 1:
(SEQ ID NO: 132)
GACATTCTAATGACCCAATCTCCACTCTCCCTGCCTGTCAGTCTTGGAGATCAA
GCCTCCTACCTGCAAAGGCCAGGCCAGTCTCCAAAGCTCCTGATCTACAAAGTT
TCCGACCGATTTTACCTGCAAAGGCCAGGCCAGTCTCCAAAGCTCCTGATCTAC
AAAGTTTCCGACCGATTTTCTGGGGTCCCAGACAGGTTCAGTGGCAGTGGATCA
GGGACAGATTTCACACTCAAGATCAGCAGAGTAGAGGCTGAGGATCTGGGAAT
TTATTACTGCTTTCAAGGTTCACATATTCCTCCCACGTTCGGAGGGGGGACCAA
GCTGGAAATCAAACGTGCGGCC
Polynucleotide encoding anti-EGFR light chain variable region 1:
(SEQ ID NO: 133)
CAGGTCCAGCTGCAGCAGTCTGGGTCTGAGATGGCGAGGCCTGGAGCTTCAGT
GAAGCTGCCCTGCAAGGCTTCTGGCGACACATTCACCAGTTACTGGATGCACTG
GGTGAAGCAGAGGCATGGACATGGCCCTGAGTGGATCGGAAATATTTATCCAG
GTAGTGGTGGTACTAACTACGCTGAGAAGTTCAAGAACAAGGTCACTCTGACTG
TAGACAGGTCCTCCCGCACAGTCTACATGCACCTCAGCAGGCTGACATCTGAGG
ACTCTGCGGTCTATTATTGTACAAGATCGGGGGGTCCCTACTTCTTTGACTACTG
GGGCCAAGGCACCACTCTCACAGTCTCCTCC
Linker:
(SEQ ID NO: 134)
GGGGS.
Linker:
(GGGGS)n,
wherein n may be an integer of 1 (SEQ ID NO: 134),
or 2 (SEQ ID NO: 135), or 3 (SEQ ID NO: 14),
or 4 (SEQ ID NO: 136), or 5 (SEQ ID NO: 137), or
6 (SEQ ID NO: 138), or 7 (SEQ ID NO: 139), or
8 (SEQ ID NO: 140), or 9 (SEQ ID NO: 141), or
10 (SEQ ID NO: 142), or 11 (SEQ ID NO: 143), or
12 (SEQ ID NO: 144), or 13 (SEQ ID NO: 145), or
14 (SEQ ID NO: 146), or 15 (SEQ ID NO: 147), or more.
EF1 alpha promoter sequence:
(SEQ ID NO: 148)
AAGGATCTGCGATCGCTCCGGTGCCCGTCAGTGGGCAGAGCGCACATCGCCCA
CAGTCCCCGAGAAGTTGGGGGGAGGGGTCGGCAATTGAACGGGTGCCTAGAGA
AGGTGGCGCGGGGTAAACTGGGAAAGTGATGTCGTGTACTGGCTCCGCCTTTTT
CCCGAGGGTGGGGGAGAACCGTATATAAGTGCAGTAGTCGCCGTGAACGTTCT
TTTTCGCAACGGGTTTGCCGCCAGAACACAGCTGAAGCTTCGAGGGGCTCGCAT
CTCTCCTTCACGCGCCCGCCGCCCTACCTGAGGCCGCCATCCACGCCGGTTGAG
TCGCGTTCTGCCGCCTCCCGCCTGTGGTGCCTCCTGAACTGCGTCCGCCGTCTAG
GTAAGTTTAAAGCTCAGGTCGAGACCGGGCCTTTGTCCGGCGCTCCCTTGGAGC
CTACCTAGACTCAGCCGGCTCTCCACGCTTTGCCTGACCCTGCTTGCTCAACTCT
ACGTCTTTGTTTCGTTTTCTGTTCTGCGCCGTTACAGATCCAAGCTGTGACCGGC
GCCTAC
A reverse sequence of SEQ ID NO: 71:
(SEQ ID NO: 151)
AVGTIVDQSAK
STAT3 association motif:
(SEQ ID NO: 152)
YXXQ
STAT3 association motif:
(SEQ ID NO: 153)
YRHQ.
A reverse sequence of SEQ ID NO: 77:
(SEQ ID NO: 154)
KLELKTGAGFTLPTSYHQQCYYVALDEAQVNSITFTFDTGSGSGTFRDPVGTYRYS
ASYILLKPSQGPKQQYWAVGTIVDQSAKCTISVRDGVSTSMSKQSQTMVIDS
Anti-BCMA sequence 2 scFv heavy chain (SEQ ID Nos.: 99 and 95)
(SEQ ID NO: 155)
ATGGGATGGAGCTCTATCATCCTCTTCTTGGTAGCAACAGCTACAGGTGTCCAC
CAGATTCAGCTGGTGCAGAGCGGCCCTGAGCTGAAGAAACCCGGCGAGACAGT
GAAGATCAGCTGCAAGGCCTCCGGCTACACCTTCACCGACTACAGCATCAACTG
GGTGAAAAGAGCCCCTGGCAAGGGCCTGAAGTGGATGGGCTGGATCAACACCG
AGACAAGAGAGCCCGCCTACGCCTACGACTTCCGGGGCAGATTCGCCTTCAGCC
TGGAAACCAGCGCCAGCACCGCCTACCTGCAGATCAACAACCTGAAGTACGAG
GACACCGCCACCTACTTTTGCGCCCTGGACTACAGCTACGCCATGGACTACTGG
GGCCAGGGCACCAGCGTGACCGTGTCCAGC
Anti-BCMA sequence 2 scFv light chain
(SEQ ID NO: 156)
GACATCGTGCTGACCCAGAGCCCCCCCAGCCTGGCCATGTCTCTGGGCAAGAGA
GCCACCATCAGCTGCCGGGCCAGCGAGAGCGTGACCATCCTGGGCAGCCACCT
GATCCACTGGTATCAGCAGAAGCCCGGCCAGCCCCCCACCCTGCTGATCCAGCT
CGCCAGCAATGTGCAGACCGGCGTGCCCGCCAGATTCAGCGGCAGCGGCAGCA
GAACCGACTTCACCCTGACCATCGACCCCGTGGAAGAGGACGACGTGGCCGTG
TACTACTGCCTGCAGAGCCGGACCATCCCCCGGACCTTTGGCGGAGGCACCAAA
CTGGAAATCAAG
Anti-CS-1 scFv heavy chain DNA sequence
(SEQ ID NO: 157)
AGCGTTACCG TGAGTACAGG CCAGGGCTGG TATGACATGG CACGTACAGC
CATCATGACC TCGCGCGCAT GTTACTACGT CGCGTCAGAT GAATCGACGC
CTTCCTCGCT GCAAATGTAT GCAACCTCCA GCAGCAAAGA TGTTACCCTG
ACCGCAAAGG ACAAGTTTAA ACAGAATTTG CGTACGGAGA GTGACTCCCC
GCACATCATG GGAATCTGGG AGTTGGGTCA GGGGCCTCGT CAGAAGGTAT
GGAACATGTG GTATACAACT TTTTCGTACG GCTCAGCAAA ATGCAGCTTG
AAAGTGTCGG CAGGTCCGCG CGTGCTGGAG GCCGGTCCGC AGCAGCTGCA
AGTCCAGTCT
Anti-CS-1 scFv light chain DNA sequence
(SEQ ID NO: 158)
AAACTTGAGT TGAAGACCGG TGCCGGCTTC ACCTTACCGA CCAGTTATCA
TCAACAATGC TATTACGTGG CCCTGGACGA AGCACAGGTG AATTCAATTA
CGTTTACGTT TGATACCGGC TCTGGCAGCG GTACATTTCG TGATCCCGTG
GGCACTTACC GCTATTCGGC GAGTTATATC TTGCTGAAAC CTTCCCAAGG
TCCGAAACAG CAGTACTGGG CGGTTGGCAC CATTGTAGAC CAATCAGCCA
AATGTACAAT CTCGGTTCGC GATGGTGTCA GTACGTCGAT GTCTAAGCAG
TCACAGACAA TGGTTATCGA T
Anti-NKG2D heavy chain DNA sequence
(SEQ ID NO: 159)
CAAGTGCAGC TGGTTGAATC CGGTGGCGGT CTGGTCAAGC CGGGCGGCTC
TTTGCGTCTG AGCTGTGCCG CGTCGGGTTT TACCTTCAGC TCTTATGGTA
TGCATTGGGT GCGTCAGGCG CCTGGCAAAG GTCTGGAGTG GGTTGCGTTC
ATCCGCTACG ATGGGTCTAA CAAATATTAT GCCGACTCAG TAAAAGGACG
CTTCACTATT AGCCGCGACA ATAGCAAAAA TACCCTGTAC CTGCAAATGA
ATAGCCTGCG CGCCGAAGAT ACCGCCGTTT ACTATTGCGC TAAAGATCGT
GGCCTGGGTG ATGGTACGTA CTTCGATTAC TGGGGTCAGG GCACCACCGT
TACCGTTAGT TCA
Anti-NKG2D light chain DNA sequence
(SEQ ID NO: 160)
CAGTCAGCGC TTACGCAGCC GGCGTCGGTG TCGGGTTCCC CGGGTCAGTC
GATCACGATC AGCTGTAGTG GGAGCAGCTC CAACATCGGT AACAACGCAG
TGAACTGGTA TCAGCAACTG CCGGGAAAAG CGCCGAAACT GCTGATTTAC
TATGATGATT TGCTGCCAAG TGGAGTTAGT GACCGCTTTT CCGGCAGTAA
ATCGGGTACC TCGGCTTTTC TGGCTATTTC GGGTCTCCAG AGCGAGGATG
AAGCTGATTA TTATTGCGCC GCATGGGATG ATAGCTTAAA TGGCCCAGTT
TTTGGCGGCG GTACTAAACT GACCGTGCTG
Linker
(SEQ ID NO: 161)
GGCGGTGGCGGTTCTGGTGGCGGTGGCTCCGGCGGTGGCGGTTCT
Anti-NKG2D Heavy Chain
(SEQ ID NO: 162)
CAAGTGCAGCTGGTTGAATCCGGTGGCGGTCTGGTCAAGCCGGGCGGCTCTTTG
CGTCTGAGCTGTGCCGCGTCGGGTTTTACCTTCAGCTCTTATGGTATGCATTGGG
TGCGTCAGGCGCCTGGCAAAGGTCTGGAGTGGGTTGCGTTCATCCGCTACGATG
GGTCTAACAAATATTATGCCGACTCAGTAAAAGGACGCTTCACTATTAGCCGCG
ACAATAGCAAAAATACCCTGTACCTGCAAATGAATAGCCTGCGCGCCGAAGAT
ACCGCCGTTTACTATTGCGCTAAAGATCGTGGCCTGGGTGATGGTACGTACTTC
GATTACTGGGGTCAGGGCACCACCGTTACCGTTAGTTCAGGTGGGGGCGGCTCT
Anti-NKG2D Light Chain
(SEQ ID NO: 163)
CAGCGCTTACGCAGCCGGCGTCGGTGTCGGGTTCCCCGGGTCAGTCGATCACGA
TCAGCTGTAGTGGGAGCAGCTCCAACATCGGTAACAACGCAGTGAACTGGTAT
CAGCAACTGCCGGGAAAAGCGCCGAAACTGCTGATTTACTATGATGATTTGCTG
CCAAGTGGAGTTAGTGACCGCTTTTCCGGCAGTAAATCGGGTACCTCGGCTTTT
CTGGCTATTTCGGGTCTCCAGAGCGAGGATGAAGCTGATTATTATTGCGCCGCA
TGGGATGATAGCTTAAATGGCCCAGTTTTTGGCGGCGGTACTAAACTGACCGTG
CTG
Anti-CS1 Heavy Chain
(SEQ ID NO: 164)
CTCCGTGACGGTGTCGACGGGCCAAGGATGGTACGATATGGCACGGACCGCGA
TTATGACATCGCGGGCGTGCTATTACGTGGCCAGCGATGAGTCGACCCCTTCCT
CTCTGCAAATGTATGCCACCTCCTCTTCAAAAGACGTGACTCTGACTGCGAAAG
ACAAATTTAAACAGAATCTGCGCACCGAAAGCGATAGCCCACATATCATGGGC
ATCTGGGAACTGGGCCAGGGCCCCCGCCAGAAAGTGTGGAACATGTGGTACAC
CACCTTCAGCTATGGTTCGGCCAAATGTTCCCTGAAGGTATCAGCCGGCCCGCG
CGTTCTTGAGGCGGGTCCGCAGCAGCTGCAGGTACAGAGC
Anti-CS1 Light Chain
(SEQ ID NO: 165)
AAACTGGAACTCAAGACGGGTGCGGGATTTACCCTCCCTACGAGCTATCACCAG
CAGTGCTATTACGTGGCGCTTGACGAAGCGCAGGTGAACTCTATTACCTTTACC
TTTGATACAGGATCAGGCAGCGGTACGTTCCGTGATCCGGTAGGTACGTACCGG
TATAGTGCAAGCTATATCCTTCTGAAACCTTCTCAGGGTCCGAAACAGCAGTAC
TGGGCGGTGGGAACGATCGTGGACCAGTCTGCCAAATGTACAATTTCAGTTCGC
GACGGAGTTAGCACCTCCATGAGCAAGCAGTCCCAAACCATGGTGATTGACTCT
Anti-BCMA Heavy Chain Sequence 2
(SEQ ID NO: 166)
CAGATTCAGCTGGTGCAGAGCGGCCCTGAGCTGAAGAAACCCGGCGAGACAGT
GAAGATCAGCTGCAAGGCCTCCGGCTACACCTTCACCGACTACAGCATCAACTG
GGTGAAAAGAGCCCCTGGCAAGGGCCTGAAGTGGATGGGCTGGATCAACACCG
AGACAAGAGAGCCCGCCTACGCCTACGACTTCCGGGGCAGATTCGCCTTCAGCC
TGGAAACCAGCGCCAGCACCGCCTACCTGCAGATCAACAACCTGAAGTACGAG
GACACCGCCACCTACTTTTGCGCCCTGGACTACAGCTACGCCATGGACTACTGG
GGCCAGGGCACCAGCGTGACCGTGTCCAGC
Anti-BCMA Light Chain Sequence 2
(SEQ ID NO: 167)
GACATCGTGCTGACCCAGAGCCCCCCCAGCCTGGCCATGTCTCTGGGCAAGAGA
GCCACCATCAGCTGCCGGGCCAGCGAGAGCGTGACCATCCTGGGCAGCCACCT
GATCCACTGGTATCAGCAGAAGCCCGGCCAGCCCCCCACCCTGCTGATCCAGCT
CGCCAGCAATGTGCAGACCGGCGTGCCCGCCAGATTCAGCGGCAGCGGCAGCA
GAACCGACTTCACCCTGACCATCGACCCCGTGGAAGAGGACGACGTGGCCGTG
TACTACTGCCTGCAGAGCCGGACCATCCCCCGGACCTTTGGCGGAGGCACCAAA
CTGGAAATCAAG
IgG1 heavy chain signal peptide
DNA
(SEQ ID NO: 168)
ATGGAATTTGGGCTGCGCTGGGTTTTCCTTGTTGCTATTTTAAAAGATGTCCAGTGT
Protein
(SEQ ID NO: 169)
MEFGLRWVFLVAILKDVQC
IgG1 FcDNA
(SEQ ID NO: 170)
GAGCCCAAATCTTGTGACAAAACTCACACATGCCCACCGTGCCCAGCACCTGAA
CTCCTGGGGGGACCGTCAGTCTTCCTCTTCCCCCCAAAACCCAAGGACACCCTC
ATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATG
CCAAGACAAAGCCGCGGGAGGAGCAGTACAACAGCACGTACCGTGTGGTCAGC
GTCCTCACCGTCCTGCACCAGGACTGGCTGAATGGCAAGGAGTACAAGTGCAA
GGTCTCCAACAAAGCCCTCCCAGCCCCCATCGAGAAAACCATCTCCAAAGCCA
AAGGGCAGCCCCGAGAACCACAGGTGTACACCCTGCCCCCATCCCGGGATGAG
CTGACCAAGAACCAGGTCAGCCTGACCTGCCTGGTCAAAGGCTTCTATCCCAGC
GACATCGCCGTGGAGTGGGAGAGCAATGGGCAGCCGGAGAACAACTACAAGA
CCACGCCTCCCGTGCTGGACTCCGACGGCTCCTTCTTCCTCTACAGCAAGCTCAC
CGTGGACAAGAGCAGGTGGCAGCAGGGGAACGTCTTCTCATGCTCCGTGATGC
ATGAGGCTCTGCACAACCACTACACACAGAAGAGCCTCTCCCTGTCTCCGGAGC
TGCAACTGGAGGAGAGCTGTGCGGAGGCGCAGGACGGGGAGCTGGACGGGCT
GTGGACGACCATCACCATCTTCATCACACTCTTCCTGTTAAGCGTGTGCTACAGT
GCCACCGTCACCTTCTTCAAGGTGAAGTGGATCTTCTCCTCGGTGGTGGACCTG
AAGCAGACCATCATCCCCGACTACAGGAACATGATCGGACAGGGGGCC
Protein encoded by IgG1 FcDNA
(SEQ ID NO: 171)
EPKSCDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQ
KSLSLSPELQLEESCAEAQDGELDGLWTTITIFITLFLLSVCYSATVTFFKVKWIFSSV
VDLKQTIIPDYRNMIGQGA

1. A polypeptide comprising:
(a) (i) an amino acid sequence of a chimeric antigen receptor (CAR) comprising:
(1) an antigen binding domain that recognizes and binds a first tumor associated antigen (TAA) on a cancer cell (first anti-TAA antigen binding domain) with the proviso that the first TAA is not a B-cell maturation antigen (BCMA);
(2) a hinge domain;
(3) a transmembrane domain; and
(4) an intracellular domain; and
(ii) a bispecific antibody comprising
(1) an antigen binding domain that recognizes and binds a second TAA on the cancer cell (second anti-TAA antigen binding domain); and
(2) an antigen binding domain that recognizes and binds NKG2D (anti-NKG2D antigen binding domain); or
(b) (i) an amino acid sequence of a CAR comprising:
(1) an antigen binding domain that recognizes and binds BCMA (anti-BCMA antigen binding domain);
(2) a hinge domain;
(3) a transmembrane domain; and
(4) an intracellular domain; and
(ii) a bispecific antibody comprising
(1) an antigen binding domain that recognizes and binds NKG2D (anti-NKG2D antigen binding domain) and
(2) an antigen binding domain that recognizes and binds CS1 (anti-CS1 antigen binding domain),
wherein the anti-NKG2D antigen binding domain comprises the following complementarity-determining regions (CDRs):
a light chain complementarity-determining region 1 (CDRL1) comprising
(SEQ ID NO: 6)
SGSSSNIGNNAVN;
a light chain complementarity-determining region 2 (CDRL2) comprising YDDLLPS (SEQ ID NO: 7);
a light chain complementarity-determining region 3 (CDRL3) comprising
(SEQ ID NO: 8)
AAWDDSLNGPV
a heavy chain complementarity-determining region 1 (CDRH1) comprising GFTFSSY (SEQ ID NO: 9);
a heavy chain complementarity-determining region 2 (CDRH2) comprising RYDGSN (SEQ ID NO: 10); and
a heavy chain complementarity-determining region 3 (CDRH3) comprising DRGLGDGTYFDY (SEQ ID NO: 11),
and wherein the anti-BCMA antigen binding domain comprises the following CDRs:
a CDRL1 comprising RASESVTILGSHLIH (SEQ ID NO: 47), SASQDISNYLN (SEQ ID NO: 48), RASESVTILGSHLIY (SEQ ID NO: 49);
a CDRL2 comprising LASNVQT (SEQ ID NO: 50), YTSNLHS (SEQ ID NO: 51), LASNVQT (SEQ ID NO: 52);
a CDRL3 comprising LQSRTIPRT (SEQ ID NO: 53), QQYRKLPWT (SEQ ID NO: 54), LQSRTIPRT (SEQ ID NO: 55);
a CDRH1 comprising GYTFTDY (SEQ ID NO: 56), GGTFSNY (SEQ ID NO: 57), GYTFRHY (SEQ ID NO: 58);
a CDRH2 comprising INTETRE (SEQ ID NO: 59), YRGHSD (SEQ ID NO: 60), NTESGV (SEQ ID NO: 61); and
a CDRH3 comprising DYSYAMDY (SEQ ID NO: 62), GAIYNGYDVLDN (SEQ ID NO: 63), DYLYSLDF (SEQ ID NO: 64):
and wherein the anti-CS1 antigen binding domain comprises the following CDRs:
a CDRL1 comprising
(SEQ ID NO: 71)
KASQDVITGVA;
a CDRL2 comprising
(SEQ ID NO: 72)
SASYRYT;
a CDRL3 comprising
(SEQ ID NO: 73)
QQHYSTPLT;
a CDRH1 comprising
(SEQ ID NO: 74)
GYSFTTY;
a CDRH2 comprising
(SEQ ID NO: 75)
HPSDSE;
and
a CDRH3 comprising
(SEQ ID NO: 76)
STMIATRAMDY.

2. The polypeptide claim 1, wherein the hinge domain comprises a CD8 α hinge domain or an IgG1 hinge domain comprising LEPKSCDKTHTCPPCPDPKGT (SEQ ID NO: 1).

3. (canceled)

4. The polypeptide of claim 1, wherein the transmembrane domain comprises a CD8 α transmembrane domain or a CD28 transmembrane domain.

5. The polypeptide of claim 1, wherein the intracellular domain comprises one or more or two or more costimulatory regions selected from a CD28 costimulatory signaling region, a 4-1BB costimulatory signaling region, an ICOS costimulatory signaling region, or an OX40 costimulatory region.

6. The polypeptide of claim 4, wherein the CAR comprises a CD28 transmembrane and cytoplasmic domain comprising
FWVLVVVGGVLACYSLLVTVAFIIFWVRSKRSRLLHSDYMNMTPRRPGPTRKHYQPYA PPRDFAAYRS (SEQ ID NO: 2) or an equivalent thereof.

7. The polypeptide of claim 1, wherein the intracellular domain further comprises a CD3 zeta signaling domain comprising
RVKFSRSADAPAYQQGQNQLYNELNLGRREEYDVLDKRRGRDPEMGGKPRRKNPQEG LYNELQKDKMAEAYSEIGMKGERRRGKGHDGLYQGLSTATKDTYDALHMQALPPR (SEQ ID NO: 3) or an equivalent thereof.

8. (canceled)

9. The polypeptide of claim 1, wherein the intracellular domain further comprises an IL2Rβ or a fragment thereof comprising an JAK-STAT activation domain.

10. The polypeptide of claim 1, further comprising a suicide gene product selected from one or more of: HSV-TK (Herpes simplex virus thymidine kinase), cytosine deaminase, nitroreductase, carboxylesterase, cytochrome P450 or PNP (Purine nucleoside phosphorylase), truncated EGFR, or inducible caspase (“iCasp”).

11. (canceled)

12. The polypeptide of claim 1, wherein the CAR further comprises a signal peptide at its N terminus, comprising
MGWSSIILFLVATATGVH (SEQ ID NO: 5), or an equivalent thereof.

13.-15. (canceled)

16. The polypeptide of claim 1, wherein the anti-NKG2D antigen binding domain comprises:
a light chain variable region comprising
QSALTQPASVSGSPGQSITISCSGSSSNIGNNAVNWYQQLPGKAPKLLIYYDDLLPSGVSD RFSGSKSGTSAFLAISGLQSEDEADYYCAAWDDSLNGPVFGGGTKLTVL (SEQ ID NO: 12), or an equivalent thereof; and/or
a heavy chain variable region comprising
QVQLVESGGGLVKPGGSLRLSCAASGFTFSSYGMHWVRQAPGKGLEWVAFIRYDGSN KYYADSVKGRFTISRDNSKNTLYLQMNSLRAEDTAVYYCAKDRGLGDGTYFDYWGQG TTVTVSS (SEQ ID NO: 13), or an equivalent thereof,
wherein the equivalent thereof recognizes and binds NKG2D.

17. The polypeptide of claim 16, wherein the equivalent of SEQ ID NO: 12 or 13 is at least 80% identical to SEQ ID NO: 12 or 13, respectively.

18. The polypeptide of claim 16, wherein the equivalent of SEQ ID NOs: 6-13 comprises the amino acid domain of C terminus to N terminus of SEQ ID NO: 6-13, respectively.

19.-37. (canceled)

38. The polynucleotide of claim 1, wherein the anti-BCMA antigen binding domain comprises
a light chain variable region comprising a sequence selected from
DIVLTQSPPSLAMSLGKRATISCRASESVTILGSHLIHWYQQKPGQPPTLLIQLASNVQTG VPARFSGSGSRTDFTLTIDPVEEDDVAVYYCLQSRTIPRTFGGGTKLEIK (SEQ ID NO: 65),
DIQMTQSPSSLSASVGDRVTITCSASQDISNYLNWYQQKPGKAPKLLIYYTSNLHSGVPS RFSGSGSGTDFTLTISSLQPEDFATYYCQQYRKLPWTFGQGTKLEIKR (SEQ ID NO: 66),
DIVLTQSPPSLAMSLGKRATISCRASESVTILGSHLIYWYQQKPGQPPTLLIQLASNVQTG VPARFSGSGSRTDFTLTIDPVEEDDVAVYYCLQSRTIPRTFGGGTKLEIK (SEQ ID NO: 67), or an equivalent of each thereof and/or
a heavy chain variable region comprising a sequence selected from
QIQLVQSGPELKKPGETVKISCKASGYTFTDYSINWVKRAPGKGLKWMGWINTETREPA YAYDFRGRFAFSLETSASTAYLQINNLKYEDTATYFCALDYSYAMDYWGQGTSVTVSS (SEQ ID NO: 68),
QVQLVQSGAEVKKPGSSVKVSCKASGGTFSNYWMHWVRQAPGQGLEWMGATYRGHS DTYYNQKFKGRVTITADKSTSTAYMELSSLRSEDTAVYYCARGAIYNGYDVLDNWGQ GTLVTVSS (SEQ ID NO: 69),
QIQLVQSGPELKKPGETVKISCKASGYTFRHYSMNWVKQAPGKGLKWMGRINTESGVP IYADDFKGRFAFSVETSASTAYLVINNLKDEDTASYFCSNDYLYSLDFWGQGTALTVSS (SEQ ID NO: 70), or an equivalent of each thereof,
wherein the equivalent thereof recognizes and bins BCMA.

39. The polypeptide of claim 38, wherein the equivalent of SEQ ID NOs: 65-70 is at least 80%, respectively.

40. The polypeptide of claim 38, wherein the equivalent of SEQ ID NOs: 47-70 comprises the amino acid sequence of C terminus to N terminus of SEQ ID NOs: 47-70, respectively.

41.-44. (canceled)

45. The polypeptide of claim 1, wherein the anti-CS1 antigen binding domain comprises
a light chain variable region comprising a sequence selected from SDIVMTQSQKSMSTSVGDRVSITCKASQDVITGVAWYQQKPGQSPKLLIYSASYRYTGV PDRFTGSGSGTDFTFTISNVQAEDLAVYYCQQHYSTPLTFGAGTKLELK (SEQ ID NO: 77)
KLELKTGAGFTLPTSYHQQCYYVALDEAQVNSITFTFDTGSGSGTFRDPVGTYRY SASYILLKPSQGPKQQYWAVGTIVDQSAKCTISVRDGVSTSMSKQSQTMVIDS (SEQ ID NO: 154),
DIVMTQSQKSMSTSVGDRVSITCKASQDVITGVAWYQQKPGQSPKLLIYSASYRYTGVP DRFTGSGSGTDFTFTISNVQAEDLAVYYCQQHYSTPLTFGAGTKLELK (SEQ ID NO: 78), or an equivalent of each thereof and/or
a heavy chain variable region comprising a sequence selected from SVTVSTGQGWYDMARTAIMTSRACYYVASDESTPSSLQMYATSSSKDVTLTAKDKFKQ NLRTESDSPHIMGIWELGQGPRQKVWNMWYTTFSYGSAKCSLKVSAGPRVLEAGPQQL QVQS (SEQ ID NO: 79),
SSVTVSTGQGWYDMARTAIMTSRACYYVASDESTPSSLQMYATSSSKDVTLTAKDKFK QNLRTESDSPHIMGIWELGQGPRQKVWNMWYTTFSYGSAKCSLKVSAGPRVLEAGPQQ LQVQ (SEQ ID NO: 149),
SQVQLQQPGAELVRPGASVKLSCKASGYSFTTYWMNWVKQRPGQGLEWIGMIHPSDSE TRLNQKFKDKATLTVDKSSSTAYMQLSSPTSEDSAVYYCARSTMIATRAMDYWGQGTS VTVS (SEQ ID NO: 80),
QVQLQQPGAELVRPGASVKLSCKASGYSFTTYWMNWVKQRPGQGLEWIGMIHPSDSE TRLNQKFKDKATLTVDKSSSTAYMQLSSPTSEDSAVYYCARSTMIATRAMDYWGQGTS VTVSS (SEQ ID NO: 150), or an equivalent of each thereof,
wherein the equivalent thereof recognizes and binds CS1.

46. The polypeptide of claim 45, wherein the equivalent of SEQ ID NOs: 77 to 80 is at least 80%, respectively.

47. The polypeptide of claim 45, wherein the equivalent of SEQ ID NOs: 71-80, 149-150 and 154 comprises the amino acid sequence of C terminus to N terminus of SEQ ID NOs: 71-80, 149-150 and 154, respectively.

48.-49. (canceled)

50. The polypeptide of claim 1, wherein the bispecific antibody further comprises a fragment crystallizable (Fc) region of an immunoglobulin, a mutant thereof, or an equivalent thereof, wherein the Fc region comprises
ESKYGPPCPPCPAPEFLGGPSVFLFPPKPKDTLMISRTPEVTCVVVDVSQEDPEVQFNWY VDGVEVHNAKTKPREEQFNSTYRVVSVLTVLHQDWLNGKEYKCKVSNKGLPSSIEKTIS KAKGQPREPQVYTLPPSQEEMTKNQVSLTCLVKGFYPSDIAVEWESNGQPENNYKTTPP VLDSDGSFFLYSRLTVDKSRWQEGNVFSCSVMHEALHNHYTQKSLSLSLGK (SEQ ID NO: 81), or an equivalent thereof that binds an Fc receptor on an immune cell or on a platelet or that binds a complement protein.

51.-52. (canceled)

53. The polypeptide of claim 50, wherein the Fc region comprises
ESKYGPPCPPCPAPEAAGGPSVFLFPPKPKDTLMISRTPEVTCVVVDVSQEDPEVQFNWY VDGVEVHNAKTKPREEQFQSTYRVVSVLTVLHQDWLNGKEYKCKVSNKGLPSSIEKTIS KAKGQPREPQVYTLPPSQEEMTKNQVSLTCLVKGFYPSDIAVEWESNGQPENNYKTTPP VLDSDGSFFLYSRLTVDKSRWQEGNVFSCSVMHEALHNHYTQKSLSLSLGK (SEQ ID NO: 82) or an Fc equivalent having mutations at a position corresponding to amino acid (aa) 16, aa 17 and aa 79 of SEQ ID NO: 81, and binding an Fc receptor on an immune cell or on a platelet or binding a complement protein.

54. The polypeptide of claim 1, wherein the bispecific antibody further comprises a peptide linker between the two antigen binding domains.

55. The polypeptide of claim 54, wherein the peptide linker comprises
(SEQ ID NO: 83)
PSGQAGAAASESLFVSNHAY.

56. The polypeptide of claim 1, wherein the bispecific antibody further comprises a signal peptide at its N terminus, optionally different from the signal peptide of the CAR, comprising a sequence selected from
(SEQ ID NO: 4)
MYRMQLLSCIALSLALVTNS.

57.-59. (canceled)

60. The polypeptide of claim 1, wherein the bispecific antibody and the CAR are contiguous sequences.

61. The polypeptide of claim 1, further comprising a cleavable peptide located between any two of the following:
the CAR;
the bispecific antibody; and
an optional suicide gene product.

62. The polypeptide of claim 61, wherein the cleavable peptide is a self-cleaving peptide.

63. The polypeptide of claim 62, wherein the self-cleaving peptide is a T2A peptide.

64. The polypeptide of claim 61, wherein cleavable peptide or self-cleaving peptide or the T2A peptide comprises
HVGSGEGRGSLLTCGDVEENPGP (SEQ ID NO: 85) or an equivalent thereof.

65.-66. (canceled)

67. A polynucleotide encoding a polypeptide of claim 1.

68-75. (canceled)

76. A vector comprising a polynucleotide of claim 67.

77.-82. (canceled)

83. An isolated or engineered cell comprising a polypeptide of claim 1.

84.-95. (canceled)

96. A method of producing a cell expressing a CAR and secreting a bispecific antibody comprising transducing or transfecting a cell or a cell population with a polynucleotide of claim 67.

97-129. (canceled)

130. A kit comprising an optional instructions for use and a polypeptide of claim 1.

131. A polynucleotide encoding a CAR of the polypeptide of claim 1.

132. A polynucleotide encoding a bispecific antibody of the polypeptide of claim 1.

133. An isolated or engineered cell comprising a polynucleotide of claim 67.

134. An isolated or engineered cell comprising a vector of claim 76.

135. A method of producing a cell expressing a CAR and secreting a bispecific antibody comprising transducing or transfecting a cell or a cell population with a vector of claim 76.


SEQUENCE LISTING (174 sequences)

SEQ ID NO: 1 - protein, 21 aa, Artificial Sequence
  Description of Artificial Sequence Synthetic peptide
LEPKSCDKTHTCPPCPDPKGT

SEQ ID NO: 2 - protein, 68 aa, Artificial Sequence
  Description of Artificial Sequence Synthetic polypeptide
FWVLVVVGGVLACYSLLVTVAFIIFWVRSKRSRLLHSDYMNMTPRRPGPTRKHYQPYAPPRDFAAYRS

SEQ ID NO: 3 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 20 aa, Artificial Sequence
  Description of Artificial Sequence Synthetic peptide
MYRMQLLSCIALSLALVTNS

SEQ ID NO: 5 - protein, 18 aa, Artificial Sequence
  Description of Artificial Sequence Synthetic peptide
MGWSSIILFLVATATGVH

SEQ ID NO: 6 - protein, 13 aa, Artificial Sequence
  Description of Artificial Sequence Synthetic peptide
SGSSSNIGNNAVN

SEQ ID NO: 7 - protein, 7 aa, Artificial Sequence
  Description of Artificial Sequence Synthetic peptide
YDDLLPS

SEQ ID NO: 8 - protein, 11 aa, Artificial Sequence
  Description of Artificial Sequence Synthetic peptide
AAWDDSLNGPV

SEQ ID NO: 9 - protein, 7 aa, Artificial Sequence
  Description of Artificial Sequence Synthetic peptide
GFTFSSY

SEQ ID NO: 10 - protein, 6 aa, Artificial Sequence
  Description of Artificial Sequence Synthetic peptide
RYDGSN

SEQ ID NO: 11 - protein, 12 aa, Artificial Sequence
  Description of Artificial Sequence Synthetic peptide
DRGLGDGTYFDY

SEQ ID NO: 12 - protein, 110 aa, Artificial Sequence
  Description of Artificial Sequence Synthetic polypeptide
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 13 - protein, 121 aa, Artificial Sequence
  Description of Artificial Sequence Synthetic polypeptide
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 14 - protein, 15 aa, Artificial Sequence
  Description of Artificial Sequence Synthetic peptide
GGGGSGGGGSGGGGS

SEQ ID NO: 15 - protein, 11 aa, Artificial Sequence
  Description of Artificial Sequence Synthetic peptide
RASQSISNNLH

SEQ ID NO: 16 - protein, 16 aa, Artificial Sequence
  Description of Artificial Sequence Synthetic peptide
KSSQSLLNSGNQKNYM

SEQ ID NO: 17 - protein, 7 aa, Artificial Sequence
  Description of Artificial Sequence Synthetic peptide
YASQSIS

SEQ ID NO: 18 - protein, 7 aa, Artificial Sequence
  Description of Artificial Sequence Synthetic peptide
GASTRES

SEQ ID NO: 19 - protein, 9 aa, Artificial Sequence
  Description of Artificial Sequence Synthetic peptide
QQSNTWPYT

SEQ ID NO: 20 - protein, 9 aa, Artificial Sequence
  Description of Artificial Sequence Synthetic peptide
QNDHSYPLT

SEQ ID NO: 21 - protein, 5 aa, Artificial Sequence
  Description of Artificial Sequence Synthetic peptide
SYWMH

SEQ ID NO: 22 - protein, 5 aa, Artificial Sequence
  Description of Artificial Sequence Synthetic peptide
NYGLH

SEQ ID NO: 23 - protein, 17 aa, Artificial Sequence
  Description of Artificial Sequence Synthetic peptide
EIDPSDSYKDYNQKFKD

SEQ ID NO: 24 - protein, 16 aa, Artificial Sequence
  Description of Artificial Sequence Synthetic peptide
VIWSGGSTDYNAAFIS

SEQ ID NO: 25 - protein, 9 aa, Artificial Sequence
  Description of Artificial Sequence Synthetic peptide
AITTTPFDF

SEQ ID NO: 26 - protein, 14 aa, Artificial Sequence
  Description of Artificial Sequence Synthetic peptide
GGIYYANHYYAMDY

SEQ ID NO: 27 - protein, 108 aa, Artificial Sequence
  Description of Artificial Sequence Synthetic polypeptide
DIVLTQSPATLSVTPGDSVSLSCRASQSISNNLHWYQQKSHESPRLLIKYASQSISGIPSRFSGSGSGTDFTLSINSVETEDFGVYFCQQSNTWPYTFGGGTKLEIKR

SEQ ID NO: 28 - protein, 114 aa, Artificial Sequence
  Description of Artificial Sequence Synthetic polypeptide
DIVMTQSPSSLSVSAGEKVTMSCKSSQSLLNSGNQKNYMAWYQQKPGQPPKLLIYGASTRESGVPDRFTGSGSGTDFTLTISSVQAEDLAVYYCQNDHSYPLTFGAGTKLELKR

SEQ ID NO: 29 - protein, 118 aa, Artificial Sequence
  Description of Artificial Sequence Synthetic polypeptide
QVQLQQPGAELVKPGASLKLSCKSSGYTFTSYWMHWVRQRPGHGLEWIGEIDPSDSYKDYNQKFKDKATLTVDRSSNTAYMHLSSLTSDDSAVYYCARAITTTPFDFWGQGTTLTVSS

SEQ ID NO: 30 - protein, 123 aa, Artificial Sequence
  Description of Artificial Sequence Synthetic polypeptide
QVQLKQSGPGLVQPSQSLSITCTVSGFSLTNYGLHWVRQSPGKGLEWLGVIWSGGSTDYNAAFISRLSISKDNSKSQVFFKMNSLQADDTAIYYCARKGGIYYANHYYAMDYWGQGTSVTVSS

SEQ ID NO: 31 - protein, 16 aa, Artificial Sequence
  Description of Artificial Sequence Synthetic peptide
RSSQNIVHNNGITYLE

SEQ ID NO: 32 - protein, 11 aa, Artificial Sequence
  Description of Artificial Sequence Synthetic peptide
RASQGIRNNLA

SEQ ID NO: 33 - protein, 7 aa, Artificial Sequence
  Description of Artificial Sequence Synthetic peptide
KVSDRFS

SEQ ID NO: 34 - protein, 7 aa, Artificial Sequence
  Description of Artificial Sequence Synthetic peptide
AASNLQS

SEQ ID NO: 35 - protein, 9 aa, Artificial Sequence
  Description of Artificial Sequence Synthetic peptide
FQGSHIPPT

SEQ ID NO: 36 - protein, 9 aa, Artificial Sequence
  Description of Artificial Sequence Synthetic peptide
LQHHSYPLT

SEQ ID NO: 37 - protein, 7 aa, Artificial Sequence
  Description of Artificial Sequence Synthetic peptide
GDTFTSY

SEQ ID NO: 38 - protein, 7 aa, Artificial Sequence
  Description of Artificial Sequence Synthetic peptide
GFTFSSY

SEQ ID NO: 39 - protein, 6 aa, Artificial Sequence
  Description of Artificial Sequence Synthetic peptide
YPGSGG

SEQ ID NO: 40 - protein, 6 aa, Artificial Sequence
  Description of Artificial Sequence Synthetic peptide
SGSGGS

SEQ ID NO: 41 - protein, 9 aa, Artificial Sequence
  Description of Artificial Sequence Synthetic peptide
SGGPYFFDY

SEQ ID NO: 42 - protein, 7 aa, Artificial Sequence
  Description of Artificial Sequence Synthetic peptide
SSGWSEY

SEQ ID NO: 43 - protein, 115 aa, Artificial Sequence
  Description of Artificial Sequence Synthetic polypeptide
DILMTQSPLSLPVSLGDQASISCRSSQNIVHNNGITYLEWYLQRPGQSPKLLIYKVSDRFSGVPDRFSGSGSGTDFTLKISRVEAEDLGIYYCFQGSHIPPTFGGGTKLEIKRAA

SEQ ID NO: 44 - protein, 112 aa, Artificial Sequence
  Description of Artificial Sequence Synthetic polypeptide
DIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKYAHNS

SEQ ID NO: 45 - protein, 118 aa, Artificial Sequence
  Description of Artificial Sequence Synthetic polypeptide
QVQLQQSGSEMARPGASVKLPCKASGDTFTSYWMHWVKQRHGHGPEWIGNIYPGSGGTNYAEKFKNKVTLTVDRSSRTVYMHLSRLTSEDSAVYYCTRSGGPYFFDYWGQGTTLTVSS

SEQ ID NO: 46 - protein, 116 aa, Artificial Sequence
  Description of Artificial Sequence Synthetic polypeptide
EVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSS

SEQ ID NO: 47 - protein, 15 aa, Artificial Sequence
  Description of Artificial Sequence Synthetic peptide
RASESVTILGSHLIH

SEQ ID NO: 48 - protein, 11 aa, Artificial Sequence
  Description of Artificial Sequence Synthetic peptide
SASQDISNYLN

SEQ ID NO: 49 - protein, 15 aa, Artificial Sequence
  Description of Artificial Sequence Synthetic peptide
RASESVTILGSHLIY

SEQ ID NO: 50 - protein, 7 aa, Artificial Sequence
  Description of Artificial Sequence Synthetic peptide
LASNVQT

SEQ ID NO: 51 - protein, 7 aa, Artificial Sequence
  Description of Artificial Sequence Synthetic peptide
YTSNLHS

SEQ ID NO: 52 - protein, 7 aa, Artificial Sequence
  Description of Artificial Sequence Synthetic peptide
LASNVQT

SEQ ID NO: 53 - protein, 9 aa, Artificial Sequence
  Description of Artificial Sequence Synthetic peptide
LQSRTIPRT

SEQ ID NO: 54 - protein, 9 aa, Artificial Sequence
  Description of Artificial Sequence Synthetic peptide
QQYRKLPWT

SEQ ID NO: 55 - protein, 9 aa, Artificial Sequence
  Description of Artificial Sequence Synthetic peptide
LQSRTIPRT

SEQ ID NO: 56 - protein, 7 aa, Artificial Sequence
  Description of Artificial Sequence Synthetic peptide
GYTFTDY

SEQ ID NO: 57 - protein, 7 aa, Artificial Sequence
  Description of Artificial Sequence Synthetic peptide
GGTFSNY

SEQ ID NO: 58 - protein, 7 aa, Artificial Sequence
  Description of Artificial Sequence Synthetic peptide
GYTFRHY

SEQ ID NO: 59 - protein, 7 aa, Artificial Sequence
  Description of Artificial Sequence Synthetic peptide
INTETRE

SEQ ID NO: 60 - protein, 6 aa, Artificial Sequence
  Description of Artificial Sequence Synthetic peptide
YRGHSD

SEQ ID NO: 61 - protein, 6 aa, Artificial Sequence
  Description of Artificial Sequence Synthetic peptide
NTESGV

SEQ ID NO: 62 - protein, 8 aa, Artificial Sequence
  Description of Artificial Sequence Synthetic peptide
DYSYAMDY

SEQ ID NO: 63 - protein, 12 aa, Artificial Sequence
  Description of Artificial Sequence Synthetic peptide
GAIYNGYDVLDN

SEQ ID NO: 64 - protein, 8 aa, Artificial Sequence
  Description of Artificial Sequence Synthetic peptide
DYLYSLDF

SEQ ID NO: 65 - protein, 111 aa, Artificial Sequence
  Description of Artificial Sequence Synthetic polypeptide
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 66 - protein, 108 aa, Artificial Sequence
  Description of Artificial Sequence Synthetic polypeptide
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 67 - protein, 111 aa, Artificial Sequence
  Description of Artificial Sequence Synthetic polypeptide
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 68 - protein, 117 aa, Artificial Sequence
  Description of Artificial Sequence Synthetic polypeptide
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 69 - protein, 121 aa, Artificial Sequence
  Description of Artificial Sequence Synthetic polypeptide
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSS

SEQ ID NO: 70 - protein, 117 aa, Artificial Sequence
  Description of Artificial Sequence Synthetic polypeptide
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 71 - protein, 11 aa, Artificial Sequence
  Description of Artificial Sequence Synthetic peptide
KASQDVITGVA

SEQ ID NO: 72 - protein, 7 aa, Artificial Sequence
  Description of Artificial Sequence Synthetic peptide
SASYRYT

SEQ ID NO: 73 - protein, 9 aa, Artificial Sequence
  Description of Artificial Sequence Synthetic peptide
QQHYSTPLT

SEQ ID NO: 74 - protein, 7 aa, Artificial Sequence
  Description of Artificial Sequence Synthetic peptide
GYSFTTY

SEQ ID NO: 75 - protein, 6 aa, Artificial Sequence
  Description of Artificial Sequence Synthetic peptide
HPSDSE

SEQ ID NO: 76 - protein, 11 aa, Artificial Sequence
  Description of Artificial Sequence Synthetic peptide
STMIATRAMDY

SEQ ID NO: 77 - protein, 108 aa, Artificial Sequence
  Description of Artificial Sequence Synthetic polypeptide
SDIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELK

SEQ ID NO: 78 - protein, 107 aa, Artificial Sequence
  Description of Artificial Sequence Synthetic polypeptide
DIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELK

SEQ ID NO: 79 - protein, 120 aa, Artificial Sequence
  Description of Artificial Sequence Synthetic polypeptide
SVTVSTGQGWYDMARTAIMTSRACYYVASDESTPSSLQMYATSSSKDVTLTAKDKFKQNLRTESDSPHIMGIWELGQGPRQKVWNMWYTTFSYGSAKCSLKVSAGPRVLEAGPQQLQVQS

SEQ ID NO: 80 - protein, 120 aa, Artificial Sequence
  Description of Artificial Sequence Synthetic polypeptide
SQVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVS

SEQ ID NO: 81 - protein, 229 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 82 - protein, 229 aa, Artificial Sequence
  Description of Artificial Sequence Synthetic polypeptide
ESKYGPPCPPCPAPEAAGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 83 - protein, 20 aa, Homo sapiens
PSGQAGAAASESLFVSNHAY

SEQ ID NO: 84 - protein, 9 aa, Artificial Sequence
  Description of Artificial Sequence Synthetic peptide
YPYDVPDYA

SEQ ID NO: 85 - protein, 23 aa, Artificial Sequence
  Description of Artificial Sequence Synthetic peptide
HVGSGEGRGSLLTCGDVEENPGP

SEQ ID NO: 86 - DNA, 54 bp, Artificial Sequence
  Description of Artificial Sequence Synthetic oligonucleotide
atggggtggtcaagcattattctgtttctggtcgctaccgctacaggcgtccat

SEQ ID NO: 87 - DNA, 60 bp, Artificial Sequence
  Description of Artificial Sequence Synthetic oligonucleotide
atgtacaggatgcaactcctgtcttgcattgcactaagtcttgcacttgtcacaaacagt

SEQ ID NO: 88 - DNA, 45 bp, Artificial Sequence
  Description of Artificial Sequence Synthetic oligonucleotide
ggtgggggcggctctggtggcggtggcagcggcggaggtggcagt

SEQ ID NO: 89 - DNA, 204 bp, Artificial Sequence
  Description of Artificial Sequence Synthetic polynucleotide
ttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 90 - DNA, 336 bp, Artificial Sequence
  Description of Artificial Sequence Synthetic polynucleotide
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 91 - DNA, 69 bp, Artificial Sequence
  Description of Artificial Sequence Synthetic oligonucleotide
cacgtgggttctggagaaggacgcggttccttgttgacgtgtggcgatgtagaggaaaatccgggtcca

SEQ ID NO: 92 - DNA, 60 bp, Artificial Sequence
  Description of Artificial Sequence Synthetic oligonucleotide
ccgagcggccaggcgggcgcggcggcatcggagtccctgtttgtgtcaaatcacgcctac

SEQ ID NO: 93 - DNA, 333 bp, Artificial Sequence
  Description of Artificial Sequence Synthetic polynucleotide
gatattgttcttactcaatcacccccaagccttgcgatgtctcttggtaaacgagcgacaattagttgtagagcttctgaaagcgtaactattcttgggtcacatcttattcattggtatcaacaaaagccgggacaaccgcctacactcttgattcaactcgcgagcaatgttcaaacgggtgtccctgcacgcttttctgggagcggttcacgaacagattttactctcacgattgatccagtcgaagaagatgatgtcgctgtatattattgtctccaaagtaggacaataccaagaacttttggtggtggtacaaaattggaaattaaa

SEQ ID NO: 94 - DNA, 324 bp, Artificial Sequence
  Description of Artificial Sequence Synthetic polynucleotide
gacatccagatgacccagagccctagctcactgagcgccagcgtgggcgacagggtgaccattacctgctccgccagccaggacatcagcaactacctgaactggtaccagcagaagcccggcaaggcccccaagctgctgatctactacacctccaacctgcactccggcgtgcccagcaggttcagcggaagcggcagcggcaccgatttcaccctgaccatctccagcctgcagcccgaggacttcgccacctactactgccagcagtacaggaagctcccctggactttcggccagggcaccaaactggagatcaagcgt

SEQ ID NO: 95 - DNA, 333 bp, Artificial Sequence
  Description of Artificial Sequence Synthetic polynucleotide
gacatcgtgctgacccagagcccccccagcctggccatgtctctgggcaagagagccaccatcagctgccgggccagcgagagcgtgaccatcctgggcagccacctgatctactggtatcagcagaagcctggccagccccccaccctgctgatccagctggctagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggaacaaagctggaaatcaag

SEQ ID NO: 96 - DNA, 333 bp, Artificial Sequence
  Description of Artificial Sequence Synthetic polynucleotide
gacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaag

SEQ ID NO: 97 - DNA, 351 bp, Artificial Sequence
  Description of Artificial Sequence Synthetic polynucleotide
cagatccagctggtgcagtccggccccgagctgaagaagcccggcgagaccgtgaagatctcctgcaaggcctccggctacaccttcaccgactactccatcaactgggtgaagcgggcccccggcaagggcctgaagtggatgggctggatcaacaccgagacccgggagcccgcctacgcctacgacttccggggccggttcgccttctccctggagacctccgcctccaccgcctacctgcagatcaacaacctgaagtacgaggacaccgccacctacttctgcgccctggactactcctacgccatggactactggggccagggcacctccgtgaccgtgtcctcc

SEQ ID NO: 98 - DNA, 363 bp, Artificial Sequence
  Description of Artificial Sequence Synthetic polynucleotide
caggtgcagctggtccagagcggcgccgaagtgaagaagcccggcagctccgtgaaagtgagctgcaaggccagcggcggcaccttcagcaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcgccaggggcgccatctacaacggctacgacgtgctggacaactggggccagggcacactagtgaccgtgtccagc

SEQ ID NO: 99 - DNA, 405 bp, Artificial Sequence
  Description of Artificial Sequence Synthetic polynucleotide
atgggatggagctctatcatcctcttcttggtagcaacagctacaggtgtccaccagattcagctggtgcagagcggccctgagctgaagaaacccggcgagacagtgaagatcagctgcaaggcctccggctacaccttccggcactacagcatgaactgggtgaaacaggcccctggcaagggcctgaagtggatgggccggatcaacaccgagagcggcgtgcccatctacgccgacgacttcaagggcagattcgccttcagcgtggaaaccagcgccagcaccgcctacctggtgatcaacaacctgaaggacgaggataccgccagctacttctgcagcaacgactacctgtacagcctggacttctggggccagggcaccgccctgaccgtgtccagc

SEQ ID NO: 100 - DNA, 351 bp, Artificial Sequence
  Description of Artificial Sequence Synthetic polynucleotide
cagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagc

SEQ ID NO: 101 - DNA, 330 bp, Artificial Sequence
  Description of Artificial Sequence Synthetic polynucleotide
cagtcagcgcttacgcagccggcgtcggtgtcgggttccccgggtcagtcgatcacgatcagctgtagtgggagcagctccaacatcggtaacaacgcagtgaactggtatcagcaactgccgggaaaagcgccgaaactgctgatttactatgatgatttgctgccaagtggagttagtgaccgcttttccggcagtaaatcgggtacctcggcttttctggctatttcgggtctccagagcgaggatgaagctgattattattgcgccgcatgggatgatagcttaaatggcccagtttttggcggcggtactaaactgaccgtgctg

SEQ ID NO: 102 - DNA, 363 bp, Artificial Sequence
  Description of Artificial Sequence Synthetic polynucleotide
caagtgcagctggttgaatccggtggcggtctggtcaagccgggcggctctttgcgtctgagctgtgccgcgtcgggttttaccttcagctcttatggtatgcattgggtgcgtcaggcgcctggcaaaggtctggagtgggttgcgttcatccgctacgatgggtctaacaaatattatgccgactcagtaaaaggacgcttcactattagccgcgacaatagcaaaaataccctgtacctgcaaatgaatagcctgcgcgccgaagataccgccgtttactattgcgctaaagatcgtggcctgggtgatggtacgtacttcgattactggggtcagggcaccaccgttaccgttagttca

SEQ ID NO: 103 - DNA, 324 bp, Artificial Sequence
  Description of Artificial Sequence Synthetic polynucleotide
gatattgtgctaactcagtctccagccaccctgtctgtgactccaggagatagcgtcagtctttcctgcagggccagccagagtattagcaacaacctacactggtatcaacaaaaatcacatgagtctccaaggcttctcatcaagtatgcttcccagtccatctctgggatcccctccaggttcagtggcagtggatcagggacagatttcactctcagtatcaacagtgtggagactgaagattttggagtgtatttctgtcaacagagtaacacctggccgtacacgttcggaggggggaccaagctggaaataaaacgg

SEQ ID NO: 104 - DNA, 342 bp, Artificial Sequence
  Description of Artificial Sequence Synthetic polynucleotide
gacattgtgatgacacagtctccatcctccctgagtgtgtcagcaggagagaaggtcactatgagctgcaagtccagtcagagtctgttaaacagtggaaatcaaaagaactatatggcctggtatcagcagaaaccagggcagcctcctaaactgttgatctacggggcatccactagggaatctggggtccctgatcgcttcacaggcagtggatctggaaccgatttcactcttaccatcagcagtgtgcaggctgaagacctggcagtttattactgtcagaatgatcatagttatccgctcacgttcggtgctgggaccaagctggagctgaaacgg

SEQ ID NO: 105 - DNA, 354 bp, Artificial Sequence
  Description of Artificial Sequence Synthetic polynucleotide
caggtccaactgcagcagcctggggctgagcttgtgaagcctggggcttcattgaagctgtcctgcaagtcttccgggtacaccttcaccagctactggatgcactgggtgaggcagaggcctggacatggccttgagtggatcggagagattgatccttctgacagttataaagactacaatcagaagttcaaggacaaggccacattgactgtggacagatcctccaacacagcctacatgcacctcagcagcctgacatctgatgactctgcggtctattattgtgcaagagcgattacgacgaccccctttgacttctggggccaaggcaccactctcacagtctcctca

SEQ ID NO: 106 - DNA, 369 bp, Artificial Sequence
  Description of Artificial Sequence Synthetic polynucleotide
caggtgcagctgaagcagtcaggacctggcctagtgcagccctcacagagcctgtccatcacctgcacagtctctggtttctcattaactaactatggtttacactgggttcgccagtctccaggaaagggcctggagtggctgggagtgatatggagtggtggaagcacagactataatgcagctttcatatccagactgagcatcagcaaggacaactccaagagccaagttttctttaaaatgaacagtctgcaggctgatgacacagccatatactactgtgccagaaaaggagggatctactatgctaaccattactatgctatggactactggggtcaaggaacctcagtcaccgtctcctca

SEQ ID NO: 107 - DNA, 321 bp, Artificial Sequence
  Description of Artificial Sequence Synthetic polynucleotide
aaacttgagttgaagaccggtgccggcttcaccttaccgaccagttatcatcaacaatgctattacgtggccctggacgaagcacaggtgaattcaattacgtttacgtttgataccggctctggcagcggtacatttcgtgatcccgtgggcacttaccgctattcggcgagttatatcttgctgaaaccttcccaaggtccgaaacagcagtactgggcggttggcaccattgtagaccaatcagccaaatgtacaatctcggttcgcgatggtgtcagtacgtcgatgtctaagcagtcacagacaatggttatcgat

SEQ ID NO: 108 - DNA, 360 bp, Artificial Sequence
  Description of Artificial Sequence Synthetic polynucleotide
agcgttaccgtgagtacaggccagggctggtatgacatggcacgtacagccatcatgacctcgcgcgcatgttactacgtcgcgtcagatgaatcgacgccttcctcgctgcaaatgtatgcaacctccagcagcaaagatgttaccctgaccgcaaaggacaagtttaaacagaatttgcgtacggagagtgactccccgcacatcatgggaatctgggagttgggtcaggggcctcgtcagaaggtatggaacatgtggtatacaactttttcgtacggctcagcaaaatgcagcttgaaagtgtcggcaggtccgcgcgtgctggaggccggtccgcagcagctgcaagtccagtct

SEQ ID NO: 109 - DNA, 63 bp, Artificial Sequence
  Description of Artificial Sequence Synthetic oligonucleotide
ctcgagcccaaatcttgtgacaaaactcacacatgcccaccgtgcccggatcccaaaggtacc

SEQ ID NO: 110 - protein, 12 aa, Artificial Sequence
  Description of Artificial Sequence Synthetic peptide
  MISC_FEATURE (1)..(12): This sequence may encompass 1-6 “Gly Ser” repeating units
GSGSGSGSGSGS

SEQ ID NO: 111 - protein, 6 aa, Artificial Sequence
  Description of Artificial Sequence Synthetic 6xHis tag
HHHHHH

SEQ ID NO: 112 - DNA, 48 bp, Artificial Sequence
  Description of Artificial Sequence Synthetic oligonucleotide
ctcgagcccaaatcttgtgacaaaactcacacatgcccaccgtgcccg

SEQ ID NO: 113 - DNA, 81 bp, Artificial Sequence
  Description of Artificial Sequence Synthetic oligonucleotide
ttttgggtgctggtggtggttggtggagtcctggcttgctatagcttgctagtaacagtggcctttattattttctgggtg

SEQ ID NO: 114 - DNA, 126 bp, Artificial Sequence
  Description of Artificial Sequence Synthetic polynucleotide
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 115 - DNA, 123 bp, Artificial Sequence
  Description of Artificial Sequence Synthetic polynucleotide
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 116 - DNA, 339 bp, Artificial Sequence
  Description of Artificial Sequence Synthetic polynucleotide
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 117 - protein, 51 aa, Homo sapiens
PAKPTTTPAPRPPTPAPTIASQPLSLRPEACRPAAGGAVHTRGLDFACDIY

SEQ ID NO: 118 - protein, 49 aa, Mus sp.
KVNSTTTKPVLRTPSPVHPTGTSQPQRPEDCRPRGSVKGTGLDFACDIY

SEQ ID NO: 119 - protein, 51 aa, Felis sp.
PVKPTTTPAPRPPTQAPITTSQRVSLRPGTCQPSAGSTVEASGLDLSCDIY

SEQ ID NO: 120 - protein, 21 aa, Homo sapiens
IYIWAPLAGTCGVLLLSLVIT

SEQ ID NO: 121 - protein, 21 aa, Mus sp.
IWAPLAGICVALLLSLIITLI

SEQ ID NO: 122 - protein, 21 aa, Rattus sp.
IWAPLAGICAVLLLSLVITLI

SEQ ID NO: 123 - protein, 42 aa, Artificial Sequence
  Description of Artificial Sequence Synthetic polypeptide
KRGRKKLLYIFKQPFMRPVQTTQEEDGCSCRFPEEEEGGCEL

SEQ ID NO: 124 - protein, 220 aa, Artificial Sequence
  Description of Artificial Sequence Synthetic polypeptide
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 125 - DNA, 105 bp, Artificial Sequence
  Description of Artificial Sequence Synthetic polynucleotide
acaaaaaagaagtattcatccagtgtgcacgaccctaacggtgaatacatgttcatgagagcagtgaacacagccaaaaaatccagactcacagatgtgacccta

SEQ ID NO: 126 - DNA, 108 bp, Artificial Sequence
  Description of Artificial Sequence Synthetic polynucleotide
agggaccagaggctgccccccgatgcccacaagccccctgggggaggcagtttccggacccccatccaagaggagcaggccgacgcccactccaccctggccaagatc

SEQ ID NO: 127 - protein, 993 aa, Homo sapiens
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 128 - protein, 952 aa, Homo sapiens
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 129 - protein, 1210 aa, Homo sapiens [sequence omitted]

SEQ ID NO: 130 - protein, 705 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGPGNESLKAMLFCLFKLSSCNQSNDGSVSHQSGSPAAQESCLGWIPSLLPSEFQLGWGGCSHLHAWPSASVIITASSCH

SEQ ID NO: 131 - protein, 1200 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPLMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 132 - DNA, 345 bp, Artificial Sequence
  Description of Artificial Sequence Synthetic polynucleotide
gacattctaatgacccaatctccactctccctgcctgtcagtcttggagatcaagcctcctacctgcaaaggccaggccagtctccaaagctcctgatctacaaagtttccgaccgattttacctgcaaaggccaggccagtctccaaagctcctgatctacaaagtttccgaccgattttctggggtcccagacaggttcagtggcagtggatcagggacagatttcacactcaagatcagcagagtagaggctgaggatctgggaatttattactgctttcaaggttcacatattcctcccacgttcggaggggggaccaagctggaaatcaaacgtgcggcc

SEQ ID NO: 133 - DNA, 354 bp, Artificial Sequence
  Description of Artificial Sequence Synthetic polynucleotide
caggtccagctgcagcagtctgggtctgagatggcgaggcctggagcttcagtgaagctgccctgcaaggcttctggcgacacattcaccagttactggatgcactgggtgaagcagaggcatggacatggccctgagtggatcggaaatatttatccaggtagtggtggtactaactacgctgagaagttcaagaacaaggtcactctgactgtagacaggtcctcccgcacagtctacatgcacctcagcaggctgacatctgaggactctgcggtctattattgtacaagatcggggggtccctacttctttgactactggggccaaggcaccactctcacagtctcctcc

SEQ ID NO: 134 - protein, 5 aa, Artificial Sequence
  Description of Artificial Sequence Synthetic peptide
GGGGS

SEQ ID NO: 135 - protein, 10 aa, Artificial Sequence
  Description of Artificial Sequence Synthetic peptide
GGGGSGGGGS

SEQ ID NO: 136 - protein, 20 aa, Artificial Sequence
  Description of Artificial Sequence Synthetic peptide
GGGGSGGGGSGGGGSGGGGS

SEQ ID NO: 137 - protein, 25 aa, Artificial Sequence
  Description of Artificial Sequence Synthetic peptide
GGGGSGGGGSGGGGSGGGGSGGGGS

SEQ ID NO: 138 - protein, 30 aa, Artificial Sequence
  Description of Artificial Sequence Synthetic polypeptide
GGGGSGGGGSGGGGSGGGGSGGGGSGGGGS

SEQ ID NO: 139 - protein, 35 aa, Artificial Sequence
  Description of Artificial Sequence Synthetic polypeptide
GGGGSGGGGSGGGGSGGGGSGGGGSGGGGSGGGGS

SEQ ID NO: 140 - protein, 40 aa, Artificial Sequence
  Description of Artificial Sequence Synthetic polypeptide
GGGGSGGGGSGGGGSGGGGSGGGGSGGGGSGGGGSGGGGS

SEQ ID NO: 141 - protein, 45 aa, Artificial Sequence
  Description of Artificial Sequence Synthetic polypeptide
GGGGSGGGGSGGGGSGGGGSGGGGSGGGGSGGGGSGGGGSGGGGS

SEQ ID NO: 142 - protein, 50 aa, Artificial Sequence
  Description of Artificial Sequence Synthetic polypeptide
GGGGSGGGGSGGGGSGGGGSGGGGSGGGGSGGGGSGGGGSGGGGSGGGGS

SEQ ID NO: 143 - protein, 55 aa, Artificial Sequence
  Description of Artificial Sequence Synthetic polypeptide
GGGGSGGGGSGGGGSGGGGSGGGGSGGGGSGGGGSGGGGSGGGGSGGGGSGGGGS

SEQ ID NO: 144 - protein, 60 aa, Artificial Sequence
  Description of Artificial Sequence Synthetic polypeptide
GGGGSGGGGSGGGGSGGGGSGGGGSGGGGSGGGGSGGGGSGGGGSGGGGSGGGGSGGGGS

SEQ ID NO: 145 - protein, 65 aa, Artificial Sequence
  Description of Artificial Sequence Synthetic polypeptide
GGGGSGGGGSGGGGSGGGGSGGGGSGGGGSGGGGSGGGGSGGGGSGGGGSGGGGSGGGGSGGGGS

SEQ ID NO: 146 - protein, 70 aa, Artificial Sequence
  Description of Artificial Sequence Synthetic polypeptide
GGGGSGGGGSGGGGSGGGGSGGGGSGGGGSGGGGSGGGGSGGGGSGGGGSGGGGSGGGGSGGGGSGGGGS

SEQ ID NO: 147 - protein, 75 aa, Artificial Sequence
  Description of Artificial Sequence Synthetic polypeptide
GGGGSGGGGSGGGGSGGGGSGGGGSGGGGSGGGGSGGGGSGGGGSGGGGSGGGGSGGGGSGGGGSGGGGSGGGGS

SEQ ID NO: 148 - DNA, 546 bp, Artificial Sequence
  Description of Artificial Sequence Synthetic polynucleotide
aaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 149 - protein, 120 aa, Artificial Sequence
  Description of Artificial Sequence Synthetic polypeptide
SSVTVSTGQGWYDMARTAIMTSRACYYVASDESTPSSLQMYATSSSKDVTLTAKDKFKQNLRTESDSPHIMGIWELGQGPRQKVWNMWYTTFSYGSAKCSLKVSAGPRVLEAGPQQLQVQ

SEQ ID NO: 150 - protein, 120 aa, Artificial Sequence
  Description of Artificial Sequence Synthetic polypeptide
QVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSS

SEQ ID NO: 151 - protein, 11 aa, Artificial Sequence
  Description of Artificial Sequence Synthetic peptide
AVGTIVDQSAK

SEQ ID NO: 152 - protein, 4 aa, Artificial Sequence
  Description of Artificial Sequence Synthetic peptide
  MOD_RES (2)..(3): Any amino acid
YXXQ

SEQ ID NO: 153 - protein, 4 aa, Artificial Sequence
  Description of Artificial Sequence Synthetic peptide
YRHQ

SEQ ID NO: 154 - protein, 108 aa, Artificial Sequence
  Description of Artificial Sequence Synthetic polypeptide
KLELKTGAGFTLPTSYHQQCYYVALDEAQVNSITFTFDTGSGSGTFRDPVGTYRYSASYILLKPSQGPKQQYWAVGTIVDQSAKCTISVRDGVSTSMSKQSQTMVIDS

SEQ ID NO: 155 - DNA, 405 bp, Artificial Sequence
  Description of Artificial Sequence Synthetic polynucleotide
atgggatggagctctatcatcctcttcttggtagcaacagctacaggtgtccaccagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagc

SEQ ID NO: 156 - DNA, 333 bp, Artificial Sequence; identical to SEQ ID NO: 96
  Description of Artificial Sequence Synthetic polynucleotide

SEQ ID NO: 157 - DNA, 360 bp, Artificial Sequence; identical to SEQ ID NO: 108
  Description of Artificial Sequence Synthetic polynucleotide

SEQ ID NO: 158 - DNA, 321 bp, Artificial Sequence; identical to SEQ ID NO: 107
  Description of Artificial Sequence Synthetic polynucleotide

SEQ ID NO: 159 - DNA, 363 bp, Artificial Sequence; identical to SEQ ID NO: 102
  Description of Artificial Sequence Synthetic polynucleotide

SEQ ID NO: 160 - DNA, 330 bp, Artificial Sequence; identical to SEQ ID NO: 101
  Description of Artificial Sequence Synthetic polynucleotide

SEQ ID NO: 161 - DNA, 45 bp, Artificial Sequence
  Description of Artificial Sequence Synthetic oligonucleotide
ggcggtggcggttctggtggcggtggctccggcggtggcggttct

SEQ ID NO: 162 - DNA, 378 bp, Artificial Sequence
  Description of Artificial Sequence Synthetic polynucleotide
caagtgcagctggttgaatccggtggcggtctggtcaagccgggcggctctttgcgtctgagctgtgccgcgtcgggttttaccttcagctcttatggtatgcattgggtgcgtcaggcgcctggcaaaggtctggagtgggttgcgttcatccgctacgatgggtctaacaaatattatgccgactcagtaaaaggacgcttcactattagccgcgacaatagcaaaaataccctgtacctgcaaatgaatagcctgcgcgccgaagataccgccgtttactattgcgctaaagatcgtggcctgggtgatggtacgtacttcgattactggggtcagggcaccaccgttaccgttagttcaggtgggggcggctct

SEQ ID NO: 163 - DNA, 326 bp, Artificial Sequence
  Description of Artificial Sequence Synthetic polynucleotide
cagcgcttacgcagccggcgtcggtgtcgggttccccgggtcagtcgatcacgatcagctgtagtgggagcagctccaacatcggtaacaacgcagtgaactggtatcagcaactgccgggaaaagcgccgaaactgctgatttactatgatgatttgctgccaagtggagttagtgaccgcttttccggcagtaaatcgggtacctcggcttttctggctatttcgggtctccagagcgaggatgaagctgattattattgcgccgcatgggatgatagcttaaatggcccagtttttggcggcggtactaaactgaccgtgctg

SEQ ID NO: 164 - DNA, 361 bp, Artificial Sequence
  Description of Artificial Sequence Synthetic polynucleotide
ctccgtgacggtgtcgacgggccaaggatggtacgatatggcacggaccgcgattatgacatcgcgggcgtgctattacgtggccagcgatgagtcgaccccttcctctctgcaaatgtatgccacctcctcttcaaaagacgtgactctgactgcgaaagacaaatttaaacagaatctgcgcaccgaaagcgatagcccacatatcatgggcatctgggaactgggccagggcccccgccagaaagtgtggaacatgtggtacaccaccttcagctatggttcggccaaatgttccctgaaggtatcagccggcccgcgcgttcttgaggcgggtccgcagcagctgcaggtacagagc

SEQ ID NO: 165 - DNA, 324 bp, Artificial Sequence
  Description of Artificial Sequence Synthetic polynucleotide
aaactggaactcaagacgggtgcgggatttaccctccctacgagctatcaccagcagtgctattacgtggcgcttgacgaagcgcaggtgaactctattacctttacctttgatacaggatcaggcagcggtacgttccgtgatccggtaggtacgtaccggtatagtgcaagctatatccttctgaaaccttctcagggtccgaaacagcagtactgggcggtgggaacgatcgtggaccagtctgccaaatgtacaatttcagttcgcgacggagttagcacctccatgagcaagcagtcccaaaccatggtgattgactct

SEQ ID NO: 166 - DNA, 351 bp, Artificial Sequence; identical to SEQ ID NO: 100
  Description of Artificial Sequence Synthetic polynucleotide

SEQ ID NO: 167 - DNA, 333 bp, Artificial Sequence; identical to SEQ ID NO: 96
  Description of Artificial Sequence Synthetic polynucleotide

SEQ ID NO: 168 - DNA, 57 bp, Artificial Sequence
  Description of Artificial Sequence Synthetic oligonucleotide
atggaatttgggctgcgctgggttttccttgttgctattttaaaagatgtccagtgt

SEQ ID NO: 169 - protein, 19 aa, Artificial Sequence
  Description of Artificial Sequence Synthetic peptide
MEFGLRWVFLVAILKDVQC

SEQ ID NO: 170 - DNA, 903 bp, Artificial Sequence
  Description of Artificial Sequence Synthetic polynucleotide
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccggagctgcaactggaggagagctgtgcggaggcgcaggacggggagctggacgggctgtggacgaccatcaccatcttcatcacactcttcctgttaagcgtgtgctacagtgccaccgtcaccttcttcaaggtgaagtggatcttctcctcggtggtggacctgaagcagaccatcatccccgactacaggaacatgatcggacagggggcc

SEQ ID NO: 171 - protein, 301 aa, Artificial Sequence
  Description of Artificial Sequence Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKWIFSSVVDLKQTIIPDYRNMIGQGA

SEQ ID NO: 172 - protein, 7 aa, Homo sapiens
ELREATS

SEQ ID NO: 173 - protein, 6 aa, Homo sapiens
ELREAT

SEQ ID NO: 174 - protein, 8 aa, Artificial Sequence
  Description of Artificial Sequence Synthetic peptide
  MOD_RES (2)..(2): Val or Ile
  MOD_RES (4)..(4): Any amino acid
DXEXNPGP